SEQUENCE LISTING (323 sequences)

SEQ ID NO: 1 - protein, 659 aa, Synthetic Construct
MFGKRRQVRKLLIWVVLLLIVYFFGLQFRAKNSAHQSSIRSFYADNKEFFDRQYSRYDEYDIIDNMNSHNELLQEQFRNGKLAAGLRGVAEEPNSDEVTDDTAIEEDEQAAMINFPKRSPQREKSLVELRKFYKNVLSIIINNKPAMPIENPRDPTPNENALKRKFGKSGIINIALHDTDPSLPILSEAYLRDSLQLSPSFIASLSKSHSAVVKAFPPSFPANAYNGTGIVFIGGQKFSWLSLLSIENLRKTGSKVPVELIIPFAHEYEPQLCEEILPKLNATCVLLQETVGIDLLKSGHLKGYQFKSLALLASSFEQVLLVDSDNIIVENPDPIFDSEVFQRTGLVLWPDFWRRVTHPDYYKIAGIKLGSERVRHVVDSYTDPSLYTSSSEDPFTDIPLHDREGAIPDGSTESGQILISKTKHCQTILLSLYYNFFGPDYYYPLFTQGASGEGDKETFLAAANYYKLPFYNIKKGVDVIGYWKPDQSAYQGCGMLQYDPIVDYQNLQTFLKTHKGSRVNKLEQSELDKPGLLSRLIPKFFFRKTFDEHQLQSHFTKDRSKIMFIHSNFPKLDPFGLKLHNYLFVDQDTHKPRIRMYADQTGLSFDFELRQWIIIHEYFCEYPDFNLKYLENANVKPQDLCMFIKEELNFLQNNPIQLT

SEQ ID NO: 2 - protein, 683 aa, Synthetic Construct
MLFGLIRHSRRQLLFLGALVTVIVLIFTLPNTSPIEANGVKSEEGSITPIIPVLESPANSLEKIVDTASEERIGGATLEEGHENNKEEQALENAERAKEKEKTEAIAAEEEKLKAAELLRQQETTREKEAAKEDDSKKPNQELVEQDTYLDDIPDDVEDNIIISEQDRKKIILPSYTPKTDPAYSKRATALKIFYNDFFIKVADSGPNTAPITKKTRKKGKSKLKGDVSSGDKYEGPVLTEDFLRFMEIYSDEFIDAVSESHSKIVNLMPESFPKGMYQGDGIVIIGGGVYSWYGLLAIRNLRDGGNTLPVELMLPSDNEYEPQLCEQILPSLNAKCIMLSDIVDQDVLKKLDFKGYQFKALSLLASSFENVLSLDSDNIPVANVSHLFDHEPFSETGLVSWPDFWRRTTNPRYYEAAGIKIGEYQVRNCLDGFVPESDFVHIGLKDIPLHDRNGTIPDASTESGQLLVNKNKHAKTLMLMFYYNFYGPGYYYPLLSQGMAGEGDKETFLAAANFFGLPFYQVKAGPGILGHHDSTGAFTGVAIVQYDPIADYELTKENFVGEKRKGIEAPKAFYGNNNKSPLFHHCNFPKLDPVKLIKEKKLIDNKTHKFNRMYGPNTKLKYDFEERQWKYTKEYLCEKKYNLLYFTEQYKNYGQGYSQERICKFSDRFLKFLSDNPIRIEG

SEQ ID NO: 3 - protein, 579 aa, Synthetic Construct
MFNSLAPMRLKKLLKVFCASVVLLAATSVVLFFHFGGQIIIPIPERTVTLSTPPANDTWQFQQFFNGYLDALLENNLSYPIPERWNHEVTNVRFFNRIGELLSESRLQELIHFSPEFIEDTSDKFDNIVEQIPAKWPYENMYRGDGYVIVGGGRHTFLALLNINALRRAGNKLPVEVVLPTYDDYEEDFCENHFPLLNARCVILEERFGDQVYPRLQLGGYQFKIFAIAASSFKNCFLLDSDNIPLRKMDKIFSSELYKNKTMITWPDFWLRSTSPHYYHNITKTPIGDKRVRYFNDFYTNPNEYYYGDEDPRSEIPFHDREGTIPDWTTESGQLVINKEVHFPAILLGLFYNFNGPMGFYPLLSQGGAGEGDKDTFVAASHYYNLPYYQVYKNCEMLYGWVDHANSGRIEHSAIVQYNPIVDYENLQSVKAKAEIILKNHEPDSRKKSSKPKSYSKTRLSTHVKGSIYSYRRLFRDSFNKANSDEMFLHCHTPKIEPYRIMEDDLTLGRNKEAKQRWYGGRKNRVRFGYDVELYIWELIDQYICDKNIQYKIFEGKDRDALCGSFMREQLGFLRSTGD

SEQ ID NO: 4 - protein, 444 aa, Synthetic Construct
MELVRLANLVNVNHPFEQSNIYRVPLFFLLSTTRPDRTTVQMAGATRINSRVVRFAIFASILVLLGFILSRGSATSYSLPSGLTSDTSQSTGSSPKSESKPSSQGSSGATELKKTYTTDGKEKATFVSLARNSDVWSLASSIRHVEDRFNHKFHYDWVFLNDEEFSDEFKRVTSALTSGKAKYGLIPKEHWSFPEWIDKERAAKTRKEMAAKKVIYGDSISYRHMCRFESGFFFRHELMQEYEWYWRVEPDIKIYCDIDYDVFKFMKDNNKMYGFTVSLPEYVATIETLWDTTRAFIKENPQYLPEDNMMDFISDDDGLSYNGCHFWSNFEVGSLSLWRSEAYLKYFDHLDKAGGFFYERWGDAPVHSIAAALFLHRDQIHFFDDVGYFHNPFNNCPVDADLREERRCMCNPKDDFTWKGYSCVPEFFTVNNMKRPKGWEAFSG

SEQ ID NO: 5 - protein, 426 aa, Synthetic Construct
SNIYRVPLFFLLSTTRPDRTTVQMAGATRINSRVVRFAIFASILVLLGFILSRGSATSYSLPSGLTSDTSQSTGSSPKSESKPSSQGSSGATELKKTYTTDGKEKATFVSLARNSDVWSLASSIRHVEDRFNHKFHYDWVFLNDEEFSDEFKRVTSALTSGKAKYGLIPKEHWSFPEWIDKERAAKTRKEMAAKKVIYGDSISYRHMCRFESGFFFRHELMQEYEWYWRVEPDIKIYCDIDYDVFKFMKDNNKMYGFTVSLPEYVATIETLWDTTRAFIKENPQYLPEDNMMDFISDDDGLSYNGCHFWSNFEVGSLSLWRSEAYLKYFDHLDKAGGFFYERWGDAPVHSIAAALFLHRDQIHFFDDVGYFHNPFNNCPVDADLREERRCMCNPKDDFTWKGYSCVPEFFTVNNMKRPKGWEAFSG

SEQ ID NO: 6 - protein, 289 aa, Synthetic Construct
MTGCFLNEVPFTDEFKERTSVLISGQAKYGLIPKEHWSYPDYIDQERAAESRRQLEDQHVVYGGLESYRHMCRFNSGFFYKHPLMLDYRYYWRVEPEIEILCDVETDLFRYMRENNKTYGFTISIHEFEKTIPTLWETTKEFMKQNPSYIAENNLMNFISDDNGKTYNLCHFWSNFEVADMDFWRSDVYEKYFKFLDDTGKFFYERWGDAPVHSLAVSLFLPKEKVHFFNEVGYKHSVYSMCPIDKDIWKNRKCYCDPNTDFTFRGYSCGRQYYKATGLTRPSNWKDYD

SEQ ID NO: 7 - protein, 396 aa, Synthetic Construct
MKVVWLACFIILAAIWYKDYQSLRGFMDDRVSKTLPINFNALKLSTNSYIPVDEHLIKPNREPNPKFVKENATLLMLCRNWELEEVLQSMRSLEDRFNGRYQYTWTFLNDVPFEKQFIQETTLMASGKTQYALISSTDWNRPSFINETRFEQNLIQSEKDDIIYGGSPSYRNMCRFNSGFFYKQKILDQYDYYFRVEPGVEYFCDLEEDPFRYMRLHDKKYGFVISLYEYENTIPTLWQTVEKFIENHPEYIHPNNSYEFLTDKEVVGPLGLVALTEQTYNLCHFWSNFEIGDLNFFRSEKYEAFFQFLDQAGGFYYERWGDAPVHSIAVGILLDKRQIHHFENIGYYHLPFSTCPQSYWSYKCNRCICKRNESIDLVPHSCLSKWWKYGGKTFLQ

SEQ ID NO: 8 - protein, 423 aa, Synthetic Construct
MMRARLSLERVNLSFITSVFLASVAVLFISLEMPKVLARDRQILKLKLGFMGSGLQKGSLETSGNIENTESNINSQTTQHIGTIGASNERANATFYTLCRNEELYQMLETVQNYEDRFNSKFKYDWVFLNDYPFTDEFKRVISHAISGEAKFGQVPASHWRFPDHIDQQKVYESMDKMDSDNTTGDYLGLPIPYAKSISYRHMCRYQSGFFYKHGLLQGYKYFWRVEPDVKLYCDIDYDVFKSMEQNGKRYGFVISMMEFEKTIESLFKEVKNYLQMKGVSRLLEDTDNLSDFVYDELSGDYTLCHFWSNFEIGDLDFFRGREYNEFFDYLDSKGGFYYERWGDAPIHSIAVSLFMQWNDVKWFSDIGYRHPPYLSCPLSEEVRLEKKCSCDPKQDFTMDAYSCTRFYQDIIRDKQKSQGSNP

SEQ ID NO: 9 - protein, 420 aa, Synthetic Construct
MMISLTKRFTKLAIFGSLSFILTTAGLWLYWDAIQYMMTSGKIPTLDFQFEDFMNRHDDIVDDMMKKYDKIMKAEVKEPNVGNLVYAPESLVDYGRENATLLMLVRNKELRTALQAIETVESQFNHKFQYPYVFLNDKEFTDKFKSTITEKVSGQVFFETIDKVTWDRPDWIDSAKESERIKVMRKYNVGYADKLSYHNMCRYYSRGFYNHPRLQQFKYYWRFEPGTHYHTSIDYDVFKFMSANDKTYGFVISLYDTERSIETLWPETLKFIEQNPQFVNKNAAWDWLTEKKQNPQKTRIANGYSTCHFWSNFEIGDMDFFRSEAYTKWVNHLDATGGFYYERWGDAPVHSIGATLFQDKSKVHWFRDIGYYHAPYYQCPNSPQSDGKCEVGKFSFPNLSDQNCLINWIEMVADNELSMY

SEQ ID NO: 10 - protein, 537 aa, Synthetic Construct
MSFRLGYIQAIVLGLVLLSVCWTIVIRPDPSSAIDLASPVTIDLENSLTNLKSFPISSRRISSNIDHVFQTGCRNVFKNKKKANAALVVLARNSELEGVQKSMFSMERHFNQWFNYPWIFLNDEEFTESFKDGVMNMTSSGVSFGVISKPDWNFSEEKDRGSTEFLRFNEFIQNQGDRGIMYGALPSYHKMCRFYSGYFFKHPLVAKLSWYWRVEPDVEFFCDLTYDPFLEMEASGKKYGFAVIIKELSNTVPNLFRHTQSFIEKYGISVDEKAWSIFTNRRSFGEKESMKLIDKIRINHLLSNFSGGIGTRLLSSLSRMNLPTSFSSKKPFFYGEEYNLCHFWSNFEIASTDLFSSPEYESYFQFLEEKKGFYQERWGDAPVHSLAVAMFLNISEIHYFRDIGYRHSNLVHCPKNAPDELQLPYVPASPEYASSAKPDKPPRVSVRDVFRSGRQTEGVNNLNRGSGCRCNCPKKYKELEDSPSCCIGRWMVLTNDKYKGEKYLDKYSMAEEVKQTLSKGEKLNVKEILKRHHKYPT

SEQ ID NO: 11 - protein, 860 aa, Synthetic Construct
MKVSKRLIPRRSRLLIMMMLLVVYQLVVLVLGLESVSEGKLASLLDLGDWDLANSSLSISDFIKLKLKGQKTYHKFDEHVFAAMARIQSNENGKLADYESTSSKTDVTIQNVELWKRLSEEEYTYEPRITLAVYLSYIHQRTYDRYATSYAPYNLRVPFSWADWIDLTALNQYLDKTKGCEAVFPRESEATMKLNNITVVDWLEGLCITDKSLQNSVNSTYAEEINSRDILSPNFHVFGYSDAKDNPQQKIFQSKSYINSKLPLPKSLIFLTDGGSYALTVDRTQNKRILKSGLLSHFFSKKKKEHNLPQDQKTFTFDPVYEFNRLKSQVKPRPISSEPSIDSALKENDYKLKLKESSFIFNYGRILSNYEERLESLNDFEKSHYESLAYSSLLEARKLPKYFGEVILKNPQDGGIHYDYRFFSGLIDKTQINHFEDETERKKIIMHRLLRTWQYFTYHNNIINWISHGSLLSWYWDGLSFPWDNDIDVQMPIMELNNFCKQFNNSLVVEDVSQGFGRYYVDCTSFLAQRTRGNGNNNIDARFIDVSSGLFIDITGLALTGSTMPKRYSNKLIKQPKKSTDSTGSTPENGLTRNLRQNLNAQVYNCRNGHFYQYSELSPLKLSIVEGALTLIPNDFVTILETEYQRRGLEKNTYAKYLYVPELRLWMSYNDIYDILQGTNSHGRPLSAKTMATIFPRLNSDINLKKFLRNDHTFKNIYSTFNVTRVHEEELKHLIVNYDQNKRKSAEYRQFLENLRFMNPIRKDLVTYESRLKALDGYNEVEELEKKQENREKERKEKKEKEEKEKKEKEEKEKKEKEEKEKKEKEEKERKEKEEKEEYEEDDNEGEQPTEQKSQQEAKE

SEQ ID NO: 12 - protein, 652 aa, Synthetic Construct
MVDLFQWLKFYSMRRLGQVAITLVLLNLFVFLGYKFTPSTVIGSPSWEPAVVPTVFNESYLDSLQFTDINVDSFLSDTNGRISVTCDSLAYKGLVKTSKKKELDCDMAYIRRKIFSSEEYGVLADLEAQDITEEQRIKKHWFTFYGSSVYLPEHEVHYLVRRVLFSKVGRADTPVISLLVAQLYDKDWNELTPHTLEIVNPATGNVTPQTFPQLIHVPIEWSVDDKWKGTEDPRVFLKPSKTGVSEPIVLFNLQSSLCDGKRGMFVTSPFRSDKVNLLDIEDKERPNSEKNWSPFFLDDVEVSKYSTGYVHFVYSFNPLKVIKCSLDTGACRMIYESPEEGRFGSELRGATPMVKLPVHLSLPKGKEVWVAFPRTRLRDCGCSRTTYRPVLTLFVKEGNKFYTELISSSIDFHIDVLSYDAKGESCSGSISVLIPNGIDSWDVSKKQGGKSDILTLTLSEADRNTVVVHVKGLLDYLLVLNGEGPIHDSHSFKNVLSTNHFKSDTTLLNSVKAAECAIFSSRDYCKKYGETRGEPARYAKQMENERKEKEKKEKEAKEKLEAEKAEMEEAVRKAQEAIAQKEREKEEAEQEKKAQQEAKEKEAEEKAAKEKEAKENEAKKKIIVEKLAKEQEEAEKLEAKKKLYQLQEEERS

SEQ ID NO: 13 - protein, 644 aa, Synthetic Construct
MRTRLNFLLLCIASVLSVIWIGVLLTWNDNNLGGISLNGGKDSAYDDLLSLGSFNDMEVDSYVTNIYDNAPVLGCTDLSYHGLLKVTPKHDLACDLEFIRAQILDIDVYSAIKDLEDKALTVKQKVEKHWFTFYGSSVFLPEHDVHYLVRRVIFSAEGKANSPVTSIIVAQIYDKNWNELNGHFLDILNPNTGKVQHNTFPQVLPIATNFVKGKKFRGAEDPRVVLRKGRFGPDPLVMFNSLTQDNKRRRIFTISPFDQFKTVMYDIKDYEMPRYEKNWVPFFLKDNQEAVHFVYSFNPLRVLKCSLDDGSCDIVFEIPKVDSMSSELRGATPMINLPQAIPMAKDKEIWVSFPRTRIANCGCSRTTYRPMLMLFVREGSNFFVELLSTSLDFGLEVLPYSGNGLPCSADHSVLIPNSIDNWEVVDSNGDDILTLSFSEADKSTSVIHIRGLYNYLSELDGYQGPEAEDEHNFQRILSDLHFDNKTTVNNFIKVQSCALDAAKGYCKEYGLTRGEAERRRRVAEERKKKEKEEEEKKKKKEKEEEEKKRIEEEKKKIEEKERKEKEKEEAERKKLQEMKKKLEEITEKLEKGQRNKEIDPKEKQREEEERKERVRKIAEKQRKEAEKKEAEKKANDKKDLKIRQ

SEQ ID NO: 14 - protein, 594 aa, Synthetic Construct
MRIRSNVLLLSTAGALALVWFAVVFSWDDKSIFGIPTPGHAVASAYDSSVTLGTFNDMEVDSYVTNIYDNAPVLGCYDLSYHGLLKVSPKHEILCDMKFIRARVLETEAYAALKDLEHKKLTEEEKIEKHWFTFYGSSVFLPDHDVHYLVRRVVFSGEGKANRPITSILVAQIYDKNWNELNGHFLNVLNPNTGKLQHHAFPQVLPIAVNWDRNSKYRGQEDPRVVLRRGRFGPDPLVMFNTLTQNNKLRRLFTISPFDQYKTVMYRTNAFKMQTTEKNWVPFFLKDDQESVHFVYSFNPLRVLNCSLDNGACDVLFELPHDFGMSSELRGATPMLNLPQAIPMADDKEIWVSFPRTRISDCGCSETMYRPMLMLFVREGTNFFAELLSSSIDFGLEVIPYTGDGLPCSSGQSVLIPNSIDNWEVTGSNGEDILSLTFSEADKSTSVVHIRGLYKYLSELDGYGGPEAEDEHNFQRILSDLHFDGKKTIENFKKVQSCALDAAKAYCKEYGVTRGEEDRLKNKEKERKIEEKRKKEEERKKKEEEKKKKEEEEKKKKEEEEEEEKRLKELKKKLKELQEELEKQKDEVKDTKAK

SEQ ID NO: 15 - protein, 488 aa, Synthetic Construct
MYHLAPRKKLLIWGGSLGFVLLLLIVASSHQRIRSTILHRTPISTLPVISQEVITADYHPTLLTGFIPTDSDDSDCADFSPSGVIYSTDKLVLHDSLKDIRDSLLKTQYKDLVTLEDEEKMNIDDILKRWYTLSGSSVWIPGMKAHLVVSRVMYLGTNGRSDPLVSFVRVQLFDPDFNELKDIALKFSDKPDGTVIFPYILPVDIPREGSRWLGPEDAKIAVNPETPDDPIVIFNMQNSVNRAMYGFYPFRPENKQVLFSIKDEEPRKKEKNWTPFFVPGSPTTVNFVYDLQKLTILKCSIITGICEKEFVSGDDGQNHGIGIFRGGSNLVPFPTSFTDKDVWVGFPKTHMESCGCSSHIYRPYLMVLVRKGDFYYKAFVSTPLDFGIDVRSWESAESTSCQTAKNVLAVNSISNWDLLDDGLDKDYMTITLSEADVVNSVLRVRGIAKFVDNLTMDDGSTTLSTSNKIDECATTGSKQYCQRYGELH

SEQ ID NO: 16 - DNA, 1554 bp, Synthetic Construct
  source 1..1554: mol_type = other DNA
atgaaaaaagtaataaagaaatatttctttttagcattagctattataatgtattcgtgtaatgaagatgaaaagtatgatatattagaaagatacactcctgaaactataacatctgacgaaatagctcctgtgcttaatttacaggcacaatatatggatagtaatagcgaaatagtacttgtaacatggatgaatccggaagatgattttttgtctaaggtggaaatctcttgctgttctgcgaatgataatcttttgggtgaacctgtgttgttggacgctgtttctaccaaagtaggttcttatcagacgtcactttctgtggaagagaggggatatgtaaagattgtagctattaatgaaaaaggagtacgctcggaagcccgtacagcagagatcctttcttcccaacaggattttgtatatagagcagattgtttgatgtcttctgtgattgaattattttttggtgggagatataatgcatggaatgagaattaccccaatgctacaggtccctattgggatggcattgcagccgtttggggacaaggtgcagcttattccggatttgttacaatgtataaggtcacaaaggaaactaataatgagaaactaagagcaaaatatgcagaaaaggaagaaacttttctaaactcaatagacatttttttgaataatggtagtggacggaaatcttttgcttatggtacttatattgggccgaatgatgagcgttattacgatgataatgtctggattggcatcgaaatggccaatttatatgaacttacagggaatgaagtttatttgcagcatgcaaatactgtttggaactttattttggaagggatagatgacgtgactggcggtggagtatattggaaagaaggtgcggtatcaaagcatacatgttccactgccccggcagctgtaatggctctaaaattataccaattgagcaagaatgaatcatatttggaaatagcaaagagtttgtattcatactgtaaagatgtattacaagatccgaatgactatttattttatgacaatgttcgcttaagtgacccttccgataagaattcggagcttaaagtatctaaggataaattcacgtataattcgggacaaccaatgttagctgctgctatgttgtatcggattacaaaagaagaacaatttctgaaagatgcccaaaatatagcacagtcgatttataaaaaatggtttaaaaactatcattcgtctatacttgatagagatataatgatattaagcgatccaaacacttggtttaatgccgttatgttcaggggattcgtagagctatataaaatagataagaacgatgtttatgtcaaagcggtgaaaaataccatggaacatgcttggcaaagcaactgtagaaatcggttgactaatctaatgagcgacgattatgcaggtgataagaaagaaggtaagtggaatataaagacacaaggtgcttttgttgaaatcttctcacttattggggaattggaacaacttggatgttttcaggagtag

SEQ ID NO: 17 - DNA, 1554 bp, Synthetic Construct
  source 1..1554: mol_type = other DNA
atgaagaaagtaattaagaaatattttttcctagccttggcaatcattatgtactcatgtaacgaagacgagaaatatgacattcttgaacgttatacccctgaaactataacctctgacgagatcgcacctgtactaaaccttcaagcccagtacatggattcaaacagtgaaatagttcttgtgacttggatgaacccagaggatgattttctgagtaaagttgagatttcttgctgcagtgctaacgataacttactgggtgagcccgtccttcttgatgccgtctcaaccaaggtcggctcctaccagacgtccctttctgtcgaagaacgtggatatgttaagatcgtagctataaatgaaaagggagttaggtctgaggctaggacggctgagattttgtcatctcaacaagacttcgtctatcgtgcagactgccttatgtctagtgtgattgaactgttctttggaggaaggtacaatgcatggaacgaaaattaccccaatgcaaccggcccttactgggatggaatcgccgctgtgtggggtcagggtgcagcctattctggtttcgtaactatgtacaaagttaccaaagaaacaaataacgaaaaactaagggctaagtatgcagaaaaggaggaaacattcctgaactctatagacatctttttaaataatggctctggcagaaagtcatttgcctacggcacgtacatcggtcctaacgacgagcgttattacgatgataatgtgtggataggtatagaaatggcaaacttatatgagctgacaggaaacgaggtgtacctacaacatgccaataccgtgtggaatttcatattagaaggcattgatgatgtaacgggaggtggcgtatactggaaggagggtgcagtttccaaacacacgtgctcaaccgcccccgcagctgtaatggctttgaaactttaccagttgtccaagaatgaatcctacttagagatcgccaaatccttgtattcctactgcaaagatgtcttgcaagatccaaacgattatcttttttacgacaacgtgaggctaagtgacccttcagataagaacagtgaactaaaagtatcaaaagacaagttcacttacaacagtggtcagcccatgcttgcagcagccatgctgtatcgtataaccaaagaagagcagtttctgaaagacgcccaaaacattgcccaatcaatatacaagaaatggttcaaaaattaccattcatcaatcttagatagggatataatgattttgtctgatccaaacacctggtttaacgcagtcatgtttaggggttttgtcgagctgtataaaatcgacaaaaatgatgtttatgttaaggcagttaagaacacaatggagcatgcttggcaatcaaactgccgtaacagacttaccaatcttatgtctgacgactatgccggagacaagaaggagggtaagtggaacattaagacccaaggagcttttgttgaaattttttctttgattggcgagttagaacagttaggctgtttccaggaatag

SEQ ID NO: 18 - DNA, 2283 bp, Synthetic Construct
  source 1..2283: mol_type = other DNA
atgaaaacacatttttcatttaaacacttgctatttcttggaggtgccgtgttgtacagcctgcaatcttctgccgttaagaatcctgtagactatgtcagcacactgataggcactcaatccaagtttgaactgtctaccggaaacacgtatccggctacggcattgccgtggggaatgaatttctggacaccgcagaccggtaaaatgggagacggttgggcgtacacgtatgatgccgacaaaatccggggattcaaacaaacacatcagcccagtccctggatgaacgactacgggcagttcgccatcatgcctatcacaggcggactggtattcgatcaagaccgacgtgccagttggttctctcacaaagcggaagttgccaaaccttattattataaggtatacctcgccgaccatgatgtaacaaccgagcttgctcctacggagcgtgccgtcatgttccgtttcacgtatccggagacaaagaatgcctacgtgattgtagacgctttcgacaaaggttcttatgtgaaagtgattccggaagaaaacaagattatcggctattcaaccaagaatagcggcggtgtgccggaaaacttcaaaaactatttcgtgattcaattcgacaaaccgttcacattcgtttccacagttttcgaaaacaacattcttccgaatgaaacagaagcaaaaggaaaccacacaggggccgtgatcggattcgccacgaaaaagggagaaatcgtacacgcacgtgttgcttcctcctttatcagccccgaacaggcggagttgaatctcaaagagcttggcaaaaacagtttcgaccaactggtagcgaacggaagagaaatctggaaccgtgaaatgagtaaaatagagatagaagacgataatatcgataatttacgcaccttctattcttgtttataccgttccatgctttttccacgcagtttctacgagatagatgctaagggacaagtcatgcattacagcccctacaacggcgaagtgcgtcccggttatatgtttaccgacaccggattctgggacacgttccgctgcctgttccctttcctcaacctgatgtatccgtcaatgaatcaaaagatgcaggagggactagtgaatacttacaaggaaagtggtttcctgccggaatgggccagtccgggacatcgggattgtatggtaggcaacaactcggcttccgtagtagccgacgcttacatcaaaggattgcgaggatatgatatcgaaactctttgggaagcattgaaacatggagcaaatgcacatcttcgcgggactgcttcaggtcgtctcggttacgaatcttacaaccaactgggatatgttgccaacaatatcggcataggacaaaacgttgcacgtacattggagtatgcttacaacgactgggcaatttatacactaggtaagaaacttggtaaaccggagaacgaaatcgacatttataagaaacacgcgctgaactacaaaaatgtctatcacccggaacgcaaactgatggttggcaaagataacaaaggcgtattcaatccgaatttcgatgcagtggactggagcggtgaattttgcgaagggaatagctggcactggagcttctgcgtattccacgacccgcaaggacttatcaacctgatgggaggcaagaaagaattcaacgcgatgatggattctgtttttgtcatctcgggtaaactgggaatggaaagccgcggcatgattcacgaaatgcgtgaaatgcaagtaatgaacatggggcaatatgcgcatggcaaccagcctattcaacacatggtatatctctacaactattcaagcgaaccctggaaagctcaatactggatacgtgagattatgaacaaactatataccgccggtcccgacggttattgcggtgacgaagacaacggacagacttccgcctggtatgtattctccgcactcggtttctatccggtttgcccgggaacagatgaatatatcataggaaccccgctctttaaatcagcgaagttacatttggagaacggaaagaccatcacgatcaaggcagataacaaccagcttgacaaccgctacatcaaggaaatgaaagtaaacgggaaatcacaaacccgtaatttccttacacatgaccagctgattaaaggtgctaatattcaatttcaaatgagccccgtgcccaataaacaacggggaaccacagaaaaagatgtaccttactctctttcgtttgaataa

SEQ ID NO: 19 - DNA, 2283 bp, Synthetic Construct
  source 1..2283: mol_type = other DNA
atgaaaacacatttttcatttaaacacttgctatttcttggaggtgccgtgttgtacagcctgcaatcttctgccgttaaaaatcccgtcgactatgtgtctacccttataggcacgcaatccaagtttgagttgtccacaggcaacacctaccctgctaccgctcttccatggggcatgaacttttggactccacagacaggaaaaatgggtgatggatgggcatatacgtacgatgctgacaagatccgtggctttaaacaaactcaccaaccatctccatggatgaacgactacggtcagtttgcaataatgccaattactggaggacttgtatttgaccaagatagacgtgctagttggttttcccacaaggcagaagtcgctaaaccatactattacaaggtctaccttgctgaccatgacgtgacaaccgaattggcccccaccgagagggccgtgatgtttaggtttacgtaccccgagacgaaaaacgcctacgttattgtagatgcctttgataagggaagttatgtcaaagtaatacctgaggaaaacaagattataggttattctacaaaaaattcaggcggcgtcccagaaaattttaagaactacttcgttattcagtttgacaaaccatttacgttcgtatcaactgtatttgaaaacaatattttgccaaacgagacagaggccaagggtaaccacacaggcgctgtgatcggcttcgcaacgaagaagggcgaaatagtacatgctagagtcgcctcttctttcatatctcctgaacaagccgagttaaacttaaaggaattgggaaaaaattcttttgatcaactggtagccaacggtagggagatttggaatcgtgagatgagtaagatcgagatcgaggatgataacattgataatttaaggacgttctattcttgtctgtatagatccatgttgtttcctaggtccttttacgagattgacgctaagggccaggtgatgcactattcaccctacaatggcgaagtacgtcctggatacatgttcacggatacgggattttgggacacgtttaggtgtctgttcccttttttgaatctgatgtatccctccatgaaccagaaaatgcaggagggccttgtaaacacttacaaggagtccggatttttaccagagtgggcaagtccaggccatcgtgattgtatggttggcaacaattcagcatcagttgtggctgatgcctatatcaaaggtttgagaggatacgatatcgagacgctgtgggaggcccttaaacacggtgccaacgctcatctaaggggtaccgcatctggcagattaggttacgagtcctacaaccaactaggctacgtggctaataatatcggtattggccagaacgttgcaagaacccttgaatacgcttacaacgactgggcaatctacactttgggtaaaaaacttggaaaacccgaaaatgaaatagacatttataagaaacacgctcttaactacaaaaacgtgtatcaccctgaaaggaagctaatggtcggtaaggacaacaagggcgtctttaaccctaatttcgatgctgtggactggtctggagagttctgcgaaggcaattcctggcattggtccttctgtgtttttcacgaccctcaaggattaattaatttgatgggtggtaagaaggagttcaatgctatgatggattccgtattcgtgatctctggtaaactgggcatggagtctcgtggtatgatccacgaaatgagagagatgcaggtaatgaacatgggacaatacgcacatggcaatcagcctatacagcatatggtatatctttataactacagttcagagccttggaaggcacaatattggattagggagatcatgaacaagctttataccgccggccctgatggatattgtggcgatgaagataacggacagaccagtgcatggtatgtgttttccgcacttggtttttaccctgtgtgccctggtacggatgagtacattatcggcacgccattattcaaatctgctaagttgcatcttgaaaacggaaagacgataacgataaaagccgacaacaaccaactggataacagatatattaaagaaatgaaggtcaacggtaagtcacaaacgagaaactttttaacccatgaccaactaattaagggagccaacatacaattccagatgagtccagtccccaataagcaacgtggaacaacagagaaggacgtgccttattctttgtccttcgagtag

SEQ ID NO: 20 - DNA, 951 bp, Synthetic Construct
  source 1..951: mol_type = other DNA
atgaaactgaaaaaccttttactaattgcccttgttgcgatcgtcttttgcggttgtcaaagtaactatcagcctacttctatcaccgttgcctcctacaatttgagaaacgccaacggtggcgattcaatcaacggaaacggttggggacaacgttacccggtcattgcccaaatagtgcaatatcacgatttcgatattttcggcacgcaggagtgctttattcatcaactgaaagatatgaaagaagcattacccggttatgattatatcggtgtaggtcgcgacgacggcaaagagaaaggtgaacattctgctattttctatcgcacagacaagtttgacgtgatagagaaaggtgatttttggttgtcggaaactcccgacgtgccgagcaaaggatgggatgccgtgttgccgcgtatttgcagttggggacacttcaaatgcaaagataccggcttcgaattccttttcttcaacctgcacatggaccatatcggcaagaaggcacgtgtggaaagtgcattcctcgtacaggacaagatgaaagaacttggcaaaggcaaagagcttccggccatcctgacgggagacttcaatgtcgaccagacccaccagtcttatgatgcttttgtgagcaaaggggtgttgtgcgactcttacgagaaggccggcttccgctatgctatcaacggcacgttcaacgacttcgacccgaacagctttacggaaagccgtatcgaccatatattcgtttctccgtctttccaagtgaaaagatatggtgtgctgactgatacttaccgcagcatcgtaggcaagggagaaaagaagcaggcgaacgattgcccggaagaaatcgacatcaagacttatcaggcgcgcactccttcagaccatttccccgtaaaggtggaactggagttcgaccagcgtcagcagaaataa

SEQ ID NO: 21 - DNA, 1158 bp, Synthetic Construct
  source 1..1158: mol_type = other DNA
atgagaaatatatgttttgtagcctgtatgttattttgccttacttccgcagtgggaaagacaccgggaaatacccgttatctttctattgccgactcgattctatctaatgtattgaatctctatcagacgaatgacggactactaacagaaacgtatcctgtcaatcccgaccaaaaaattacttatctggcgggcggaacgcagcagaacggaacgctgaaggcttcttttctatggccgtattccgggatgatgtcgggttgtgtggctttatacaaagcgaccggaaacaagaagtacaaaaagattctcgagaaaagaattctaccgggaatggagcagtattgggataacagtcgcttgccggcctgttatcagtcataccccaccaagtacgggcagcacggacgttattatgacgataacatctggattgcactggattactgcgattattaccaactgactcacaagcctgcatctttggaaaaagccgttgcattgtatcaatatatctacagtggatggagcgatgagataggcggtggcatcttttggtgtgaacagcagaaggaagcgaagcatacttgttccaatgcaccgtctactgtgctcggtgtcaagttgtaccggctgacgaaggatgccaaatacctcgaaaaagcaaaagagacgtatgcctggacgaaaaagcatctgtgcgaccctaccgaccatctttactgggataacatcaacctgaaagggaaagtttccaaagagaagtacgcctacaacagtggacagatgattcaggcgggtgtattgctctatgaggaaacgggagatgaacagtatttgcgcgatgcacagcagacagccgcaggaactgatgcttttttccgcacaaaagccgacaagaaagacccgactgtcaaagtgcataaagacatggcctggtttaacgtgatcttattcagaggactgaaagctctgtataagattgacaagaatccggcgtatgtcaatgcgatggtggaaaatgcgcttcacgcctgggaaaactaccgggatgaaaacggattattaggcagggattggtcgggacataacaaggagcagtataaatggctgctcgacaatgcctgtcttattgaattctttgcagagatttaa

SEQ ID NO: 22 - DNA, 1158 bp, Synthetic Construct
  source 1..1158: mol_type = other DNA
atgagaaacatctgctttgtcgcctgtatgctgttctgtctgaccagtgctgtgggcaagactcctggaaacacgaggtacctatctattgccgactctatcctttccaacgtgttgaacctttaccaaactaacgatggtcttctgaccgaaacttatcctgttaaccccgaccagaagataacctatttggctggcggcacacaacagaatggcaccctgaaggcatcttttttgtggccttattctggcatgatgtccggatgcgttgcattgtataaagccactggcaacaaaaagtataaaaagatacttgagaaaaggattttaccaggaatggagcagtactgggacaatagtcgtttaccagcatgttatcaatcataccctactaaatacggccagcacggaagatactatgacgataatatctggatcgccttagattactgcgactattaccagttaacccacaaacccgcctctctggagaaagccgtagctctatatcagtacatctattctggttggtcagatgagattggcggaggcatattttggtgtgagcaacaaaaagaggccaagcacacgtgctccaatgccccttccactgtattaggtgtaaaactgtataggcttacaaaagacgccaaatatctggaaaaagctaaagagacgtatgcttggaccaagaaacatctttgcgaccctacagatcatttgtactgggataatataaacttgaaaggaaaggtttctaaagaaaaatacgcctataatagtggtcaaatgattcaggccggcgttctgttgtatgaggaaacaggcgatgagcaatatcttcgtgatgctcaacaaacagccgctggcacagacgcatttttcagaacgaaggcagacaagaaagacccaactgtcaaggtacataaggacatggcctggtttaacgtaattttatttagaggcctgaaggcattatataaaatagacaagaaccccgcctatgtaaatgctatggtagagaatgccctgcatgcctgggaaaattacagagacgagaatggacttctaggaagagattggagtggtcacaacaaagaacaatacaaatggctattagataacgcctgtctaattgagttcttcgcagagatttag

SEQ ID NO: 23 - DNA, 1446 bp, Synthetic Construct
  source 1..1446: mol_type = other DNA
atgaatataactaaagccttttgtttgtccatagcactcttgggcgctagcaatatgcaggctataacgaacagtgattttgtcatccaacaagataataccaaaatcaacaactatcagacgaaccgtccggaaacatcgaaacgtctgtttgtctcacaagctgtggaacaacagattgcgcatatcaagcaactgctgacgaatgcccgcttagcatggatgttcgaaaactgtttcccgaacacactggatactactgttcattttgacggtaaagacgatacgtttgtttatacaggtgacatccacgccatgtggttgcgcgattcgggtgcacaagtatggccttacgtgcaactcgccaacaaagacgcagaactgaaaaaaatgctcgctggcgttatcaaacgtcagttcaagtgtatcaatatcgacccgtatgccaatgctttcaacatgaattccgaaggcggcgaatggatgagtgaccttacggacatgaagcccgaactgcacgaacgcaaatgggaaatcgactcgctctgttatcctatccgtctcgcttatcattactggaagacgacgggagatgccagtatattctccgacgaatggcttacagccatcgccaaggttctgaaaacgtttaaggaacagcaacgaaaagaagatccgaaaggtccttatcgtttccaacgcaaaacggaacgtgcactcgatacgatgaccaatgacggctggggcaatcctgtaaagccggtcggactgattgcttctgctttccgtccttcggatgatgctacaactttccagtttctcgttccgtccaacttctttgctgtaacttcattgcgcaaagctgccgaaattctgaatacggtcaacaagaaacctgatttagctaaagaatgtactacactgtctaacgaagtggaaacagccctgaaaaagtatgcggtttacaatcatccgaaatatggcaaaatctatgctttcgaagtggacggtttcggcaatcaactgttaatggatgatgccaatgtgccgagtctcattgccctgccttatcttggggatgtgaaagtgaacgatcctatttatcagaatacccgtaagtttgtatggagcgaagataatccttacttcttcaaaggtactgccggcgaaggaattggcggtccgcacatcggatatgatatgatttggcccatgagtattatgatgaaagcattcaccagtcaaaacgacgcagaaatcaagacctgcatcaaaatgctgatggatacggatgccggaacagggttcatgcatgaatctttccacaagaacgacccgaaaaactttactcgttcctggtttgcatggcaaaatacgctgtttggagaactaatcctaaaactcgtgaatgaaggaaaggtagacttactgaatagtatccaatag

SEQ ID NO: 24 - DNA, 1446 bp, Synthetic Construct
  source 1..1446: mol_type = other DNA
atgaatattactaaggccttttgcctaagtatcgcattattaggagcctctaatatgcaagccattaccaatagtgactttgttattcagcaggacaacacaaaaatcaataattaccagacaaatcgtccagagacatcaaaaaggttgttcgtgtctcaggcagtcgagcagcaaatcgctcacatcaagcaacttctgacaaacgcaaggcttgcctggatgttcgagaactgctttccaaacactttagacacgacggtccacttcgacggaaaggacgatacattcgtttataccggcgatatccacgctatgtggctaagagactccggagcacaggtttggccctacgtccaactggccaataaagatgccgagctgaaaaaaatgctggctggagtcattaaaagacaattcaaatgcattaacattgatccttatgcaaatgcattcaatatgaattcagaaggcggcgagtggatgtccgatttgacagatatgaaacccgagcttcatgagcgtaaatgggagatcgacagtctttgctaccccattagactggcatatcactattggaagacaacaggagacgcttccatatttagtgacgagtggttaacggcaatagccaaagtcctaaagacatttaaggagcagcagcgtaaagaggacccaaagggtccatatagatttcaaagaaagacagagagagccttagataccatgacgaacgacggatggggtaatcctgtcaagcctgtaggtctgattgcatccgcctttaggccatcagatgatgctacgacatttcaattcttagtgccaagtaatttctttgccgtgacttctcttaggaaagctgccgagatacttaacacggtaaacaagaaaccagaccttgccaaagaatgcactacattgtcaaatgaagtagaaacggcactaaaaaaatatgccgtctacaatcatcccaaatacggcaaaatctatgcttttgaagtcgatggcttcggaaaccaactattaatggatgacgctaacgttccctctctaatagccctaccttatcttggcgatgtaaaagtgaacgacccaatctaccagaatactagaaagtttgtctggagtgaggacaatccttacttcttcaagggtaccgcaggagaaggcatcggcggtcctcatattggttacgatatgatttggcctatgtctatcatgatgaaggccttcacatctcagaatgatgcagagataaaaacatgtatcaaaatgttgatggacactgatgccggcacaggttttatgcatgagtcctttcacaaaaacgacccaaaaaatttcaccagatcctggtttgcttggcagaacacgttgttcggagagttaattctaaaattggtaaacgaaggtaaagtcgatttattgaacagtatccaatag

SEQ ID NO: 25 - DNA, 3597 bp, Synthetic Construct
  source 1..3597: mol_type = other DNA
atgaacaaaaaagtaattgccgtagccctcgcccttgccttagcaggaggaagctatgcacaagatgacaccgcgaagaaaaaggtgaaagcctatatggtgtcggacgcccacctcgacacccagtggaactgggacatccagacaacaatcaacgaatatgtctggaataccattagtcagaacttatttctgctaaagaaatatcccgaatacgttttcaactttgaagggggagtgaagtatgcgtggatgaaggaatactatcccgaacagtatgaagagatgaagaaattcatcgaggaaggccgctggcatatcgccggaagtagctgggaagcaagtgatgtgttggttccttccgtcgaagcctccatccgtaacatcatgctcggacagacgtactaccggcaagagttcggaaaagaaggaacggatatcttcctgccggactgcttcggattcggatggacgcttcccaccattgccgcacactgcggactgatcggcttctcttcacagaagctggactggcgtaatcatcccttctatggaaagagcaagcatccgtttaccatcggactctggaagggcattgacggcaaacaagtaatgctagcccacggatatgactacggacgcaaatggaacaacgaagatctctcgaagaataaagatctggaaaaattagcccaacgtactccgctcaatacggtctaccgctattatggaacaggggatatcggtggctctcctactctgggttcggtacgttctgtagaacagggaatcaaaggtgatggcccggtagaggtgatcagtgctaccagcgatcagttgttcaaagattatctgccgttcaacaatcacccggaactgccggtatttgacggagagttattgatggatgttcacggaacaggttgctatacttcgcaggcagccatgaagctgtacaaccggcaaaacgaacagttgggcgatgcagcagaaagagcggcggtcgctgccgaatggttgggtactgccagctatccgcaacacacgctgacggaggcatggaaacgtttcatcttccatcaattccatgatgacctgacgggaacgagtatcccccgtgcctatgagttctcatggaacgatgaactgatctctctaaaacaattctcacaagtactgacttcttccgtcaacgccattgccggacagatggatacacgcgtgaaaggaacgcctgtcgttctttataatgcaaacgctttcccggtatcggacttgacagagatcatcctcgaacagcctaaaaccccgaaaggcttcactgtatacaatgcacaaggcaagaaagtcgcttcgcaaatgatcggttacgagaacggacgtgctcacatcctggttgcagcgtcactgcccgcaaacagttatgcagtgtacgatgtccgcaccggaggatctgaaaaaacgatctctccttcagccgcctcagccatcgaaaactccgtctacaaaatcacactggataaaaacggagatatcatctcactgaccgacaagcgcaacaacaaagaactcgtaaaagatggaaaagcgattcgcctggcactcttcaccgaaaacaagtcgtacgcatggcctgcatgggaaatcctgaaagagaccatcgaccgtgaacctgtctccatcacagacggcgcaaagatcactttagtggaaaacggcgcactccgtaaagcactctgcattgagaagaagtatggcaaatcgctcttcaagcaatacatccgcctctacgaaggcagccgtgccgaccgcatagatttctataacgaaatagactggcagtcaacaaacacactgctgaaagcagagtttcctctgaatatagaaaatgaaaaggctacttacgatctgggaatcggcagcgtggaaagaggtaataatgtacagaccgcttacgaagtatatgcgcagcaatgggcagacctgaccgataagaacaacagctacggtgtatcgatcctaaatgacagtaaatatggctgggataaaccggataacaacacgatccgtctgactcttctccatacaccggaaacaaaaggaaattacgcttatcaggatcaccaggacttcggcttccatacatttacttatagcctcacaggacataacggagcacttgacaaacccgccaccgccatcaaagctgaaattctgaatcagccgatcaaagccttcagcagtccgaaacatgccggaacactaggtaaagaatttgcttttgtacgttcaagcaacgatcaagtcgttatcaaagcgctgaaaaaagcggaagtatccgatgaatatgtagtacgtgtatatgaaacaggaggcgcagctccgcaacaggcagccatcaccttcgccggtgaaatagagaaggcagtacttgcagacggtacggaaaaagagatcggcagtgctgacttcaacaagaaccagctgaatgtatccatcgctccctacagcatacagacatttaaagtgaagctgaagaaaaaagctgatcttcaagctccggcatgcgcttatcttcctttggactatgatcgcagatgtttcagttggaatgctttccgcaaagaagggaacttcgaatcgggcaacagctatgcagcagaacttctccccgactccatcctgaaagccgacggcattcctttccgcttgggagagaaagaaattgccaatggtttgacttgcaaaggcaatgtacttcagttgccaaccggacattcttacaaccgtatctatttcctggcagcctctgccggtgaagatgcagttgctaccttcagcaccggtaacaactcacaggaaatcaccgtaccttcctataccggttttatcggtcagtgggagcatctgggacatacggaaggcttcctgaaagatgcagaaatcgcttatgtcggcactcaccgtcatgcttctgacaaagatgaggcttatgagtttacgtatatgttcaagtttggcatggatattcctaaaggagcgactacggttactttgccggatcatgcagatatcgtattatttgccgcaacgctggttaatgagaagtatccggcagtaactccggcctcggaattgttccgcacagccttgaaagcagacaatggagaagaagcgacgactaaaacaaacctgttgaaacaagccaaactaatcaaatgttccggtgaaaccaacgaaaaagaagttgcaagatatgccgtagacggtgatgtgaagacgaaatggtgtgatacaagcacggctcccaactacattgacttcgacttcggaaaggaacagacgatccgtggatggaagttggtaaatgccggaaatgaaggcagcgtctttatcactcatacctgcttcttacaaggcagaaacagtccggacgaagaatggaaaacgattgatgaactgagtgataacaagaaaaacacggtagttcgccagtttaagccgacttcggtacgttacgtcagactgctggttacacaatctacacaaaacaacagtctgaaggctgcaagaatctacgagttggaggtttattga

SEQ ID NO: 26 - DNA, 1152 bp, Synthetic Construct
  source 1..1152: mol_type = other DNA
atgaaatcaacctttttatttctggttactacaaccatgatgacttgtaccgccttgggacaaccttccaacgacaaaaagaacgtattacccgactgggcgttcggaggcttcgaacgaccacagggagctaatccggtgatatctcctatagagaacacgaaattctattgtccgatgacacaggattacgttgcatgggaatccaatgacactttcaatccggctgctaccctgcatgacggcaagattgtcgtgctgtatcgggcagaagataaatccggtgtcggtatcggtcaccgtacctcacgtctcggatacgccacttcgagcgacggcattcacttcaagcgggaaaagaccccggtattttatccggataacgatactcaaaagaaactggaatggccgggcggatgcgaagacccgcgtatcgccgtcacagcagaaggactgtatgtgatgacctatacgcaatggaaccgccacattccgcgtctggcaatagccacttcccgcaatctgaaagactggacaaagcacggtcccgcttttgccaaagcgtatgacggcaagttcttcaatttaggatgcaagtccggctccattctgaccgaagttgtcaatgggaaacaggtgatcaagaaaatcgacggaaaatacttcatgtattggggagaggaacatgtgtttgccgccacttccgaagatttagtcaactggactccatacgtaaatacggacggctcgctgagaaaactgttttcaccccgtgacggacacttcgacagccagctgacggaatgcggtcctccagctatttatactccaaagggaatcgtacttctgtataatggtaaaaacagtgcaagcagaggcgacaaacgctataccgccaatgtttacgctgccggacaagccctcttcgacgccaatgacccgacccgtttcatcacccgtctcgacgaaccgttcttccgcccgatggatagtttcgaaaagagcgggcagtatgtagacggaacggtgttcatcgaagggatggtttattataaggataaatggtatctgtattatggttgcgcagattccaaggtgggtatggctatctacaatccgaagaaacctgctgccgcagatccgctgccctaa

SEQ ID NO: 27 - DNA, 1152 bp, Synthetic Construct
  source 1..1152: mol_type = other DNA
atgaagtctacctttctattcctagtgacgactaccatgatgacttgcaccgctcttggacagccctccaacgacaaaaagaacgtcttacccgactgggcatttggtggctttgaacgtccacaaggcgctaatccagttatttcccccatagaaaatactaaattttattgccctatgacgcaggactacgtagcctgggaatcaaacgacacctttaatcctgccgcaactctgcacgatggcaaaatcgtggtgttgtatagagccgaagacaaatccggcgtcggcatcggacataggacatcaagattgggatacgccacgtcctctgacggtatacatttcaaaagagagaagacccctgtcttttatcccgacaatgatacgcagaaaaaacttgaatggcctggcggttgtgaggatccaaggattgcagtgacggcagagggactttatgttatgacttacacccaatggaatagacatatacctcgtctagcaatcgcaacctctaggaaccttaaagattggacgaaacatggccccgcttttgctaaagcctacgacggaaagtttttcaatttaggctgtaagagtggcagtattttgacagaagtggtcaatggtaaacaggtgatcaagaaaatcgatggtaagtattttatgtattggggtgaggaacacgttttcgcagctacttctgaagacctggtgaactggacaccctacgttaatacagatggaagtctaaggaagttattttcacctcgtgacggtcacttcgactcccaactaacggaatgtggcccacccgccatttatacgcctaagggcatcgtactgctgtataacggtaaaaatagtgccagtagaggcgataaaagatacaccgctaacgtatacgcagccggccaagctctattcgatgctaacgaccctaccaggttcataactagattggacgagccctttttcaggccaatggattcatttgagaaatcaggccagtacgtagatggcacggtttttattgagggcatggtttattacaaggataaatggtatctttattatggttgtgctgattctaaagttggtatggcaatatataatcccaagaagccagcagctgcagatccacttccctaa

SEQ ID NO: 28 - DNA, 1446 bp, Synthetic Construct
  source 1..1446: mol_type = other DNA
atgaatataaccaaaacactttgcctctgcgcagcactttcgggcgctgccggcgtgcaagcaatggaaaaccgcgaatttgtgacccagcaagacaatacccgggtcaataattaccagaccaaccgtcccgaagcctccaagcgcttattcgtatcgcaggaagtggaacgacagattgaccacatcaagcaactactgaccaatgcgaaactggcatggatgttcgagaactgttttccgaacacactggacactaccgttcacttcgacggaaaagaggacacttttgtatacaccggagacatccacgccatgtggctccgcgactccggtgcgcaggtatggccctatgtgcagcttgccaataaagaccccgaactgaaaaagatgctggcaggagtcatcaaccgccagtttaaatgtatcaatatcgacccgtacgccaacgccttcaacatgaactccgaaggaggcgaatggatgagcgacctgacggacatgaaaccggaacttcacgaacgcaaatgggaaatcgactctctctgctacccgatccgcctggcataccattactggaaaacaacgggcgatgccagcgtattctccgacgaatggctgcaggccattgcaaatgtgctgaagactttcaaggaacagcagcgtaaggacgacgcgaaaggtccgtacagattccagcgtaagaccgaacgcgcactcgacaccatgaccaatgacggttggggcaatccggtgaaacctgtcggactgattgcttccgctttccgcccttcggatgacgctacgactttccagttcctcgttccttccaacttctttgccgttacttccttgcgcaaagctgccgaaattctgaacaccgtgaacaggaaaccggcgctggccaaagaatgtaccgcactggcggatgaagtagaaaaagcattaaagaaatatgctgtctgcaaccatccgaaatacggtaagatttatgctttcgaggtagatggcttcggcaatcagctactgatggacgacgccaacgtgccgagtctcatcgctttgccttatctgggtgacgtcaaagtgactgatccgatttatcagaatacccgcaagtttgtatggagcgaagacaatccttacttcttcaaaggcagtgccggggaaggtatcggaggtccgcatatcggatatgacatgatatggcccatgagtatcatgatgaaagccttcaccagccagaatgacgcagaaatcaaaacttgcatcaaaatgctgatggatacggatgcaggtaccggcttcatgcacgaatcattcaacaaaaacgacccgaaaaactttacccgtgcatggtttgcatggcagaatacgttgttcggagagctgatcctcaaactggtcaatgaaggcaaagtggacttattgaacagcattcagtag

SEQ ID NO: 29 - DNA, 1446 bp, Synthetic Construct
  source 1..1446: mol_type = other DNA
atgaatattacgaaaactttgtgcttgtgtgcagcactaagtggcgcagccggagttcaggcaatggagaaccgtgagtttgttactcaacaggataatacaagagtcaataactatcaaacgaaccgtcccgaggcatctaaaagattattcgtaagtcaagaagtggaaaggcagatagaccatataaaacagttattgaccaatgccaaattagcatggatgttcgaaaattgcttccccaatactctggacacgaccgtacatttcgatggtaaagaagatacattcgtttacaccggagacattcacgctatgtggctaagagactcaggcgctcaggtatggccatacgttcagctagctaataaggatcccgagctgaaaaagatgctagctggtgttattaatcgtcagtttaaatgtatcaatatagatccctatgctaacgcatttaatatgaactccgagggcggtgaatggatgtctgatctgacagatatgaaacccgaactgcacgaaaggaaatgggaaattgatagtctgtgctacccaatcagactggcatatcattattggaagactaccggtgatgcttccgtattttccgatgaatggctacaggccatagcaaatgtattaaaaactttcaaagagcaacagaggaaggacgacgcaaagggaccctatagatttcaaaggaagacggaaagagctttagacactatgactaacgacggctggggaaatccagtcaagccagtgggtctaatcgcatccgcatttaggccctcagatgacgcaactacgttccagttcctggtcccttcaaacttcttcgcagtcacgtctttaaggaaagcagctgagatactaaatacggtgaacagaaagcctgccttggctaaagagtgcacagcactggcagatgaggtagagaaagccttgaagaaatacgcagtgtgcaatcatcccaagtatggcaagatatacgccttcgaagtagacggctttggtaatcaactattgatggatgatgctaatgtccctagtttaatagcactaccttatttaggcgacgtaaaagtgacggacccaatttaccaaaataccagaaaattcgtctggtccgaagataatccctactttttcaaaggttcagcaggagaaggtatcggaggaccccatattggttacgacatgatatggcccatgagtataatgatgaaggcatttacgagtcagaatgacgcagagatcaaaacctgcataaagatgctgatggacactgatgctggcacgggttttatgcacgagtcttttaataaaaacgatccaaaaaattttacccgtgcctggttcgcttggcagaacaccttgtttggagagttgatactgaagttggtaaatgaaggtaaagtggatctactgaactccattcaatag

SEQ ID NO: 30 - DNA, 1164 bp, Synthetic Construct
  source 1..1164: mol_type = other DNA
atgagaaatatatgttttgtagcctgtatgttgttttgcctcgcttccgcttccggaaaaacggtgaaaaatcatcctttcgtgtccattgccgactctatcctcgacaatgttctgaatttatatcagacggaagacgggctgcttaccgaaacatatcccgtgaatcccgaccagaaaatcacttatctggcaggcggagcacagcagaacggaaccttgaaggcctcctttctgtggccctactcagggatgatgtccggttgcgtagccatgtaccaggctaccggagacaagaagtacaagacgatactggaaaagcgcatcctgccgggactggaacagtactgggacggagaacgccttccggcatgctatcagtcgtaccctgtcaaatacggtcagcatggacgctactacgatgacaacatctggatcgcactggattattgcgactactaccgcctcacaaagaaggccgactatctgaaaaaggccattgccctgtacgaatacatctacagcggctggagtgacgaactgggcggaggaatcttctggtgcgaacagcagaaagaagcgaagcatacctgctccaatgccccgtcaacagtactcggcgtcaagctataccgtctgacgaaggacaaaaagtatctgaacaaggccaaggaaacttacgcatggaccagaaaacacttgtgtgatcccgacgacttcctttactgggacaatatcaacctgaaagggaaagtctcgaaagacaagtacgcctacaacagtggacaaatgattcaggcaggtgtattactgtacgaagagacaggagacaaggattacttgcgcgatgcccagaagacagccgcgggaaccgatgcctttttccgttcgaaagcagataagaaagacccgtcagtcaaggtacacaaggatatgtcgtggtttaacgtgattctgttcagaggcttcaaggcgctggagaagattgaccacaacccgacttatgtccgtgcgatggcagagaacgcgctccacgcatggagaaactaccgggatgccaacggattactgggcagagactggtcaggacataacgaggaaccttataaatggctgctcgataatgcctgcctgatcgagctgttcgctgaaatcgagaaataa

SEQ ID NO: 31 - DNA, 1164 bp, Synthetic Construct
  source 1..1164: mol_type = other DNA
atgcgtaacatttgttttgtcgcttgtatgctgttttgtcttgcatccgcctctggaaaaactgtcaaaaatcatccatttgtatccattgccgactccatactagataacgtactaaacctataccaaacagaagacggcctattaactgaaacatatcctgtcaaccctgaccagaaaatcacctatttggcaggcggcgctcagcagaacggaaccctaaaggcatcctttctttggccttactccggtatgatgtccggctgtgtggccatgtaccaagctaccggagacaaaaagtacaaaaccatactagagaagcgtatcttaccaggattagaacaatactgggatggtgagcgtttgcccgcatgttaccaatcctatcccgtgaaatacggacaacacggcaggtactatgacgacaacatttggattgcattggactattgtgattattaccgtctaacaaagaaagcagactatctgaaaaaagccattgctctatatgaatacatatacagtggctggagtgatgagttaggtggcggcatcttttggtgtgagcagcaaaaggaagccaagcacacgtgctccaatgcaccctccacggtcttaggtgttaagctttacaggctaacgaaggacaagaaatacttaaataaggctaaggagacttacgcctggactagaaagcatctttgcgaccccgacgactttttatattgggataatattaacttaaagggaaaagtttccaaagataaatatgcatacaactctggccaaatgatccaggccggagtactactatacgaagaaactggcgataaagactaccttagggatgcccaaaaaacggccgctggtacggacgcctttttccgtagtaaagcagacaaaaaagatccatcagtcaaagttcacaaagatatgtcttggttcaacgtcatcctattcagaggttttaaagctctagagaagattgaccacaacccaacttatgtgcgtgccatggcagagaatgcacttcacgcttggcgtaactatagagatgcaaacggacttctgggcagggactggagtggccataatgaagagccatacaagtggctactggataatgcctgtctaatagaattattcgcagagatcgagaaataa

SEQ ID NO: 32 - DNA, 948 bp, Synthetic Construct
  source 1..948: mol_type = other DNA
atgaaactaagaaaccttttatttatcgttcttgcagcgatagtcttctgcaactgtcagagctatcagcctacttcgctcaccgttgcctcctacaacctgagaaatgccaacggttccgactccgcccgtggagacggatggggacagcgttatccggtgattgcccagatggtgcaatatcacgatttcgatattttcggcacacaggaatgcttccttcaccaactgaaagacatgaaagaagcccttcccggttatgactatatcggcgtaggccgcgacgacggtaaagacaaaggcgaacactccgctatcttctaccgcaccgacaaattcgacatcgtagaaaaaggagatttctggctgtcggaaactccggacgtgccgagcaaaggctgggatgccgtattgcctcgtatttgcagctgggggcacttcaaatgcaaagataccggtttcgagtttctgttcttcaatctccacatggaccacatcggcaagaaagcccgtgtggagagcgctttcctcgtacaggaaaagatgaaagagctgggaagaggcaagaatctgccggctatcctgacgggagacttcaacgtcgaccagacccaccagtcctacgacgcatttgtcagcaaaggcgtcctctgtgattcttacgagaagtgcgactaccgatatgcgctcaacggaactttcaacaacttcgatccgaacagttttaccgaaagccgcatcgaccatatcttcgtttcaccttctttccacgtcaagagatacggtgtgctgacagatacctatcggagtgtacgggaaaacagtaaaaaggaggacgtgagagattgtccggaagagatcaccattaaggcttatgaagcacgtacaccatccgaccatttccctgtaaaagtggaactggtgtttgaccaacgtcagcaaaaataa

SEQ ID NO: 33 - DNA, 2283 bp, Synthetic Construct
  source 1..2283: mol_type = other DNA
atgaaaacacatttttcatttaaacacctgttatttattggaggtgcggtgttgtacagcatgcaaatttctgccgtcaagaatccggtagactatgtcagcacgctggtaggaacgcagtccaagtttgagttatcgaccggaaatacctatccggctacggcactgccgtggggaatgaacttctggacaccgcaaaccggtaaaatgggcgacggttgggcatatacctacaatgccgacaaaatccggggcttcaaacaaacacatcaacccagcccgtggatgaacgactacggtcagttttccatcatgccgatcacaggcggactggtattcgaccaggaccaacgtgccagctggttctcgcacaaggcggaggttgccaaaccttattattataaggtatatctcgcagaccacgacgttactacggaactcgttccgacggagcgtgccgctatgttccgtttcacgtatccggaaaccaagaacgcttatgtcgttatcgacgcattcgacaaaggctcttatgtaaaggtgattccggaagaaaacaagatcatcggttattctaccaagaacagcggcggagtgccggagaacttcaagaattattttgtcatccagttcgacaagccgtttacctttacttccggcgtgaaagagaacaacattctcccgaacgaaacagaagttcagggcaaccataccggagcgatcatcggattcgctacccagaaaggggagatcgttcacgcacgtgtagcttcttcttttatcagttatgagcaggcggaactgaatctcaaagaattgggcaaggatagtttcgaccagctggtcactaaaggaaaagacatctggaaccgtgaaatgagcaaagtagatgtggaagacgataatatcgacaatctgcgcactttctattcctgcctctatcgttcgatgctgttcccacgcagcttttatgaaatagacgccaaaggacaggtcgtacactacagcccttacaacggaaaagtgctaccgggctatatgtttacggataccggcttctgggatacgttccgctgtctgttcccattcttgaacctgatgtatccgtccatgaatcagaagatgcaggaaggactggtcaatgcgtaccttgaaagcggattccttccggaatgggcaagtcccggacaccgtgactgtatggtcggcaacaactccgcttccgtagtagccgacgcctatatcaaaggactgcgcggatatgacatcgaaacactttgggaagcattgaaacatgacgcaaacgcccatctccgcggcacagcttcgggccgccttgcatacgacgcctacaacaaactgggttatgtccccaacaatatcggtataggacagaatgttgcccgtacgctggaatatgcgtacaacgactggaccatctacacgctaggcaagaaactgggcaaaccggcaagcgaaatcgacatcttcaaacaacgtgcactcaactacaagaacgtctaccacccgaaacgcaaactgatggtaggcaaagacgacaaaggtgtgttcaaccccaaatttgatgcagtagactggagcggcgagttctgcgaaggtaacagctggcactggagtttctgcgtattccatgatccgcaaggactgatcgacctgatgggaggcaagaaagaattcaacaacatgatggattccgtctttgtcattccgggcaaacagggtatggaaagccgtggcatgatccacgaaatgcgtgaaatgcaggtaatgaacatgggacagtacgctcacggcaaccagcctatccagcacatggtttatctttacaactattcgggagaaccgtggaaggcccagcattgggttcgtgaaatcatggacaagctctacacggcaggccccgacggatattgcggtgacgaagacaacggtcagacttctgcctggtatgtcttctcggctttaggattctaccccgtttgtccgggaacagatcagtacattctgggaactccccttttcaagtcagccaagctgcatctggaaaatggaaaaaccgtcacaataaaagcaagcaacaataacaccgacaaccgttatgtgaaggatatgaaggtaaatggcaaggcattcacccgcaattatctgacgcacgaccaattactgaaaggagcgaatatccagtatcagatgagtcctacgccgaacaaacagcggggaacgactgaaaaagatattccctattccctttcatttgaataa

SEQ ID NO: 34 - DNA, 3102 bp, Synthetic Construct
  source 1..3102: mol_type = other DNA
atgaaaaatacacagaaatatttcatattacttttgatgttgatattacttgttccaagcaatatatggggacaagaaacaaaaaaggaaattatagtcaaaggtgtagtggaagatgatttagggccgataattggtgcgtcagtcgttgctaaaaaccaggcaggtgtgggagtaatcacaaatactgaaggtaagttttctttgaaagtgggaccttatgatgtattggtagtgacttttgttggttatcagccatatgagctgcctgttctgaaaatgaatgatcccaataatgtaactataaagttattggaagatgttggcaaaattgatgaagtggtaattacagccagtggacttcaacaaaagaaaactctgactggggcaataaccaatgttgatgtaaaacagttgaatgctgtaggaagtagtagtctttctaattcattggctggtgtggttcccggtattatagccatgcagcgtagtggtgaaccgggtgaaaatacatctgaattctggattcgaggtattagtacctttggtgcaaaatcaggagccttagttcttatcgacggagtagaacgaaattttgatgagattttgccgcaagacattgaatcgttctcagtactgaaagatgcatcagcaactgcaatatatggtcagcgcggtgcaaatggagttatcttaattaccaccaagcgtggggaaaaaggtaaggtgaaaattaatgtaaaagcaggatttgactggaatactcctgtaaaagtgccagagtatgcaagtggttatgattgggcgcgtttagccaatgaggctcggttaggacgctatgattccccgatttatactcctgaagaattggagataattagatcaggtttagatactgatttatatcctaatattgattggagggatttaatgttgaagagtggtgcacctcgctattatgctaatattagtttttcaggtggtagtgataatgtacgttattatgtctctggacaatataccagtgaacaaggacgttacaaaacgtttagctctgaaaataagtacaataccaatacgacttatgaacgatataattatcgtgctaacgtagacatgaacataactaagacaacagtactgaaagttagtgtaggtggatggttggtgaataggactacgcctactagaagtactggtgacatatgggaggattttgccaaatttactcctttgtctactcctcgtaaatggtctacaggacaatggccgagagtggatgggcaagatactcctgaatatcatatgacacaaagaggatatcatacgaaatgggagagtaaggtggaaactagtgtaaagttagagcaggatcttaagtttattacgcccggtttgaagtttgaaggagtatttgcttttgatacttataatgagaatataataaaacgggagaaaaaagaggaagtatgggaagcccaaaaatatagagatgaaaatggtaaattgattttgaaaagagtggtcaatagaagtccgatgaatcaaaataaggaagttaggggtgataaacgatactattttcaggcgtcattagattataaccgtttatttgctcatgcacatcgtgtcggtgtttttggcatggtataccaagaggaaaagacggatgttaatttcgactccagtgatttgattggttctattcctcgtcgtaatttggcttattccggtcgttttacttatgcttataaagataaataccttgctgaatttaactggggatgtactggttcagagaattttgaacatggaaaacaatttggtttctttcctgctgtttctgccggttatgtaatttctgaagaggcttttatgaaaaaagcattgccatggatagatcaatttaagatcagagcttcttatggtgaggtaggtaatgatgtattggatggtcgtcgattcccttatgtgtctcttatagatactgatgatggaggatcatattcatttggggaatttggaacaaatagagtgcaagcctaccgtattagaactttggggactcctaatttgacttgggagatagctaagaaatatgatgtaggtgttgacttttctttttttaatgggaaaattagtggtgctttagattggtttttggataaacgtgatgacatctttatgcagcgtaaacatatgccattgactaccgggcttgctgatcagactccaatggccaatgtcggaaagatgaagtcttatggatgggaaggaaatataggatttactcaatctattggtcaggtgaatctccaacttcgtgccaactttacttatcagactactgatatcatagataaggatgaagcagccaatgagttatggtataaaatggataaaggctttcagttaaatcaatcgcgtggattgattgctttaggattatttaaagatcaagatgaaatagaccgtagtccgaaacagacaagtaacagacctatccttcccggtgatattaaatacaaagatgtaaatggtgatggagttattaatgatgatgatattgtgcctttaggatatcgggaagttccgggattacagtatggtgtcggtttaagtgctaattggagaaattggaatttgagtgtacttttccaaggaacaggtaaatgtgatttctttattggcggtaatgggcctcatgctttccgtagtgaacgttatggtaatattttacaggcaatggtcgatggtaatcgttggatacccaaagaaatatcaggcacgactgctactgaaaatccaaatgcggattggccgcttttgacatatggcaataatgataataataatagaaaatcaacattttggttgtatgaaagaaaatatttgcgattacggaatgttgaagtcagctatgattttccacaaacttggacgcgtaaattttttgtaagtaacttacgtctaggctttgttggacaaaatttgttgacatgggctccttttaaaatgtgggatccggaagggactagagaggacggatctaactatccgataaataaaacattctcatgttatcttcaaataagcttttaa

SEQ ID NO: 35 - DNA, 1638 bp, Synthetic Construct
  source 1..1638: mol_type = other DNA
atgaaaattgtgaagtatatagttatcgtatcattatttagtatttctgcatgtagtgatgatgatgataaaaaaaacaatgagcgacctgggaatcttgtagagttacaggttgatgtaaatgagattaatattgcgcaaggagatacccgtactgtaaacattacgtcaggtaatggggaatatgttgcgacttcggctaatgaagaagtagttgtcgcagaaatagatggaaatgtggtgaaactaaccgctgttgaggggcataataatgctcaaggagttgtttatgttagcgataagtatttccaacgcactaaaattctagttaatacggcggcagaatttgaattgaagttgaataaaactttgtttacgctttattctcaagtggaaggatctgatgaagctctcatcaagatctatacaggaaatggaggttattctcttgaagtgattgatgataaaaattgtattgaagttgatcaatctacgcttgaagacacagaatcatttatggtgaaaggcattgctcaaggtaatgctgagattaagattactgaccaaaaaggaaaagaagcttttgtgaatctgaatgtaattgctcctaagcaaattacgactgatgctgacgaaaagggcgttctgataaattctaatcaaggatcacaacaagtgaagattcttacaggtaatggagaatataaggttcttgatgctggtgatgcaaagatcattcgtttggaagtttatggtaatgtggtaacggtgaccggaagaaaggccggagagacttcatttactttgactgatgcaaaaggacaagtttcacagactattcatgtaaagatcgctcctgagaagcgttggtatatgaatttaggaaaagagtatgcagtttggactcactttgcagagatgactggtgagggactagaggctgtgaaagttgaaactaacggctttaaacttaaaaaaatgacttgggagctagttgctcgtatcgatggaactaattggctacagacctttatgggtaaggaaggctattttattcttcgcggtggtgattgggaaaataataagggtagacagatggagttggtaggtatagatgataaactaaaactgagaactggacatggagcctttgaactcggaaaatggtctcatattgctttagttgtagattgttcgaaaggtaaggatgattacaatgaaaaatacaagctttatgttaatggtaaacaagtaaagtgggacgatagccgcaaaaccgatatggactattctgagattgatctttgtgcaggtaatgacgggggtagagtatcaatcggaagagctagtgacaacagatgctttcttgatggtgctatactcgaagcacgtatctggacggtttgtcgtacagaggaacaacttaaggctaatgcatgggagcttcatgaacaaaatcccgaagggttattagggcgctgggatttctcggctggagctccgacatcttatattgaggatggtaccaattcggatcatgagttgctgatgcatatttcgaagtatgatagctggaatgccacagaatttcctatgagcagatttggggaagctcccattgaagtaccttttaaataa

SEQ ID NO: 36 - DNA, 1578 bp, Synthetic Construct
  source 1..1578: mol_type = other DNA
atgaaagcaatattcaagctgttgatattgaactttttgactctgtttatctttccgtcttgcagtgatgatgataagtcaaagtctgaattgaatgaccccatcagtggcaatatttctccggtaggttcatttgcggtagaagctaccaataacgagaatgaacttctggtgaaatggaccaatcccagtaatcgcgacgtggatatggtagaactctcttacagggacgtggaagcgagtttgtctcgtgctaccgacttctcgccgggacatatcataatacaagtagagcgtgatgtcacacaggaatatatgttgaaggttccttattttgctacttacgaagtttctgccgtagctatcagcaaagccggcaagcgatcggtacccgaaagccgtgtggtgatgccttatcatgaaaaggtggacgagccggaactgaaactgccggaaatgctggaccgtgcacattcttacatgacttctgtcattggatattatttcggcaagagttccagaagctgctggcgtagtaattatccttatgatggaaaaggttattgggatggtgatgcgttggtctggggacaaggcggtgggctttcggcatttgttgctatgcgtgatgcaaccaaagagagcgaagtggagaatctttacggtgcaatggatgatatgatgttcaaaggaatacagtatttctgtcagctggatcgtggaatcctggcttattcctgctacccggctgccggtaacgaacgtttttacgatgataacgtatggatcgggctcgatatggtcgactggtatacggaaacgaaagagatgcgttatctgacacaggcaaaggtggtatggcgctacctgatcgatcacggttgggatgagacttgcggaggaggtgtacactggagggagttgaacgaacacactaccagcaagcactcttgctctaccggacctactgctgtgatgggctgtaagatgtatctggcaactcaggaacaggaatatctcgactgggcgatcaaatgttacgactatatgctggatgtattgcaagacaagtccgatcatttattctatgacaatgtacgcccgaataaggatgatcccaatctgccgggtgatcttgaaaagaacaagtattcctacaactccggacaaccattgcaggcggcctgtctcttatataagattaccggcgaacagaaatatctggatgaagcgtatgcgattgctgaaagctgtcataagaaatggtttatgccctatcgttccaaagagctgaatcttactttcaatatccttgctccgggacacgcttggttcaatacgatcatgtgccgtggattctttgaactttattctatagacaatgaccgtaaatatatcgatgatatcgaaaagtcaatgattcatgcgtggagcagtagctgtcatcagggtaataacttgctgaatgacgatgatctgagagggggaactaccaagaccggttgggaaatactccatcagggagcattggttgaattgtatgcccggttggcagtattggaacgtgaaaaccgatag

SEQ ID NO: 37 - DNA, 1578 bp, Synthetic Construct
  source 1..1578: mol_type = other DNA
atgaaagccatttttaaacttctaattttaaatttcttaactcttttcattttcccatcctgttctgatgatgataaatccaaatctgaattgaacgatcctatttctggcaatatttctcccgtaggaagttttgctgtcgaggctacaaacaatgaaaatgagcttcttgtcaagtggaccaatcccagtaaccgtgatgtggacatggtagagcttagttacagagacgtcgaagcatctctttcccgtgcaactgacttcagtcccggacacatcatcatacaagttgaaagggatgtaacacaagaatatatgcttaaggttccctattttgctacctatgaggtctccgcagttgcaataagtaaggccggaaagaggtccgttcccgaaagtagggtagtcatgccttatcacgagaaggtggatgaacctgagttgaagctgcccgagatgctggacagagcacattcctacatgacatctgtaataggatactactttggtaaaagtagtcgttcctgttggcgttctaactatccatatgacggtaagggctactgggacggagatgctttagtgtggggtcagggaggaggattaagtgcatttgtagcaatgcgtgatgctaccaaggaatcagaggtagagaatctatatggtgctatggacgatatgatgttcaagggtatccaatacttctgtcaactagatagaggtatactggcatattcttgttatcctgccgctggaaatgagaggttttacgatgataatgtttggattggtctagatatggtggactggtatacggaaaccaaagagatgagataccttacgcaagcaaaggttgtatggcgttatttaattgatcacggatgggacgagacatgcggtggcggcgtacattggagagaactgaatgaacatactacttcaaaacactcatgcagtactggccccactgctgtaatgggttgcaagatgtatcttgctacgcaggaacaagaatacttggactgggcaattaagtgttacgattatatgttggacgtactacaagataaatcagaccacttgttttatgacaacgtcaggccaaataaagatgatcctaatttaccaggcgacctagagaagaataagtacagttataattccggccaacctctgcaggccgcttgtttactatataaaattacgggtgagcaaaagtacttggatgaagcttatgcaatcgccgaaagttgtcacaagaaatggtttatgccatatagaagtaaagagctaaatctaactttcaacatccttgcccccggacatgcttggtttaatactatcatgtgccgtggctttttcgaactatattcaatagataatgatcgtaaatacattgatgacatagaaaaatcaatgatacacgcctggagttcctcctgccaccagggaaacaatctgttaaatgacgacgacctgaggggtggtacgaccaagacgggctgggaaattcttcaccaaggagcactggtcgagttatacgcaagactggcagttcttgagagggagaaccgatag

SEQ ID NO: 38 - DNA, 2268 bp, Synthetic Construct
  source 1..2268: mol_type = other DNA
atgatgatgaacagattgaatataaaaagaacagtcggctcctgtttgatggcgatggcgtttttttcgtgtacccatacggatcagacgcccacgaaagactttgtcgattatgtaaatccatatatcggcaatatcagccatctgctggtgcctacttacccaaccgtacatctgccgaactcgatgctcagggtctatccggaaaggggagactatacatcggacagggtaaacggccttccggtcgtggtgaccagtcatagaggcagctcggcttttaacctgagtccggtgcagggagaggtatcccgaccgattgtatcttactcctatgatttggagaatattaccccctatagttattccgtatacctggatgaggctgatatacaggttgagtatgccccttcacatcaggctggtatttatcatatcagttttgggacggaaggtgataatgctctggtggtgaatacgaagaacggaaagctggtcgctgaagaaaaaggagtcagtggctatcaggttattgacaacactcctaccaaaatctatctgtatctcgaaaccagtcaactacctttacgcaaaggggtactggcagatggaaaagttgatatggaaagtaaggaaggcagtgccatcgctttgtattatggaagcgagaagaacctgaatctacgttacggaatttcctttatcagtgccgagcaggcaaagaagaatctgcaacgtgacatcaccacctatgatgtaaaggcggtggcggatgccggacgcaggatatggaacaagacattgggcaagattgtgatagaaggcggttcggaagacgaaaaagaaatcttctacacttccctttatcgtacctacgaacgcatgatcaatctttcggaggacgggaaatattacagtgctttcgatggcaagattcatgaagatggcggagtacctttttatacagatgactggatatgggatacttaccgggctacacatccgttgcgtatcttgatagaaccgcagaaggaactcgatatgattcgttcatatatacggatggcagaacagtcggacagaagatggatgcctaccttccccgaggtgaccggagacagtcaccggatgaatggcaatcatgcagtggcagttatctgggatgcttattgcaaaggattgaaagactttgatctggaggctgcttatgaagcctgcaaaggagcgattacagaaaaaacgttgttgccctggctgagatgtccgttgacggagctcgataagttctatcaggaaaaaggatttttccctgcactgaaccctggcgaagaagaaacttgcaaggctgttcattcgttcgagagacgacaagcggttgcggttatgttgggtaactgttacgataattggtgtctggcacagatagccagaacattaaacaagaccgatgactataagaagtttatgaggatgtcttatacgtaccggaatgtttataatgcggaaacgggtttctttcatcccaagaacaaggacggaaagtttatcgaaccgtttgactatcgatattcgggaggacagggggcacgtggctattatggtgaaaacaacggttggatctatcgttgggatgtgcagcacaatccggcggatttgattgccttgatgggtggacaggcttcatttatcgagagattgaatcagacattcaatgaaccgttggggcggagcaagtttgatttctatcatcagttgccggaccataccggtaatgtcggccagttctctatggcaaatgaaccttgtctgcatattccttatttgtataactatgccggtcagccgtggatgacacaaaaaaggattcgcgttttgctgaaccagtggttccgtaatgacttgatgggcgttcccggtgatgaagacggaggtggaatgactgcatttgtggtattctccatgatgggcttttatccggtaactcccggttctccaacttataatatcggcagtccggtattccaatccgcaaagatggaggtaggtgacggacattattttgagatcatagcggagaattatgcgccggaccataagtacatccagtcggctaccttgaatggaacgccgtggaataagccgtggttcagccatgcggatattcaaaacggcggacgtctggttttgcagatgggagataagcccaataagaagtgggggatagcttcggatgccgtgccgccctcttcagagagtttgccggaataa

SEQ ID NO: 39 - DNA, 1149 bp, Synthetic Construct
  source 1..1149: mol_type = other DNA
atgaggaaagaacttgtttttgttttattggcattatttctgtgtgccggctgtaacggtaacaaaaagaaaatgaacggtgaacacgatttggatgcggcaaacattacgttggatgaccatacgatcagtttttattataattggtatggaaatccgtcagtggatggagaaatgaagcactggatgcacccgatagcccttgctccgggacattcgggagatgtcggtgccatatccggacttaatgatgacatcgcctgtaatttttatccggagctcggaacgtacagcagcaatgatcctgaaatcattcggaaacatatccggatgcatataaaagcgaatgtcggtgtactgtctgtcacttggtggggagaaagcgattatggcaaccaaagtgtgtctctcctgctggatgaggctgcaaaagtaggggcaaaggtgtgctttcatatagagccttttaatggacgcagcccgcaaacggtaagggagaatattcaatatatagtggatacttatggtgatcatccggctttttaccgtacgcacggcaaacctcttttctttatctatgattcttatctgatcaaacctgccgagtgggcgaagttgtttgctgccgggggagagataagtgtgcgtaataccaagtacgacggtctttttattggtctgacattgaaggaaagcgagttgcccgacattgagacagcgtgcatggatggcttttacacttactttgccgcaacaggtttcacaaatgcttctactccggccaactggaaatccatgcagcaatgggcaaaggcacataataaattgtttattccgagtgtcggtccgggatatattgatacccggattcgtccttggaacggaagtaccacccgagaccgtgagaatggaaaatattacgatgatatgtataaagctgccatagaaagcggtgcttcttatatttcgattacgtctttcaatgaatggcatgaaggaactcagatagagccggctgtctcaaagaagtgcgatgcttttgaatatttggattataaaccattggctgatgattactatttgataagaactgcctattgggtagatgaattccggaaagcaagatctgcttcggaagatgttcaataa

SEQ ID NO: 40 - DNA, 1149 bp, Synthetic Construct
  source 1..1149: mol_type = other DNA
atgaggaaagaacttgtttttgttttattggcattatttctgtgtgccggctgcaatggaaataaaaaaaaaatgaatggcgagcacgacttggacgctgccaatattacgcttgatgaccatacaatctctttttattacaattggtacggtaacccatcagttgacggcgagatgaagcactggatgcaccccatagcactggcccccggtcactccggagatgttggtgcaatatctggtttgaatgatgatattgcatgcaacttctaccctgaactaggaacatactcctctaacgatcctgaaattattcgtaaacacattagaatgcatataaaggctaatgtaggcgtgctatctgttacctggtggggcgagtccgactatggaaatcagtccgttagtctactattagatgaagctgccaaggtaggtgccaaagtatgcttccacatagaaccattcaacggacgttccccccaaacggtgcgtgagaacatccaatacatagtagacacctatggtgaccaccccgccttttatcgtactcacggcaaacctttatttttcatttacgactcttatttgatcaaacccgcagaatgggccaaattgtttgccgccggcggtgaaatatctgttcgtaatacgaagtatgatggcttgtttatcggccttacattaaaagaatctgagctacccgatatagaaactgcctgcatggacggattctacacctacttcgcagctactggatttacgaatgcttcaacgccagccaattggaaaagtatgcaacagtgggctaaagcacacaacaaacttttcatcccttctgttggcccaggatacatagacacaaggataaggccatggaacggttctacaactcgtgacagagagaacggaaagtactacgatgatatgtacaaagctgccatagagtccggagcctcttatatatctatcacctcctttaatgaatggcatgagggcacacaaatagagcctgccgtatccaagaagtgcgacgctttcgagtaccttgactacaaacctttggccgatgactactatctaataaggaccgcttactgggtggatgaatttaggaaagccaggtctgcctccgaggatgtgcagtaa

SEQ ID NO: 41 - protein, 517 aa, Synthetic Construct
MKKVIKKYFFLALAIIMYSCNEDEKYDILERYTPETITSDEIAPVLNLQAQYMDSNSEIVLVTWMNPEDDFLSKVEISCCSANDNLLGEPVLLDAVSTKVGSYQTSLSVEERGYVKIVAINEKGVRSEARTAEILSSQQDFVYRADCLMSSVIELFFGGRYNAWNENYPNATGPYWDGIAAVWGQGAAYSGFVTMYKVTKETNNEKLRAKYAEKEETFLNSIDIFLNNGSGRKSFAYGTYIGPNDERYYDDNVWIGIEMANLYELTGNEVYLQHANTVWNFILEGIDDVTGGGVYWKEGAVSKHTCSTAPAAVMALKLYQLSKNESYLEIAKSLYSYCKDVLQDPNDYLFYDNVRLSDPSDKNSELKVSKDKFTYNSGQPMLAAAMLYRITKEEQFLKDAQNIAQSIYKKWFKNYHSSILDRDIMILSDPNTWFNAVMFRGFVELYKIDKNDVYVKAVKNTMEHAWQSNCRNRLTNLMSDDYAGDKKEGKWNIKTQGAFVEIFSLIGELEQLGCFQE

SEQ ID NO: 42 - protein, 760 aa, Synthetic Construct
MKTHFSFKHLLFLGGAVLYSLQSSAVKNPVDYVSTLIGTQSKFELSTGNTYPATALPWGMNFWTPQTGKMGDGWAYTYDADKIRGFKQTHQPSPWMNDYGQFAIMPITGGLVFDQDRRASWFSHKAEVAKPYYYKVYLADHDVTTELAPTERAVMFRFTYPETKNAYVIVDAFDKGSYVKVIPEENKIIGYSTKNSGGVPENFKNYFVIQFDKPFTFVSTVFENNILPNETEAKGNHTGAVIGFATKKGEIVHARVASSFISPEQAELNLKELGKNSFDQLVANGREIWNREMSKIEIEDDNIDNLRTFYSCLYRSMLFPRSFYEIDAKGQVMHYSPYNGEVRPGYMFTDTGFWDTFRCLFPFLNLMYPSMNQKMQEGLVNTYKESGFLPEWASPGHRDCMVGNNSASVVADAYIKGLRGYDIETLWEALKHGANAHLRGTASGRLGYESYNQLGYVANNIGIGQNVARTLEYAYNDWAIYTLGKKLGKPENEIDIYKKHALNYKNVYHPERKLMVGKDNKGVFNPNFDAVDWSGEFCEGNSWHWSFCVFHDPQGLINLMGGKKEFNAMMDSVFVISGKLGMESRGMIHEMREMQVMNMGQYAHGNQPIQHMVYLYNYSSEPWKAQYWIREIMNKLYTAGPDGYCGDEDNGQTSAWYVFSALGFYPVCPGTDEYIIGTPLFKSAKLHLENGKTITIKADNNQLDNRYIKEMKVNGKSQTRNFLTHDQLIKGANIQFQMSPVPNKQRGTTEKDVPYSLSFE

SEQ ID NO: 43 - protein, 316 aa, Synthetic Construct
MKLKNLLLIALVAIVFCGCQSNYQPTSITVASYNLRNANGGDSINGNGWGQRYPVIAQIVQYHDFDIFGTQECFIHQLKDMKEALPGYDYIGVGRDDGKEKGEHSAIFYRTDKFDVIEKGDFWLSETPDVPSKGWDAVLPRICSWGHFKCKDTGFEFLFFNLHMDHIGKKARVESAFLVQDKMKELGKGKELPAILTGDFNVDQTHQSYDAFVSKGVLCDSYEKAGFRYAINGTFNDFDPNSFTESRIDHIFVSPSFQVKRYGVLTDTYRSIVGKGEKKQANDCPEEIDIKTYQARTPSDHFPVKVELEFDQRQQK

SEQ ID NO: 44 - protein, 385 aa, Synthetic Construct
MRNICFVACMLFCLTSAVGKTPGNTRYLSIADSILSNVLNLYQTNDGLLTETYPVNPDQKITYLAGGTQQNGTLKASFLWPYSGMMSGCVALYKATGNKKYKKILEKRILPGMEQYWDNSRLPACYQSYPTKYGQHGRYYDDNIWIALDYCDYYQLTHKPASLEKAVALYQYIYSGWSDEIGGGIFWCEQQKEAKHTCSNAPSTVLGVKLYRLTKDAKYLEKAKETYAWTKKHLCDPTDHLYWDNINLKGKVSKEKYAYNSGQMIQAGVLLYEETGDEQYLRDAQQTAAGTDAFFRTKADKKDPTVKVHKDMAWFNVILFRGLKALYKIDKNPAYVNAMVENALHAWENYRDENGLLGRDWSGHNKEQYKWLLDNACLIEFFAEI

SEQ ID NO: 45 - protein, 481 aa, Synthetic Construct
MNITKAFCLSIALLGASNMQAITNSDFVIQQDNTKINNYQTNRPETSKRLFVSQAVEQQIAHIKQLLTNARLAWMFENCFPNTLDTTVHFDGKDDTFVYTGDIHAMWLRDSGAQVWPYVQLANKDAELKKMLAGVIKRQFKCINIDPYANAFNMNSEGGEWMSDLTDMKPELHERKWEIDSLCYPIRLAYHYWKTTGDASIFSDEWLTAIAKVLKTFKEQQRKEDPKGPYRFQRKTERALDTMTNDGWGNPVKPVGLIASAFRPSDDATTFQFLVPSNFFAVTSLRKAAEILNTVNKKPDLAKECTTLSNEVETALKKYAVYNHPKYGKIYAFEVDGFGNQLLMDDANVPSLIALPYLGDVKVNDPIYQNTRKFVWSEDNPYFFKGTAGEGIGGPHIGYDMIWPMSIMMKAFTSQNDAEIKTCIKMLMDTDAGTGFMHESFHKNDPKNFTRSWFAWQNTLFGELILKLVNEGKVDLLNSIQ

SEQ ID NO: 46 - protein, 1198 aa, Synthetic Construct
MNKKVIAVALALALAGGSYAQDDTAKKKVKAYMVSDAHLDTQWNWDIQTTINEYVWNTISQNLFLLKKYPEYVFNFEGGVKYAWMKEYYPEQYEEMKKFIEEGRWHIAGSSWEASDVLVPSVEASIRNIMLGQTYYRQEFGKEGTDIFLPDCFGFGWTLPTIAAHCGLIGFSSQKLDWRNHPFYGKSKHPFTIGLWKGIDGKQVMLAHGYDYGRKWNNEDLSKNKDLEKLAQRTPLNTVYRYYGTGDIGGSPTLGSVRSVEQGIKGDGPVEVISATSDQLFKDYLPFNNHPELPVFDGELLMDVHGTGCYTSQAAMKLYNRQNEQLGDAAERAAVAAEWLGTASYPQHTLTEAWKRFIFHQFHDDLTGTSIPRAYEFSWNDELISLKQFSQVLTSSVNAIAGQMDTRVKGTPVVLYNANAFPVSDLTEIILEQPKTPKGFTVYNAQGKKVASQMIGYENGRAHILVAASLPANSYAVYDVRTGGSEKTISPSAASAIENSVYKITLDKNGDIISLTDKRNNKELVKDGKAIRLALFTENKSYAWPAWEILKETIDREPVSITDGAKITLVENGALRKALCIEKKYGKSLFKQYIRLYEGSRADRIDFYNEIDWQSTNTLLKAEFPLNIENEKATYDLGIGSVERGNNVQTAYEVYAQQWADLTDKNNSYGVSILNDSKYGWDKPDNNTIRLTLLHTPETKGNYAYQDHQDFGFHTFTYSLTGHNGALDKPATAIKAEILNQPIKAFSSPKHAGTLGKEFAFVRSSNDQVVIKALKKAEVSDEYVVRVYETGGAAPQQAAITFAGEIEKAVLADGTEKEIGSADFNKNQLNVSIAPYSIQTFKVKLKKKADLQAPACAYLPLDYDRRCFSWNAFRKEGNFESGNSYAAELLPDSILKADGIPFRLGEKEIANGLTCKGNVLQLPTGHSYNRIYFLAASAGEDAVATFSTGNNSQEITVPSYTGFIGQWEHLGHTEGFLKDAEIAYVGTHRHASDKDEAYEFTYMFKFGMDIPKGATTVTLPDHADIVLFAATLVNEKYPAVTPASELFRTALKADNGEEATTKTNLLKQAKLIKCSGETNEKEVARYAVDGDVKTKWCDTSTAPNYIDFDFGKEQTIRGWKLVNAGNEGSVFITHTCFLQGRNSPDEEWKTIDELSDNKKNTVVRQFKPTSVRYVRLLVTQSTQNNSLKAARIYELEVY

SEQ ID NO: 47 - protein, 383 aa, Synthetic Construct
MKSTFLFLVTTTMMTCTALGQPSNDKKNVLPDWAFGGFERPQGANPVISPIENTKFYCPMTQDYVAWESNDTFNPAATLHDGKIVVLYRAEDKSGVGIGHRTSRLGYATSSDGIHFKREKTPVFYPDNDTQKKLEWPGGCEDPRIAVTAEGLYVMTYTQWNRHIPRLAIATSRNLKDWTKHGPAFAKAYDGKFFNLGCKSGSILTEVVNGKQVIKKIDGKYFMYWGEEHVFAATSEDLVNWTPYVNTDGSLRKLFSPRDGHFDSQLTECGPPAIYTPKGIVLLYNGKNSASRGDKRYTANVYAAGQALFDANDPTRFITRLDEPFFRPMDSFEKSGQYVDGTVFIEGMVYYKDKWYLYYGCADSKVGMAIYNPKKPAAADPLP

SEQ ID NO: 48 - protein, 481 aa, Synthetic Construct
MNITKTLCLCAALSGAAGVQAMENREFVTQQDNTRVNNYQTNRPEASKRLFVSQEVERQIDHIKQLLTNAKLAWMFENCFPNTLDTTVHFDGKEDTFVYTGDIHAMWLRDSGAQVWPYVQLANKDPELKKMLAGVINRQFKCINIDPYANAFNMNSEGGEWMSDLTDMKPELHERKWEIDSLCYPIRLAYHYWKTTGDASVFSDEWLQAIANVLKTFKEQQRKDDAKGPYRFQRKTERALDTMTNDGWGNPVKPVGLIASAFRPSDDATTFQFLVPSNFFAVTSLRKAAEILNTVNRKPALAKECTALADEVEKALKKYAVCNHPKYGKIYAFEVDGFGNQLLMDDANVPSLIALPYLGDVKVTDPIYQNTRKFVWSEDNPYFFKGSAGEGIGGPHIGYDMIWPMSIMMKAFTSQNDAEIKTCIKMLMDTDAGTGFMHESFNKNDPKNFTRAWFAWQNTLFGELILKLVNEGKVDLLNSIQ

SEQ ID NO: 49 - protein, 387 aa, Synthetic Construct
MRNICFVACMLFCLASASGKTVKNHPFVSIADSILDNVLNLYQTEDGLLTETYPVNPDQKITYLAGGAQQNGTLKASFLWPYSGMMSGCVAMYQATGDKKYKTILEKRILPGLEQYWDGERLPACYQSYPVKYGQHGRYYDDNIWIALDYCDYYRLTKKADYLKKAIALYEYIYSGWSDELGGGIFWCEQQKEAKHTCSNAPSTVLGVKLYRLTKDKKYLNKAKETYAWTRKHLCDPDDFLYWDNINLKGKVSKDKYAYNSGQMIQAGVLLYEETGDKDYLRDAQKTAAGTDAFFRSKADKKDPSVKVHKDMSWFNVILFRGFKALEKIDHNPTYVRAMAENALHAWRNYRDANGLLGRDWSGHNEEPYKWLLDNACLIELFAEIEK

SEQ ID NO: 50 - protein, 315 aa, Synthetic Construct
MKLRNLLFIVLAAIVFCNCQSYQPTSLTVASYNLRNANGSDSARGDGWGQRYPVIAQMVQYHDFDIFGTQECFLHQLKDMKEALPGYDYIGVGRDDGKDKGEHSAIFYRTDKFDIVEKGDFWLSETPDVPSKGWDAVLPRICSWGHFKCKDTGFEFLFFNLHMDHIGKKARVESAFLVQEKMKELGRGKNLPAILTGDFNVDQTHQSYDAFVSKGVLCDSYEKCDYRYALNGTFNNFDPNSFTESRIDHIFVSPSFHVKRYGVLTDTYRSVRENSKKEDVRDCPEEITIKAYEARTPSDHFPVKVELVFDQRQQK

SEQ ID NO: 51 - protein, 760 aa, Synthetic Construct
MKTHFSFKHLLFIGGAVLYSMQISAVKNPVDYVSTLVGTQSKFELSTGNTYPATALPWGMNFWTPQTGKMGDGWAYTYNADKIRGFKQTHQPSPWMNDYGQFSIMPITGGLVFDQDQRASWFSHKAEVAKPYYYKVYLADHDVTTELVPTERAAMFRFTYPETKNAYVVIDAFDKGSYVKVIPEENKIIGYSTKNSGGVPENFKNYFVIQFDKPFTFTSGVKENNILPNETEVQGNHTGAIIGFATQKGEIVHARVASSFISYEQAELNLKELGKDSFDQLVTKGKDIWNREMSKVDVEDDNIDNLRTFYSCLYRSMLFPRSFYEIDAKGQVVHYSPYNGKVLPGYMFTDTGFWDTFRCLFPFLNLMYPSMNQKMQEGLVNAYLESGFLPEWASPGHRDCMVGNNSASVVADAYIKGLRGYDIETLWEALKHDANAHLRGTASGRLAYDAYNKLGYVPNNIGIGQNVARTLEYAYNDWTIYTLGKKLGKPASEIDIFKQRALNYKNVYHPKRKLMVGKDDKGVFNPKFDAVDWSGEFCEGNSWHWSFCVFHDPQGLIDLMGGKKEFNNMMDSVFVIPGKQGMESRGMIHEMREMQVMNMGQYAHGNQPIQHMVYLYNYSGEPWKAQHWVREIMDKLYTAGPDGYCGDEDNGQTSAWYVFSALGFYPVCPGTDQYILGTPLFKSAKLHLENGKTVTIKASNNNTDNRYVKDMKVNGKAFTRNYLTHDQLLKGANIQYQMSPTPNKQRGTTEKDIPYSLSFE

SEQ ID NO: 52 - protein, 1033 aa, Synthetic Construct
MKNTQKYFILLLMLILLVPSNIWGQETKKEIIVKGVVEDDLGPIIGASVVAKNQAGVGVITNTEGKFSLKVGPYDVLVVTFVGYQPYELPVLKMNDPNNVTIKLLEDVGKIDEVVITASGLQQKKTLTGAITNVDVKQLNAVGSSSLSNSLAGVVPGIIAMQRSGEPGENTSEFWIRGISTFGAKSGALVLIDGVERNFDEILPQDIESFSVLKDASATAIYGQRGANGVILITTKRGEKGKVKINVKAGFDWNTPVKVPEYASGYDWARLANEARLGRYDSPIYTPEELEIIRSGLDTDLYPNIDWRDLMLKSGAPRYYANISFSGGSDNVRYYVSGQYTSEQGRYKTFSSENKYNTNTTYERYNYRANVDMNITKTTVLKVSVGGWLVNRTTPTRSTGDIWEDFAKFTPLSTPRKWSTGQWPRVDGQDTPEYHMTQRGYHTKWESKVETSVKLEQDLKFITPGLKFEGVFAFDTYNENIIKREKKEEVWEAQKYRDENGKLILKRVVNRSPMNQNKEVRGDKRYYFQASLDYNRLFAHAHRVGVFGMVYQEEKTDVNFDSSDLIGSIPRRNLAYSGRFTYAYKDKYLAEFNWGCTGSENFEHGKQFGFFPAVSAGYVISEEAFMKKALPWIDQFKIRASYGEVGNDVLDGRRFPYVSLIDTDDGGSYSFGEFGTNRVQAYRIRTLGTPNLTWEIAKKYDVGVDFSFFNGKISGALDWFLDKRDDIFMQRKHMPLTTGLADQTPMANVGKMKSYGWEGNIGFTQSIGQVNLQLRANFTYQTTDIIDKDEAANELWYKMDKGFQLNQSRGLIALGLFKDQDEIDRSPKQTSNRPILPGDIKYKDVNGDGVINDDDIVPLGYREVPGLQYGVGLSANWRNWNLSVLFQGTGKCDFFIGGNGPHAFRSERYGNILQAMVDGNRWIPKEISGTTATENPNADWPLLTYGNNDNNNRKSTFWLYERKYLRLRNVEVSYDFPQTWTRKFFVSNLRLGFVGQNLLTWAPFKMWDPEGTREDGSNYPINKTFSCYLQISF

SEQ ID NO: 53 - protein, 545 aa, Synthetic Construct
MKIVKYIVIVSLFSISACSDDDDKKNNERPGNLVELQVDVNEINIAQGDTRTVNITSGNGEYVATSANEEVVVAEIDGNVVKLTAVEGHNNAQGVVYVSDKYFQRTKILVNTAAEFELKLNKTLFTLYSQVEGSDEALIKIYTGNGGYSLEVIDDKNCIEVDQSTLEDTESFMVKGIAQGNAEIKITDQKGKEAFVNLNVIAPKQITTDADEKGVLINSNQGSQQVKILTGNGEYKVLDAGDAKIIRLEVYGNVVTVTGRKAGETSFTLTDAKGQVSQTIHVKIAPEKRWYMNLGKEYAVWTHFAEMTGEGLEAVKVETNGFKLKKMTWELVARIDGTNWLQTFMGKEGYFILRGGDWENNKGRQMELVGIDDKLKLRTGHGAFELGKWSHIALVVDCSKGKDDYNEKYKLYVNGKQVKWDDSRKTDMDYSEIDLCAGNDGGRVSIGRASDNRCFLDGAILEARIWTVCRTEEQLKANAWELHEQNPEGLLGRWDFSAGAPTSYIEDGTNSDHELLMHISKYDSWNATEFPMSRFGEAPIEVPFK

SEQ ID NO: 54 - protein, 525 aa, Synthetic Construct
MKAIFKLLILNFLTLFIFPSCSDDDKSKSELNDPISGNISPVGSFAVEATNNENELLVKWTNPSNRDVDMVELSYRDVEASLSRATDFSPGHIIIQVERDVTQEYMLKVPYFATYEVSAVAISKAGKRSVPESRVVMPYHEKVDEPELKLPEMLDRAHSYMTSVIGYYFGKSSRSCWRSNYPYDGKGYWDGDALVWGQGGGLSAFVAMRDATKESEVENLYGAMDDMMFKGIQYFCQLDRGILAYSCYPAAGNERFYDDNVWIGLDMVDWYTETKEMRYLTQAKVVWRYLIDHGWDETCGGGVHWRELNEHTTSKHSCSTGPTAVMGCKMYLATQEQEYLDWAIKCYDYMLDVLQDKSDHLFYDNVRPNKDDPNLPGDLEKNKYSYNSGQPLQAACLLYKITGEQKYLDEAYAIAESCHKKWFMPYRSKELNLTFNILAPGHAWFNTIMCRGFFELYSIDNDRKYIDDIEKSMIHAWSSSCHQGNNLLNDDDLRGGTTKTGWEILHQGALVELYARLAVLERENR

SEQ ID NO: 55 - protein, 755 aa, Synthetic Construct
MMMNRLNIKRTVGSCLMAMAFFSCTHTDQTPTKDFVDYVNPYIGNISHLLVPTYPTVHLPNSMLRVYPERGDYTSDRVNGLPVVVTSHRGSSAFNLSPVQGEVSRPIVSYSYDLENITPYSYSVYLDEADIQVEYAPSHQAGIYHISFGTEGDNALVVNTKNGKLVAEEKGVSGYQVIDNTPTKIYLYLETSQLPLRKGVLADGKVDMESKEGSAIALYYGSEKNLNLRYGISFISAEQAKKNLQRDITTYDVKAVADAGRRIWNKTLGKIVIEGGSEDEKEIFYTSLYRTYERMINLSEDGKYYSAFDGKIHEDGGVPFYTDDWIWDTYRATHPLRILIEPQKELDMIRSYIRMAEQSDRRWMPTFPEVTGDSHRMNGNHAVAVIWDAYCKGLKDFDLEAAYEACKGAITEKTLLPWLRCPLTELDKFYQEKGFFPALNPGEEETCKAVHSFERRQAVAVMLGNCYDNWCLAQIARTLNKTDDYKKFMRMSYTYRNVYNAETGFFHPKNKDGKFIEPFDYRYSGGQGARGYYGENNGWIYRWDVQHNPADLIALMGGQASFIERLNQTFNEPLGRSKFDFYHQLPDHTGNVGQFSMANEPCLHIPYLYNYAGQPWMTQKRIRVLLNQWFRNDLMGVPGDEDGGGMTAFVVFSMMGFYPVTPGSPTYNIGSPVFQSAKMEVGDGHYFEIIAENYAPDHKYIQSATLNGTPWNKPWFSHADIQNGGRLVLQMGDKPNKKWGIASDAVPPSSESLPE

SEQ ID NO: 56 - protein, 382 aa, Synthetic Construct
MRKELVFVLLALFLCAGCNGNKKKMNGEHDLDAANITLDDHTISFYYNWYGNPSVDGEMKHWMHPIALAPGHSGDVGAISGLNDDIACNFYPELGTYSSNDPEIIRKHIRMHIKANVGVLSVTWWGESDYGNQSVSLLLDEAAKVGAKVCFHIEPFNGRSPQTVRENIQYIVDTYGDHPAFYRTHGKPLFFIYDSYLIKPAEWAKLFAAGGEISVRNTKYDGLFIGLTLKESELPDIETACMDGFYTYFAATGFTNASTPANWKSMQQWAKAHNKLFIPSVGPGYIDTRIRPWNGSTTRDRENGKYYDDMYKAAIESGASYISITSFNEWHEGTQIEPAVSKKCDAFEYLDYKPLADDYYLIRTAYWVDEFRKARSASEDVQ

SEQ ID NO: 57 - protein, 985 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLDKREAEAMKKVIKKYFFLALAIIMYSCNEDEKYDILERYTPETITSDEIAPVLNLQAQYMDSNSEIVLVTWMNPEDDFLSKVEISCCSANDNLLGEPVLLDAVSTKVGSYQTSLSVEERGYVKIVAINEKGVRSEARTAEILSSQQDFVYRADCLMSSVIELFFGGRYNAWNENYPNATGPYWDGIAAVWGQGAAYSGFVTMYKVTKETNNEKLRAKYAEKEETFLNSIDIFLNNGSGRKSFAYGTYIGPNDERYYDDNVWIGIEMANLYELTGNEVYLQHANTVWNFILEGIDDVTGGGVYWKEGAVSKHTCSTAPAAVMALKLYQLSKNESYLEIAKSLYSYCKDVLQDPNDYLFYDNVRLSDPSDKNSELKVSKDKFTYNSGQPMLAAAMLYRITKEEQFLKDAQNIAQSIYKKWFKNYHSSILDRDIMILSDPNTWFNAVMFRGFVELYKIDKNDVYVKAVKNTMEHAWQSNCRNRLTNLMSDDYAGDKKEGKWNIKTQGAFVEIFSLIGELEQLGCFQEGSSGSSGSSGSSGSSGSSGSSGSSEAAAREAAAREAAAREAAARGGGGSGGGGSGGGGSQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 58 - protein, 368 aa, Synthetic Construct
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 59 - protein, 390 aa, Synthetic Construct
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 60 - protein, 465 aa, Synthetic Construct
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 61 - protein, 487 aa, Synthetic Construct
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 62 - protein, 279 aa, Synthetic Construct
ASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 63 - protein, 298 aa, Synthetic Construct
MSRISILAVAAALVASATAASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 64 - protein, 320 aa, Synthetic Construct
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 65 - protein, 338 aa, Synthetic Construct
MKLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 66 - protein, 1142 aa, Synthetic Construct
ITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 67 - protein, 1161 aa, Synthetic Construct
MVNISIVAGIVALATSAAAITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 68 - protein, 631 aa, Synthetic Construct
ININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 69 - protein, 650 aa, Synthetic Construct
MFTFLKIILWLFSLALASAININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 70 - protein, 1587 aa, Synthetic Construct
QIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 71 - protein, 1609 aa, Synthetic Construct
MNSFASLGLIYSVVNLLTRVEAQIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 72 - protein, 409 aa, Synthetic Construct
MLTKVISLAILTASAFADSGEFTLWNLSPGDPYDSTFWGVSEGLIVPVEPGVTFVITDDLQLKTTDDQFVTVGEDSALGLGAEGSVEFSIINEDGITSLYYNGELVTAYICEGAEPQIYLTGSEEDPECVSYTVAVIGVDGEAPPTFPEEDDETTTTDDPTDEPTDEPTDEPTDEPTDEPTDEPTDEPTDEPTDEPTDEPTDEPTDEPTDEPTDEPTDEPTDEPTEEPTEEPTEEPTDEPTPPPPHWGNETVTATKTEYETTKVTITSCEETKCYETTSDAWVSTCTTEIGGKVTKIVTWCPIPSTPGPKPPKPTKPTETKPTTVPAPTTKKPETPTTKKPETPAPEKPEKTTTVIPPPTTEKPSTLSTSSVTGSVTIPTITATGGAGSNFNLGGLTVGVAGIAMALFV

SEQ ID NO: 73 - protein, 471 aa, Synthetic Construct
MFEKSKFVVSFLLLLQLFCVLGVHGQESGNGTTSDTAYACDIGATPFDGFNATIYQYQASDDNSIQDPVFMSTGYLQRNQLHSTTGVTNPGFNIFTAGVATTTLYGIPNVNYQNMLLELKGYFRADASGNYGLSLRNIDDSAILFFGRETAFECCNENLIPLDEAPTDYSLFTIKEGEASTNPDSYTYTQYLEAGRYYPVRTFFANIRTRAVFNFTMTLPDGSELTDFQNYIFQFGALNQQQCQAEIVTRENYTTTTEPWTGTFEATTTVIPSGTEPGTVIVQTPYSTIDSTSTWTGTFTTFTTDADGSTIAVVPSSTIDDHFASTETVLTDTAISTTVITVTSCGTSKCTKTTALTGVTQRTLTIDDRTTVVTTYCPLPTDVATIKTASVSGSEVVQTIYTAKHSQAVSYVHPSTVTITREVCDAQTCTQATIVTGEILQTTVVDSGSTTVVPKYVPVETHEPTFELSTL

SEQ ID NO: 74 - protein, 233 aa, Synthetic Construct
MQFTFASTSVVVSLIAALAKPAVATPPACLLACAAEVVKESSDCDALNNIQCICENEGSAIHACLESTCPDGLSSTALQSFEDVCESVGTEANLDESSSSQSSSSSSSSESSSSSVSSSSSSASSSSETSSSVTSSSVTSSSTAVSSSTESSSSVEPSTSHSSSHSSSEVSSTVAPTTSVAPTTSSITTSSTSLTSATTSSVTISIEPTSDAADKVIIPGLAGLVGALAVGLI

SEQ ID NO: 75 - protein, 203 aa, Synthetic Construct
MQYRSLFLGSALLAAANAAVYNTTVTDVVSELETTVLTITSCAEDKCITSKSTGLITTSTLTKHGVVTVVTTVCDLPSTTKSYVPPAKTTTIPPPEKTTTTVPPPAKTTTTVPPPAKTTSTVPPPAKTSSHHESTITVTVPSSTSTKKIETESTTYHFVTQTTTARNITPPAITTQSHGAAGMNAANFVGLGAAAVAAAALVL

SEQ ID NO: 76 - protein, 439 aa, Synthetic Construct
MSLLLFLVLGAFLLSSVKAADIGAFRLRVYTPGRFTNGALNFNNWGYQYLDASSSNGQLFAGYATVTSVTTFLAPDDEGFVWGSSLGGYPGFLGIGAGATAFHLTGIPGDALSWYIEDNILKTSSPTYVCSRNDGDVVVGIEANTRWLAMHDTSQLPPNYYCFQADYEIVALWYIPDTTSTWTGTETSTTTDDDGSVIELVPTPLPDTTSTWTGTFTTFTTDDDGSVIELVPTPLPDSTSTWTGTYTTFTTDEDGSTIAVVPSSTIDSTSTWTGTYTTFTTDEDGSTIAVVPSSTIDSTSTWTGTYTTFTTDEDGSTIAVYHHLLSTPHPPGLVLTPRSLPMRMEVLLLWYHHLLSTLHPPGLVLTPRSLPMRMEVLLLWYHRLLSTPHPGLVLTPRSLPMRMEVLLLYHHLLSTPHPPGLVLTPRSLPMRMEVLLLWY

SEQ ID NO: 77 - protein, 2140 aa, Synthetic Construct
MKLQLQSFVFFLLSAVNVLADDSYGCSIATSPRSTGFVANLYEFPNMAISNAELKTYVRYRYKEGRLYDTISNIISPYFYYQGQGANSAYGTLYGRPNVYLYNFSMELKGYFRPPITGQYTIDFNGANVDDAAMVFFGKAGAFDCCNSDYILPEQSAEYSLYSVYPHTATDQILSATIYLEAGKYYPLRVTYTNIGNIGSLDLRVVLPSGASITSLGAFVYQFPNNLSPGTCTPDVEYFTTTTQAWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVIIETPESYVTTTQPWTGTYETTYTVPPSGTEPGIVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGIVIIETPESYVTTTQPWTGTYETTYTVPPSGTEPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPSGTEPGTVIVETPDVPGSYVTTTQPWTGTYETTHTVPPTGTEPGTVVVETPDVPGSYVTTTQPWTGTYETTHTVPPTGTEPGTVVVETPDVPGSYVTTTQPWTGTYETTYTVPPSGTEPGTVIVETPDVPGSYVTTTQPWTGTYETTHTVPPTGTEPGTVVVETPDVPGSYVTTTQPWTGVYKTTYTVPPSGTIPGTVIIETPFGYFNTSSISTKTDKRTITSVVPCSQCSESKTQYITPTGPGDVTVIISQPPSKITLSSPEDKTKTDFITSTGSIGGGSPPSHPNDKPGIITTPTQPIGGGNPSDIPSAISSVSSGGNSRASVPSFSTSSAISVQVSSLYDENSGSTFEVSLLFSVVSGFFLTLMV

SEQ ID NO: 78 - protein, 1149 aa, Synthetic Construct
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 79 - protein, 715 aa, Synthetic Construct
MTKFTILLLVLLKFYSILAIEVDGSANGQPLAHPIVVEVHEATKWITHTSPWTGTPEAIRTVTGETPYEQKIARYDEFNPRLANREIIDCVAFCCGDATSSPSITEPESTATELPESYVTINRPWSLSWIPDVPPGSPYWSTSTIPPSGTEPGTVIIYFYLYDDARKRREINFGSTQPYHGRPKLLGSIEKRELCQCDAVCCLGDLSCEVYVTTTQPWTGTYETTYTITPTGSEPGTVIIETPELYVTTTQPWTGTYETTYTITPTGSEPGTVIIETPESYVTTTQPWTGTYETTYTITPTGSEPGTVIIETPESYVTTTQPWTGTYETTYTITPTGSEPGTVIIETPESYVTTTQPWTGTYETTYTVPPSGTEPGAVIIETPELYVTTTQPWTGTYETTYTITPTGSEPGTVIIETPESYVTTTQPWTGTYETTYTVPPSGTEPGTVIIETPELYVTTTQPWTGTYETTYTITPTGSEPGTVIVEIPVSYVNSTQISTSTYDTTDTVLSSGVEPGTIAIETPIVYLNTSVSAFSRPWTKIDTVTQFSSCAVCSKPETITVTPENPIDTVTIIISQPQSTSQSNTPTSFKANSTSAFSRFDEDSIPVFGSYSYEITVNIDVNTEDDTTTNLNADTTIIIGSLSAIRTVAGSSSNYHASNISPTINSQKTASSVVVHSDSSATVYQFSPSNGAPWLSVQISTLLSVVGTLLAAVLL

SEQ ID NO: 80 - protein, 2498 aa, Synthetic Construct
MNFRYLLILPIYASIVLGQVGDFQLLLNAKEPIRNSPSLLSSNYGNLTLPAMANGALESHFDYGNAYVGDDQITVVYHLPDEHGQINAYRQDTDEYIGYLGLVTDDYGEYTYLSVIMPGVQYDQTTSVNWYIENEELKSTSINVQPLLGCYYKNPPQYSWYWASIDEPGNIASSNFVCEPCKVYVDFVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADTTSVWTGSYTTWTTDEDGTVIEQVPTPSADTPSADTTSVWTGSYTTWTTDEDGTVIEQVPTPSADTTSVWTGSYTTWTTDEDGTVIEQVPTPSADTPSADTTSVWTGSYTTWTTEVGDGGSSTVVELVPTESSTSTNVMQTPVPSSGVSDGVSVFNGFNVEVFHYPADNYELANEISFLSYGYENLGLVTTVTGVSDINFDTDSNWPYYIDRDALGNTGSYVNATIEYEGFFRAPVDGEYVFSFSSTDYNSILFVGSPAAADQALQKREVQFLKPETSPDYVLLFNNTRDLGKTVSTTQYLLADQYYPLRVVIAAISQHALLDFQIKLPNGASLTQYQGYVYNFALEGSESTTVIGDKTSTWTGSYTTWTTDSDGSTIVVVPPATITADKTSTWTGSYTTWTTDSDGSTVVICPSITSDHNDKPSESTLTDSSISTTVVTVTSCDIEKCTKTTALTGVRETTLTTGGTTTVVTTYCPLPTDIVTVKTTSIDGSEVLQTIYTAKPNHVVPDVQTSTVTITREVCDAFTCTHATIVTGEILKTTTLADTHYTTVVPVYVPLETYQPAVELSTLETVLKSSDLASGPVVTAGSVQPSYQSGGVAESSLTVSEFEAHSTSDTVSQPSTISLQTGEANALKWSSFFGAALVPLVNVFFV

SEQ ID NO: 81 - protein, 2812 aa, Synthetic Construct
MQNTNDKLIIRTFYSISTIHGLLSINIFSDTRVYKFAIYSTDAVSLEPRTKNNMSLVTVLACFIIFAAHAFGQDTFYMLKVRTLTPNGYPLADSLSNPMQYWDLYYVPGGPRRLESSFVNWQPTTAAPINQFYCRLGTDGHMTGYNRVTGSVIGKLSFGTNAATALAFGSYDGDPSYPPQAFSISSSVSGTMTYLNVHYVNARSITWYSTTTATGETNVYINVASTGYTGDRTTYQAELWVEPFVPNIPVDTTTSIWTGSQTSYTTEVGENGGSTVIELIPTPPADATSTWTGTYTTRTTDADGSVIEQIPTPSADTTSVWTGTYTTWTTDADGSVIEQIPTPSADTTSVWTGTYTTWTTDADGSVIEQIPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVIELVPSDTETATNIVETPVPSSGVSDGVSVFDGFNVEVFHYPADNYELANEIGFLSYGYENLGLVTNATGVSDINFDTDSNWPYYIDRDALGNTGSYVNATIEYEGFFRAPVDGEYVFSFSNTDYNSILFVGSPAAAGQALQKRRVQFLKPETSPDHVLLFNNTRDLGQTISTTQYLLADQYYPLRVVIAAISQHALLDFQIKLPNGALLTQYQGYVYNFALEGSESTTVIGDKTSTWTGSYTTWTTDSDGSTVVVVPSATITADKTSTWTGSYTTWTTDSDGSTIVICPSITSDHNDKPSESTLTDGSISTTVVTVTSCDIEKCTKTTALTGVTETTLTTGGTTTVVTTYCPLPTDIVTVKTTSISGSEVLQTIYTAKPSHVVPNVHTLTVTITREVCDAFTCTQATIVTGEILKTTTLADTHSTTVVPVYVPLESYQSAVELSTLETVLKSSDFASGSAVTAGSAQPSYQSGGVAESSLTGSELEAHSTSDTVSQPSTISPQTGEANALRWSSFFGAALVPLVNVFFV

SEQ ID NO: 82 - protein, 860 aa, Synthetic Construct
MTKLTILLSVLLQLFSVLAEVPKKTEWSSHTTYWTSTLEALRTVTPTGTERAVIGEAPYEYKLIGNDQFDPGLNAKREIIDCEAVCCGAVPTSDPLKRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTQPWTGTYETTYTIPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTKPWTGTYETTHTVPASGTEPGTVIIETPIKYLNTSISASTSTWTKINTVTQFISCPVCTIPKTITVTPKISNETVTIIISQPHGTSSRTTTVVKTDGASVSSHSYKTALTTDVKPEEKTSTKLGTVTTVSGSHSAIDTVTGSLSDYHASSIPHTVKSEEKASSTVTHTISSSTVYQVSPSNGASWLSVRLNTALSIIGTLFAAVFI

SEQ ID NO: 83 - protein, 1436 aa, Synthetic Construct
MSKTKNGGSEFVHIAYVFHIEASTPSDYINMIQIVLFPHQAQITKRMNLVTLLVCNLLCVSLTLGQGVYRLKFPALVVTGRESVGTTVVNYDFLVGNTGQYGDLGEFFYDGEPYYCWNSTDSQPLSCSSSSSLLISTQNVTISHPDEDGTVYAYAERDGGLLGRFTVGSVSADWPQWAVIVYSTSSSAHPSSWYVDDNKLKLTSGLGPNNSTTLQACYFTQSSGRDRYAISLEGSPAYTGQVSCQATEFDLEFIPPSADTTSIWDGSYTTWTTDSNGIVVEQIPTPSADTTSIWTGSETSWTTDSDGTVIELVPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDREGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGDHTTWTTEVGGDGSSIVVELVPSETGTATNVVQTPVPSSGISDGVSALDGFNVEVFHYPADNYELANEISFLSYGYENLGLVTTATGVSDINFDTDSNWPSYIDRNALGNTGSYVNATIKYEGFFRAPVDGDYEFSFSNIDYNSILFVGSAAADQALRKREAQFLKPETSPNHILFFNNSRDVGQTISTTQYLSADSYYPLRVVIAAVSQHALLDFQIKLPNGVSLTQFQGYVYNFALEGAESTTVIGDKTSTWTGTYTTWTTDSEGSTIVLCPSIISDHNGKPADTTLTDGSISTTVVTVTSCDIKKCTKTTALTGVTQKTLTVKGTTTVVTAYCPLPTDVATVKTISVGGSEVLQTVYTAKPSHIVPDVQTLTVTITREVCDALTCIPATIVTGEILKTTTLADTHSTTVIPVYVPLETHQPALDLITLETVLKSSDFANGPAITSVSVESLSHQSGVVVSEFDSDSTSGAVSQPSSAVSLQTGKASALKWSPFLGAAVISLFNVFFV

SEQ ID NO: 84 - protein, 2435 aa, Synthetic Construct
MNLFTILAWGFLYVPLVLGEGYYSLNFDARVPIALGILGSSYQKYTIMADRSLLGGSNIDLDVTFSGIIELLTNRVHIVVSLPDADGRVSVYDMYSGTSLGYLSFVCSLTTCEVHAVSSSSGATTWTLDGNQLIPTSPSTVYACYRSLVGLLAQYTLNDRTSITAQCEQTNLYVELAIPAFPETTAVWTGTYTTWTTDESGSVIEQMPTPSADTTTTWTGTYTTWTTDADGSVIEQIPTPPADTTSVWTGTYTTRTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSIWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSVWTGTYTTWTTDAAGTVIEVIPSGTSISSDVIPTPLPTSGVDIDTIPYDAFNVAVYHYPADNYELANNLGFLTSGYEGLGQVTTATSVGNINFDTSSGWPYYIESNALGNTGSYVNATIEYVGFFQAPANGNYELSFSNIDYNAILFLGSPATDSSLAKREVQFLKPETSSEYVLFFDHGKDAGQTVSTTQYLSAGLYYPLRIVLAAVSERAQLDFQITLPDGRVLDQYQGYVYNFAHEGIESATSSAHETSWSRFTNSTIYSHSSTIGIITSSTDAPHSVINPTAIETTSTDTSISTVAVTTSICDTKDCVKTTVITPNSPLPTQTVSLTTTTIDRSEVVQTAHSAVPSQFAPDAHPSAVTITREQCDAYSCSQATIVSGKVLQTTTVSDSTTVVPLDTPQLSVEASTLETRLKSTQSSRAPTVTVQTSQSSRHSEDVTESSVHVSEFDAQSTSATSASALQAPSSISLQTGGANTLRLSAFLGTALLPMLNVLFI

SEQ ID NO: 85 - protein, 135 aa, Synthetic Construct
MQFSIVATLALAGSALAAYSNVTYTYETTITDVVTELTTYCPEPTTFVHKNKTITVTAPTTLTITDCPCTISKTTKITTDVPPTTHSTPHTTTTHVPSTSTPAPTHSVSTISHGGAAKAGVAGLAGVAAAAAYFL

SEQ ID NO: 86 - protein, 219 aa, Synthetic Construct
MLLTSLLQVFACCLVLPAQVTAFYYYTSGAERKCFHKELSKGTLFQATYKAQIYDDQLQNYRDAGAQDFGVLIDIEETFDDNHLVVHQKGSASGDLTFLASDSGEHKICIQPEAGGWLIKAKTKIDVEFQVGSDEKLDSKGKATIDILHAKVNVLNSKIGEIRREQKLMRDREATFRDASEAVNSRAMWWIVIQLIVLAVTCGWQMKHLGKFFVKQKIL

SEQ ID NO: 87 - protein, 215 aa, Synthetic Construct
MIKSTIALPSFFIVLILALVNSVAASSSYAPVAISLPAFSKECLYYDMVTEDDSLAVGYQVLTGGNFEIDFDITAPDGSVITSEKQKKYSDFLLKSFGVGKYTFCFSNNYGTALKKVEITLEKEKTLTDEHEADVNNDDIIANNAVEEIDRNLNKITKTLNYLRAREWRNMSTVNSTESRLTWLSILIIIIIAVISIAQVLLIQFLFTGRQKNYV

SEQ ID NO: 88 - protein, 203 aa, Synthetic Construct [sequence omitted]

SEQ ID NO: 89 - protein, 211 aa, Synthetic Construct [sequence omitted]

SEQ ID NO: 90 - protein, 225 aa, Synthetic Construct
MSNLCVLFFQFFFLAQFFAEASPLTFELNKGRKECLYTLTPEIDCTISYYFAVQQGESNDFDVNYEIFAPDDKNKPIIERSGERQGEWSFIGQHKGEYAICFYGGKAHDKIVDLDFKYNCERQDDIRNERRKARKAQRNLRDSKTDPLQDSVENSIDTIERQLHVLERNIQYYKSRNTRNHHTVCSTEHRIVMFSIYGILLIIGMSCAQIAILEFIFRESRKHNV

SEQ ID NO: 91 - protein, 212 aa, Synthetic Construct
MKYNIVHGICLLFAITQAVGAVHFYAKSGETKCFYEHLSRGNLLIGDLDLYVEKDGLFEEDPESSLTITVDETFDNDHRVLNQKNSHTGDVTFTALDTGEHRFCFTPFYSKKSATLRVFIELEIGNVEALDSKKKEDMNSLKGRVGQLTQRLSSIRKEQDAIREKEAEFRNQSESANSKIMTWSVFQLLILLGTCAFQLRYLKNFFVKQKVV

SEQ ID NO: 92 - protein, 186 aa, Synthetic Construct
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 93 - protein, 186 aa, Synthetic Construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 94 - protein, 186 aa, Synthetic Construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 95 - protein, 210 aa, Synthetic Construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 96 - protein, 210 aa, Synthetic Construct
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 97 - protein, 208 aa, Synthetic Construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 98 - protein, 186 aa, Synthetic Construct
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 99 - protein, 208 aa, Synthetic Construct
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 100 - protein, 239 aa, Synthetic Construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 101 - protein, 185 aa, Synthetic Construct
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 102 - protein, 237 aa, Synthetic Construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 103 - protein, 128 aa, Synthetic Construct
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 104 - protein, 195 aa, Synthetic Construct
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 105 - protein, 339 aa, Synthetic Construct
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 106 - protein, 238 aa, Synthetic Construct
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 107 - protein, 284 aa, Synthetic Construct
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 108 - protein, 186 aa, Synthetic Construct
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 109 - protein, 210 aa, Synthetic Construct
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 110 - protein, 205 aa, Synthetic Construct
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 111 - protein, 171 aa, Synthetic Construct
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 112 - protein, 207 aa, Synthetic Construct
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 113 - protein, 207 aa, Synthetic Construct
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 114 - protein, 207 aa, Synthetic Construct
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 115 - protein, 207 aa, Synthetic Construct
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 116 - protein, 207 aa, Synthetic Construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 117 - protein, 207 aa, Synthetic Construct
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 118 - protein, 207 aa, Synthetic Construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 119 - protein, 207 aa, Synthetic Construct
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 120 - protein, 206 aa, Synthetic Construct
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 121 - protein, 213 aa, Synthetic Construct
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 122 - protein, 207 aa, Synthetic Construct
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 123 - protein, 207 aa, Synthetic Construct
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 124 - protein, 207 aa, Synthetic Construct
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 125 - protein, 207 aa, Synthetic Construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 126 - protein, 282 aa, Synthetic Construct
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 127 - protein, 193 aa, Synthetic Construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 128 - protein, 190 aa, Synthetic Construct
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 129 - protein, 199 aa, Synthetic Construct
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 130 - protein, 275 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 131 - protein, 273 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 132 - protein, 188 aa, Synthetic Construct
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 133 - protein, 206 aa, Synthetic Construct
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 134 - protein, 272 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 135 - protein, 187 aa, Synthetic Construct
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 136 - protein, 402 aa, Synthetic Construct
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 137 - protein, 388 aa, Synthetic Construct
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 138 - protein, 386 aa, Synthetic Construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 139 - protein, 474 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 140 - protein, 389 aa, Synthetic Construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 141 - protein, 419 aa, Synthetic Construct
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 142 - protein, 406 aa, Synthetic Construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 143 - protein, 451 aa, Synthetic Construct
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 144 - protein, 386 aa, Synthetic Construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 145 - protein, 386 aa, Synthetic Construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 146 - protein, 417 aa, Synthetic Construct
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 147 - protein, 383 aa, Synthetic Construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 148 - protein, 386 aa, Synthetic Construct
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 149 - protein, 411 aa, Synthetic Construct
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 150 - protein, 383 aa, Synthetic Construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 151 - protein, 408 aa, Synthetic Construct
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 152 - protein, 383 aa, Synthetic Construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 153 - protein, 386 aa, Synthetic Construct
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 154 - protein, 386 aa, Synthetic Construct
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 155 - protein, 386 aa, Synthetic Construct
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 156 - protein, 386 aa, Synthetic Construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 157 - protein, 386 aa, Synthetic Construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 158 - protein, 386 aa, Synthetic Construct
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 159 - protein, 386 aa, Synthetic Construct
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 160 - protein, 386 aa, Synthetic Construct
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 161 - protein, 386 aa, Synthetic Construct
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 162 - protein, 386 aa, Synthetic Construct
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 163 - protein, 386 aa, Synthetic Construct
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 164 - protein, 386 aa, Synthetic Construct
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 165 - protein, 391 aa, Synthetic Construct
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 166 - protein, 386 aa, Synthetic Construct
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 167 - protein, 386 aa, Synthetic Construct
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 168 - protein, 386 aa, Synthetic Construct
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 169 - protein, 386 aa, Synthetic Construct
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 170 - protein, 385 aa, Synthetic Construct
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 171 - protein, 386 aa, Synthetic Construct
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 172 - protein, 386 aa, Synthetic Construct
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 173 - protein, 386 aa, Synthetic Construct
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 174 - protein, 385 aa, Synthetic Construct
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 175 - protein, 386 aa, Synthetic Construct
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 176 - protein, 386 aa, Synthetic Construct
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 177 - protein, 386 aa, Synthetic Construct
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 178 - protein, 386 aa, Synthetic Construct
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 179 - protein, 386 aa, Synthetic Construct
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 180 - protein, 386 aa, Synthetic Construct
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 181 - protein, 386 aa, Synthetic Construct
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 182 - protein, 386 aa, Synthetic Construct
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 183 - protein, 386 aa, Synthetic Construct
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 184 - protein, 381 aa, Synthetic Construct
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 185 - protein, 391 aa, Synthetic Construct
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 186 - protein, 383 aa, Synthetic Construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 187 - protein, 386 aa, Synthetic Construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 188 - protein, 392 aa, Synthetic Construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 189 - protein, 386 aa, Synthetic Construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 190 - protein, 395 aa, Synthetic Construct
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 191 - protein, 386 aa, Synthetic Construct
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 192 - protein, 386 aa, Synthetic Construct
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 193 - protein, 385 aa, Synthetic Construct
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 194 - protein, 386 aa, Synthetic Construct
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 195 - protein, 383 aa, Synthetic Construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 196 - protein, 386 aa, Synthetic Construct
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 197 - protein, 386 aa, Synthetic Construct
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 198 - protein, 2174 aa, Synthetic Construct
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 199 - protein, 386 aa, Synthetic Construct
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 200 - protein, 2104 aa, Synthetic Construct
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 201 - protein, 386 aa, Synthetic Construct
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 202 - protein, 420 aa, Synthetic Construct
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 203 - protein, 386 aa, Synthetic Construct
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 204 - protein, 386 aa, Synthetic Construct
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 205 - protein, 386 aa, Synthetic Construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 206 - protein, 386 aa, Synthetic Construct
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 207 - protein, 386 aa, Synthetic Construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 208 - protein, 399 aa, Synthetic Construct
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 209 - protein, 389 aa, Synthetic Construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 210-299 - skipped (90 entries)

SEQ ID NO: 300 - protein, 474 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 301 - protein, 389 aa, Synthetic Construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 302 - protein, 474 aa, Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 303 - protein, 426 aa, Synthetic Construct
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 304 - protein, 388 aa, Synthetic Construct; identical to SEQ ID NO: 137

SEQ ID NO: 305 - protein, 1185 aa, Synthetic Construct
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 306 - protein, 129 aa, Synthetic Construct [sequence omitted]

SEQ ID NO: 307 - protein, 129 aa, Synthetic Construct
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 308 - protein, 130 aa, Synthetic Construct
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 309 - protein, 129 aa, Synthetic Construct
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 310 - protein, 449 aa, Synthetic Construct
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 311 - protein, 139 aa, Synthetic Construct
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 312 - protein, 450 aa, Synthetic Construct
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 313 - protein, 455 aa, Synthetic Construct
GLVAADRITGGKDFRDIESKFALRTPEDTAEDTCHLIPGVTESVANCHFNHSSKTFVVIHGWTVTGMYESWVPKLVAALYKREPDSNVIVVDWLSRAQQHYPVSAGYTKLVGQDVAKFMNWMADEFNYPLGNVHLLGYSLGAHAAGIAGSLTSKKVNRITGLDPAGPNFEYAEAPSRLSPDDADFVDVLHTFTRGSPGRSIGIQKPVGHVDIYPNGGTFQPGCNIGEALRVIAERGLGDVDQLVKCSHERSVHLFIDSLLNEENPSKAYRCNSKEAFEKGLCLSCRKNRCNNMGYEINKVRAKRSSKMYLKTRSQMPYKVFHYQVKIHFSGTESNTYTNQAFEISLYGTVAESENIPFTLPEVSTNKTYSFLLYTEVDIGELLMLKLKWISDSYFSWSNWWSSPGFDIGKIRVKAGETQKKVIFCSREKMSYLQKGKSPVIFVKCHDKSLNRKSG

SEQ ID NO: 314 - protein, 685 aa, Synthetic Construct
APKKGVRWCVISTAEYSKCRQWQSKIRRTNPMFCIRRASPTDCIRAIAAKRADAVTLDGGLVFEADQYKLRPVAAEIYGTEENPQTYYYAVAVVKKGFNFQLNQLQGRKSCHTGLGRSAGWNIPIGLLRRFLDWAGPPEPLQKAVAKFFSQSCVPCADGNAYPNLCQLCIGKGKDKCACSSQEPYFGYSGAFNCLHKGIGDVAFVKESTVFENLPQKADRDKYELLCPDNTRKPVEAFRECHLARVPSHAVVARSVNGKENSIWELLYQSQKKFGKSNPQEFQLFGSPGQQKDLLFRDATIGFLKIPSKIDSKLYLGLPYLTAIQGLRETAAEVEARQAKVVWCAVGPEELRKCRQWSSQSSQNLNCSLASTTEDCIVQVLKGEADAMSLDGGFIYTAGKCGLVPVLAENQKSRQSSSSDCVHRPTQGYFAVAVVRKANGGITWNSVRGTKSCHTAVDRTAGWNIPMGLLVNQTGSCKFDEFFSQSCAPGSQPGSNLCALCVGNDQGVDKCVPNSNERYYGYTGAFRCLAENAGDVAFVKDVTVLDNTNGQNTEEWARELRSDDFELLCLDGTRKPVTEAQNCHLAVAPSHAVVSRKEKAAQVEQVLLTEQAQFGRYGKDCPDKFCLFRSETKNLLFNDNTEVLAQLQGKTTYEKYLGSEYVTAIANLKQCSVSPLLEACAFMMR

SEQ ID NO: 315 - protein, 689 aa, Synthetic Construct
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 316 - protein, 5 aa, Synthetic Construct
GGGGS

SEQ ID NO: 317 - skipped

SEQ ID NO: 318 - protein, 20 aa, Synthetic Construct
EAAAREAAAREAAAREAAAR

SEQ ID NO: 319 - protein, 59 aa, Synthetic Construct
GSSGSSGSSGSSGSSGSSGSSGSSEAAAREAAAREAAAREAAARGGGGSGGGGSGGGGS

SEQ ID NO: 320 - protein, 24 aa, Synthetic Construct
GSSGSSGSSGSSGSSGSSGSSGSS

SEQ ID NO: 321 - protein, 15 aa, Synthetic Construct
GGGGSGGGGSGGGGS

SEQ ID NO: 322 - protein, 547 aa, Synthetic Construct
MPHKRTPSSSLLYARIPGISFENSPVFDFLSPFGPAPNQWVARYIIIIFAILIRLAVGLGSYSGFNTPPMYGDFEAQRHWMEITQHLSIEKWYFYDLQYWGLDYPPLTAFHSYFFGKLGSFINPAWFALDVSRGFESVDLKSYMRATAILSELLCFIPAVIWYCRWMGLNYFNQNAIEQTIIASAILFNPSLIIIDHGHFQYNSVMLGFALLSILNLLYDNFALAAIFFVLSISFKQMALYYSPIMFFYMLSVSCWPLKNFNLLRLATISIAVLLTFATLLLPFVLVDGMSQIGQILFRVFPFSRGLFEDKVANFWCTTNILVKYKQLFTDKTLTRISLVATLIAISPSCFIIFTHPKKVLLPWAFAACSWAFYLFSFQVHEKSVLVPLMPTTLLLVEKDLDIISMVCWISNIAFFSMWPLLKRDGLALEYFVLGILSNWLIGNLNWISKWLVPSFLIPGPTLSKKVPKRDTKTVVHTHWFWGSVTFVSYLGATVIQFVDWLYLPPAKYPDLWVILNTTLSFACFGLFWLWINYNLYILRDFKLKDA

SEQ ID NO: 323 - protein, 742 aa, Synthetic Construct
MVTINDQGYITVNDRVLKLIKSLLIVLIFISITIAAVSSRLFSVIRFESIIHEFDPWFNFRATKYLVHNGFYKFLNWFDDKTWYPLGRVTGGTLYPGLMVTSAVIHNLLAKIGLPIDIRNICVMLAPAFSSLTAIAMYFLTLELTNDSESIANGTAKATAALFSAIFMGITPGYISRSVAGSYDNEAIAITLLMVTFYFWIKAVKLGSIFYSSVTALFYFYMVSAWGGYVFITNLIPLHVFVLLLMGRFTHKIYVSYTTWYVLGTLMSMQIPFVGFLPIRSNDHMAPLGVFGLIQLVLIGDFFKSQLSRKVFIKLAIASGVVIGILGVVGLVLATKIGLIAPWTGRFYSLWDTNYAKIHIPIIASVSEHQPTPWASFFFDLNFLIWLFPVGVWFCFQELTDGAVFVIIYSVLASYFAGVMVRLILTLAPIVCVCGAIAITKLFEVYSDFTDVVKGKSGNFFTLFSKLAVLGSFGFYLFFYVKHCTWVTENAYSSPSVVLASHAADGSQILIDDYREAYYWLRMNTPEDAKVMAWWDYGYQIGGMADRTTFVDNNTWNNTHIATVGKAMAVSEEKSEVIMRQLGVDYILVIFGGVLGYSGDDINKFLWMVRISEGIWPEEVSERGYFTPRGEYKIDDNAAQAMKDSMLYKMSFYRFGELFPSGDAIDRVRGQRLSRSYAESIDLNIVEEVFTSENWLVRLYKLKEPDNLGRSLLTLKDNEKKLATKKGRRLRVNKKPSLDLRV

(19) United States
(12) Patent Application Publication
(10) Pub. No.: US 2024/0209328 A1
(43) Pub. Date: Jun. 27, 2024

(54) PROTEIN COMPOSITIONS AND METHODS OF PRODUCTION

(71) Applicant: Clara Foods Co., Daly City, CA (US)

(72) Inventors: Logan HURST, Daly City, CA (US); Weixi ZHONG, Daly City, CA (US)

(73) Assignee: Clara Foods Co., Daly City, CA (US)

(21) Appl. No.: 18/419,747

(22) Filed: Jan. 23, 2024

Related U.S. Application Data
(63) Continuation of application No. PCT/US2022/038095, filed on Jul. 22, 2022.
(60) Provisional application No. 63/225,355, filed on Jul. 23, 2021.
(60) Provisional application No. 63/356,944, filed on Jun. 29, 2022.

(51) Int. Cl. C12N 9/10; A61K 8/64; C07K 14/395; C07K 14/465; C12N 9/24; C12N 15/81

(52) U.S. Cl. CPC C12N 9/1051; A61K 8/64; C07K 14/395; C07K 14/465; C12N 9/2402; C12N 15/815; C07K 2319/02

(57) ABSTRACT

Provided are systems and methods for production of recombinant proteins in engineered microorganisms while reducing impurities produced in the culture.

### CROSS-REFERENCE

This application is a continuation of International Patent Application No. PCT/US2022/038095, filed Jul. 22, 2022, which claims the benefit of and priority to U.S. Provisional Patent Application No. 63/225,355, filed Jul. 23, 2021, and U.S. Provisional Patent Application No. 63/356,944, filed Jun. 29, 2022, each of which is herein incorporated by reference in its entirety.

### SEQUENCE LISTING

The instant application contains a Sequence Listing which has been submitted electronically in XML format and is hereby incorporated by reference in its entirety. Said XML copy, created on Aug. 15, 2022, is named 41960-730601.xml, and is 354,444 bytes in size.

### BACKGROUND

In industrial protein production, a goal towards cost reduction is to maximize expression of the protein product in the recombinant organism. Methylotrophic yeasts such as Pichia sp. are an important production system for proteins. Despite their widespread use, high yield expression, particularly for expression of heterologous animal-derived proteins remains a challenge. This hurdle is particularly apparent in larger scale fermentation settings. While increasing the number of integrated copies can lead to increases in protein expression, there appear to be limitations to the amount of transcript produced with increasing copy number.

There is a growing demand for animal-free proteins, particularly in food product-based ingredients. For example, an observable trend of preference for health-conscious fast food options has seen egg white demand at all-time highs in recent years. Aside from an increasingly health conscious consumer base, aversion to the inhumane aspects of the industrial hatchery may fuel acceptance and ultimately preference of animal-free egg white alternatives over factory-farmed eggs. Thus, there is a need for novel methods for high-yield industrial production of food proteins, e.g., alternative animal-free egg proteins.

### SUMMARY

In some aspects, provided herein is a recombinant host cell for manufacturing a heterologous protein of interest. In some embodiments, the host cell may be a yeast and may be engineered to underexpress two mannosyl transferases: beta-mannosyl transferase 1 (BMT1) and beta-mannosyl transferase 2 (BMT2) or functional homologues thereof wherein the underexpression may be compared to the host cell prior to genetic manipulation, wherein the host cell may be engineered to express a heterologous protein of interest and a heterologous mannosidase.

In some embodiments, the underexpression may be achieved by independently for each mannosyl transferase protein knocking-out the polynucleotide encoding the mannosyl transferase protein or a homologue thereof from the genome of said host cell, disrupting the polynucleotide encoding the mannosyl transferase protein or a homologue thereof in the host cell, disrupting a promoter which may be operably linked with said polynucleotide encoding the mannosyl transferase protein or a homologue thereof, replacing the promoter which may be operably linked with said polynucleotide encoding the mannosyl transferase protein or a homologue thereof with another promoter which has lower promoter activity, or disrupting expression control sequences of the mannosyl transferase protein or a homologue thereof, wherein the functional homologue has at least 70% sequence identity to an amino acid sequence of a mannosyl transferase. In some embodiments, the host cell may be Pichia pastoris.

In some embodiments, the BMT1 protein may comprise an amino acid sequence that may be at least 70% identical, at least 75% identical, at least 80% identical, at least 85% identical, at least 90% identical, or at least 95% identical, to SEQ ID NO: 12.

In some embodiments, the BMT2 protein may comprise an amino acid sequence that may be at least 70% identical, at least 75% identical, at least 80% identical, at least 85% identical, at least 90% identical, or at least 95% identical, to SEQ ID NO: 13.

In some embodiments, the recombinant host cell may be engineered to express at least 10% less BMT1 relative to a host cell which has not been engineered to underexpress BMT1.

In some embodiments, the recombinant host cell may be engineered to express at least 10%, 15%, 20%, 30%, 40%, 50%, 60%, 70%, 80%, 90%, or 95% less BMT1 relative to a host cell which has not been engineered to underexpress BMT1.

In some embodiments, the recombinant host cell may be engineered to knockout BMT1, wherein the knockout leads to no activity of BMT1 in the recombinant host cell.

In some embodiments, the recombinant host cell may be engineered to express at least 10% less BMT2 relative to a host cell which has not been engineered to underexpress BMT2.

In some embodiments, the recombinant host cell may be engineered to express at least 10%, 15%, 20%, 30%, 40%, 50%, 60%, 70%, 80%, 90%, or 95% less BMT2 relative to a host cell which has not been engineered to underexpress BMT2.

In some embodiments, the recombinant host cell may be engineered to knock out BMT2, wherein the knockout leads to no activity of BMT2 in the recombinant host cell.

In some embodiments, the recombinant host cell produces a reduced size of exopolysaccharides relative to a host cell not engineered to underexpress BMT1 and BMT2.

In some embodiments, the recombinant host cell may be further engineered to underexpress alpha-1,2-mannosyltransferase MNN2.

In some embodiments, the MNN2 protein may comprise an amino acid sequence that may be at least 70% identical, at least 75% identical, at least 80% identical, at least 85% identical, at least 90% identical, or at least 95% identical, to one of SEQ ID NO: 1.

In some embodiments, the recombinant host cell may be engineered to express at least 10%, 15%, 20%, 30%, 40%, 50%, 60%, 70%, 80%, 90%, or 95% less MNN2 relative to a host cell which has not been engineered to underexpress MNN2.

In some embodiments, the recombinant host cell may be further engineered to underexpress MNNF1.

In some embodiments, the MNNF1 protein may comprise an amino acid sequence that may be at least 70% identical, at least 75% identical, at least 80% identical, at least 85% identical, at least 90% identical, or at least 95% identical, to one of SEQ ID NO: 2.

In some embodiments, the recombinant host cell may be engineered to express at least 10%, 15%, 20%, 30%, 40%, 50%, 60%, 70%, 80%, 90%, or 95% less MNNF1 relative to a host cell which has not been engineered to underexpress MNNF1.

In some embodiments, the recombinant host cell may be further engineered to underexpress MNNF2.

In some embodiments, the MNNF2 protein may comprise an amino acid sequence that may be at least 70% identical, at least 75% identical, at least 80% identical, at least 85% identical, at least 90% identical, or at least 95% identical, to one of SEQ ID NO: 3.

In some embodiments, the recombinant host cell may be engineered to express at least 10%, 15%, 20%, 30%, 40%, 50%, 60%, 70%, 80%, 90%, or 95% less MNNF2 relative to a host cell which has not been engineered to underexpress MNNF2.

In some embodiments, the recombinant host cell may be further engineered to underexpress one or more enzymes in addition to BMT1 and BMT2.

In some embodiments, the one or more enzymes may comprise an amino acid sequence that may be at least 70% identical, at least 75% identical, at least 80% identical, at least 85% identical, at least 90% identical, or at least 95% identical, to one of SEQ ID NOs: 4-11, 14-15, and 72-85.

In some embodiments, the recombinant host cell may be engineered to express at least 10%, 15%, 20%, 30%, 40%, 50%, 60%, 70%, 80%, 90%, or 95% less one or more enzymes relative to a host cell which has not been engineered to underexpress said one or more enzymes.

In some embodiments, the recombinant host cell recombinantly expresses a mannosidase from a species different from the recombinant host cell.

In some embodiments, the mannosidase may be from a genus different from the recombinant host cell.

In some embodiments, the mannosidase may comprise an amino acid sequence that may be at least 70% identical, at least 75% identical, at least 80% identical, at least 85% identical, at least 90% identical, or at least 95% identical, to one of SEQ ID NOs: 41-56.

In some embodiments, the mannosidase may be expressed on the surface of the recombinant host cell.

In some embodiments, the recombinant host cell expresses a surface-displayed fusion protein may comprise a catalytic domain of a mannosidase and an anchoring domain of a glycosylphosphatidylinositol (GPI)-anchored protein, wherein the anchoring domain may comprise at least about 200 amino acids and/or at least about 30% of the residues in the anchoring domain are serines or threonines.

In some embodiments, the anchoring domain may comprise at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In some embodiments, the serines or threonines in the anchoring domain are capable of being O-mannosylated.

In some embodiments, a fusion protein having an anchoring domain may comprise at least about 325 amino acids provides greater enzymatic activity relative to a fusion protein having an anchoring domain may comprise less than about 300 amino acids.

In some embodiments, a fusion protein having an anchoring domain may comprise at least about 300 amino acids provides greater enzymatic activity relative to a fusion protein having an anchoring domain may comprise less than about 250 amino acids.

In some embodiments, the fusion protein may comprise the anchoring domain of the GPI anchored protein.

In some embodiments, the fusion protein may comprise the GPI anchored protein without its native signal peptide.

In some embodiments, the GPI anchored protein may be not native to the recombinant host cell.

In some embodiments, the GPI anchored protein may be naturally expressed by a S. cerevisiae cell and the recombinant host cell may be not a S. cerevisiae cell.

In some embodiments, the GPI anchored protein may be selected from Tir4, Dan1, Dan4, Sag1, Fig2, and Sed1.

In some embodiments, the anchoring domain of the GPI anchored protein may comprise an amino acid sequence that may be at least 70% identical, at least 75% identical, at least 80% identical, at least 85% identical, at least 90% identical, or at least 95% identical, to one of SEQ ID NO: 57 to SEQ ID NO: 71.

In some embodiments, the anchoring domain of the GPI anchored protein may comprise an amino acid sequence of one of SEQ ID NO: 57 to SEQ ID NO: 71.

In some embodiments, the recombinant host cell may comprise a genomic modification that expresses the fusion protein and/or may comprise an extrachromosomal modification that expresses the fusion protein.

In some embodiments, the fusion protein may comprise a portion of the mannosidase in addition to its catalytic domain.

In some embodiments, the fusion protein may comprise substantially the entire amino acid sequence of the mannosidase.

In some embodiments, the fusion protein, the catalytic domain may be N-terminal to the anchoring domain.

In some embodiments, the fusion protein may comprise a linker between the catalytic domain and the anchoring domain.

In some embodiments, the fusion protein may comprise a linker having an amino acid sequence that may be at least 95% identical to any one of SEQ ID NOs: 316-321.

In some embodiments, upon translation, the fusion protein may comprise a signal peptide and/or a secretory signal.

In some embodiments, the recombinant host cell may comprise two or more fusion proteins, three or more fusion proteins, or four fusion proteins.

In some embodiments, the recombinant host cell may comprise a mutation in its AOX1 gene and/or its AOX2 gene.

In some embodiments, the recombinant host cell may comprise a genomic modification that overexpresses a secreted heterologous protein of interest and/or may comprise an extrachromosomal modification that overexpresses a secreted protein of interest.

In some embodiments, the secreted protein of interest may be an animal protein.

In some embodiments, the animal protein may be an egg protein.

In some embodiments, the egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In some embodiments, the genomic modification and/or the extrachromosomal modification that overexpresses the secreted recombinant protein may comprise an inducible promoter.

In some embodiments, the inducible promoter may be an AOX1, DAK2, PEX11, FLD1, FGH1, DAS1, DAS2, CAT1, MDH3, HAC1, BIP, RAD30, RVS161-2, MPP10, THP3, TLR, GBP2, PMP20, SHB17, PEX8, PEX4, or TKL3 promoter.

In some embodiments, the genomic modification and/or the extrachromosomal modification that overexpresses a secreted recombinant protein may comprise an AOX1, TDH3, MOX, RPS25A, or RPL2A terminator.

In some embodiments, the genomic modification and/or the extrachromosomal modification that overexpresses a secreted recombinant protein encodes a signal peptide and/or a secretory signal.

In some embodiments, the genomic modification and/or the extrachromosomal modification that overexpresses a secreted recombinant protein may comprise codons that are optimized for the species of the recombinant host cell.

In some embodiments, the secreted recombinant protein may be designed to be secreted from the cell and/or may be capable of being secreted from the cell.

In some embodiments, the additional genomic modification reduces the number of native cell wall proteins expressed by the recombinant host cell, thereby allowing additional space for localization of the surface-displayed fusion protein.

In some embodiments, the recombinant host cell may comprise a further genomic modification that overexpresses a protein related to the p24 complex.

In some embodiments, the recombinant host cell may comprise a further genomic modification may comprise that overexpresses more than one protein related to the p24 complex.

In some embodiments, the protein related to the p24 complex may be selected from Erp1, Erp2, Erp3, Erp5, Emp24, and Erv25.

In some embodiments, the protein related to the p24 complex may comprise the amino acid sequence of any one of SEQ ID NO: 86 to SEQ ID NO: 91.

In some aspects, described herein are methods for expressing a heterologous protein of interest. In some embodiments, the method may comprise obtaining a recombinant host cell described herein and culturing the recombinant host cell under conditions that allow expression of the heterologous protein of interest.

In some embodiments, the isolated heterologous protein of interest may be expressed according to the methods described herein.

In some aspects, provided herein is a method for expressing a heterologous protein of interest. In some embodiments, the method may comprise having of a reduced level of exopolysaccharides, the method may comprise obtaining a recombinant host cell described herein and culturing the recombinant host cell under conditions that allow expression of the heterologous protein of interest.

In some aspects, provided herein is a method for expressing a heterologous protein of interest having of a reduced level of exopolysaccharides. The method may comprise: obtaining a host cell that may be a yeast and may be engineered to underexpress two mannosyl transferases: beta-mannosyl transferase 1 (BMT1) and beta-mannosyl transferase 2 (BMT2) or functional homologues thereof wherein the underexpression may be compared to the host cell prior to genetic manipulation, wherein the host cell may be engineered to express a heterologous protein of interest and a heterologous mannosidase; and culturing the recombinant host cell under conditions that allow expression of the heterologous protein of interest

In some embodiments, the BMT1 protein may comprise an amino acid sequence that may be at least 70% identical, at least 75% identical, at least 80% identical, at least 85% identical, at least 90% identical, or at least 95% identical, to SEQ ID NO: 12 and the BMT2 protein may comprise an amino acid sequence that may be at least 70% identical, at least 75% identical, at least 80% identical, at least 85% identical, at least 90% identical, or at least 95% identical, to SEQ ID NO: 13.

In some embodiments, the recombinant host cell may be further engineered to underexpress one or more enzymes may comprise an amino acid sequence of one of SEQ ID NOs: 1-11, 14-15, and 72-85.

In some embodiments, the recombinant host cell recombinantly expresses a mannosidase from a species different than from the recombinant host cell.

In some embodiments, the mannosidase may comprise an amino acid sequence that may be at least 70% identical, at least 75% identical, at least 80% identical, at least 85% identical, at least 90% identical, or at least 95% identical, to one of SEQ ID NOs: 41-56.

In some embodiments, the mannosidase may be expressed on the surface of the recombinant host cell.

In some embodiments, the recombinant host cell expresses a surface-displayed fusion protein may comprise a catalytic domain of a mannosidase and an anchoring domain of a glycosylphosphatidylinositol (GPI)-anchored protein, wherein the anchoring domain may comprise at least about 200 amino acids and/or at least about 30% of the residues in the anchoring domain are serines or threonines.

In some embodiments, the heterologous protein of interest may be secreted from the recombinant host cell.

In some embodiments, the secreted heterologous protein of interest may be an animal protein.

In some embodiments, the animal protein may be an egg protein.

In some embodiments, the egg protein may be selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In some embodiments, the recombinant host cell may comprise a further genomic modification that overexpresses a protein related to the p24 complex.

In some aspects, provided herein is a method for manufacturing a recombinant host cell for manufacturing a heterologous protein of interest having of a reduced level of exopolysaccharides. In some embodiments, the method comprises: obtaining a yeast cell engineered to express a heterologous protein of interest and/or a heterologous mannosidase; and modifying the yeast cell to underexpress two mannosyl transferases: beta-mannosyl transferase 1 (BMT1) and beta-mannosyl transferase 2 (BMT2) or functional homologues thereof.

In some aspects, provided herein is a method for manufacturing a recombinant host cell for manufacturing a heterologous protein of interest having of a reduced level of exopolysaccharides. The method may comprise: obtaining a yeast cell engineered to underexpress two mannosyl transferases: beta-mannosyl transferase 1 (BMT1) and beta-mannosyl transferase 2 (BMT2) or functional homologues thereof and engineered to express a heterologous mannosidase; and modifying the yeast cell to express a heterologous protein of interest.

In some aspects, provided herein is a method for manufacturing a recombinant host cell for manufacturing a heterologous protein of interest having of a reduced level of exopolysaccharides. In some embodiments, the method comprising: obtaining a yeast cell engineered to underexpress two mannosyl transferases: beta-mannosyl transferase 1 (BMT1) and beta-mannosyl transferase 2 (BMT2) or functional homologues thereof and engineered to express a heterologous protein of interest; and modifying the yeast cell to express a heterologous mannosidase.

In some aspects, provided herein is a method for manufacturing a recombinant host cell for manufacturing a heterologous protein of interest having of a reduced level of exopolysaccharides. In some embodiments, the method comprising: obtaining a yeast cell, modifying the yeast cell engineered to underexpress two mannosyl transferases: beta-mannosyl transferase 1 (BMT1) and beta-mannosyl transferase 2 (BMT2) or functional homologues thereof and engineered to express a heterologous protein of interest; modifying the yeast cell to express a heterologous protein of interest; and modifying the yeast cell to express a heterologous mannosidase.

In some aspects, provided herein are recombinant host cells for manufacturing a heterologous protein of interest. In some embodiments, the host cell may be a yeast cell. The host cell may be engineered to underexpress at least one polynucleotide encoding a mannosyl transferase or a functional homologue thereof compared to the host cell prior to genetic manipulation to achieve underexpression, wherein the host cell is engineered to express a heterologous protein of interest.

In some embodiments, the underexpression may be achieved by knocking-out the polynucleotide encoding the mannosyl transferase protein or a homologue thereof from the genome of said host cell, disrupting the polynucleotide encoding the mannosyl transferase protein or a homologue thereof in the host cell, disrupting a promoter which may be operably linked with said polynucleotide encoding the mannosyl transferase protein or a homologue thereof, replacing the promoter which may be operably linked with said polynucleotide encoding the mannosyl transferase protein or a homologue thereof with another promoter which has lower promoter activity, or disrupting expression control sequences of the mannosyl transferase protein or a homologue thereof, wherein the functional homologue has at least 70% sequence identity to an amino acid sequence of a mannosyl transferase.

In some embodiments, the host cell may be Pichia pastoris.

In some embodiments, the recombinant host cell expresses a mannosidase.

In some embodiments, the mannosidase may be heterologous to the host cell.

In some embodiments, the mannosidase may be expressed on the surface of the recombinant host cell.

In some embodiments, the protein of interest may be a nutritional protein.

In some embodiments, the mannosyl transferase may be a beta-mannosyl transferase.

In some embodiments, the beta-mannosyl transferase may be a protein sequence selected from the group consisting of XP_002493882.1, XP_002493883.1, XP_002490760.1, and XP_002493902.1.

In some embodiments, the mannosyl transferase may be a protein sequence selected from the group consisting of XP_002492593.1, XP_002490149.1, and XP_002493020.1.

In some embodiments, the host cell may be Pichia pastoris.

In some embodiments, the recombinant host cell expresses a mannosidase.

In some embodiments, the mannosidase may be heterologous to the host cell.

In some embodiments, the mannosidase may be expressed on the surface of the recombinant host cell.

In some embodiments, the protein of interest may be a nutritional protein.

In some aspects, provided herein are recombinant host cells for manufacturing a heterologous protein of interest. In some embodiments, the host cell may be a yeast cell. The host cell may be engineered to underexpress at least one polynucleotide encoding a protein from the Oligosaccharide Transferase complex or a functional homologue thereof compared to the host cell prior to genetic manipulation to achieve underexpression, wherein the host cell is engineered to express a heterologous protein of interest.

In some embodiments, the underexpression may be achieved by knocking-out the polynucleotide encoding a protein from the Oligosaccharide Transferase complex or a homologue thereof from the genome of said host cell, disrupting the polynucleotide encoding a protein from the Oligosaccharide Transferase complex or a homologue thereof in the host cell, disrupting a promoter which may be operably linked with said polynucleotide encoding a protein from the Oligosaccharide Transferase complex or a homologue thereof, replacing the promoter which may be operably linked with said polynucleotide encoding a protein from the Oligosaccharide Transferase complex or a homologue thereof with another promoter which has lower promoter activity, or disrupting expression control sequences of a protein from the Oligosaccharide Transferase complex or a homologue thereof, wherein the functional homologue has at least 70% sequence identity to an amino acid sequence of a protein from the Oligosaccharide Transferase complex.

In some embodiments, the host cell may be Pichia pastoris.

In some embodiments, the recombinant host cell expresses a mannosidase.

In some embodiments, the mannosidase may be heterologous to the host cell.

In some embodiments, the mannosidase may be expressed on the surface of the recombinant host cell.

In some embodiments, the protein of interest may be a nutritional protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the shift in the size of exopolysaccharides using gel electrophoresis after disruption of BMT1 and BMT2 genes which suggests that EPS is a form of mannan polysaccharide.

FIG. 2 illustrates the growth of P. pastoris strains using mannose as a sole carbon source.

FIG. 3 illustrates a chromatogram of purified EPS from the parent strain following 2 days of incubation with cells that express surface-displayed mannosidases. The size of the pure EPS byproduct is unchanged following incubation with cells.

FIG. 4 illustrates a chromatogram of EPS isolated from Strain 1 cells that express surface-displayed mannosidase enzymes. Strains show no discernable decrease in the concentration of EPS or size of the byproduct molecule.

FIG. 5 illustrates a chromatogram of EPS isolated from Strain 2 cell that express the surface-displayed mannosidase enzymes both cause a right shift in the elution profile of the EPS, suggesting a significant change in the size of the polysaccharide molecule.

FIG. 6 illustrates size exclusion chromatography of EPS samples. Strain 3 is Strain 1 after the deletion of 5 native P. pastoris mannosyltransferases.

FIG. 7 illustrates a general schematic for mannosidase surface display.

FIG. 8 illustrates size exclusion chromatography of EPS samples. By coupling the deletion of native mannosyltransferases with the expression of a surface-displayed B. thetaiotaomicron mannosidase, Strain 4 is able to reduce the size of the EPS byproduct.

FIG. 9 illustrates that disruption of native mannosyltransferases is important for B. theta enzymes to recognize mannan as a substrate for cleavage. The strains with deletions and mannosidase elicits the right-shift in the EPS elution profile.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

High-yielding recombinant protein expression is a cornerstone of various industries such as therapeutic proteins, food industry, cosmetics, etc. Recombinant protein expression though is almost always accompanied by impurities produced by the host cell. Each host cell generates and secretes proteins, carbohydrates, small molecules and polymers that must be separated from the protein of interest (POI) to produce a pure protein composition. The present invention addresses this need. The systems and methods provide high-titer expression of recombinant proteins in large scale production and are particularly useful for expressing pure heterologous animal derived proteins in a microbial host.

The present invention is concerned with the manipulation of genes related to the production of glycans in host cells. It has been surprisingly found that the manipulated host has an increased capacity to produce a significantly lower amount of exopolysaccharide impurities therefore reducing the amount of impurities produced by the cell while maintaining high-yield of recombinant proteins of interest.

In a first aspect, the preset invention provides a recombinant host cell for manufacturing a protein of interest, wherein the host cell is engineered to underexpress at least one, such as at least 2, or at least 3, polynucleotides encoding a mannosyl transferase, or a functional homologue thereof, wherein the functional homologue has at least 30% sequence identity to an amino acid sequence of these proteins.

For the purpose of the present invention the term “protein” is also meant to encompass functional homologues of the proteins described.

### Knockout (KO) Proteins

Yeast cells commonly produce highly complex and branched polysaccharides for various purposes such as enforcement for their cell walls. These complex polysaccharides include mannans with β-1,2-mannosyl linkages. It has not yet been suggested that an alteration in the mannan production pathways may lead to an increased purity of a recombinant protein produced in a yeast or other host cell. Inventors of the current application have discovered for the first time that the underexpression of one or more proteins in the mannosyl transferase pathway and/or the oligosaccharyltransferase (OST) pathway may lead to a reduction in size or amount of the glycans produced by the first cell thereby reducing exopolysaccharide impurities associated with recombinant proteins produced by host cells.

In some embodiments, a host cell engineered to underexpress one or more KO proteins reduces a concentration of exopolysaccharides produced by the host cell. A decrease in exopolysaccharide concentration can be determined when the exopolysaccharide concentration obtained from an engineered host cell is compared to the concentration obtained from a host cell prior to engineering, i.e., from a non-engineered host cell.

In some embodiments, a host cell engineered to underexpress one or more KO proteins alters the type of exopolysaccharides produced by the host cell. An alteration in exopolysaccharide concentration can be determined when the exopolysaccharide mass and/or form obtained from an engineered host cell is compared to the mass and/or form obtained from a host cell prior to engineering, i.e., from a non-engineered host cell.

In some embodiments, one or more proteins from the mannosyl transferase pathway are underexpressed in a host cell. The underexpression of one or more proteins from the mannosyl transferase pathway may lead to a reduced production of mannans in the host cell.

In one exemplary embodiment, one or more enzymes responsible for forming β-1,2-mannosyl linkages in cell wall mannan may be the KO proteins and may be underexpressed in a host cell. In this example, the mannan structure of the yeast may be altered to produce a reduced amount of the β-1,2-mannosyl linkages. Examples of such proteins include but are not limited to proteins encoded by genes such as BMT2 (SEQ ID NO: 13, XP_002493882.1), BMT1 (SEQ ID NO: 12, XP_002493883.1), BMT3 (SEQ ID NO: 14, XP_002490760.1), and BMT4 (SEQ ID NO: 15, XP_002493902.1), which code for enzymes responsible for forming β-1,2-mannosyl linkages.

In some embodiments, the host cell may be engineered to underexpress at least one mannosyl transferase enzyme, such as BMT1, BMT2, BMT3 or BMT4. In some embodiments, the host cell may be engineered to underexpress at least two mannosyl transferase enzymes. In some embodiments, the host cell may be engineered to underexpress at least three mannosyl transferase enzymes. In some embodiments, the host cell may be engineered to underexpress at least four mannosyl transferase enzymes.

In another exemplary embodiment, a host cell may be engineered to express a less complex mannan structure by underexpressing one or more KO proteins. In this example, a protein from the mannosyl transferase pathway, for instance a mannosyl transferase protein may be underexpressed to produce a linear mannan structure with «-1,6-linked mannose units. The α-1,6-linked mannose units may provide for an easier separation from the recombinantly produced POI. Examples of such proteins include but are not limited to proteins encoded by genes such as MNN2 (SEQ ID NO: 1, XP_002492593.1), MNN2 5 homolog 1 (SEQ ID NO: 2, XP_002490149.1), and MNN2 5 homolog 2 (SEQ ID NO: 3, XP_002493020.1).

In some embodiments, the host cell may be engineered to underexpress two mannosyl transferase enzymes. In one exemplary embodiment, the host cell may be engineered to underexpress BMT1 and BMT2. In one exemplary embodiment, the host cell may be engineered to underexpress one or more enzymes in addition to BMT1 and BMT2. In one example, the host cell may be engineered to underexpress one or more enzymes such as MNN2, MNN2/5 homolog 1 or MNN 2/5 homolog 2 in addition to BMT1 and BMT2.

In yet another exemplary embodiment, the one or more proteins underexpressed in a host cell may include proteins such as KTR1 (SEQ ID NO: 4, XP_002492424/GQ68_03227T0), KTR1 (alternative start site, SEQ ID NO: 5), KRE2 (SEQ ID NO: 6, XP_002492423/GQ68_03226T0) variant 1, KTR2 (SEQ ID NO: 7, XP_002492102/GQ68_00148T0), KTR3 (SEQ ID NO: 8, XP_002489479/GQ68_02855T0), KTR4 (SEQ ID NO: 9, XP_002490162/GQ68_02152T0), KTR5 (SEQ ID NO: 10, XP_002491999/GQ68_00252 T0), MNN4 (SEQ ID NO: 11, XP_002490538/GQ68_01768T0). Exemplary sequences for proteins that can be underexpressed are provided in Table 1. In some cases, the KO protein sequence may be at least 70% identical, at least 75% identical, at least 80% identical, at least 85% identical, at least 90% identical, or at least 95% identical, or at least 99% identical to one or more sequences in Table 1. In some exemplary embodiments, the host cell may be engineered to underexpress one or more enzymes such as KTR1, KRE2, KTR2, KTR3, KTR4, KTR5 and/or MNN4 in addition to BMT1 and BMT2.

In yet another exemplary embodiment, one or more proteins from the Asparagine Linked Glycolysis (ALG) pathway may be underexpressed in a host cell. In one more exemplary embodiment, one or more proteins from the Oligosaccharyltransferase (OST) may be underexpressed in the host cell. In one or more exemplary embodiments, the proteins in the ALG or OST pathway that may be underexpressed may include a protein with at least 70% identity, at least 75% identity, at least 80% identity, at least 85% identity, at least 90% identity, or at least 95% identity, or at least 99% identity to one or more sequences in Table 7.

In some embodiments, a host cell engineered to underexpress one or more KO proteins described herein does not negatively impact a yield of the POI produced by the host cell. In some embodiments, a host cell engineered to underexpress one or more KO proteins described herein increases a yield of the POI produced by the host cell. The term “yield” refers to the amount of POI or model protein(s) as described herein, which is, for example, harvested from the engineered host cell, and increased yields can be due to increased amounts of production or secretion of the POI by the host cell. Yield may be presented by mg POI/g biomass (measured as dry cell weight or wet cell weight) of a host cell. The term “titer” when used herein refers similarly to the amount of produced POI or model protein, presented as mg POI/L culture supernatant. An increase in yield can be determined when the yield obtained from an engineered host cell is compared to the yield obtained from a host cell prior to engineering, i.e., from a non-engineered host cell.

In some embodiments, the host cell is engineered to express at least 10%, 15%, 20%, 30%, 40%, 50%, 60%, 70%, 80%, 90%, or 95% less KO protein relative to a host cell which has not been engineered to underexpress said KO protein. In some embodiments, the host cell is engineered to knock out the KO protein, wherein the knockout leads to no activity of the KO protein in the host cell.

In some embodiments, the host cell is engineered to express at most 10%, 15%, 20%, 30%, 40%, 50%, 60%, 70%, 80%, 90%, or 95% KO protein relative to a host cell which has not been engineered to underexpress said KO protein.

### Host Cell

As used herein, a “host cell” refers to a cell which is capable of protein expression and optionally protein secretion. Such host cell is applied in the methods of the present invention. For that purpose, for the host cell to express a polypeptide, a nucleotide sequence encoding the polypeptide is present or introduced in the cell. Host cells provided by the present invention can be prokaryotes or eukaryotes. As will be appreciated by one of skill in the art, a prokaryotic cell lacks a membrane-bound nucleus, while a eukaryotic cell has a membrane-bound nucleus. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Examples of yeast cells include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces mandanus), the Candida genus (e.g. Candida utifis, Candida cacaoi), the Geotrichum genus (e.g. Geotrichum fermentans), as well as Hansenula polymorpha and Yarrowia fipolytica.

The genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris and Komagataella phaffii. Therefore, Pichia pastoris is synonymous for both Komagataella pastoris and Komagataella phaffii.

In some embodiments, the host cell is a Pichia pastoris, Hansenula polymorpha, Trichoderma reesei, Saccharomyces cerevisiae, Kluyveromyces lactis, Yarrowia lipolytica, Pichia methanolica, Candida boidinii, and Komagataella, and Schizosaccharomyces pombe.

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

As used herein, “engineered” host cells are host cells which have been manipulated using genetic engineering, i.e. by human intervention. When a host cell is “engineered to underexpress” a given protein, the host cell is manipulated such that the host cell has no longer the capability to express the protein described or a functional homologue thereof such as a non-engineered host cell.

“Prior to engineering” when used in the context of host cells of the present invention means that such host cells are not engineered such that a polynucleotide encoding a knockout (KO) protein or functional homologue thereof is underexpressed. Said term thus also means that host cells do not underexpress a polynucleotide encoding a KO protein or functional homologue thereof or are not engineered to underexpress a polynucleotide encoding a KO protein or functional homologue thereof.

The term “underexpression” includes any method that prevents or reduces the functional expression of a KO protein or functional homologues thereof. This results in the incapability or reduction to exert its known function. Means of underexpression may include gene silencing (e.g. RNAi genes antisense), knocking-out, altering expression level, altering expression pattern, by mutagenizing the gene sequence, disrupting the sequence, insertions, additions, mutations, modifying expression control sequences, and the like.

As mentioned herein, a host cell of the present invention is preferably engineered to underexpress a polynucleotide encoding a protein having an amino acid as defined herein. This includes that, if a host cell may have more than one copy of such a polynucleotide, also the other copies of such a polynucleotide are underexpressed. For example, a host cell of the present invention may not only be haploid, but it may be diploid, tetraploid or even more -ploid. Accordingly, in a preferred embodiment all copies of such a polynucleotide are underexpressed, such as two, three, four, five, six or even more copies.

The terms “underexpress,” “underexpressing,” “underexpressed” and “underexpression” in the present invention refer to an expression of a gene product or a polypeptide at a level less than the expression of the same gene product or polypeptide prior to a genetic alteration of the host cell or in a comparable host which has not been genetically altered. “Less than” includes, e.g., 10%, 20%, 30%, 40%, 50%, 60%, 70%, 80, 90% or more. No expression of the gene product or a polypeptide is also encompassed by the term “underexpression.”

### Features of Methods of the Present Disclosure

In some embodiments, the protein product having a reduced quantity of the exopolysaccharide impurities comprises an at least 50% reduction in exopolysaccharide impurities quantity relative to the composition comprising a recombinant protein of interest and exopolysaccharide impurities. In some cases, the POI product has an at least 75% reduction, at least 80% reduction, at least 90% reduction, or at least 95% reduction in exopolysaccharide impurities quantity relative to the composition comprising a recombinant POI and exopolysaccharide impurities.

In various embodiments, less than about 10% of the weight of the POI product comprises the exopolysaccharide impurities. In some cases, less than about 5% of the weight of the POI product comprises the exopolysaccharide impurities.

In embodiments, the exopolysaccharide impurities (EPS) is generally inseparable from the recombinant POI when using commonly used protein purification methods such as size exclusion chromatography.

In some embodiments, the EPS component is naturally a component of a recombinant cell's cell wall. In some cases, the EPS present in the composition comprising the recombinant POI was secreted from the recombinant cell rather than being incorporated into the recombinant cell's cell wall.

In various embodiments, the EPS has an apparent size of about 13 kDa to about 27 kDa as characterized by a size exclusion chromatography column.

In embodiments, the EPS comprises mannose. In some cases, the EPS further comprises N-acetylglucosamine and/or glucose.

In some embodiments, the EPS comprises about 91 mol % mannose, about 5 mol % N-acetylglucosamine, and about 3 mol % glucose as analyzed by gas chromatography in tandem with mass spectrometry. EPS can be quantified using a method using a pb binding column. An analytical HyperREZ XP Pb++ column (8 um, 300× 7.7 mm, Thermofisher Sci.) can be used for the measurement, which is eluted with water on UltiMate 3000 system (Thermofisher Sci.) operated at a flow rate of 0.6 mL/min and monitored with a refractive index detector.

In various embodiments, the EPS comprises an α(1,6)-linked backbone with α(1,2)-linked branches and/or α(1,3)-linked branches.

In embodiments, the EPS is a mannan.

In some embodiments, the recombinant cell is a cell that expresses and/or secretes EPS and is selected from a fungal cell, such as filamentous fungus or a yeast, a bacterial cell, a plant cell, an insect cell, or a mammalian cell.

### Methods of Underexpression

Preferably, underexpression is achieved by knocking-out the polynucleotide encoding the KO protein in the host cell. A gene can be knocked out by deleting the entire or partial coding sequence. Methods of making gene knockouts are known in the art, e.g., see Kuhn and Wurst (Eds.) Gene Knockout Protocols (Methods in Molecular Biology) Humana Press (Mar. 27, 2009). A gene can also be knocked out by removing part or all of the gene sequence. Alternatively, a gene can be knocked-out or inactivated by the insertion of a nucleotide sequence, such as a resistance gene. Alternatively, a gene can be knocked-out or inactivated by inactivating its promoter.

In an embodiment, underexpression is achieved by disrupting the polynucleotide encoding the gene in the host cell.

A “disruption” is a change in a nucleotide or amino acid sequence, which resulted in the addition, deletion, or substitution of one or more nucleotides or amino acid residues, as compared to the original sequence prior to the disruption.

An “insertion” or “addition” is a change in a nucleic acid or amino acid sequence in which one or more nucleotides or amino acid residues have been added as compared to the original sequence prior to the disruption.

A “deletion” is defined as a change in either nucleotide or amino acid sequence in which one or more nucleotides or amino acid residues, respectively, have been removed (i.e., are absent). A deletion encompasses deletion of the entire sequence, deletion of part of the coding sequence, or deletion of single nucleotides or amino acid residues.

A “substitution” generally refers to replacement of nucleotides or amino acid residues with other nucleotides or amino acid residues. “Substitution” can be performed by site-directed mutation, generation of random mutations, and gapped-duplex approaches (See e.g., U.S. Pat. No. 4,760,025; Moring et al., Biotech. (1984) 2:646; and Kramer et al., Nucleic Acids Res., (1984) 12:9441). Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et ai, 1990, Nucleic Acids Res. 18: 7349-4966. Site-directed mutagenesis can also be accomplished in vivo by methods known in the art. See, e.g., U.S. Patent Application Publication No. 2004/0171 154; Storici et ai, 2001, Nature Biotechnol. 19: 773-776; Kren et ai, 1998, Nat. Med. 4: 285-290; and Calissano and Macino, 1996, Fungal Genet. Newslett. 43: 15-16. Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips. Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al, 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127). Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide. Semisynthetic gene construction is accomplished by combining aspects of synthetic gene construction, and/or site-directed mutagenesis, and/or random mutagenesis, and/or shuffling. Semisynthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled. Alternatively, homologues can be obtained from a natural source such as by screening cDNA libraries of closely or distantly related microorganisms.

Preferably, disruption results in a frame shift mutation, early stop codon, point mutations of critical residues, translation of a nonsense or otherwise non-functional protein product.

In another embodiment, underexpression is achieved by disrupting the promoter which is operably linked with said polypeptide encoding the KO protein. A promoter directs the transcription of a downstream gene. The promoter is necessary, together with other expression control sequences such as ribosomal binding sites, transcriptional start and stop sequences, translational start and stop sequences, and enhancer or activator sequences, to express a given gene. Therefore, it is also possible to disrupt any of the expression control sequence to hinder the expression of the polypeptide encoding the KO protein.

A nucleic acid is “operably linked” when it is placed into a functional relationship with another nucleic acid sequence on the same nucleic acid molecule. For example, a promoter is operably linked with a coding sequence of a recombinant gene when it is capable of effecting the expression of that coding sequence.

In another embodiment, underexpression is achieved by post-transcriptional gene silencing (PTGS). A technique commonly used in the art, PTGS reduces the expression level of a gene via expression of a heterologous RNA sequence, frequently antisense to the gene requiring disruption (Lechtreck et al., J. Cell Sci (2002). 115:1511-1522; Smith et al., Nature (2000). 407:319-320; Furhmann et al., J. Cell Sci (2001). 114:3857-3863; Rohr et al., Plant J (2004). 40(4):611-21. Post-transcriptional gene silencing is a biological process in which RNA molecules inhibit gene expression, typically by causing the destruction of specific mRNA molecules using small RNAs including microRNA (miRNA), small interfering RNA (siRNA), or antisense RNA. Gene silencing can occur either through the blocking of transcription (in the case of gene-binding), the degradation of the mRNA transcript (e.g. by small interfering RNA (siRNA) or RNase-H dependent antisense), or through the blocking of either mRNA translation, pre-mRNA splicing sites, or nuclease cleavage sites used for maturation of other functional RNAs, including miRNA (e.g. by Morpholino oligos or other RNase-H independent antisense). These small RNAs can bind to other specific messenger RNA (mRNA) molecules and decrease their activity, for example by preventing an mRNA from producing a protein. Exemplary siRNA molecules have a length from about 10-50 or more nucleotides. The small RNA molecules comprise at least one strand that has a sequence that is “sufficiently complementary” to a target mRNA sequence to direct target-specific RNA interference (RNAi). Small interfering RNAs can originate from inside the cell or can be exogenously introduced into the cell. Once introduced into the cell, exogenous siRNAs are processed by the RNA-induced silencing complex (RISC). The siRNA is complementary to the target mRNA to be silenced, and the RISC uses the siRNA as a template for locating the target mRNA. After the RISC localizes to the target mRNA, the RNA can be cleaved by a ribonuclease. The strand has a sequence sufficient to trigger the destruction of the target mRNA by the RNAi machinery or process is commonly referred to as an antisense strand in the context of a ds-siRNA molecule. The siRNA molecule can be designed such that every residue is complementary to a residue in the target molecule. PTGS is found in many organisms. For yeast cells, the fission yeast, Schizosaccharomyces pombe, has an active RNAi pathway involved in heterochromatin formation and centromeric silencing (Raponi et al., Nucl. Acids Res. (2003) 31(15): 4481-4489). Some budding yeasts, including Saccharomyces cerevisiae, Candida albicans and Kluyveromyces polysporus were also found to have such RNAi pathway (Bartel et la., Science Express doi:10.1126/science. 1176945, published online 10 Sep. 2009). “Underexpression” can be achieved with any known techniques in the art which lowers gene expression. For example, the promoter which is operably linked with the polypeptide encoding the KO protein can be replaced with another promoter which has lower promoter activity. Promoter activity may be assessed by its transcriptional efficiency. This may be determined directly by measurement of the amount of mRNA transcription from the promoter, e.g. by Northern Blotting, quantitative PCR or indirectly by measurement of the amount of gene product expressed from the promoter.

Underexpression may in another embodiment be achieved by intervening in the folding of the expressed KO protein so that the KO protein is not properly folded to become functional. For example, mutation can be introduced to remove a disulfide bond formation of the KO protein or to disruption the formation of an alpha helices and beta sheets.

### Protein of Interest

The term “protein of interest” (POI) as used herein refers to a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of the promoter sequence. In general, the proteins of interest referred to herein may be produced by methods of recombinant expression well known to a person skilled in the art.

There is no limitation with respect to the protein of interest (POI). The POI is usually a eukaryotic or prokaryotic polypeptide, variant or derivative thereof. The POI can be any eukaryotic or prokaryotic protein. The protein can be a naturally secreted protein or an intracellular protein, i.e. a protein which is not naturally secreted. The present invention also includes biologically active fragments of proteins. In another embodiment, a POI may be an amino acid chain or present in a complex, such as a dimer, trimer, hetero-dimer, multimer or oligomer.

The protein of interest may be a protein used as nutritional, dietary, digestive, supplements, such as in food products, feed products, or cosmetic products. The food products may be, for example, bouillon, desserts, cereal bars, confectionery, sports drinks, dietary products or other nutrition products. Preferably, the protein of interest is a food additive. In some embodiments, the protein of interest if an animal-protein. In some exemplary embodiments, the protein of interest in an egg-white protein. In some examples, the protein of interest may include one or more proteins such as ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Exemplary POI sequences are provided in Table 5. In some cases, the POI sequence may be at least 70% identical, at least 75% identical, at least 80% identical, at least 85% identical, at least 90% identical, or at least 95% identical, or at least 99% identical to one or more sequences in Table 5.

In some cases, the protein of interest may be secreted from the host cell.

In some cases, a POI is produced in a host cell that has been engineered to express or overexpress one or more advantageous protein of interest (APOI). An APOI may be a protein that alters the type or form of glycans produced by the host cell. An APOI may be a protein that reduces glycan production by the host cell. An APOI may be a protein that reduces a type of glycan produced by the host cell. In some embodiments, APOIs may comprise hydrolase enzymes. In one example, APOIs may include mannosyl hydrolases and/or mannosidases. In some examples, the APOIs may comprise one or more helper factor proteins. Examples of such helper factor proteins may include proteins with SEQ ID NOs: 86-91.

One or more APOIs may be secreted from the host cell using a secretion signal. One or more APOIs may be expressed on the surface of the host cell. APOIs may be expressed on the surface of a host cell using conventional methods of surface display, including but not limited to chimeric linkages of the APOIs with surface display enzymes such as Sed1 (any one of SEQ ID NOs: 64-65), Tir4 (any one of SEQ ID NO: 58-61), Dan1 (any one of SEQ ID NOs: 62-63). Other surface display proteins that may be used are described in Table 4.

APOIs produced in the host cell may be proteins homologous to the host cell. Alternatively, APOIs produced in the host cell may be heterologous to the host cell. In one example, an APOI comprises a mannosidase such as produced by organisms including the common human gut microbe Bacteroides thetaiotaomicron. Exemplary APOIs include proteins with nucleotide sequences in Table 2 (SEQ ID NOs: 16-40) or protein sequences in Table 3 (SEQ ID NOs: 41-56, 86-91). In some cases, the APOI sequence may be at least 70% identical, at least 75% identical, at least 80% identical, at least 85% identical, at least 90% identical, or at least 95% identical, or at least 99% identical to one or more sequences in Table 2 or 3.

In one example, an APOI is a mannosidase which is capable of degrade any of the free altered mannan or exopolysaccharide structures into mannose monosaccharides which the cell can naturally import to use for carbon recovery.

### Surface Display of APOIs

APOIs or the advantageous proteins of interest such as a mannosidase can be displayed on the surface of the host cell. The APOIs displayed on the surface of the cell may be part of a fusion protein.

In some embodiments, an engineered eukaryotic cell may express a surface-displayed fusion protein comprising a catalytic domain of an APOI and an anchoring domain of a glycosylphosphatidylinositol (GPI)-anchored protein. In some cases, the anchoring domain comprises at least about 200 amino acids and/or at least about 30% of the residues in the anchoring domain are serines or threonines.

In some embodiments, the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In some embodiments, the serines or threonines in the anchoring domain are capable of being O-mannosylated.

In some embodiments, a fusion protein having an anchoring domain comprising at least about 325 amino acids provides greater enzymatic activity relative to a fusion protein having an anchoring domain comprising less than about 300 amino acids.

In some embodiments, a fusion protein having an anchoring domain comprising at least about 300 amino acids provides greater enzymatic activity relative to a fusion protein having an anchoring domain comprising less than about 250 amino acids.

In some embodiments, the fusion protein comprises the anchoring domain of the GPI anchored protein.

In some embodiments, the fusion protein comprises the GPI anchored protein without its native signal peptide.

In some embodiments, the GPI anchored protein is not native to the engineered eukaryotic cell.

In some embodiments, the GPI anchored protein is naturally expressed by a S. cerevisiae cell and the engineered eukaryotic cell is not a S. cerevisiae cell.

In some embodiments, the GPI anchored protein is selected from Tir4, Dan1, Dan4, Sag1, Fig2, or Sed1.

In some embodiments, the anchoring domain of the GPI anchored protein comprises an amino acid sequence that is at least 70% identical, at least 75% identical, at least 80% identical, at least 85% identical, at least 90% identical, or at least 95% identical, to one or more sequences in Table 4.

In some embodiments, the anchoring domain of the GPI anchored protein comprises an amino acid sequence of one or more sequences in Table 4.

In some embodiments, the fusion protein comprises a portion of the APOI in addition to its catalytic domain.

In some embodiments, the fusion protein comprises substantially the entire amino acid sequence of the APOI.

In some embodiments, the fusion protein, the catalytic domain is N-terminal to the anchoring domain.

In some embodiments, the fusion protein comprises a linker between the catalytic domain and the anchoring domain.

In some embodiments, upon translation, the fusion protein comprises a signal peptide and/or a secretory signal.

In some embodiments, the engineered eukaryotic cell comprises two or more fusion proteins, three or more fusion proteins, or four fusion proteins.

In some embodiments, the two or more fusion proteins comprise different enzyme types.

In some embodiments, the two or more fusion proteins comprise the same enzyme type.

In some embodiments, the two of the three or more fusion proteins or two of the four or more fusion proteins comprise different enzyme types.

In some embodiments, the two of the three or more fusion proteins or two of the four or more fusion proteins comprise the same enzyme type. In some embodiments, the three of the three or more fusion proteins or three of the four or more fusion proteins comprise different enzyme types. In some embodiments, the three of the three or more fusion proteins or three of the four or more fusion proteins comprise the same enzyme type. In some embodiments, the each of the two or more, three or more, or four fusion proteins comprise different enzyme types. In some embodiments, the each of the two or more, three or more, or four fusion proteins comprise the same enzyme type.

### Expression of Proteins

Expression of a recombinant protein such as the POI or the APOI can be provided by an expression vector, a plasmid, a nucleic acid integrated into the host genome or other means. For example, a vector for expression can include: (a) a promoter element, (b) a signal peptide, (c) a heterologous protein sequence, and (d) a terminator element.

Expression vectors that can be used for expression of a recombinant POI or APOI include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aid in the amplification of the vector prior to transformation into the host microorganism, a replication origin (c) may be contained in the vector (such as PUC_ORIC and PUC (DNA2.0)). To aide in the selection of microorganism stably transformed with the expression vector, the vector may also include a selection marker (f) such as URA3 gene and Zeocin resistance gene (ZeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (c), (f), and (g). Other expression elements and vector element known to one of skill in the art can be used in combination or substituted for the elements described herein.

Exemplary promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof. Illustrative inducible promoters include methanol-induced promoters, e.g., DAS1 and pPEX11.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native a recombinant POI or APOI.

Any nucleic acid sequence that encodes a recombinant POI or APOI can be used as (c). Preferably such sequence is codon optimized for the species/genus/kingdom of the host cell.

Exemplary transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

Exemplary selectable markers (f) may include but are not limited to: an antibiotic resistance gene (e.g. zeocin, ampicillin, blasticidin, kanamycin, nourseothricin, chloroamphenicol, tetracycline, triclosan, ganciclovir, and any combination thereof), an auxotrophic marker (e.g. ade1, arg4, his4, ura3, met2, and any combination thereof).

In one example, a vector for expression in Pichia sp. can include an AOX1 promoter operably linked to a signal peptide (alpha mating factor) that is fused in frame with a nucleic acid sequence encoding a recombinant POI or APOI, and a terminator element (AOX1 terminator) immediately downstream of the nucleic acid sequence encoding a recombinant POI or APOI.

In another example, a vector comprising a DAS1 promoter is operably linked to a signal peptide (alpha mating factor) that is fused in frame with a nucleic acid sequence encoding a recombinant POI or APOI and a terminator element (AOX1 terminator) immediately downstream of a recombinant POI or APOI.

A recombinant protein described herein may be secreted from the one or more host cells. In some embodiments, a recombinant POI protein is secreted from the host cell. The secreted a recombinant POI may be isolated and purified by methods such as centrifugation, fractionation, filtration, affinity purification and other methods for separating protein from cells, liquid and solid media components and other cellular products and byproducts. In some embodiments, a recombinant POI is produced in a Pichia Sp. and secreted from the host cells into the culture media. The secreted a recombinant POI is then separated from other media components for further use.

In some cases, multiple vectors comprising the gene sequence of a POI and/or APOI may be transfected into one or more host cells. A host cell may comprise more than one copy of the gene encoding the POI and/or APOI. A single host cell may comprise 2, 3, 4, 5, 6, 7, 8, 9, 10, 11, 12, 13, 14, 15, 16, 17, 18, 19 or 20 copies of the POI and/or APOI. A single host cell may comprise one or more vectors for the expression of the POI and/or APOI. A single host cell may comprise 2, 3, 4, 5, 6, 7, 8, 9 or 10 vectors for the POI and/or APOI expression. Each vector in the host cell may drive the expression of POI using the same promoter. Alternatively, different promoters may be used in different vectors for POI expression.

A recombinant POI or APOI may be recombinantly expressed in one or more host cells. As used herein, a “host” or “host cell” denotes here any protein production host selected or genetically modified to produce a desired product. Exemplary hosts include fungi, such as filamentous fungi, as well as bacteria, yeast, plant, insect, and mammalian cells. A host cell can be an organism that is approved as generally regarded as safe by the U.S. Food and Drug Administration.

A host cell may be transformed to include one or more expression cassettes. As examples, a host cell may be transformed to express one expression cassette, two expression cassettes, three expression cassettes or more expression cassettes. In one example, a host cell is transformed express a first expression cassette that encodes a first POI and express a second expression cassette that encodes a second POI.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

TABLE 1
Exemplary proteins for underexpression

| SEQ | | |
|---|---|---|
| ID | Sequence | |
| NO. | Info | Amino acid sequence |
| 1 | MNN2 | MFGKRRQVRKLLIWVVLLLIVYFFGLQFRA |
| | (XP_002492593/ | KNSAHQSSIRSFYADNKEFFDRQYSRYDEY |
| | GQ68_03403T0) | DIIDNMNSHNELLQEQFRNGKLAAGLRGVA |
| | | EEPNSDEVTDDTAIEEDEQAAMINFPKRSP |
| | | QREKSLVELRKFYKNVLSIIINNKPAMPIE |
| | | NPRDPTPNENALKRKFGKSGIINIALHDTD |
| | | PSLPILSEAYLRDSLQLSPSFIASLSKSHS |
| | | AVVKAFPPSFPANAYNGTGIVFIGGQKFSW |
| | | LSLLSIENLRKTGSKVPVELIIPFAHEYEP |
| | | QLCEEILPKLNATCVLLQETVGIDLLKSGH |
| | | LKGYQFKSLALLASSFEQVLLVDSDNIIVE |
| | | NPDPIFDSEVFQRTGLVLWPDFWRRVTHPD |
| | | YYKIAGIKLGSERVRHVVDSYTDPSLYTSS |
| | | SEDPFTDIPLHDREGAIPDGSTESGQILIS |
| | | KTKHCQTILLSLYYNFFGPDYYYPLFTQGA |
| | | SGEGDKETFLAAANYYKLPFYNIKKGVDVI |
| | | GYWKPDQSAYQGCGMLQYDPIVDYQNLQTF |
| | | LKTHKGSRVNKLEQSELDKPGLLSRLIPKF |
| | | FFRKTFDEHQLQSHFTKDRSKIMFIHSNFP |
| | | KLDPFGLKLHNYLFVDQDTHKPRIRMYADQ |
| | | TGLSFDFELRQWIIIHEYFCEYPDFNLKYL |
| | | ENANVKPQDLCMFIKEELNFLQNNPIQLT |
| 2 | MNN2/5 | MLFGLIRHSRRQLLFLGALVTVIVLIFTLP |
| | homolog | NTSPIEANGVKSEEGSITPIIPVLESPANS |
| | 1-MNNF1 | LEKIVDTASEERIGGATLEEGHENNKEEQA |
| | (XP_002490149/ | LENAERAKEKEKTEAIAAEEEKLKAAELLR |
| | GQ68_02166T0) | QQETTREKEAAKEDDSKKPNQELVEQDTYL |
| | | DDIPDDVEDNIIISEQDRKKIILPSYTPKT |
| | | DPAYSKRATALKIFYNDFFIKVADSGPNTA |
| | | PITKKTRKKGKSKLKGDVSSGDKYEGPVLT |
| | | EDFLRFMEIYSDEFIDAVSESHSKIVNLMP |
| | | ESFPKGMYQGDGIVIIGGGVYSWYGLLAIR |
| | | NLRDGGNTLPVELMLPSDNEYEPQLCEQIL |
| | | PSLNAKCIMLSDIVDQDVLKKLDFKGYQFK |
| | | ALSLLASSFENVLSLDSDNIPVANVSHLFD |
| | | HEPFSETGLVSWPDFWRRTTNPRYYEAAGI |
| | | KIGEYQVRNCLDGFVPESDFVHIGLKDIPL |
| | | HDRNGTIPDASTESGQLLVNKNKHAKTLML |
| | | MFYYNFYGPGYYYPLLSQGMAGEGDKETFL |
| | | AAANFFGLPFYQVKAGPGILGHHDSTGAFT |
| | | GVAIVQYDPIADYELTKENFVGEKRKGIEA |
| | | PKAFYGNNNKSPLFHHCNFPKLDPVKLIKE |
| | | KKLIDNKTHKFNRMYGPNTKLKYDFEERQW |
| | | KYTKEYLCEKKYNLLYFTEQYKNYGQGYSQ |
| | | ERICKFSDRFLKFLSDNPIRIEG |
| 3 | MNN2/5 | MFNSLAPMRLKKLLKVFCASVVLLAATSVV |
| | homolog | LFFHFGGQIIIPIPERTVTLSTPPANDTWQ |
| | 2-MNNF2 | FQQFFNGYLDALLENNLSYPIPERWNHEVT |
| | (XP_002493020/ | NVRFFNRIGELLSESRLQELIHFSPEFIED |
| | GQ68_03863T0) | TSDKFDNIVEQIPAKWPYENMYRGDGYVIV |
| | | GGGRHTFLALLNINALRRAGNKLPVEVVLP |
| | | TYDDYEEDFCENHFPLLNARCVILEERFGD |
| | | QVYPRLQLGGYQFKIFAIAASSFKNCFLLD |
| | | SDNIPLRKMDKIFSSELYKNKTMITWPDFW |
| | | LRSTSPHYYHNITKTPIGDKRVRYFNDFYT |
| | | NPNEYYYGDEDPRSEIPFHDREGTIPDWTT |
| | | ESGQLVINKEVHFPAILLGLFYNFNGPMGF |
| | | YPLLSQGGAGEGDKDTFVAASHYYNLPYYQ |
| | | VYKNCEMLYGWVDHANSGRIEHSAIVQYNP |
| | | IVDYENLQSVKAKAEIILKNHEPDSRKKSS |
| | | KPKSYSKTRLSTHVKGSIYSYRRLFRDSFN |
| | | KANSDEMFLHCHTPKIEPYRIMEDDLTLGR |
| | | NKEAKQRWYGGRKNRVRFGYDVELYIWELI |
| | | DQYICDKNIQYKIFEGKDRDALCGSFMREQ |
| | | LGFLRSTGD |
| 4 | KTR1 | MELVRLANLVNVNHPFBQSNIYRVPLFFLL |
| | (XP_002492424/ | STTRPDRTTVQMAGATRINSRVVRFAIFAS |
| | GQ68_03227T0) | ILVLLGFILSRGSATSYSLPSGLTSDTSQS |
| | | TGSSPKSESKPSSQGSSGATELKKTYTTDG |
| | | KEKATFVSLARNSDVWSLASSIRHVEDRFN |
| | | HKFHYDWVFLNDEEFSDEFKRVTSALTSGK |
| | | AKYGLIPKEHWSFPEWIDKERAAKTRKEMA |
| | | AKKVIYGDSISYRHMCRFESGFFFRHELMQ |
| | | EYEWYWRVEPDIKIYCDIDYDVFKFMKDNN |
| | | KMYGFTVSLPEYVATIETLWDTTRAFIKEN |
| | | PQYLPEDNMMDFISDDDGLSYNGCHFWSNF |
| | | EVGSLSLWRSEAYLKYFDHLDKAGGFFYER |
| | | WGDAPVHSIAAALFLHRDQIHFFDDVGYFH |
| | | NPFNNCPVDADLREERRCMCNPKDDFTWKG |
| | | YSCVPEFFTVNNMKRPKGWEAFSG |
| 5 | KTR1 | SNIYRVPLFFLLSTTRPDRTTVQMAGATRI |
| | (alternative | NSRVVRFAIFASILVLLGFILSRGSATSYS |
| | startsite) | LPSGLTSDTSQSTGSSPKSESKPSSQGSSG |
| | | ATELKKTYTTDGKEKATFVSLARNSDVWSL |
| | | ASSIRHVEDRFNHKFHYDWVFLNDEEFSDE |
| | | FKRVTSALTSGKAKYGLIPKEHWSFPEWID |
| | | KERAAKTRKEMAAKKVIYGDSISYRHMCRF |
| | | ESGFFFRHELMQEYEWYWRVEPDIKIYCDI |
| | | DYDVFKFMKDNNKMYGFT |
| | | VSLPEYVATIETLWDTTRAFIKENPQYLPE |
| | | DNMMDFISDDDGLSYNGCHFWSNFEVGSLS |
| | | LWRSEAYLKYFDHLDKAGGFFYERWGDAPV |
| | | HSIAAALFLHRDQIHFFDDVGYFHNPFNNC |
| | | PVDADLREERRCMCNPKDDFTWKGYSCVPE |
| | | FFTVNNMKRPKGWEAFSG |
| 6 | KRE2 | MTGCFLNEVPFTDEFKERTSVLISGQAKYG |
| | (XP_002492423/ | LIPKEHWSYPDYIDQERAAESRRQLEDQHV |
| | GQ68_03226T0) | VYGGLESYRHMCRFNSGFFYKHPLMLDYRY |
| | variant 1 | YWRVEPEIEILCDVETDLFRYMRENNKTYG |
| | | FTISIHEFEKTIPTLWETTKEFMKQNPSYI |
| | | AENNLMNFISDDNGKTYNLCHFWSNFEVAD |
| | | MDFWRSDVYEKYFKFLDDTGKFFYERWGDA |
| | | PVHSLAVSLFLPKEKVHFFNEVGYKHSVYS |
| | | MCPIDKDIWKNRKCYCDPNTDFTFRGYSCG |
| | | RQYYKATGLTRPSNWKDYD |
| 7 | KTR2 | MKVVWLACFIILAAIWYKDYQSLRGFMDDR |
| | (XP_002492102/ | VSKTLPINFNALKLSTNSYIPVDEHLIKPN |
| | GQ68_00148T0) | REPNPKFVKENATLLMLCRNWELEEVLQSM |
| | | RSLEDRFNGRYQYTWTFLNDVPFEKQFIQE |
| | | TTLMASGKTQYALISSTDWNRPSFINETRF |
| | | EQNLIQSEKDDIIYGGSPSYRNMCRFNSGF |
| | | FYKQKILDQYDYYFRVEPGVEYFCDLEEDP |
| | | FRYMRLHDKKYGFVISLYEYENTIPTLWQT |
| | | VEKFIENHPEYIHPNNSYEFLTDKEVVGPL |
| | | GLVALTEQTYNLCHFWSNFEIGDLNFFRSE |
| | | KYEAFFQFLDQAGGFYYERWGDAPVHSIAV |
| | | GILLDKRQIHHFENIGYYHLPFSTCPQSYW |
| | | SYKCNRCICKRNESIDLVPHSCLSKWWKYG |
| | | GKTFLQ |
| 8 | KTR3 | MMRARLSLERVNLSFITSVFLASVAVLFIS |
| | (XP_002489479/ | LEMPKVLARDRQILKLKLGFMGSGLQKGSL |
| | GQ68_02855T0) | ETSGNIENTESNINSQTTQHIGTIGASNER |
| | | ANATFYTLCRNEELYQMLETVQNYEDRFNS |
| | | KFKYDWVFLNDYPFTDEFKRVISHAISGEA |
| | | KFGQVPASHWRFPDHIDQQKVYESMDKMDS |
| | | DNTTGDYLGLPIPYAKSISYRHMCRYQSGF |
| | | FYKHGLLQGYKYFWRVEPDVKLYCDIDYDV |
| | | FKSMEQNGKRYGFVISMMEFEKTIESLFKE |
| | | VKNYLQMKGVSRLLEDTDNLSDFVYDELSG |
| | | DYTLCHFWSNFEIGDLDFFRGREYNEFFDY |
| | | LDSKGGFYYERWGDAPIHSIAVSLFMQWND |
| | | VKWFSDIGYRHPPYLSCPLSEEVRLEKKCS |
| | | CDPKQDFTMDAYSCTRFYQDIIRDKQKSQG |
| | | SNP |
| 9 | KTR4 | MMISLTKRFTKLAIFGSLSFILTTAGLWLY |
| | (XP_002490162/ | WDAIQYMMTSGKIPTLDFQFEDFMNRHDDI |
| | GQ68_02152T0) | VDDMMKKYDKIMKAEVKEPNVGNLVYAPES |
| | | LVDYGRENATLLMLVRNKELRTALQAIETV |
| | | ESQFNHKFQYPYVFLNDKEFTDKFKSTITE |
| | | KVSGQVFFETIDKVTWDRPDWIDSAKESER |
| | | IKVMRKYNVGYADKLSYHNMCRYYSRGFYN |
| | | HPRLQQFKYYWRFEPGTHYHTSIDYDVFKF |
| | | MSANDKTYGFVISLYDTERSIETLWPETLK |
| | | FIEQNPQFVNKNAAWDWLTEKKQNPQKTRI |
| | | ANGYSTCHFWSNFEIGDMDFFRSEAYTKWV |
| | | NHLDATGGFYYERWGDAPVHSIGATLFQDK |
| | | SKVHWFRDIGYYHAPYYQCPNSPQSDGKCE |
| | | VGKFSFPNLSDQNCLINWIEMVADNELSMY |
| 10 | KTR5 | MSFRLGYIQAIVLGLVLLSVCWTIVIRPDP |
| | (XP_002491999/ | SSAIDLASPVTIDLENSLTNLKSFPISSRR |
| | GQ68_00252T0) | ISSNIDHVFQTGCRNVFKNKKKANAALVVL |
| | | ARNSELEGVQKSMFSMERHFNQWFNYPWIF |
| | | LNDEEFTESFKDGVMNMTSSGVSFGVISKP |
| | | DWNFSEEKDRGSTEFLRFNEFIQNQGDRGI |
| | | MYGALPSYHKMCRFYSGYFFKHPLVAKLSW |
| | | YWRVEPDVEFFCDLTYDPFLEMEASGKKYG |
| | | FAVIIKELSNTVPNLFRHTQSFIEKYGISV |
| | | DEKAWSIFTNRRSFGEKESMKLIDKIRINH |
| | | LLSNFSGGIGTRLLSSLSRMNLPTSFSSKK |
| | | PFFYGEEYNLCHFWSNFEIASTDLFSSPEY |
| | | ESYFQFLEEKKGFYQERWGDAPVHSLAVAM |
| | | FLNISEIHYFRDIGYRHSNLVHCPKNAPDE |
| | | LQLPYVPASPEYASSAKPDKPPRVSVRDVF |
| | | RSGRQTEGVNNLNRGSGCRCNCPKKYKELE |
| | | DSPSCCIGRWMVLTNDKYKGEKYLDKYSMA |
| | | EEVKQTLSKGEKLNVKEILKRHHKYPT |
| 11 | MNN4 | MKVSKRLIPRRSRLLIMMMLLVVYQLVVLV |
| | (XP_002490538/ | LGLESVSEGKLASLLDLGDWDLANSSLSIS |
| | GQ68_01768T0) | DFIKLKLKGQKTYHKFDEHVFAAMARIQSN |
| | | ENGKLADYESTSSKTDVTIQNVELWKRLSE |
| | | EEYTYEPRITLAVYLSYIHQRTYDRYATSY |
| | | APYNLRVPFSWADWIDLTALNQYLDKTKGC |
| | | EAVFPRESEATMKLNNITVVDWLEGLCITD |
| | | KSLQNSVNSTYAEEINSRDILSPNFHVFGY |
| | | SDAKDNPQQKIFQSKSYINSKLPLPKSLIF |
| | | LTDGGSYALTVDRTQNKRILKSGLLSHFFS |
| | | KKKKEHNLPQDQKTFTFDPVYEFNRLKSQV |
| | | KPRPISSEPSIDSALKENDYKLKLKESSFI |
| | | FNYGRILSNYEERLESLNDFEKSHYESLAY |
| | | SSLLEARKLPKYFGEVILKNPQDGGIHYDY |
| | | RFFSGLIDKTQINHFEDETERKKIIMHRLL |
| | | RTWQYFTYHNNIINWISHGSLLSWYWDGLS |
| | | FPWDNDIDVQMPIMELNNFCKQFNNSLVVE |
| | | DVSQGFGRYYVDCTSFLAQRTRGNGNNNID |
| | | ARFIDVSSGLFIDITGLALTGSTMPKRYSN |
| | | KLIKQPKKSTDSTGSTPENGLTRNLRQNLN |
| | | AQVYNCRNGHFYQYSELSPLKLSIVEGALT |
| | | LIPNDFVTILETEYQRRGLEKNTYAKYLYV |
| | | PELRLWMSYNDIYDILQGTNSHGRPLSAKT |
| | | MATIFPRLNSDINLKKFLRNDHTFKNIYST |
| | | FNVTRVHEEELKHLIVNYDQNKRKSAEYRQ |
| | | FLENLRFMNPIRKDLVTYESRLKALDGYNE |
| | | VEELEKKQENREKERKEKKEKEEKEKKEKE |
| | | EKEKKEKEEKEKKEKEEKERKEKEEKEEYE |
| | | EDDNEGEQPTEQKSQQEAKE |
| 12 | BMT1 | MVDLFQWLKFYSMRRLGQVAITLVLLNLFV |
| | (XP_002493883/ | FLGYKFTPSTVIGSPSWEPAVVPTVFNESY |
| | GQ68_04782T0) | LDSLQFTDINVDSFLSDTNGRISVTCDSLA |
| | | YKGLVKTSKKKELDCDMAYIRRKIFSSEEY |
| | | GVLADLEAQDITEEQRIKKHWFTFYGSSVY |
| | | LPEHEVHYLVRRVLFSKVGRADTPVISLLV |
| | | AQLYDKDWNELTPHTLEIVNPATGNVTPQT |
| | | FPQLIHVPIEWSVDDKWKGTEDPRVFLKPS |
| | | KTGVSEPIVLFNLQSSLCDGKRGMFVTSPF |
| | | RSDKVNLLDIEDKERPNSEKNWSPFFLDDV |
| | | EVSKYSTGYVHFVYSFNPLKVIKCSLDTGA |
| | | CRMIYESPEEGRFGSELRGATPMVKLPVHL |
| | | SLPKGKEVWVAFPRTRLRDCGCSRTTYRPV |
| | | LTLFVKEGNKFYTELISSSIDFHIDVLSYD |
| | | AKGESCSGSISVLIPNGIDSWDVSKKQGGK |
| | | SDILTLTLSEADRNTVVVHVKGLLDYLLVL |
| | | NGEGPIHDSHSFKNVLSTNHFKSDTTLLNS |
| | | VKAAECAIFSSRDYCKKYGETRGEPARYAK |
| | | QMENERKEKEKKEKEAKEKLEAEKAEMEEA |
| | | VRKAQEAIAQKEREKEEAEQEKKAQQEAKE |
| | | KEAEEKAAKEKEAKENEAKKKIIVEKLAKE |
| | | QEEAEKLEAKKKLYQLQEEERS |
| 13 | BMT2 | MRTRLNFLLLCIASVLSVIWIGVLLTWNDN |
| | (XP_002493882/ | NLGGISLNGGKDSAYDDLLSLGSENDMEVD |
| | GQ68_04781T0) | SYVTNIYDNAPVLGCTDLSYHGLLKVTPKH |
| | | DLACDLEFIRAQILDIDVYSAIKDLEDKAL |
| | | TVKQKVEKHWFTFYGSSVFLPEHDVHYLVR |
| | | RVIFSAEGKANSPVTSIIVAQIYDKNWNEL |
| | | NGHFLDILNPNTGKVQHNTFPQVLPIATNF |
| | | VKGKKFRGAEDPRVVLRKGRFGPDPLVMFN |
| | | SLTQDNKRRRIFTISPFDQFKTVMYDIKDY |
| | | EMPRYEKNWVPFFLKDNQEAVHFVYSFNPL |
| | | RVLKCSLDDGSCDIVFEIPKVDSMSSELRG |
| | | ATPMINLPQAIPMAKDKEIWVSFPRTRIAN |
| | | CGCSRTTYRPMLMLFVREGSNFFVELLSTS |
| | | LDFGLEVLPYSGNGLPCSADHSVLIPNSID |
| | | NWEVVDSNGDDILTLSFSEADKSTSVIHIR |
| | | GLYNYLSELDGYQGPEAEDEHNFQRILSDL |
| | | HFDNKTTVNNFIKVQSCALDAAKGYCKEYG |
| | | LTRGEAERRRRVAEERKKKEKEEEEKKKKK |
| | | EKEEEEKKRIEEEKKKIEEKERKEKEKEEA |
| | | ERKKLQEMKKKLEEITEKLEKGQRNKEIDP |
| | | KEKQREEEERKERVRKIAEKQRKEAEKKEA |
| | | EKKANDKKDLKIRQ |
| 14 | BMT3 | MRIRSNVLLLSTAGALALVWFAVVFSWDDK |
| | (XP_002490760/ | SIFGIPTPGHAVASAYDSSVTLGTFNDMEV |
| | GQ68_01534T0) | DSYVTNIYDNAPVLGCYDLSYHGLLKVSPK |
| | | HEILCDMKFIRARVLETEAYAALKDLEHKK |
| | | LTEEEKIEKHWFTFYGSSVFLPDHDVHYLV |
| | | RRVVFSGEGKANRPITSILVAQIYDKNWNE |
| | | LNGHFLNVLNPNTGKLQHHAFPQVLPIAVN |
| | | WDRNSKYRGQEDPRVVLRRGRFGPDPLVME |
| | | NTLTQNNKLRRLFTISPFDQYKTVMYRTNA |
| | | FKMQTTEKNWVPFFLKDDQESVHFVYSFNP |
| | | LRVLNCSLDNGACDVLFELPHDFGMSSELR |
| | | GATPMLNLPQAIPMADDKEIWVSFPRTRIS |
| | | DCGCSETMYRPMLMLFVREGTNFFAELLSS |
| | | SIDFGLEVIPYTGDGLPCSSGQSVLIPNSI |
| | | DNWEVTGSNGEDILSLTFSEADKSTSVVHI |
| | | RGLYKYLSELDGYGGPEAEDEHNFQRILSD |
| | | LHFDGKKTIENFKKVQSCALDAAKAYCKEY |
| | | GVTRGEEDRLKNKEKERKIEEKRKKEEERK |
| | | KKEEEKKKKEEEEKKKKEEEEEEEKRLKEL |
| | | KKKLKELQEELEKQKDEVKDTKAK |
| 15 | BMT4 | MYHLAPRKKLLIWGGSLGFVLLLLIVASSH |
| | (XP_002493902/ | QRIRSTILHRTPISTLPVISQEVITADYHP |
| | GQ68_04802T0) | TLLTGFIPTDSDDSDCADFSPSGVIYSTDK |
| | | LVLHDSLKDIRDSLLKTQYKDLVTLEDEEK |
| | | MNIDDILKRWYTLSGSSVWIPGMKAHLVVS |
| | | RVMYLGTNGRSDPLVSFVRVQLFDPDFNEL |
| | | KDIALKFSDKPDGTVIFPYILPVDIPREGS |
| | | RWLGPEDAKIAVNPETPDDPIVIFNMQNSV |
| | | NRAMYGFYPFRPENKQVLFSIKDEEPRKKE |
| | | KNWTPFFVPGSPTTVNFVYDLQKLTILKCS |
| | | IITGICEKEFVSGDDGQNHGIGIFRGGSNL |
| | | VPFPTSFTDKDVWVGFPKTHMESCGCSSHI |
| | | YRPYLMVLVRKGDFYYKAFVSTPLDFGIDV |
| | | RSWESAESTSCQTAKNVLAVNSISNWDLLD |
| | | DGLDKDYMTITLSEADVVNSVLRVRGIAKF |
| | | VDNLTMDDGSTTLSTSNKIDECATTGSKQY |
| | | CQRYGELH |
| 72 | CCW12homolog | MLTKVISLAILTASAFADSGEFTLWNLSPG |
| | (GQ68_04433) | DPYDSTFWGVSEGLIVPVEPGVTFVITDDL |
| | (PAS_chr4_ | QLKTTDDQFVTVGEDSALGLGAEGSVEFSI |
| | 0151) | INEDGITSLYYNGELVTAYICEGAEPQIYL |
| | | TGSEEDPECVSYTVAVIGVDGEAPPTFPEE |
| | | DDETTTTDDPTDEPTDEPTDEPTDEPTDEP |
| | | TDEPTDEPTDEPTDEPTDEPTDEPTDEPTD |
| | | EPTDEPTDEPTDEPTEEPTEEPTEEPTDEP |
| | | TPPPPHWGNETVTATKTEYETTKVTITSCE |
| | | ETKCYETTSDAWVSTCTTEIGGKVTKIVTW |
| | | CPIPSTPGPKPPKPTKPTETKPTTVPAPTT |
| | | KKPETPTTKKPETPAPEKPEKTTTVIPPPT |
| | | TEKPSTLSTSSVTGSVTIPTITATGGAGSN |
| | | FNLGGLTVGVAGIAMALFV |
| 73 | CCW12homolog | MFEKSKFVVSFLLLLQLFCVLGVHGQESGN |
| | GQ68_01574 | GTTSDTAYACDIGATPFDGFNATIYQYQAS |
| | (chr1) | DDNSIQDPVFMSTGYLQRNQLHSTTGVTNP |
| | | GFNIFTAGVATTTLYGIPNVNYQNMLLELK |
| | | GYFRADASGNYGLSLRNIDDSAILFFGRET |
| | | AFECCNENLIPLDEAPTDYSLFTIKEGEAS |
| | | TNPDSYTYTQYLEAGRYYPVRTFFANIRTR |
| | | AVFNFTMTLPDGSELTDFQNYIFQFGALNQ |
| | | QQCQAEIVTRENYTTTTEPWTGTFEATTTV |
| | | IPSGTEPGTVIVQTPYSTIDSTSTWTGTFT |
| | | TFTTDADGSTIAVVPSSTIDDHFASTETVL |
| | | TDTAISTTVITVTSCGTSKCTKTTALTGVT |
| | | QRTLTIDDRTTVVTTYCPLPTDVATIKTAS |
| | | VSGSEVVQTIYTAKHSQAVSYVHPSTVTIT |
| | | REVCDAQTCTQATIVTGEILQTTVVDSGST |
| | | TVVPKYVPVETHEPTFELSTL |
| 74 | CCW14homolog | MQFTFASTSVVVSLIAALAKPAVATPPACL |
| | GQ68_01658 | LACAAEVVKESSDCDALNNIQCICENEGSA |
| | (PAS_chr1- | IHACLESTCPDGLSSTALQSFEDVCESVGT |
| | 4_0510) | EANLDESSSSQSSSSSSSSESSSSSVSSSS |
| | | SSASSSSETSSSVTSSSVTSSSTAVSSSTE |
| | | SSSSVEPSTSHSSSHSSSEVSSTVAPTTSV |
| | | APTTSSITTSSTSLTSATTSSVTISIEPTS |
| | | DAADKVIIPGLAGLVGALAVGLI |
| 75 | CCW22homologs | MQYRSLFLGSALLAAANAAVYNTTVTDVVS |
| | GQ68_02511 | ELETTVLTITSCAEDKCITSKSTGLITTST |
| | (chr 1) | LTKHGVVTVVTTVCDLPSTTKSYVPPAKTT |
| | | TIPPPEKTTTTVPPPAKTTTTVPPPAKTTS |
| | | TVPPPAKTSSHHESTITVTVPSSTSTKKIE |
| | | TESTTYHFVTQTTTARNITPPAITTQSHGA |
| | | AGMNAANFVGLGAAAVAAAALVL |
| 76 | CCW22homolog | MSLLLFLVLGAFLLSSVKAADIGAFRLRVY |
| | GQ68_03003 | TPGRFINGALNFNNWGYQYLDASSSNGQLF |
| | (chr 3) | AGYATVTSVTTFLAPDDEGFVWGSSLGGYP |
| | | GFLGIGAGATAFHLTGIPGDALSWYIEDNI |
| | | LKTSSPTYVCSRNDGDVVVGIEANTRWLAM |
| | | HDTSQLPPNYYCFQADYEIVALWYIPDTTS |
| | | TWTGTETSTTTDDDGSVIELVPTPLPDTTS |
| | | TWTGTFTTFTTDDDGSVIELVPTPLPDSTS |
| | | TWTGTYTTFTTDEDGSTIAVVPSSTIDSTS |
| | | TWTGTYTTFTTDEDGSTIAVVPSSTIDSTS |
| | | TWTGTYTTFTTDEDGSTIAVYHHLLSTPHP |
| | | PGLVLTPRSLPMRMEVLLLWYHHLLSTLHP |
| | | PGLVLTPRSLPMRMEVLLLWYHRLLSTPHP |
| | | GLVLTPRSLPMRMEVLLLYHHLLSTPHPPG |
| | | LVLTPRSLPMRMEVLLLWY |
| 77 | FLO5 homolog | MKLQLQSFVFFLLSAVNVLADDSYGCSIAT |
| | GQ68_04296 | SPRSTGFVANLYEFPNMAISNAELKTYVRY |
| | (chr 4) | RYKEGRLYDTISNIISPYFYYQGQGANSAY |
| | | GTLYGRPNVYLYNFSMELKGYFRPPITGQY |
| | | TIDENGANVDDAAMVFFGKAGAFDCCNSDY |
| | | ILPEQSAEYSLYSVYPHTATDQILSATIYL |
| | | EAGKYYPLRVTYTNIGNIGSLDLRVVLPSG |
| | | ASITSLGAFVYQFPNNLSPGTCTPDVEYFT |
| | | TTTQAWTGTYETTYTVPPSGTQPGTVIIET |
| | | PESYVTTTQPWTGTYETTYTVPPTGTEPGT |
| | | VIIETPESYVTTTQPWTGTYETTYTVPPSG |
| | | TEPGTVIIETPESYVTTTQPWTGTYETTYT |
| | | VPPSGTEPGTVIIETPESYVTTTQPWTGTY |
| | | ETTYTVPPSGTEPGIVIIETPESYVTTTQP |
| | | WTGTYETTYTVPPSGTEPGTVVIETPEITD |
| | | CEAVCCGAVPTSDPLRRRDVCDCETFCCPG |
| | | DTNCETYVTTTQPWTGTYETTYTVPPSGTE |
| | | PGTVIIETPESYVTTTQPWTGTYETTYTVP |
| | | PSGTEPGIVIIETPESYVTTTQPWTGTYET |
| | | TYTVPPTGTEPGTVIIETPESYVTTTQPWT |
| | | GTYETTYTVPPSGTEPGIVIIETPESYVTT |
| | | TQPWTGTYETTYTVPPSGTEPGTVIIETPE |
| | | SYVTTTQPWTGTYETTYTVPPTGTEPGTVI |
| | | IETPESYVTTTQPWTGTYETTYTVPPSGTE |
| | | PGIVIIETPESYVTTTQPWTGTYETTYTVP |
| | | PTGTEPGTVIIETPESYVTTTQPWTGTYET |
| | | TYTVPPTGTEPGTVIIETPESYVTTTQPWT |
| | | GTYETTYTVPPSGTEPGTVIIETPESYVTT |
| | | TQPWTGTYETTYTVPPSGTEPGTVVIETPE |
| | | ITDCEAVCCGAVPTSDPLRRRDVCDCETFC |
| | | CPGDTNCETYVTTTQPWTGTYETTYTVPPS |
| | | GTEPGTVIIETPESYVTTTQPWTGTYETTY |
| | | TVPPTGTEPGTVIIETPESYVTTTQPWTGT |
| | | YETTYTVPPSGTQPGTVIIETPESYVTTTQ |
| | | PWTGTYETTYTVPPTGTEPGTVIIETPESY |
| | | VTTTQPWTGTYETTYTVPPSGTEPGTVIIE |
| | | TPESYVTTTQPWTGTYETTYTVPPSGTQPG |
| | | TVIIETPESYVTTTQPWTGTYETTYTVPPT |
| | | GTEPGTVIIETPESYVTTTQPWTGTYETTY |
| | | TVPPSGTEPGIVIIETPESYVTTTQPWTGT |
| | | YETTYTVPPTGTEPGTVIIETPESYVTTTQ |
| | | PWTGTYETTYTVPPTGTEPGTVIIETPESY |
| | | VTTTQPWTGTYETTYTVPPSGTEPGTVIIE |
| | | TPESYVTTTQPWTGTYETTYTVPPSGTEPG |
| | | TVVIETPEITDCEAVCCGAVPTSDPLRRRD |
| | | VCDCETFCCPGDTNCETYVTTTQPWTGTYE |
| | | TTYTVPPSGTEPGTVIIETPESYVTTTQPW |
| | | TGTYETTYTVPPTGTEPGTVIIETPESYVT |
| | | TTQPWTGTYETTYTVPPSGTQPGTVIIETP |
| | | ESYVTTTQPWTGTYETTYTVPPTGTEPGTV |
| | | IIETPESYVTTTQPWTGTYETTYTVPPSGT |
| | | EPGTVIIETPESYVTTTQPWTGTYETTYTV |
| | | PPSGTQPGTVIIETPESYVTTTQPWTGTYE |
| | | TTYTVPPTGTEPGTVIIETPESYVTTTQPW |
| | | TGTYETTYTVPPSGTEPGIVIIETPESYVT |
| | | TTQPWTGTYETTYTVPPTGTEPGTVIIETP |
| | | ESYVTTTQPWTGTYETTYTVPPSGTEPGTV |
| | | IIETPESYVTTTQPWTGTYETTYTVPPSGT |
| | | QPGTVIIETPESYVTTTQPWTGTYETTYTV |
| | | PPSGTEPGTVIVETPDVPGSYVTTTQPWTG |
| | | TYETTHTVPPTGTEPGTVVVETPDVPGSYV |
| | | TTTQPWTGTYETTHTVPPTGTEPGTVVVET |
| | | PDVPGSYVTTTQPWTGTYETTYTVPPSGTE |
| | | PGTVIVETPDVPGSYVTTTQPWTGTYETTH |
| | | TVPPTGTEPGTVVVETPDVPGSYVTTTQPW |
| | | TGVYKTTYTVPPSGTIPGTVIIETPFGYFN |
| | | TSSISTKTDKRTITSVVPCSQCSESKTQYI |
| | | TPTGPGDVTVIISQPPSKITLSSPEDKTKT |
| | | DFITSTGSIGGGSPPSHPNDKPGIITTPTQ |
| | | PIGGGNPSDIPSAISSVSSGGNSRASVPSF |
| | | STSSAISVQVSSLYDENSGSTFEVSLLFSV |
| | | VSGFFLTLMV |
| 78 | FLO5 homolog | MKFPVPLLFLLQLFFIIATQGDESGNGDES |
| | GQ68_03011 | DTAYGCDITSNAFDGFDATIYEYNANDLKL |
| | (PAS_chr3_ | IRDPVFMSTGYLGRNVLNKISGVTVPGFNI |
| | 1145) | WNPRSRTATVYGVQNVNYYNMVLELKGYFK |
| | | AAVSGDYKLTLSNIDDSSMLFFGKNTAFQC |
| | | CDTGSIPVDQAPTDYSLFTIKPSNQVNSEV |
| | | ISSTQYLEAGKYYPVRIVFVNALERALFNF |
| | | KLTIPSGTVLDDFQDYIYQFGALDENSCYE |
| | | TTVSKITEWTTYTTPWTGTFETTRTITPTG |
| | | TEGTVVIETPESYVTTTQPWTGTYETTYTV |
| | | PPTGTEPGTVIIETPEIIDCEAVCCGPFLT |
| | | AFSFRKREECQCENICCPGDTNCETYVTTT |
| | | QPWTGTYETTYTVPPTGTEPGTVIIETPES |
| | | YVTTTQPWTGTYETTYTVPPTGTEPGTVII |
| | | ETPESYVTTTQPWTGTYETTYTVPPSGTEP |
| | | GTVVIETPEIVDCEAYCCASVAIKKRELCQ |
| | | CENFCCSWDQSCQTYVTTTQPWTGTYETTY |
| | | TVPPTGTEPGTVIIETPESYVTTTQPWTGT |
| | | YETTYTVPPTGTEPGTVIIETPESYVTTTQ |
| | | PWTGTYETTYTVPPTGTEPGTVIIETPEII |
| | | DCEAVCCGPFLTAFSFRKREECQCENICCP |
| | | GDTNCETYVTTTQPWTGTYETTYTVPPTGT |
| | | EPGTVIIETPESYVTTTQPWTGTYETTYTV |
| | | PPTGTEPGTVIIETPESYVTTTQPWTGTYE |
| | | TTYTVPPTGTEPGTVIIETPEIINCEAVCC |
| | | GPFLTAFSFRKREECQCENICCPGDTNCET |
| | | YVTTTQPWTGTYETTYTVPPTGTEPGTVII |
| | | ETPESYVTTTQPWTGTYETTYTVPSTGTEP |
| | | GTVIIETPESYVTTTQPWTGTYETTFTVPP |
| | | TGTEPGTVVIETPESYVTTTQPWTGTYETT |
| | | YSVPPSGTEPGTVVIETPEASTARTKFTTV |
| | | TSSWTGVFTTTKTLPASGTEPATIVIQTPT |
| | | GYFNTSSLVSTRTKTNVDTVTRVIPCPICT |
| | | APKTITVVPEEPNESVSVIISQPQSSSTDT |
| | | TLSKPDSVRVISQPETASQMDTSLSKTDSA |
| | | VISTETAGNNIIPLAGSHSYNTIVTTVTDS |
| | | PQVAQSTTATSSSNVHLTISTQTTTPSLVY |
| | | SSSLSTVHQVSPSNGGFRSSITVHPLLSVI |
| | | GAIFGALFM |
| 79 | FLO5 homolog | MTKFTILLLVLLKFYSILAIEVDGSANGQP |
| | GQ68_03079 | LAHPIVVEVHEATKWITHTSPWTGTPEAIR |
| | (chr 3) | TVTGETPYEQKIARYDEFNPRLANREIIDC |
| | | VAFCCGDATSSPSITEPESTATELPESYVT |
| | | INRPWSLSWIPDVPPGSPYWSTSTIPPSGT |
| | | EPGTVIIYFYLYDDARKRREINFGSTQPYH |
| | | GRPKLLGSIEKRELCQCDAVCCLGDLSCEV |
| | | YVTTTQPWTGTYETTYTITPTGSEPGTVII |
| | | ETPELYVTTTQPWTGTYETTYTITPTGSEP |
| | | GTVIIETPESYVTTTQPWTGTYETTYTITP |
| | | TGSEPGTVIIETPESYVTTTQPWTGTYETT |
| | | YTITPTGSEPGTVIIETPESYVTTTQPWTG |
| | | TYETTYTVPPSGTEPGAVIIETPELYVTTT |
| | | QPWTGTYETTYTITPTGSEPGTVIIETPES |
| | | YVTTTQPWTGTYETTYTVPPSGTEPGTVII |
| | | ETPELYVTTTQPWTGTYETTYTITPTGSEP |
| | | GTVIVEIPVSYVNSTQISTSTYDTTDTVLS |
| | | SGVEPGTIAIETPIVYLNTSVSAFSRPWTK |
| | | IDTVTQFSSCAVCSKPETITVTPENPIDTV |
| | | TIIISQPQSTSQSNTPTSFKANSTSAFSRF |
| | | DEDSIPVFGSYSYEITVNIDVNTEDDTTTN |
| | | LNADTTIIIGSLSAIRTVAGSSSNYHASNI |
| | | SPTINSQKTASSVVVHSDSSATVYQFSPSN |
| | | GAPWLSVQISTLLSVVGTLLAAVLL |
| 80 | FLO5 homolog | MNFRYLLILPIYASIVLGQVGDFQLLLNAK |
| | GQ68_04277 | EPIRNSPSLLSSNYGNLTLPAMANGALESH |
| | (chr 4) | FDYGNAYVGDDQITVVYHLPDEHGQINAYR |
| | | QDTDEYIGYLGLVTDDYGEYTYLSVIMPGV |
| | | QYDQTTSVNWYIENEELKSTSINVQPLLGC |
| | | YYKNPPQYSWYWASIDEPGNIASSNFVCEP |
| | | CKVYVDFVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADATSVWTGDHTTWTTDD |
| | | DGNVIEQIPTPSADITSMWTGSETSWTTDA |
| | | DGTVIELVPTPSADITSMWTGSETSWTTDA |
| | | DGTVIELVPTPSADITSMWTGSETSWTTDA |
| | | DGTVIELVPTPSADTTSVWTGSYTTWTTDE |
| | | DGTVIEQVPTPSADTPSADTTSVWTGSYTT |
| | | WTTDEDGTVIEQVPTPSADTTSVWTGSYTT |
| | | WTTDEDGTVIEQVPTPSADTPSADTTSVWT |
| | | GSYTTWTTEVGDGGSSTVVELVPTESSTST |
| | | NVMQTPVPSSGVSDGVSVFNGFNVEVFHYP |
| | | ADNYELANEISFLSYGYENLGLVTTVTGVS |
| | | DINFDTDSNWPYYIDRDALGNTGSYVNATI |
| | | EYEGFFRAPVDGEYVFSFSSTDYNSILFVG |
| | | SPAAADQALQKREVQFLKPETSPDYVLLFN |
| | | NTRDLGKTVSTTQYLLADQYYPLRVVIAAI |
| | | SQHALLDFQIKLPNGASLTQYQGYVYNFAL |
| | | EGSESTTVIGDKTSTWTGSYTTWTTDSDGS |
| | | TIVVVPPATITADKTSTWTGSYTTWTTDSD |
| | | GSTVVICPSITSDHNDKPSESTLTDSSIST |
| | | TVVTVTSCDIEKCTKTTALTGVRETTLTTG |
| | | GTTTVVTTYCPLPTDIVTVKTTSIDGSEVL |
| | | QTIYTAKPNHVVPDVQTSTVTITREVCDAF |
| | | TCTHATIVTGEILKTTTLADTHYTTVVPVY |
| | | VPLETYQPAVELSTLETVLKSSDLASGPVV |
| | | TAGSVQPSYQSGGVAESSLTVSEFEAHSTS |
| | | DTVSQPSTISLQTGEANALKWSSFFGAALV |
| | | PLVNVFFV |
| 81 | FLO5 homolog | MQNTNDKLIIRTFYSISTIHGLLSINIFSD |
| | GQ68_01371 | TRVYKFAIYSTDAVSLEPRTKNNMSLVTVL |
| | (chr 1) | ACFIIFAAHAFGQDTFYMLKVRTLTPNGYP |
| | | LADSLSNPMQYWDLYYVPGGPRRLESSFVN |
| | | WQPTTAAPINQFYCRLGTDGHMTGYNRVTG |
| | | SVIGKLSFGTNAATALAFGSYDGDPSYPPQ |
| | | AFSISSSVSGTMTYLNVHYVNARSITWYST |
| | | TTATGETNVYINVASTGYTGDRTTYQAELW |
| | | VEPFVPNIPVDTTTSIWTGSQTSYTTEVGE |
| | | NGGSTVIELIPTPPADATSTWTGTYTTRTT |
| | | DADGSVIEQIPTPSADTTSVWTGTYTTWTT |
| | | DADGSVIEQIPTPSADTTSVWTGTYTTWTT |
| | | DADGSVIEQIPTPSADTTATWTGTETSYTT |
| | | DVGEDGSSTVIELVPTPSADTTATWTGTET |
| | | SYTTDVGEDGSSTVVELVPTPSADTTATWT |
| | | GTETSYTTDVGEDGSSTVIELVPTPSADTT |
| | | ATWTGTETSYTTDVGEDGSSTVIELVPTPS |
| | | ADTTATWTGTETSYTTDVGEDGSSTVVELV |
| | | PTPSADTTATWTGTETSYTTDVGEDGSSTV |
| | | IELVPTPSADTTATWTGTETSYTTDVGEDG |
| | | SSTVIELVPTPSADTTATWTGTETSYTTDV |
| | | GEDGSSTVIELVPTPSADTTATWTGTETSY |
| | | TTDVGEDGSSTVIELVPTPSADTTATWTGT |
| | | ETSYTTDVGEDGSSTVIELVPTPSADTTAT |
| | | WTGTETSYTTDVGEDGSSTVIELVPTPSAD |
| | | TTATWTGTETSYTTDVGEDGSSTVIELVPT |
| | | PSADTTATWTGTETSYTTDVGEDGSSTVIE |
| | | LVPTPSADTTATWTGTETSYTTDVGEDGSS |
| | | TVIELVPTPSADTTATWTGTETSYTTDVGE |
| | | DGSSTVIELVPTPSADTTATWTGTETSYTT |
| | | DVGEDGSSTVIELVPTPSADTTATWTGTET |
| | | SYTTDVGEDGSSTVIELVPTPTPSADTTAT |
| | | WTGTETSYTTDVGEDGSSTVIELVPTPSAD |
| | | TTATWTGTETSYTTDVGEDGSSTVIELVPT |
| | | PSADTTATWTGTETSYTTDVGEDGSSTVIE |
| | | LVPTPSADTTATWTGTETSYTTDVGEDGSS |
| | | TVIELVPTPTPSADTTATWTGTETSYTTDV |
| | | GEDGSSTVIELVPTPSADTTATWTGTETSY |
| | | TTDVGEDGSSTVIELVPTPSADTTATWTGT |
| | | ETSYTTDVGEDGSSTVVELVPTPTPSADTT |
| | | ATWTGTETSYTTDVGEDGSSTVVELVPTPS |
| | | ADTTATWTGTETSYTTDVGEDGSSTVIELV |
| | | PTPSADTTATWTGTETSYTTDVGEDGSSTV |
| | | VELVPTPSADTTATWTGTETSYTTDVGEDG |
| | | SSTVVELVPTPTADTTATWTGTETSYTTDV |
| | | GEDGSSTVIELVPTPSADTTATWTGTETSY |
| | | TTDVGEDGSSTVVELVPTPSADTTATWTGT |
| | | ETSYTTDVGEDGSSTVVELVPTPSADTTAT |
| | | WTGTETSYTTDVGEDGSSTVIELVPTPSAD |
| | | TTATWTGTETSYTTDVGEDGSSTVVELVPT |
| | | PSADTTATWTGTETSYTTDVGEDGSSTVVE |
| | | LVPTPTADTTATWTGTETSYTTDVGEDGSS |
| | | TVIELVPTPSADTTATWTGTETSYTTDVGE |
| | | DGSSTVVELVPTPSADTTATWTGTETSYTT |
| | | DVGEDGSSTVIELVPTPSADTTATWTGTET |
| | | SYTTDVGEDGSSTVIELVPTPSADTTATWT |
| | | GTETSYTTDVGEDGSSTVIELVPTPTPSAD |
| | | TTATWTGTETSYTTDVGEDGSSTVIELVPT |
| | | PSADTTATWTGTETSYTTDVGEDGSSTVIE |
| | | LVPTPTPSADTTATWTGTETSYTTDVGEDG |
| | | SSTVVELVPTPSADTTATWTGTETSYTTDV |
| | | GEDGSSTVIELVPTPSADTTATWTGTETSY |
| | | TTDVGEDGSSTVVELVPTPSADTTATWTGT |
| | | ETSYTTDVGEDGSSTVIELVPTPSADTTAT |
| | | WTGTETSYTTDVGEDGSSTVIELVPTPSAD |
| | | TTATWTGTETSYTTDVGEDGSSTVIELVPT |
| | | PSADTTATWTGTETSYTTDVGEDGSSTVIE |
| | | LVPTPSADTTATWTGTETSYTTDVGEDGSS |
| | | TVIELVPTPSADTTATWTGTETSYTTDVGE |
| | | DGSSTVIELVPTPSADTTATWTGTETSYTT |
| | | DVGEDGSSTVIELVPTPSADTTATWTGTET |
| | | SYTTDVGEDGSSTVIELVPTPSADTTATWT |
| | | GTETSYTTDVGEDGSSTVIELVPTPSADTT |
| | | ATWTGTETSYTTDVGEDGSSTVIELVPTPS |
| | | ADTTATWTGTETSYTTDVGEDGSSTVVELV |
| | | PTPTPSADTTATWTGTETSYTTDVGEDGSS |
| | | TVIELVPSDTETATNIVETPVPSSGVSDGV |
| | | SVFDGFNVEVFHYPADNYELANEIGFLSYG |
| | | YENLGLVTNATGVSDINFDTDSNWPYYIDR |
| | | DALGNTGSYVNATIEYEGFFRAPVDGEYVF |
| | | SFSNTDYNSILFVGSPAAAGQALQKRRVQF |
| | | LKPETSPDHVLLFNNTRDLGQTISTTQYLL |
| | | ADQYYPLRVVIAAISQHALLDFQIKLPNGA |
| | | LLTQYQGYVYNFALEGSESTTVIGDKTSTW |
| | | TGSYTTWTTDSDGSTVVVVPSATITADKTS |
| | | TWTGSYTTWTTDSDGSTIVICPSITSDHND |
| | | KPSESTLTDGSISTTVVTVTSCDIEKCTKT |
| | | TALTGVTETTLTTGGTTTVVTTYCPLPTDI |
| | | VTVKTTSISGSEVLQTIYTAKPSHVVPNVH |
| | | TLTVTITREVCDAFTCTQATIVTGEILKTT |
| | | TLADTHSTTVVPVYVPLESYQSAVELSTLE |
| | | TVLKSSDFASGSAVTAGSAQPSYQSGGVAE |
| | | SSLTGSELEAHSTSDTVSQPSTISPQTGEA |
| | | NALRWSSFFGAALVPLVNVFFV |
| 82 | FLO5 homolog | MTKLTILLSVLLQLFSVLAEVPKKTEWSSH |
| | GQ68_04678 | TTYWTSTLEALRTVTPTGTERAVIGEAPYE |
| | (PAS_chr4_ | YKLIGNDQFDPGLNAKREIIDCEAVCCGAV |
| | 0363) | PTSDPLKRRDVCECENVCCPGDDCETYVTT |
| | | TQPWTGTYETTYTVPPSGTEPGTVVIETPE |
| | | ITDCEAVCCGAVPTSDPLRRRDVCECENVC |
| | | CPGDDCETYVTTTQPWTGTYETTYTVPPSG |
| | | TEPGTVVIETPEITDCEAVCCGAVPTSDPL |
| | | RRRDVCECENVCCPGDDCETYVTTTQPWTG |
| | | TYETTYTVPPTGTEPGTVVIETPVTYVTTT |
| | | QPWTGTYETTYTVPPTGTEPGTVVIETPEI |
| | | TDCEAVCCGAVPTSDPLRRRDVCECENVCC |
| | | PGDDCETYVTTTQPWTGTYETTYTVPPTGT |
| | | EPGTVVIETPVTYVTTTQPWTGTYETTYTV |
| | | PPTGTEPGTVVIETPVTYVTTTQPWTGTYE |
| | | TTYTVPPTGTEPGTVVIETPVTYVTTTQPW |
| | | TGTYETTYTIPPTGTEPGTVVIETPEITDC |
| | | EAVCCGAVPTSDPLRRRDVCECENVCCPGD |
| | | DCETYVTTTQPWTGTYETTYTVPPTGTEPG |
| | | TVVIETPVTYVTTTQPWTGTYETTYTVPPT |
| | | GTEPGTVVIETPVTYVTTTQPWTGTYETTY |
| | | TVPPTGTEPGTVVIETPVTYVTTTKPWTGT |
| | | YETTHTVPASGTEPGTVIIETPIKYLNTSI |
| | | SASTSTWTKINTVTQFISCPVCTIPKTITV |
| | | TPKISNETVTIIISQPHGTSSRTTTVVKTD |
| | | GASVSSHSYKTALTTDVKPEEKTSTKLGTV |
| | | TTVSGSHSAIDTVTGSLSDYHASSIPHTVK |
| | | SEEKASSTVTHTISSSTVYQVSPSNGASWL |
| | | SVRLNTALSIIGTLFAAVFI |
| 83 | FLO5 homolog | MSKTKNGGSEFVHIAYVFHIEASTPSDYIN |
| | GQ68_04282 | MIQIVLFPHQAQITKRMNLVTLLVCNLLCV |
| | (chr 4) | SLTLGQGVYRLKFPALVVTGRESVGTTVVN |
| | | YDFLVGNTGQYGDLGEFFYDGEPYYCWNST |
| | | DSQPLSCSSSSSLLISTQNVTISHPDEDGT |
| | | VYAYAERDGGLLGRFTVGSVSADWPQWAVI |
| | | VYSTSSSAHPSSWYVDDNKLKLTSGLGPNN |
| | | STTLQACYFTQSSGRDRYAISLEGSPAYTG |
| | | QVSCQATEFDLEFIPPSADTTSIWDGSYTT |
| | | WTTDSNGIVVEQIPTPSADTTSIWTGSETS |
| | | WTTDSDGTVIELVPTPSADATSIWTGDHTT |
| | | WTTDSEGNVIEQIPTPSADTTSIWTGSETS |
| | | WTTDSDGTVIELVPTPSADATSIWTGDHTT |
| | | WTTDSEGNVIEQIPTPSADATSIWTGDHTT |
| | | WTTDREGNVIEQIPTPSADTTSIWTGSETS |
| | | WTTDSDGTVIELVPTPSADATSIWTGDHTT |
| | | WTTDSEGNVIEQIPTPSADATSIWTGSETS |
| | | WTTDSDGTVIELVPTPSADATSIWTGDHTT |
| | | WTTDSEGNVIEQIPTPSADATSIWTGDHTT |
| | | WTTDSEGNVIEQIPTPSADTTSIWTGSETS |
| | | WTTDSDGTVIELVPTPSADATSIWTGDHTT |
| | | WTTDSEGNVIEQIPTPSADATSIWTGDHTT |
| | | WTTDSEGNVIEQIPTPSADTTSIWTGSETS |
| | | WTTDSDGTVIELVPTPSADATSIWTGDHTT |
| | | WTTDSEGNVIEQIPTPSADATSIWTGDHTT |
| | | WTTDSEGNVIEQIPTPSADTTSIWTGSETS |
| | | WTTDSDGTVIELVPTPSADATSIWTGDHTT |
| | | WTTDSEGNVIEQIPTPSADATSIWTGSETS |
| | | WTTDSDGTVIELVPTPSADATSIWTGDHTT |
| | | WTTDSEGNVIEQIPTPSADATSIWTGDHTT |
| | | WTTDSEGNVIEQIPTPSADTTSIWTGDHTT |
| | | WTTEVGGDGSSIVVELVPSETGTATNVVQT |
| | | PVPSSGISDGVSALDGFNVEVFHYPADNYE |
| | | LANEISFLSYGYENLGLVTTATGVSDINFD |
| | | TDSNWPSYIDRNALGNTGSYVNATIKYEGF |
| | | FRAPVDGDYEFSFSNIDYNSILFVGSAAAD |
| | | QALRKREAQFLKPETSPNHILFENNSRDVG |
| | | QTISTTQYLSADSYYPLRVVIAAVSQHALL |
| | | DFQIKLPNGVSLTQFQGYVYNFALEGAEST |
| | | TVIGDKTSTWTGTYTTWTTDSEGSTIVLCP |
| | | SIISDHNGKPADTTLTDGSISTTVVTVTSC |
| | | DIKKCTKTTALTGVTQKTLTVKGTTTVVTA |
| | | YCPLPTDVATVKTISVGGSEVLQTVYTAKP |
| | | SHIVPDVQTLTVTITREVCDALTCIPATIV |
| | | TGEILKTTTLADTHSTTVIPVYVPLETHQP |
| | | ALDLITLETVLKSSDFANGPAITSVSVESL |
| | | SHQSGVVVSEFDSDSTSGAVSQPSSAVSLQ |
| | | TGKASALKWSPFLGAAVISLFNVFFV |
| 84 | FLO5 homolog | MNLFTILAWGFLYVPLVLGEGYYSLNFDAR |
| | GQ68_03013 | VPIALGILGSSYQKYTIMADRSLLGGSNID |
| | (PAS_chr3_0015) | LDVTFSGIIELLTNRVHIVVSLPDADGRVS |
| | | VYDMYSGTSLGYLSFVCSLTTCEVHAVSSS |
| | | SGATTWTLDGNQLIPTSPSTVYACYRSLVG |
| | | LLAQYTLNDRTSITAQCEQTNLYVELAIPA |
| | | FPETTAVWTGTYTTWTTDESGSVIEQMPTP |
| | | SADTTTTWTGTYTTWTTDADGSVIEQIPTP |
| | | PADTTSVWTGTYTTRTTDADGSVIEQIPTP |
| | | SADTTSIWTGTYTTWTTDADGSVIEQIPTP |
| | | SADTTSVWTGTYTTWTTDADGSVIEQIPTP |
| | | SADTTSVWTGTYTTWTTDADGSVIEQIPTP |
| | | SADTTSVWTGTYTTWTTDADGSVIEQIPTP |
| | | STDTTLAPSADTTSIWTGTYTTWTTDADGS |
| | | VIEQIPTPSADTTSIWTGTYTTWTTDADGS |
| | | VIEQIPTPSADTTSVWTGTYTTWTTDADGS |
| | | VIEQIPTPSTDTTLAPSADTTSIWTGTYTT |
| | | WTTDADGSVIEQIPTPSADTTSVWTGTYTT |
| | | WTTDADGSVIEQIPTPSADTTSVWTGTYTT |
| | | WTTDADGSVIEQIPTPSADTTSVWTGTYTT |
| | | WTTDADGSVIEQIPTPSADTTSVWTGTYTT |
| | | WTTDADGSVIEQIPTPSADTTSVWTGTYTT |
| | | WTTDADGSVIEQIPTPSADTTSIWTGTYTT |
| | | WTTDADGSVIEQIPTPSADTTSVWTGTYTT |
| | | WTTDADGSVIEQIPTPSADTTLAPSADTTS |
| | | IWTGTYTTWTTDADGSVIEQIPTPSADTTS |
| | | IWTGTYTTWTTDADGSVIEQIPTPSADTTS |
| | | VWTGTYTTWTTDADGSVIEQIPTPSADTTS |
| | | VWTGTYTTWTTDADGSVIEQIPTPSADTTS |
| | | VWTGTYTTWTTDADGSVIEQIPTPSADTTS |
| | | VWTGTYTTWTTDADGSVIEQIPTPSADTTS |
| | | VWTGTYTTWTTDADGSVIEQIPTPSADTTS |
| | | VWTGTYTTWTTDADGSVIEQIPTPSTDTTL |
| | | APSADTTSIWTGTYTTWTTDADGSVIEQIP |
| | | TPSADTTSVWTGTYTTWTTDADGSVIEQIP |
| | | TPSADTTSVWTGTYTTWTTDADGSVIEQIP |
| | | TPSADTTSVWTGTYTTWTTDADGSVIEQIP |
| | | TPSTDTTLAPSADTTSIWTGTYTTWTTDAD |
| | | GSVIEQIPTPSADTTSIWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQSPTPSAYTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQIPTPSADTTSVWTGTYTTWTTDAD |
| | | GSVIEQSPTPSAYTTSVWTGTYTTWTTDAD |
| | | GSVIEQIPTPSADTTLAPSADTTSIWTGTY |
| | | TTWTTDADGSVIEQIPTPSADTTSIWTGTY |
| | | TTWTTDADGSVIEQIPTPSADTTSVWTGTY |
| | | TTWTTDADGSVIEQIPTPSADTTSVWTGTY |
| | | TTWTTDADGSVIEQIPTPSADTTSVWTGTY |
| | | TTWTTDADGSVIEQIPTPSTDTTLAPSADT |
| | | TSIWTGTYTTWTTDADGSVIEQIPTPSADT |
| | | TSVWTGTYTTWTTDADGSVIEQIPTPSTDT |
| | | TLAPSADTTSIWTGTYTTWTTDADGSVIEQ |
| | | IPTPSADTTSVWTGTYTTWTTDADGSVIEQ |
| | | IPTPSADTTSVWTGTYTTWTTDADGSVIEQ |
| | | IPTPSADTTSVWTGTYTTWTTDADGSVIEQ |
| | | IPTPSADTTLAPSADTTSIWTGTYTTWTTD |
| | | ADGSVIEQIPTPSADTTSVWTGTYTTWTTD |
| | | AAGTVIEVIPSGTSISSDVIPTPLPTSGVD |
| | | IDTIPYDAFNVAVYHYPADNYELANNLGFL |
| | | TSGYEGLGQVTTATSVGNINFDTSSGWPYY |
| | | IESNALGNTGSYVNATIEYVGFFQAPANGN |
| | | YELSFSNIDYNAILFLGSPATDSSLAKREV |
| | | QFLKPETSSEYVLFFDHGKDAGQTVSTTQY |
| | | LSAGLYYPLRIVLAAVSERAQLDFQITLPD |
| | | GRVLDQYQGYVYNFAHEGIESATSSAHETS |
| | | WSRFTNSTIYSHSSTIGIITSSTDAPHSVI |
| | | NPTAIETTSTDTSISTVAVTTSICDTKDCV |
| | | KTTVITPNSPLPTQTVSLTTTTIDRSEVVQ |
| | | TAHSAVPSQFAPDAHPSAVTITREQCDAYS |
| | | CSQATIVSGKVLQTTTVSDSTTVVPLDTPQ |
| | | LSVEASTLETRLKSTQSSRAPTVTVQTSQS |
| | | SRHSEDVTESSVHVSEFDAQSTSATSASAL |
| | | QAPSSISLQTGGANTLRLSAFLGTALLPML |
| | | NVLFI |
| 85 | SED1 homolog | MQFSIVATLALAGSALAAYSNVTYTYETTI |
| | (GQ68_01572) | TDVVTELTTYCPEPTTFVHKNKTITVTAPT |
| | | TLTITDCPCTISKTTKITTDVPPTTHSTPH |
| | | TTTTHVPSTSTPAPTHSVSTISHGGAAKAG |
| | | VAGLAGVAAAAAYFL |

TABLE 2
Exemplary advantageous proteins (Nucleotides)

| SEQ | | |
|---|---|---|
| ID | Sequence | |
| NO. | Info | Nucleotide sequence |
| 16 | BT2623 | Native nucleotide: |
| | Bacteroides | ATGAAAAAAGTAATAAAGAAATATT |
| | thetaiotaomicron | TCTTTTTAGCATTAGCTATTATAAT |
| | mannan | GTATTCGTGTAATGAAGATGAAAAG |
| | utilization | TATGATATATTAGAAAGATACACTC |
| | genes | CTGAAACTATAACATCTGACGAAAT |
| | | AGCTCCTGTGCTTAATTTACAGGCA |
| | | CAATATATGGATAGTAATAGCGAAA |
| | | TAGTACTTGTAACATGGATGAATCC |
| | | GGAAGATGATTTTTTGTCTAAGGTG |
| | | GAAATCTCTTGCTGTTCTGCGAATG |
| | | ATAATCTTTTGGGTGAACCTGTGTT |
| | | GTTGGACGCTGTTTCTACCAAAGTA |
| | | GGTTCTTATCAGACGTCACTTTCTG |
| | | TGGAAGAGAGGGGATATGTAAAGAT |
| | | TGTAGCTATTAATGAAAAAGGAGTA |
| | | CGCTCGGAAGCCCGTACAGCAGAGA |
| | | TCCTTTCTTCCCAACAGGATTTTGT |
| | | ATATAGAGCAGATTGTTTGATGTCT |
| | | TCTGTGATTGAATTATTTTTTGGTG |
| | | GGAGATATAATGCATGGAATGAGAA |
| | | TTACCCCAATGCTACAGGTCCCTAT |
| | | TGGGATGGCATTGCAGCCGTTTGGG |
| | | GACAAGGTGCAGCTTATTCCGGATT |
| | | TGTTACAATGTATAAGGTCACAAAG |
| | | GAAACTAATAATGAGAAACTAAGAG |
| | | CAAAATATGCAGAAAAGGAAGAAAC |
| | | TTTTCTAAACTCAATAGACATTTTT |
| | | TTGAATAATGGTAGTGGACGGAAAT |
| | | CTTTTGCTTATGGTACTTATATTGG |
| | | GCCGAATGATGAGCGTTATTACGAT |
| | | GATAATGTCTGGATTGGCATCGAAA |
| | | TGGCCAATTTATATGAACTTACAGG |
| | | GAATGAAGTTTATTTGCAGCATGCA |
| | | AATACTGTTTGGAACTTTATTTTGG |
| | | AAGGGATAGATGACGTGACTGGCGG |
| | | TGGAGTATATTGGAAAGAAGGTGCG |
| | | GTATCAAAGCATACATGTTCCACTG |
| | | CCCCGGCAGCTGTAATGGCTCTAAA |
| | | ATTATACCAATTGAGCAAGAATGAA |
| | | TCATATTTGGAAATAGCAAAGAGTT |
| | | TGTATTCATACTGTAAAGATGTATT |
| | | ACAAGATCCGAATGACTATTTATTT |
| | | TATGACAATGTTCGCTTAAGTGACC |
| | | CTTCCGATAAGAATTCGGAGCTTAA |
| | | AGTATCTAAGGATAAATTCACGTAT |
| | | AATTCGGGACAACCAATGTTAGCTG |
| | | CTGCTATGTTGTATCGGATTACAAA |
| | | AGAAGAACAATTTCTGAAAGATGCC |
| | | CAAAATATAGCACAGTCGATTTATA |
| | | AAAAATGGTTTAAAAACTATCATTC |
| | | GTCTATACTTGATAGAGATATAATG |
| | | ATATTAAGCGATCCAAACACTTGGT |
| | | TTAATGCCGTTATGTTCAGGGGATT |
| | | CGTAGAGCTATATAAAATAGATAAG |
| | | AACGATGTTTATGTCAAAGCGGTGA |
| | | AAAATACCATGGAACATGCTTGGCA |
| | | AAGCAACTGTAGAAATCGGTTGACT |
| | | AATCTAATGAGCGACGATTATGCAG |
| | | GTGATAAGAAAGAAGGTAAGTGGAA |
| | | TATAAAGACACAAGGTGCTTTTGTT |
| | | GAAATCTTCTCACTTATTGGGGAAT |
| | | TGGAACAACTTGGATGTTTTCAGGA |
| | | GTAG |
| 17 | BT2623 | ATGAAGAAAGTAATTAAGAAATATT |
| | (codon | TTTTCCTAGCCTTGGCAATCATTAT |
| | optimized) | GTACTCATGTAACGAAGACGAGAAA |
| | Bacteroides | TATGACATTCTTGAACGTTATACCC |
| | thetaiotaomicron | CTGAAACTATAACCTCTGACGAGAT |
| | mannan | CGCACCTGTACTAAACCTTCAAGCC |
| | utilization | CAGTACATGGATTCAAACAGTGAAA |
| | genes | TAGTTCTTGTGACTTGGATGAACCC |
| | | AGAGGATGATTTTCTGAGTAAAGTT |
| | | GAGATtTCTTGCTGCAGTGCTAACG |
| | | ATAACT |
| | | TACTGGGTGAGCCCGTCCTTCTTGA |
| | | TGCCGTCTCAACCAAGGTCGGCTCC |
| | | TACCAGACGTCCCTTTCTGTCGAAG |
| | | AACGTGGATATGTTAAGATCGTAGC |
| | | TATAAATGAAAAGGGAGTTAGGTCT |
| | | GAGGCTAGGACGGCTGAGATTTTGT |
| | | CATCTCAACAAGACTTCGTCTATCG |
| | | TGCAGACTGCCTTATGTCTAGTGTG |
| | | ATTGAACTGTTCTTTGGAGGAAGGT |
| | | ACAATGCATGGAACGAAAATTACCC |
| | | CAATGCAACCGGCCCTTACTGGGAT |
| | | GGAATCGCCGCTGTGTGGGGTCAGG |
| | | GTGCAGCCTATTCTGGTTTCGTAAC |
| | | TATGTACAAAGTTACCAAAGAAACA |
| | | AATAACGAAAAACTAAGGGCTAAGT |
| | | ATGCAGAAAAGGAGGAAACATTCCT |
| | | GAACTCTATAGACATCTTTTTAAAT |
| | | AATGGCTCTGGCAGAAAGTCATTTG |
| | | CCTACGGCACGTACATCGGTCCTAA |
| | | CGACGAGCGTTATTACGATGATAAT |
| | | GTGTGGATAGGTATAGAAATGGCAA |
| | | ACTTATATGAGCTGACAGGAAACGA |
| | | GGTGTACCTACAACATGCCAATACC |
| | | GTGTGGAATTTCATATTAGAAGGCA |
| | | TTGATGATGTAACGGGAGGTGGCGT |
| | | ATACTGGAAGGAGGGTGCAGTTTCC |
| | | AAACACACGTGCTCAACCGCCCCCG |
| | | CAGCTGTAATGGCTTTGAAACTTTA |
| | | CCAGTTGTCCAAGAATGAATCCTAC |
| | | TTAGAGATCGCCAAATCCTTGTATT |
| | | CCTACTGCAAAGATGTCTTGCAAGA |
| | | TCCAAACGATTATCTTTTTTACGAC |
| | | AACGTGAGGCTAAGTGACCCTTCAG |
| | | ATAAGAACAGTGAACTAAAAGTATC |
| | | AAAAGACAAGTTCACTTACAACAGT |
| | | GGTCAGCCCATGCTTGCAGCAGCCA |
| | | TGCTGTATCGTATAACCAAAGAAGA |
| | | GCAGTTTCTGAAAGACGCCCAAAAC |
| | | ATTGCCCAATCAATATACAAGAAAT |
| | | GGTTCAAAAATTACCATTCATCAAT |
| | | CTTAGATAGGGATATAATGATTTTG |
| | | TCTGATCCAAACACCTGGTTTAACG |
| | | CAGTCATGTTTAGGGGTTTTGTCGA |
| | | GCTGTATAAAATCGACAAAAATGAT |
| | | GTTTATGTTAAGGCAGTTAAGAACA |
| | | CAATGGAGCATGCTTGGCAATCAAA |
| | | CTGCCGTAACAGACTTACCAATCTT |
| | | ATGTCTGACGACTATGCCGGAGACA |
| | | AGAAGGAGGGTAAGTGGAACATTAA |
| | | GACCCAAGGAGCTTTTGTTGAAATT |
| | | TTTTCTTTGATTGGCGAGTTAGAAC |
| | | AGTTAGGCTGTTTCCAGGAATAG |
| 18 | BT2629 | ATGAAAACATCTTTAAACACTTGCT |
| | | ATTTCTTGGAGGTGCCGTGTTGTAC |
| | | AGCCTGCAATCTTCTGCCGTTAAGA |
| | | ATCCTGTAGACTATGTCAGCACACT |
| | | GATAGGCACTCAATCCAAGTTTGAA |
| | | CTGTCTACCGGAAACACGTATCCGG |
| | | CTACGGCATTGCCGTGGGGAATGAA |
| | | TTTCTGGACACCGCAGACCGGTAAA |
| | | ATGGGAGACGGTTGGGCGTACACGT |
| | | ATGATGCCGACAAAATCCGGGGATT |
| | | CAAACAAACACATCAGCCCAGTCCC |
| | | TGGATGAACGACTACGGGCAGTTCG |
| | | CCATCATGCCTATCACAGGCGGACT |
| | | GGTATTCGATCAAGACCGACGTGCC |
| | | AGTTGGTTCTCTCACAAAGCGGAAG |
| | | TTGCCAAACCTTATTATTATAAGGT |
| | | ATACCTCGCCGACCATGATGTAACA |
| | | ACCGAGCTTGCTCCTACGGAGCGTG |
| | | CCGTCATGTTCCGTTTCACGTATCC |
| | | GGAGACAAAGAATGCCTACGTGATT |
| | | GTAGACGCTTTCGACAAAGGTTCTT |
| | | ATGTGAAAGTGATTCCGGAAGAAAA |
| | | CAAGATTATCGGCTATTCAACCAAG |
| | | AATAGCGGCGGTGTGCCGGAAAACT |
| | | TCAAAAACTATTTCGTGATTCAATT |
| | | CGACAAACCGTTCACATTCGTTTCC |
| | | ACAGTTTTCGAAAACAACATTCTTC |
| | | CGAATGAAACAGAAGCAAAAGGAAA |
| | | CCACACAGGGGCCGTGATCGGATTC |
| | | GCCACGAAAAAGGGAGAAATCGTAC |
| | | ACGCACGTGTTGCTTCCTCCTTTAT |
| | | CAGCCCCGAACAGGCGGAGTTGAAT |
| | | CTCAAAGAGCTTGGCAAAAACAGTT |
| | | TCGACCAACTGGTAGCGAACGGAAG |
| | | AGAAATCTGGAACCGTGAAATGAGT |
| | | AAAATAGAGATAGAAGACGATAATA |
| | | TCGATAATTTACGCACCTTCTATTC |
| | | TTGTTTATACCGTTCCATGCTTTTT |
| | | CCACGCAGTTTCTACGAGATAGATG |
| | | CTAAGGGACAAGTCATGCATTACAG |
| | | CCCCTACAACGGCGAAGTGCGTCCC |
| | | GGTTATATGTTTACCGACACCGGAT |
| | | TCTGGGACACGTTCCGCTGCCTGTT |
| | | CCCTTTCCTCAACCTGATGTATCCG |
| | | TCAATGAATCAAAAGATGCAGGAGG |
| | | GACTAGTGAATACTTACAAGGAAAG |
| | | TGGTTTCCTGCCGGAATGGGCCAGT |
| | | CCGGGACATCGGGATTGTATGGTAG |
| | | GCAACAACTCGGCTTCCGTAGTAGC |
| | | CGACGCTTACATCAAAGGATTGCGA |
| | | GGATATGATATCGAAACTCTTTGGG |
| | | AAGCATTGAAACATGGAGCAAATGC |
| | | ACATCTTCGCGGGACTGCTTCAGGT |
| | | CGTCTCGGTTACGAATCTTACAACC |
| | | AACTGGGATATGTTGCCAACAATAT |
| | | CGGCATAGGACAAAACGTTGCACGT |
| | | ACATTGGAGTATGCTTACAACGACT |
| | | GGGCAATTTATACACTAGGTAAGAA |
| | | ACTTGGTAAACCGGAGAACGAAATC |
| | | GACATTTATAAGAAACACGCGCTGA |
| | | ACTACAAAAATGTCTATCACCCGGA |
| | | ACGCAAACTGATGGTTGGCAAAGAT |
| | | AACAAAGGCGTATTCAATCCGAATT |
| | | TCGATGCAGTGGACTGGAGCGGTGA |
| | | ATTTTGCGAAGGGAATAGCTGGCAC |
| | | TGGAGCTTCTGCGTATTCCACGACC |
| | | CGCAAGGACTTATCAACCTGATGGG |
| | | AGGCAAGAAAGAATTCAACGCGATG |
| | | ATGGATTCTGTTTTTGTCATCTCGG |
| | | GTAAACTGGGAATGGAAAGCCGCGG |
| | | CATGATTCACGAAATGCGTGAAATG |
| | | CAAGTAATGAACATGGGGCAATATG |
| | | CGCATGGCAACCAGCCTATTCAACA |
| | | CATGGTATATCTCTACAACTATTCA |
| | | AGCGAACCCTGGAAAGCTCAATACT |
| | | GGATACGTGAGATTATGAACAAACT |
| | | ATATACCGCCGGTCCCGACGGTTAT |
| | | TGCGGTGACGAAGACAACGGACAGA |
| | | CTTCCGCCTGGTATGTATTCTCCGC |
| | | ACTCGGTTTCTATCCGGTTTGCCCG |
| | | GGAACAGATGAATATATCATAGGAA |
| | | CCCCGCTCTTTAAATCAGCGAAGTT |
| | | ACATTTGGAGAACGGAAAGACCATC |
| | | ACGATCAAGGCAGATAACAACCAGC |
| | | TTGACAACCGCTACATCAAGGAAAT |
| | | GAAAGTAAACGGGAAATCACAAACC |
| | | CGTAATTTCCTTACACATGACCAGC |
| | | TGATTAAAGGTGCTAATATTCAATT |
| | | TCAAATGAGCCCCGTGCCCAATAAA |
| | | CAACGGGGAACCACAGAAAAAGATG |
| | | TACCTTACTCTCTTTCGTTTGAATA |
| | | A |
| 19 | BT2629 | ATGAAAACACATTTTTCATTTAAAC |
| | (codon | ACTTGCTATTTCTTGGAGGTGCCGT |
| | optimized) | GTTGTACAGCCTGCAATCTTCTGCC |
| | | GTTAAAAATCCCGTCGACTATGTGT |
| | | CTACCCTTATAGGCACGCAATCCAA |
| | | GTTTGAGTTGTCCACAGGCAACACC |
| | | TACCCTGCTACCGCTCTTCCATGGG |
| | | GCATGAACTTTTGGACTCCACAGAC |
| | | AGGAAAAATGGGTGATGGATGGGCA |
| | | TATACGTACGATGCTGACAAGATCC |
| | | GTGGCTTTAAACAAACTCACCAACC |
| | | ATCTCCATGGATGAACGACTACGGT |
| | | CAGTTTGCAATAATGCCAATTACTG |
| | | GAGGACTTGTATTTGACCAAGATAG |
| | | ACGTGCTAGTTGGTTTTCCCACAAG |
| | | GCAGAAGTCGCTAAACCATACTATT |
| | | ACAAGGTCTACCTTGCTGACCATGA |
| | | CGTGACAACCGAATTGGCCCCCACC |
| | | GAGAGGGCCGTGATGTTTAGGTTTA |
| | | CGTACCCCGAGACGAAAAACGCCTA |
| | | CGTTATTGTAGATGCCTTTGATAAG |
| | | GGAAGTTATGTCAAAGTAATACCTG |
| | | AGGAAAACAAGATTATAGGTTATTC |
| | | TACAAAAAATTCAGGCGGCGTCCCA |
| | | GAAAATTTTAAGAACTACTTCGTTA |
| | | TTCAGTTTGACAAACCATTTACGTT |
| | | CGTATCAACTGTATTTGAAAACAAT |
| | | ATTTTGCCAAACGAGACAGAGGCCA |
| | | AGGGTAACCACACAGGCGCTGTGAT |
| | | CGGCTTCGCAACGAAGAAGGGCGAA |
| | | ATAGTACATGCTAGAGTCGCCTCTT |
| | | CTTTCATATCTCCTGAACAAGCCGA |
| | | GTTAAACTTAAAGGAATTGGGAAAA |
| | | AATTCTTTTGATCAACTGGTAGCCA |
| | | ACGGTAGGGAGATTTGGAATCGTGA |
| | | GATGAGTAAGATCGAGATCGAGGAT |
| | | GATAACATTGATAATTTAAGGACGT |
| | | TCTATTCTTGTCTGTATAGATCCAT |
| | | GTTGTTTCCTAGGTCCTTTTACGAG |
| | | ATTGACGCTAAGGGCCAGGTGATGC |
| | | ACTATTCACCCTACAATGGCGAAGT |
| | | ACGTCCTGGATACATGTTCACGGAT |
| | | ACGGGATTTTGGGACACGTTTAGGT |
| | | GTCTGTTCCCTTTTTTGAATCTGAT |
| | | GTATCCCTCCATGAACCAGAAAATG |
| | | CAGGAGGGCCTTGTAAACACTTACA |
| | | AGGAGTCCGGATTTTTACCAGAGTG |
| | | GGCAAGTCCAGGCCATCGTGATTGT |
| | | ATGGTTGGCAACAATTCAGCATCAG |
| | | TTGTGGCTGATGCCTATATCAAAGG |
| | | TTTGAGAGGATACGATATCGAGACG |
| | | CTGTGGGAGGCCCTTAAACACGGTG |
| | | CCAACGCTCATCTAAGGGGTACCGC |
| | | ATCTGGCAGATTAGGTTACGAGTCC |
| | | TACAACCAACTAGGCTACGTGGCTA |
| | | ATAATATCGGTATTGGCCAGAACGT |
| | | TGCAAGAACCCTTGAATACGCTTAC |
| | | AACGACTGGGCAATCTACACTTTGG |
| | | GTAAAAAACTTGGAAAACCCGAAAA |
| | | TGAAATAGACATTTATAAGAAACAC |
| | | GCTCTTAACTACAAAAACGTGTATC |
| | | ACCCTGAAAGGAAGCTAATGGTCGG |
| | | TAAGGACAACAAGGGCGTCTTTAAC |
| | | CCTAATTTCGATGCTGTGGACTGGT |
| | | CTGGAGAGTTCTGCGAAGGCAATTC |
| | | CTGGCATTGGTCCTTCTGTGTTTTT |
| | | CACGACCCTCAAGGATTAATTAATT |
| | | TGATGGGTGGTAAGAAGGAGTTCAA |
| | | TGCTATGATGGATTCCGTATTCGTG |
| | | ATCTCTGGTAAACTGGGCATGGAGT |
| | | CTCGTGGTATGATCCACGAAATGAG |
| | | AGAGATGCAGGTAATGAACATGGGA |
| | | CAATACGCACATGGCAATCAGCCTA |
| | | TACAGCATATGGTATATCTTTATAA |
| | | CTACAGTTCAGAGCCTTGGAAGGCA |
| | | CAATATTGGATTAGGGAGATCATGA |
| | | ACAAGCTTTATACCGCCGGCCCTGA |
| | | TGGATATTGTGGCGATGAAGATAAC |
| | | GGACAGACCAGTGCATGGTATGTGT |
| | | TTTCCGCACTTGGTTTTTACCCTGT |
| | | GTGCCCTGGTACGGATGAGTACATT |
| | | ATCGGCACGCCATTATTCAAATCTG |
| | | CTAAGTTGCATCTTGAAAACGGAAA |
| | | GACGATAACGATAAAAGCCGACAAC |
| | | AACCAACTGGATAACAGATATATTA |
| | | AAGAAATGAAGGTCAACGGTAAGTC |
| | | ACAAACGAGAAACTTTTTAACCCAT |
| | | GACCAACTAATTAAGGGAGCCAACA |
| | | TACAATTCCAGATGAGTCCAGTCCC |
| | | CAATAAGCAACGTGGAACAACAGAG |
| | | AAGGACGTGCCTTATTCTTTGTCCT |
| | | TCGAGTAG |
| 20 | BT2630 | ATGAAACTGAAAAACCTTTTACTAA |
| | | TTGCCCTTGTTGCGATCGTCTTTTG |
| | | CGGTTGTCAAAGTAACTATCAGCCT |
| | | ACTTCTATCACCGTTGCCTCCTACA |
| | | ATTTGAGAAACGCCAACGGTGGCGA |
| | | TTCAATCAACGGAAACGGTTGGGGA |
| | | CAACGTTACCCGGTCATTGCCCAAA |
| | | TAGTGCAATATCACGATTTCGATAT |
| | | TTTCGGCACGCAGGAGTGCTTTATT |
| | | CATCAACTGAAAGATATGAAAGAAG |
| | | CATTACCCGGTTATGATTATATCGG |
| | | TGTAGGTCGCGACGACGGCAAAGAG |
| | | AAAGGTGAACATTCTGCTATTTTCT |
| | | ATCGCACAGACAAGTTTGACGTGAT |
| | | AGAGAAAGGTGATTTTTGGTTGTCG |
| | | GAAACTCCCGACGTGCCGAGCAAAG |
| | | GATGGGATGCCGTGTTGCCGCGTAT |
| | | TTGCAGTTGGGGACACTTCAAATGC |
| | | AAAGATACCGGCTTCGAATTCCTTT |
| | | TCTTCAACCTGCACATGGACCATAT |
| | | CGGCAAGAAGGCACGTGTGGAAAGT |
| | | GCATTCCTCGTACAGGACAAGATGA |
| | | AAGAACTTGGCAAAGGCAAAGAGCT |
| | | TCCGGCCATCCTGACGGGAGACTTC |
| | | AATGTCGACCAGACCCACCAGTCTT |
| | | ATGATGCTTTTGTGAGCAAAGGGGT |
| | | GTTGTGCGACTCTTACGAGAAGGCC |
| | | GGCTTCCGCTATGCTATCAACGGCA |
| | | CGTTCAACGACTTCGACCCGAACAG |
| | | CTTTACGGAAAGCCGTATCGACCAT |
| | | ATATTCGTTTCTCCGTCTTTCCAAG |
| | | TGAAAAGATATGGTGTGCTGACTGA |
| | | TACTTACCGCAGCATCGTAGGCAAG |
| | | GGAGAAAAGAAGCAGGCGAACGATT |
| | | GCCCGGAAGAAATCGACATCAAGAC |
| | | TTATCAGGCGCGCACTCCTTCAGAC |
| | | CATTTCCCCGTAAAGGTGGAACTGG |
| | | AGTTCGACCAGCGTCAGCAGAAATA |
| | | A |
| 21 | BT2631 | ATGAGAAATATATGTTTTGTAGCCT |
| | | GTATGTTATTTTGCCTTACTTCCGC |
| | | AGTGGGAAAGACACCGGGAAATACC |
| | | CGTTATCTTTCTATTGCCGACTCGA |
| | | TTCTATCTAATGTATTGAATCTCTA |
| | | TCAGACGAATGACGGACTACTAACA |
| | | GAAACGTATCCTGTCAATCCCGACC |
| | | AAAAAATTACTTATCTGGCGGGCGG |
| | | AACGCAGCAGAACGGAACGCTGAAG |
| | | GCTTCTTTTCTATGGCCGTATTCCG |
| | | GGATGATGTCGGGTTGTGTGGCTTT |
| | | ATACAAAGCGACCGGAAACAAGAAG |
| | | TACAAAAAGATTCTCGAGAAAAGAA |
| | | TTCTACCGGGAATGGAGCAGTATTG |
| | | GGATAACAGTCGCTTGCCGGCCTGT |
| | | TATCAGTCATACCCCACCAAGTACG |
| | | GGCAGCACGGACGTTATTATGACGA |
| | | TAACATCTGGATTGCACTGGATTAC |
| | | TGCGATTATTACCAACTGACTCACA |
| | | AGCCTGCATCTTTGGAAAAAGCCGT |
| | | TGCATTGTATCAATATATCTACAGT |
| | | GGATGGAGCGATGAGATAGGCGGTG |
| | | GCATCTTTTGGTGTGAACAGCAGAA |
| | | GGAAGCGAAGCATACTTGTTCCAAT |
| | | GCACCGTCTACTGTGCTCGGTGTCA |
| | | AGTTGTACCGGCTGACGAAGGATGC |
| | | CAAATACCTCGAAAAAGCAAAAGAG |
| | | ACGTATGCCTGGACGAAAAAGCATC |
| | | TGTGCGACCCTACCGACCATCTTTA |
| | | CTGGGATAACATCAACCTGAAAGGG |
| | | AAAGTTTCCAAAGAGAAGTACGCCT |
| | | ACAACAGTGGACAGATGATTCAGGC |
| | | GGGTGTATTGCTCTATGAGGAAACG |
| | | GGAGATGAACAGTATTTGCGCGATG |
| | | CACAGCAGACAGCCGCAGGAACTGA |
| | | TGCTTTTTTCCGCACAAAAGCCGAC |
| | | AAGAAAGACCCGACTGTCAAAGTGC |
| | | ATAAAGACATGGCCTGGTTTAACGT |
| | | GATCTTATTCAGAGGACTGAAAGCT |
| | | CTGTATAAGATTGACAAGAATCCGG |
| | | CGTATGTCAATGCGATGGTGGAAAA |
| | | TGCGCTTCACGCCTGGGAAAACTAC |
| | | CGGGATGAAAACGGATTATTAGGCA |
| | | GGGATTGGTCGGGACATAACAAGGA |
| | | GCAGTATAAATGGCTGCTCGACAAT |
| | | GCCTGTCTTATTGAATTCTTTGCAG |
| | | AGATTTAA |
| 22 | BT2631 | ATGAGAAACATCTGCTTTGTCGCCT |
| | (codon | GTATGCTGTTCTGTCTGACCAGTGC |
| | optimized) | TGTGGGCAAGACTCCTGGAAACACG |
| | | AGGTACCTATCTATTGCCGACTCTA |
| | | TCCTTTCCAACGTGTTGAACCTTTA |
| | | CCAAACTAACGATGGTCTTCTGACC |
| | | GAAACTTATCCTGTTAACCCCGACC |
| | | AGAAGATAACCTATTTGGCTGGCGG |
| | | CACACAACAGAATGGCACCCTGAAG |
| | | GCATCTTTTTTGTGGCCTTATTCTG |
| | | GCATGATGTCCGGATGCGTTGCATT |
| | | GTATAAAGCCACTGGCAACAAAAAG |
| | | TATAAAAAGATACTTGAGAAAAGGA |
| | | TTTTACCAGGAATGGAGCAGTACTG |
| | | GGACAATAGTCGTTTACCAGCATGT |
| | | TATCAATCATACCCTACTAAATACG |
| | | GCCAGCACGGAAGATACTATGACGA |
| | | TAATATCTGGATCGCCTTAGATTAC |
| | | TGCGACTATTACCAGTTAACCCACA |
| | | AACCCGCCTCTCTGGAGAAAGCCGT |
| | | AGCTCTATATCAGTACATCTATTCT |
| | | GGTTGGTCAGATGAGATTGGCGGAG |
| | | GCATATTTTGGTGTGAGCAACAAAA |
| | | AGAGGCCAAGCACACGTGCTCCAAT |
| | | GCCCCTTCCACTGTATTAGGTGTAA |
| | | AACTGTATAGGCTTACAAAAGACGC |
| | | CAAATATCTGGAAAAAGCTAAAGAG |
| | | ACGTATGCTTGGACCAAGAAACATC |
| | | TTTGCGACCCTACAGATCATTTGTA |
| | | CTGGGATAATATAAACTTGAAAGGA |
| | | AAGGTTTCTAAAGAAAAATACGCCT |
| | | ATAATAGTGGTCAAATGATTCAGGC |
| | | CGGCGTTCTGTTGTATGAGGAAACA |
| | | GGCGATGAGCAATATCTTCGTGATG |
| | | CTCAACAAACAGCCGCTGGCACAGA |
| | | CGCATTTTTCAGAACGAAGGCAGAC |
| | | AAGAAAGACCCAACTGTCAAGGTAC |
| | | ATAAGGACATGGCCTGGTTTAACGT |
| | | AATTTTATTTAGAGGCCTGAAGGCA |
| | | TTATATAAAATAGACAAGAACCCCG |
| | | CCTATGTAAATGCTATGGTAGAGAA |
| | | TGCCCTGCATGCCTGGGAAAATTAC |
| | | AGAGACGAGAATGGACTTCTAGGAA |
| | | GAGATTGGAGTGGTCACAACAAAGA |
| | | ACAATACAAATGGCTATTAGATAAC |
| | | GCCTGTCTAATTGAGTTCTTCGCAG |
| | | AGATTTAG |
| 23 | BT2632 | ATGAATATAACTAAAGCCTTTTGTT |
| | | TGTCCATAGCACTCTTGGGCGCTAG |
| | | CAATATGCAGGCTATAACGAACAGT |
| | | GATTTTGTCATCCAACAAGATAATA |
| | | CCAAAATCAACAACTATCAGACGAA |
| | | CCGTCCGGAAACATCGAAACGTCTG |
| | | TTTGTCTCACAAGCTGTGGAACAAC |
| | | AGATTGCGCATATCAAGCAACTGCT |
| | | GACGAATGCCCGCTTAGCATGGATG |
| | | TTCGAAAACTGTTTCCCGAACACAC |
| | | TGGATACTACTGTTCATTTTGACGG |
| | | TAAAGACGATACGTTTGTTTATACA |
| | | GGTGACATCCACGCCATGTGGTTGC |
| | | GCGATTCGGGTGCACAAGTATGGCC |
| | | TTACGTGCAACTCGCCAACAAAGAC |
| | | GCAGAACTGAAAAAAATGCTCGCTG |
| | | GCGTTATCAAACGTCAGTTCAAGTG |
| | | TATCAATATCGACCCGTATGCCAAT |
| | | GCTTTCAACATGAATTCCGAAGGCG |
| | | GCGAATGGATGAGTGACCTTACGGA |
| | | CATGAAGCCCGAACTGCACGAACGC |
| | | AAATGGGAAATCGACTCGCTCTGTT |
| | | ATCCTATCCGTCTCGCTTATCATTA |
| | | CTGGAAGACGACGGGAGATGCCAGT |
| | | ATATTCTCCGACGAATGGCTTACAG |
| | | CCATCGCCAAGGTTCTGAAAACGTT |
| | | TAAGGAACAGCAACGAAAAGAAGAT |
| | | CCGAAAGGTCCTTATCGTTTCCAAC |
| | | GCAAAACGGAACGTGCACTCGATAC |
| | | GATGACCAATGACGGCTGGGGCAAT |
| | | CCTGTAAAGCCGGTCGGACTGATTG |
| | | CTTCTGCTTTCCGTCCTTCGGATGA |
| | | TGCTACAACTTTCCAGTTTCTCGTT |
| | | CCGTCCAACTTCTTTGCTGTAACTT |
| | | CATTGCGCAAAGCTGCCGAAATTCT |
| | | GAATACGGTCAACAAGAAACCTGAT |
| | | TTAGCTAAAGAATGTACTACACTGT |
| | | CTAACGAAGTGGAAACAGCCCTGAA |
| | | AAAGTATGCGGTTTACAATCATCCG |
| | | AAATATGGCAAAATCTATGCTTTCG |
| | | AAGTGGACGGTTTCGGCAATCAACT |
| | | GTTAATGGATGATGCCAATGTGCCG |
| | | AGTCTCATTGCCCTGCCTTATCTTG |
| | | GGGATGTGAAAGTGAACGATCCTAT |
| | | TTATCAGAATACCCGTAAGTTTGTA |
| | | TGGAGCGAAGATAATCCTTACTTCT |
| | | TCAAAGGTACTGCCGGCGAAGGAAT |
| | | TGGCGGTCCGCACATCGGATATGAT |
| | | ATGATTTGGCCCATGAGTATTATGA |
| | | TGAAAGCATTCACCAGTCAAAACGA |
| | | CGCAGAAATCAAGACCTGCATCAAA |
| | | ATGCTGATGGATACGGATGCCGGAA |
| | | CAGGGTTCATGCATGAATCTTTCCA |
| | | CAAGAACGACCCGAAAAACTTTACT |
| | | CGTTCCTGGTTTGCATGGCAAAATA |
| | | CGCTGTTTGGAGAACTAATCCTAAA |
| | | ACTCGTGAATGAAGGAAAGGTAGAC |
| | | TTACTGAATAGTATCCAATAG |
| 24 | BT2632 | ATGAATATTACTAAGGCCTTTTGCC |
| | (codon | TAAGTATCGCATTATTAGGAGCCTC |
| | optimized) | TAATATGCAAGCCATTACCAATAGT |
| | | GACTTTGTTATTCAGCAGGACAACA |
| | | CAAAAATCAATAATTACCAGACAAA |
| | | TCGTCCAGAGACATCAAAAAGGTTG |
| | | TTCGTGTCTCAGGCAGTCGAGCAGC |
| | | AAATCGCTCACATCAAGCAACTTCT |
| | | GACAAACGCAAGGCTTGCCTGGATG |
| | | TTCGAGAACTGCTTTCCAAACACTT |
| | | TAGACACGACGGTCCACTTCGACGG |
| | | AAAGGACGATACATTCGTTTATACC |
| | | GGCGATATCCACGCTATGTGGCTAA |
| | | GAGACTCCGGAGCACAGGTTTGGCC |
| | | CTACGTCCAACTGGCCAATAAAGAT |
| | | GCCGAGCTGAAAAAAATGCTGGCTG |
| | | GAGTCATTAAAAGACAATTCAAATG |
| | | CATTAACATTGATCCTTATGCAAAT |
| | | GCATTCAATATGAATTCAGAAGGCG |
| | | GCGAGTGGATGTCCGATTTGACAGA |
| | | TATGAAACCCGAGCTTCATGAGCGT |
| | | AAATGGGAGATCGACAGTCTTTGCT |
| | | ACCCCATTAGACTGGCATATCACTA |
| | | TTGGAAGACAACAGGAGACGCTTCC |
| | | ATATTTAGTGACGAGTGGTTAACGG |
| | | CAATAGCCAAAGTCCTAAAGACATT |
| | | TAAGGAGCAGCAGCGTAAAGAGGAC |
| | | CCAAAGGGTCCATATAGATTTCAAA |
| | | GAAAGACAGAGAGAGCCTTAGATAC |
| | | CATGACGAACGACGGATGGGGTAAT |
| | | CCTGTCAAGCCTGTAGGTCTGATTG |
| | | CATCCGCCTTTAGGCCATCAGATGA |
| | | TGCTACGACATTTCAATTCTTAGTG |
| | | CCAAGTAATTTCTTTGCCGTGACTT |
| | | CTCTTAGGAAAGCTGCCGAGATACT |
| | | TAACACGGTAAACAAGAAACCAGAc |
| | | CTTGCCAAAGAATGCACTACATTGT |
| | | CAAATGAAGTAGAAACGGCACTAAA |
| | | AAAATATGCCGTCTACAATCATCCC |
| | | AAATACGGCAAAATCTATGCTTTTG |
| | | AAGTCGATGGCTTCGGAAACCAACT |
| | | ATTAATGGATGACGCTAACGTTCCC |
| | | TCTCTAATAGCCCTACCTTATCTTG |
| | | GCGATGTAAAAGTGAACGACCCAAT |
| | | CTACCAGAATACTAGAAAGTTTGTC |
| | | TGGAGTGAGGACAATCCTTACTTCT |
| | | TCAAGGGTACCGCAGGAGAAGGCAT |
| | | CGGCGGTCCTCATATTGGTTACGAT |
| | | ATGATTTGGCCTATGTCTATCATGA |
| | | TGAAGGCCTTCACATCTCAGAATGA |
| | | TGCAGAGATAAAAACATGTATCAAA |
| | | ATGTTGATGGACACTGATGCCGGCA |
| | | CAGGTTTTATGCATGAGTCCTTTCA |
| | | CAAAAACGACCCAAAAAATTTCACC |
| | | AGATCCTGGTTTGCTTGGCAGAACA |
| | | CGTTGTTCGGAGAGTTAATTCTAAA |
| | | ATTGGTAAACGAAGGTAAAGTCGAT |
| | | TTATTGAACAGTATCCAATAG |
| 25 | BT3774 | ATGAACAAAAAAGTAATTGCCGTAG |
| | | CCCTCGCCCTTGCCTTAGCAGGAGG |
| | | AAGCTATGCACAAGATGACACCGCG |
| | | AAGAAAAAGGTGAAAGCCTATATGG |
| | | TGTCGGACGCCCACCTCGACACCCA |
| | | GTGGAACTGGGACATCCAGACAACA |
| | | ATCAACGAATATGTCTGGAATACCA |
| | | TTAGTCAGAACTTATTTCTGCTAAA |
| | | GAAATATCCCGAATACGTTTTCAAC |
| | | TTTGAAGGGGGAGTGAAGTATGCGT |
| | | GGATGAAGGAATACTATCCCGAACA |
| | | GTATGAAGAGATGAAGAAATTCATC |
| | | GAGGAAGGCCGCTGGCATATCGCCG |
| | | GAAGTAGCTGGGAAGCAAGTGATGT |
| | | GTTGGTTCCTTCCGTCGAAGCCTCC |
| | | ATCCGTAACATCATGCTCGGACAGA |
| | | CGTACTACCGGCAAGAGTTCGGAAA |
| | | AGAAGGAACGGATATCTTCCTGCCG |
| | | GACTGCTTCGGATTCGGATGGACGC |
| | | TTCCCACCATTGCCGCACACTGCGG |
| | | ACTGATCGGCTTCTCTTCACAGAAG |
| | | CTGGACTGGCGTAATCATCCCTTCT |
| | | ATGGAAAGAGCAAGCATCCGTTTAC |
| | | CATCGGACTCTGGAAGGGCATTGAC |
| | | GGCAAACAAGTAATGCTAGCCCACG |
| | | GATATGACTACGGACGCAAATGGAA |
| | | CAACGAAGATCTCTCGAAGAATAAA |
| | | GATCTGGAAAAATTAGCCCAACGTA |
| | | CTCCGCTCAATACGGTCTACCGCTA |
| | | TTATGGAACAGGGGATATCGGTGGC |
| | | TCTCCTACTCTGGGTTCGGTACGTT |
| | | CTGTAGAACAGGGAATCAAAGGTGA |
| | | TGGCCCGGTAGAGGTGATCAGTGCT |
| | | ACCAGCGATCAGTTGTTCAAAGATT |
| | | ATCTGCCGTTCAACAATCACCCGGA |
| | | ACTGCCGGTATTTGACGGAGAGTTA |
| | | TTGATGGATGTTCACGGAACAGGTT |
| | | GCTATACTTCGCAGGCAGCCATGAA |
| | | GCTGTACAACCGGCAAAACGAACAG |
| | | TTGGGCGATGCAGCAGAAAGAGCGG |
| | | CGGTCGCTGCCGAATGGTTGGGTAC |
| | | TGCCAGCTATCCGCAACACACGCTG |
| | | ACGGAGGCATGGAAACGTTTCATCT |
| | | TCCATCAATTCCATGATGACCTGAC |
| | | GGGAACGAGTATCCCCCGTGCCTAT |
| | | GAGTTCTCATGGAACGATGAACTGA |
| | | TCTCTCTAAAACAATTCTCACAAGT |
| | | ACTGACTTCTTCCGTCAACGCCATT |
| | | GCCGGACAGATGGATACACGCGTGA |
| | | AAGGAACGCCTGTCGTTCTTTATAA |
| | | TGCAAACGCTTTCCCGGTATCGGAC |
| | | TTGACAGAGATCATCCTCGAACAGC |
| | | CTAAAACCCCGAAAGGCTTCACTGT |
| | | ATACAATGCACAAGGCAAGAAAGTC |
| | | GCTTCGCAAATGATCGGTTACGAGA |
| | | ACGGACGTGCTCACATCCTGGTTGC |
| | | AGCGTCACTGCCCGCAAACAGTTAT |
| | | GCAGTGTACGATGTCCGCACCGGAG |
| | | GATCTGAAAAAACGATCTCTCCTTC |
| | | AGCCGCCTCAGCCATCGAAAACTCC |
| | | GTCTACAAAATCACACTGGATAAAA |
| | | ACGGAGATATCATCTCACTGACCGA |
| | | CAAGCGCAACAACAAAGAACTCGTA |
| | | AAAGATGGAAAAGCGATTCGCCTGG |
| | | CACTCTTCACCGAAAACAAGTCGTA |
| | | CGCATGGCCTGCATGGGAAATCCTG |
| | | AAAGAGACCATCGACCGTGAACCTG |
| | | TCTCCATCACAGACGGCGCAAAGAT |
| | | CACTTTAGTGGAAAACGGCGCACTC |
| | | CGTAAAGCACTCTGCATTGAGAAGA |
| | | AGTATGGCAAATCGCTCTTCAAGCA |
| | | ATACATCCGCCTCTACGAAGGCAGC |
| | | CGTGCCGACCGCATAGATTTCTATA |
| | | ACGAAATAGACTGGCAGTCAACAAA |
| | | CACACTGCTGAAAGCAGAGTTTCCT |
| | | CTGAATATAGAAAATGAAAAGGCTA |
| | | CTTACGATCTGGGAATCGGCAGCGT |
| | | GGAAAGAGGTAATAATGTACAGACC |
| | | GCTTACGAAGTATATGCGCAGCAAT |
| | | GGGCAGACCTGACCGATAAGAACAA |
| | | CAGCTACGGTGTATCGATCCTAAAT |
| | | GACAGTAAATATGGCTGGGATAAAC |
| | | CGGATAACAACACGATCCGTCTGAC |
| | | TCTTCTCCATACACCGGAAACAAAA |
| | | GGAAATTACGCTTATCAGGATCACC |
| | | AGGACTTCGGCTTCCATACATTTAC |
| | | TTATAGCCTCACAGGACATAACGGA |
| | | GCACTTGACAAACCCGCCACCGCCA |
| | | TCAAAGCTGAAATTCTGAATCAGCC |
| | | GATCAAAGCCTTCAGCAGTCCGAAA |
| | | CATGCCGGAACACTAGGTAAAGAAT |
| | | TTGCTTTTGTACGTTCAAGCAACGA |
| | | TCAAGTCGTTATCAAAGCGCTGAAA |
| | | AAAGCGGAAGTATCCGATGAATATG |
| | | TAGTACGTGTATATGAAACAGGAGG |
| | | CGCAGCTCCGCAACAGGCAGCCATC |
| | | ACCTTCGCCGGTGAAATAGAGAAGG |
| | | CAGTACTTGCAGACGGTACGGAAAA |
| | | AGAGATCGGCAGTGCTGACTTCAAC |
| | | AAGAACCAGCTGAATGTATCCATCG |
| | | CTCCCTACAGCATACAGACATTTAA |
| | | AGTGAAGCTGAAGAAAAAAGCTGAT |
| | | CTTCAAGCTCCGGCATGCGCTTATC |
| | | TTCCTTTGGACTATGATCGCAGATG |
| | | TTTCAGTTGGAATGCTTTCCGCAAA |
| | | GAAGGGAACTTCGAATCGGGCAACA |
| | | GCTATGCAGCAGAACTTCTCCCCGA |
| | | CTCCATCCTGAAAGCCGACGGCATT |
| | | CCTTTCCGCTTGGGAGAGAAAGAAA |
| | | TTGCCAATGGTTTGACTTGCAAAGG |
| | | CAATGTACTTCAGTTGCCAACCGGA |
| | | CATTCTTACAACCGTATCTATTTCC |
| | | TGGCAGCCTCTGCCGGTGAAGATGC |
| | | AGTTGCTACCTTCAGCACCGGTAAC |
| | | AACTCACAGGAAATCACCGTACCTT |
| | | CCTATACCGGTTTTATCGGTCAGTG |
| | | GGAGCATCTGGGACATACGGAAGGC |
| | | TTCCTGAAAGATGCAGAAATCGCTT |
| | | ATGTCGGCACTCACCGTCATGCTTC |
| | | TGACAAAGATGAGGCTTATGAGTTT |
| | | ACGTATATGTTCAAGTTTGGCATGG |
| | | ATATTCCTAAAGGAGCGACTACGGT |
| | | TACTTTGCCGGATCATGCAGATATC |
| | | GTATTATTTGCCGCAACGCTGGTTA |
| | | ATGAGAAGTATCCGGCAGTAACTCC |
| | | GGCCTCGGAATTGTTCCGCACAGCC |
| | | TTGAAAGCAGACAATGGAGAAGAAG |
| | | CGACGACTAAAACAAACCTGTTGAA |
| | | ACAAGCCAAACTAATCAAATGTTCC |
| | | GGTGAAACCAACGAAAAAGAAGTTG |
| | | CAAGATATGCCGTAGACGGTGATGT |
| | | GAAGACGAAATGGTGTGATACAAGC |
| | | ACGGCTCCCAACTACATTGACTTCG |
| | | ACTTCGGAAAGGAACAGACGATCCG |
| | | TGGATGGAAGTTGGTAAATGCCGGA |
| | | AATGAAGGCAGCGTCTTTATCACTC |
| | | ATACCTGCTTCTTACAAGGCAGAAA |
| | | CAGTCCGGACGAAGAATGGAAAACG |
| | | ATTGATGAACTGAGTGATAACAAGA |
| | | AAAACACGGTAGTTCGCCAGTTTAA |
| | | GCCGACTTCGGTACGTTACGTCAGA |
| | | CTGCTGGTTACACAATCTACACAAA |
| | | ACAACAGTCTGAAGGCTGCAAGAAT |
| | | CTACGAGTTGGAGGTTTATTGA |
| 26 | BT3780 | ATGAAATCAACCTTTTTATTTCTGG |
| | | TTACTACAACCATGATGACTTGTAC |
| | | CGCCTTGGGACAACCTTCCAACGAC |
| | | AAAAAGAACGTATTACCCGACTGGG |
| | | CGTTCGGAGGCTTCGAACGACCACA |
| | | GGGAGCTAATCCGGTGATATCTCCT |
| | | ATAGAGAACACGAAATTCTATTGTC |
| | | CGATGACACAGGATTACGTTGCATG |
| | | GGAATCCAATGACACTTTCAATCCG |
| | | GCTGCTACCCTGCATGACGGCAAGA |
| | | TTGTCGTGCTGTATCGGGCAGAAGA |
| | | TAAATCCGGTGTCGGTATCGGTCAC |
| | | CGTACCTCACGTCTCGGATACGCCA |
| | | CTTCGAGCGACGGCATTCACTTCAA |
| | | GCGGGAAAAGACCCCGGTATTTTAT |
| | | CCGGATAACGATACTCAAAAGAAAC |
| | | TGGAATGGCCGGGCGGATGCGAAGA |
| | | CCCGCGTATCGCCGTCACAGCAGAA |
| | | GGACTGTATGTGATGACCTATACGC |
| | | AATGGAACCGCCACATTCCGCGTCT |
| | | GGCAATAGCCACTTCCCGCAATCTG |
| | | AAAGACTGGACAAAGCACGGTCCCG |
| | | CTTTTGCCAAAGCGTATGACGGCAA |
| | | GTTCTTCAATTTAGGATGCAAGTCC |
| | | GGCTCCATTCTGACCGAAGTTGTCA |
| | | ATGGGAAACAGGTGATCAAGAAAAT |
| | | CGACGGAAAATACTTCATGTATTGG |
| | | GGAGAGGAACATGTGTTTGCCGCCA |
| | | CTTCCGAAGATTTAGTCAACTGGAC |
| | | TCCATACGTAAATACGGACGGCTCG |
| | | CTGAGAAAACTGTTTTCACCCCGTG |
| | | ACGGACACTTCGACAGCCAGCTGAC |
| | | GGAATGCGGTCCTCCAGCTATTTAT |
| | | ACTCCAAAGGGAATCGTACTTCTGT |
| | | ATAATGGTAAAAACAGTGCAAGCAG |
| | | AGGCGACAAACGCTATACCGCCAAT |
| | | GTTTACGCTGCCGGACAAGCCCTCT |
| | | TCGACGCCAATGACCCGACCCGTTT |
| | | CATCACCCGTCTCGACGAACCGTTC |
| | | TTCCGCCCGATGGATAGTTTCGAAA |
| | | AGAGCGGGCAGTATGTAGACGGAAC |
| | | GGTGTTCATCGAAGGGATGGTTTAT |
| | | TATAAGGATAAATGGTATCTGTATT |
| | | ATGGTTGCGCAGATTCCAAGGTGGG |
| | | TATGGCTATCTACAATCCGAAGAAA |
| | | CCTGCTGCCGCAGATCCGCTGCCCT |
| | | AA |
| 27 | BT3780 | ATGAAGTCTACCTTTCTATTCCTAG |
| | (codon | TGACGACTACCATGATGACTTGCAC |
| | optimized) | CGCTCTTGGACAGCCCTCCAACGAC |
| | | AAAAAGAACGTCTTACCCGACTGGG |
| | | CATTTGGTGGCTTTGAACGTCCACA |
| | | AGGCGCTAATCCAGTTATTTCCCCC |
| | | ATAGAAAATACTAAATTTTATTGCC |
| | | CTATGACGCAGGACTACGTAGCCTG |
| | | GGAATCAAACGACACCTTTAATCCT |
| | | GCCGCAACTCTGCACGATGGCAAAA |
| | | TCGTGGTGTTGTATAGAGCCGAAGA |
| | | CAAATCCGGCGTCGGCATCGGACAT |
| | | AGGACATCAAGATTGGGATACGCCA |
| | | CGTCCTCTGACGGTATACATTTCAA |
| | | AAGAGAGAAGACCCCTGTCTTTTAT |
| | | CCCGACAATGATACGCAGAAAAAAC |
| | | TTGAATGGCCTGGCGGTTGTGAGGA |
| | | TCCAAGGATTGCAGTGACGGCAGAG |
| | | GGACTTTATGTTATGACTTACACCC |
| | | AATGGAATAGACATATACCTCGTCT |
| | | AGCAATCGCAACCTCTAGGAACCTT |
| | | AAAGATTGGACGAAACATGGCCCCG |
| | | CTTTTGCTAAAGCCTACGACGGAAA |
| | | GTTTTTCAATTTAGGCTGTAAGAGT |
| | | GGCAGTATTTTGACAGAAGTGGTCA |
| | | ATGGTAAACAGGTGATCAAGAAAAT |
| | | CGATGGTAAGTATTTTATGTATTGG |
| | | GGTGAGGAACACGTTTTCGCAGCTA |
| | | CTTCTGAAGACCTGGTGAACTGGAC |
| | | ACCCTACGTTAATACAGATGGAAGT |
| | | CTAAGGAAGTTATTTTCACCTCGTG |
| | | ACGGTCACTTCGACTCCCAACTAAC |
| | | GGAATGTGGCCCACCCGCCATTTAT |
| | | ACGCCTAAGGGCATCGTACTGCTGT |
| | | ATAACGGTAAAAATAGTGCCAGTAG |
| | | AGGCGATAAAAGATACACCGCTAAC |
| | | GTATACGCAGCCGGCCAAGCTCTAT |
| | | TCGATGCTAACGACCCTACCAGGTT |
| | | CATAACTAGATTGGACGAGCCCTTT |
| | | TTCAGGCCAATGGATTCATTTGAGA |
| | | AATCAGGCCAGTACGTAGATGGCAC |
| | | GGTTTTTATTGAGGGCATGGTTTAT |
| | | TACAAGGATAAATGGTATCTTTATT |
| | | ATGGTTGTGCTGATTCTAAAGTTGG |
| | | TATGGCAATATATAATCCCAAGAAG |
| | | CCAGCAGCTGCAGATCCACTTCCCT |
| | | AA |
| 28 | BT3781 | ATGAATATAACCAAAACACTTTGCC |
| | | TCTGCGCAGCACTTTCGGGCGCTGC |
| | | CGGCGTGCAAGCAATGGAAAACCGC |
| | | GAATTTGTGACCCAGCAAGACAATA |
| | | CCCGGGTCAATAATTACCAGACCAA |
| | | CCGTCCCGAAGCCTCCAAGCGCTTA |
| | | TTCGTATCGCAGGAAGTGGAACGAC |
| | | AGATTGACCACATCAAGCAACTACT |
| | | GACCAATGCGAAACTGGCATGGATG |
| | | TTCGAGAACTGTTTTCCGAACACAC |
| | | TGGACACTACCGTTCACTTCGACGG |
| | | AAAAGAGGACACTTTTGTATACACC |
| | | GGAGACATCCACGCCATGTGGCTCC |
| | | GCGACTCCGGTGCGCAGGTATGGCC |
| | | CTATGTGCAGCTTGCCAATAAAGAC |
| | | CCCGAACTGAAAAAGATGCTGGCAG |
| | | GAGTCATCAACCGCCAGTTTAAATG |
| | | TATCAATATCGACCCGTACGCCAAC |
| | | GCCTTCAACATGAACTCCGAAGGAG |
| | | GCGAATGGATGAGCGACCTGACGGA |
| | | CATGAAACCGGAACTTCACGAACGC |
| | | AAATGGGAAATCGACTCTCTCTGCT |
| | | ACCCGATCCGCCTGGCATACCATTA |
| | | CTGGAAAACAACGGGCGATGCCAGC |
| | | GTATTCTCCGACGAATGGCTGCAGG |
| | | CCATTGCAAATGTGCTGAAGACTTT |
| | | CAAGGAACAGCAGCGTAAGGACGAC |
| | | GCGAAAGGTCCGTACAGATTCCAGC |
| | | GTAAGACCGAACGCGCACTCGACAC |
| | | CATGACCAATGACGGTTGGGGCAAT |
| | | CCGGTGAAACCTGTCGGACTGATTG |
| | | CTTCCGCTTTCCGCCCTTCGGATGA |
| | | CGCTACGACTTTCCAGTTCCTCGTT |
| | | CCTTCCAACTTCTTTGCCGTTACTT |
| | | CCTTGCGCAAAGCTGCCGAAATTCT |
| | | GAACACCGTGAACAGGAAACCGGCG |
| | | CTGGCCAAAGAATGTACCGCACTGG |
| | | CGGATGAAGTAGAAAAAGCATTAAA |
| | | GAAATATGCTGTCTGCAACCATCCG |
| | | AAATACGGTAAGATTTATGCTTTCG |
| | | AGGTAGATGGCTTCGGCAATCAGCT |
| | | ACTGATGGACGACGCCAACGTGCCG |
| | | AGTCTCATCGCTTTGCCTTATCTGG |
| | | GTGACGTCAAAGTGACTGATCCGAT |
| | | TTATCAGAATACCCGCAAGTTTGTA |
| | | TGGAGCGAAGACAATCCTTACTTCT |
| | | TCAAAGGCAGTGCCGGGGAAGGTAT |
| | | CGGAGGTCCGCATATCGGATATGAC |
| | | ATGATATGGCCCATGAGTATCATGA |
| | | TGAAAGCCTTCACCAGCCAGAATGA |
| | | CGCAGAAATCAAAACTTGCATCAAA |
| | | ATGCTGATGGATACGGATGCAGGTA |
| | | CCGGCTTCATGCACGAATCATTCAA |
| | | CAAAAACGACCCGAAAAACTTTACC |
| | | CGTGCATGGTTTGCATGGCAGAATA |
| | | CGTTGTTCGGAGAGCTGATCCTCAA |
| | | ACTGGTCAATGAAGGCAAAGTGGAC |
| | | TTATTGAACAGCATTCAGTAG |
| 29 | BT3781 | ATGAATATTACGAAAACTTTGTGCT |
| | (codon | TGTGTGCAGCACTAAGTGGCGCAGC |
| | optimized) | CGGAGTTCAGGCAATGGAGAACCGT |
| | | GAGTTTGTTACTCAACAGGATAATA |
| | | CAAGAGTCAATAACTATCAAACGAA |
| | | CCGTCCCGAGGCATCTAAAAGATTA |
| | | TTCGTAAGTCAAGAAGTGGAAAGGC |
| | | AGATAGACCATATAAAACAGTTATT |
| | | GACCAATGCCAAATTAGCATGGATG |
| | | TTCGAAAATTGCTTCCCCAATACTC |
| | | TGGACACGACCGTACATTTCGATGG |
| | | TAAAGAAGATACATTCGTTTACACC |
| | | GGAGACATTCACGCTATGTGGCTAA |
| | | GAGACTCAGGCGCTCAGGTATGGCC |
| | | ATACGTTCAGCTAGCTAATAAGGAT |
| | | CCCGAGCTGAAAAAGATGCTAGCTG |
| | | GTGTTATTAATCGTCAGTTTAAATG |
| | | TATCAATATAGATCCCTATGCTAAC |
| | | GCATTTAATATGAACTCCGAGGGCG |
| | | GTGAATGGATGTCTGATCTGACAGA |
| | | TATGAAACCCGAACTGCACGAAAGG |
| | | AAATGGGAAATTGATAGTCTGTGCT |
| | | ACCCAATCAGACTGGCATATCATTA |
| | | TTGGAAGACTACCGGTGATGCTTCC |
| | | GTATTTTCCGATGAATGGCTACAGG |
| | | CCATAGCAAATGTATTAAAAACTTT |
| | | CAAAGAGCAACAGAGGAAGGACGAC |
| | | GCAAAGGGACCCTATAGATTTCAAA |
| | | GGAAGACGGAAAGAGCTTTAGACAC |
| | | TATGACTAACGACGGCTGGGGAAAT |
| | | CCAGTCAAGCCAGTGGGTCTAATCG |
| | | CATCCGCATTTAGGCCCTCAGATGA |
| | | CGCAACTACGTTCCAGTTCCTGGTC |
| | | CCTTCAAACTTCTTCGCAGTCACGT |
| | | CTTTAAGGAAAGCAGCTGAGATACT |
| | | AAATACGGTGAACAGAAAGCCTGCC |
| | | TTGGCTAAAGAGTGCACAGCACTGG |
| | | CAGATGAGGTAGAGAAAGCCTTGAA |
| | | GAAATACGCAGTGTGCAATCATCCC |
| | | AAGTATGGCAAGATATACGCCTTCG |
| | | AAGTAGACGGCTTTGGTAATCAACT |
| | | ATTGATGGATGATGCTAATGTCCCT |
| | | AGTTTAATAGCACTACCTTATTTAG |
| | | GCGACGTAAAAGTGACGGACCCAAT |
| | | TTACCAAAATACCAGAAAATTCGTC |
| | | TGGTCCGAAGATAATCCCTACTTTT |
| | | TCAAAGGTTCAGCAGGAGAAGGTAT |
| | | CGGAGGACCCCATATTGGTTACGAC |
| | | ATGATATGGCCCATGAGTATAATGA |
| | | TGAAGGCATTTACGAGTCAGAATGA |
| | | CGCAGAGATCAAAACCTGCATAAAG |
| | | ATGCTGATGGACACTGATGCTGGCA |
| | | CGGGTTTTATGCACGAGTCTTTTAA |
| | | TAAAAACGATCCAAAAAATTTTACC |
| | | CGTGCCTGGTTCGCTTGGCAGAACA |
| | | CCTTGTTTGGAGAGTTGATACTGAA |
| | | GTTGGTAAATGAAGGTAAAGTGGAT |
| | | CTACTGAACTCCATTCAATAG |
| 30 | BT3782 | ATGAGAAATATATGTTTTGTAGCGG |
| | | TATGTTGTTTTGCCTCGCTTCCCCT |
| | | TCCGGAAAAACGGTGAAAAATCATC |
| | | CTTTCGTGTCCATTGCCGACTCTAT |
| | | CCTCGACAATGTTCTGAATTTATAT |
| | | CAGACGGAAGACGGGCTGCTTACCG |
| | | AAACATATCCCGTGAATCCCGACCA |
| | | GAAAATCACTTATCTGGCAGGCGGA |
| | | GCACAGCAGAACGGAACCTTGAAGG |
| | | CCTCCTTTCTGTGGCCCTACTCAGG |
| | | GATGATGTCCGGTTGCGTAGCCATG |
| | | TACCAGGCTACCGGAGACAAGAAGT |
| | | ACAAGACGATACTGGAAAAGCGCAT |
| | | CCTGCCGGGACTGGAACAGTACTGG |
| | | GACGGAGAACGCCTTCCGGCATGCT |
| | | ATCAGTCGTACCCTGTCAAATACGG |
| | | TCAGCATGGACGCTACTACGATGAC |
| | | AACATCTGGATCGCACTGGATTATT |
| | | GCGACTACTACCGCCTCACAAAGAA |
| | | GGCCGACTATCTGAAAAAGGCCATT |
| | | GCCCTGTACGAATACATCTACAGCG |
| | | GCTGGAGTGACGAACTGGGCGGAGG |
| | | AATCTTCTGGTGCGAACAGCAGAAA |
| | | GAAGCGAAGCATACCTGCTCCAATG |
| | | CCCCGTCAACAGTACTCGGCGTCAA |
| | | GCTATACCGTCTGACGAAGGACAAA |
| | | AAGTATCTGAACAAGGCCAAGGAAA |
| | | CTTACGCATGGACCAGAAAACACTT |
| | | GTGTGATCCCGACGACTTCCTTTAC |
| | | TGGGACAATATCAACCTGAAAGGGA |
| | | AAGTCTCGAAAGACAAGTACGCCTA |
| | | CAACAGTGGACAAATGATTCAGGCA |
| | | GGTGTATTACTGTACGAAGAGACAG |
| | | GAGACAAGGATTACTTGCGCGATGC |
| | | CCAGAAGACAGCCGCGGGAACCGAT |
| | | GCCTTTTTCCGTTCGAAAGCAGATA |
| | | AGAAAGACCCGTCAGTCAAGGTACA |
| | | CAAGGATATGTCGTGGTTTAACGTG |
| | | ATTCTGTTCAGAGGCTTCAAGGCGC |
| | | TGGAGAAGATTGACCACAACCCGAC |
| | | TTATGTCCGTGCGATGGCAGAGAAC |
| | | GCGCTCCACGCATGGAGAAACTACC |
| | | GGGATGCCAACGGATTACTGGGCAG |
| | | AGACTGGTCAGGACATAACGAGGAA |
| | | CCTTATAAATGGCTGCTCGATAATG |
| | | CCTGCCTGATCGAGCTGTTCGCTGA |
| | | AATCGAGAAATAA |
| 31 | BT3782 | ATGCGTAACTTGTTTTGTCGCTTGT |
| | (codon | ATGCTGTTTTGTCTTGCATCCGCCT |
| | optimized) | CTGGAAAAACTGTCAAAAATCATCC |
| | | ATTTGTATCCATTGCCGACTCCATA |
| | | CTAGATAACGTACTAAACCTATACC |
| | | AAACAGAAGACGGCCTATTAACTGA |
| | | AACATATCCTGTCAACCCTGACCAG |
| | | AAAATCACCTATTTGGCAGGCGGCG |
| | | CTCAGCAGAACGGAACCCTAAAGGC |
| | | ATCCTTTCTTTGGCCTTACTCCGGT |
| | | ATGATGTCCGGCTGTGTGGCCATGT |
| | | ACCAAGCTACCGGAGACAAAAAGTA |
| | | CAAAACCATACTAGAGAAGCGTATC |
| | | TTACCAGGATTAGAACAATACTGGG |
| | | ATGGTGAGCGTTTGCCCGCATGTTA |
| | | CCAATCCTATCCCGTGAAATACGGA |
| | | CAACACGGCAGGTACTATGACGACA |
| | | ACATTTGGATTGCATTGGACTATTG |
| | | TGATTATTACCGTCTAACAAAGAAA |
| | | GCAGACTATCTGAAAAAAGCCATTG |
| | | CTCTATATGAATACATATACAGTGG |
| | | CTGGAGTGATGAGTTAGGTGGCGGC |
| | | ATCTTTTGGTGTGAGCAGCAAAAGG |
| | | AAGCCAAGCACACGTGCTCCAATGC |
| | | ACCCTCCACGGTCTTAGGTGTTAAG |
| | | CTTTACAGGCTAACGAAGGACAAGA |
| | | AATACTTAAATAAGGCTAAGGAGAC |
| | | TTACGCCTGGACTAGAAAGCATCTT |
| | | TGCGACCCCGACGACTTTTTATATT |
| | | GGGATAATATTAACTTAAAGGGAAA |
| | | AGTTTCCAAAGATAAATATGCATAC |
| | | AACTCTGGCCAAATGATCCAGGCCG |
| | | GAGTACTACTATACGAAGAAACTGG |
| | | CGATAAAGACTACCTTAGGGATGCC |
| | | CAAAAAACGGCCGCTGGTACGGACG |
| | | CCTTTTTCCGTAGTAAAGCAGACAA |
| | | AAAAGATCCATCAGTCAAAGTTCAC |
| | | AAAGATATGTCTTGGTTCAACGTCA |
| | | TCCTATTCAGAGGTTTTAAAGCTCT |
| | | AGAGAAGATTGACCACAACCCAACT |
| | | TATGTGCGTGCCATGGCAGAGAATG |
| | | CACTTCACGCTTGGCGTAACTATAG |
| | | AGATGCAAACGGACTTCTGGGCAGG |
| | | GACTGGAGTGGCCATAATGAAGAGC |
| | | CATACAAGTGGCTACTGGATAATGC |
| | | CTGTCTAATAGAATTATTCGCAGAG |
| | | ATCGAGAAATAA |
| 32 | BT3783 | ATGAAACTAAGAAACCTTTTATTTA |
| | | TCGTTCTTGCAGCGATAGTCTTCTG |
| | | CAACTGTCAGAGCTATCAGCCTACT |
| | | TCGCTCACCGTTGCCTCCTACAACC |
| | | TGAGAAATGCCAACGGTTCCGACTC |
| | | CGCCCGTGGAGACGGATGGGGACAG |
| | | CGTTATCCGGTGATTGCCCAGATGG |
| | | TGCAATATCACGATTTCGATATTTT |
| | | CGGCACACAGGAATGCTTCCTTCAC |
| | | CAACTGAAAGACATGAAAGAAGCCC |
| | | TTCCCGGTTATGACTATATCGGCGT |
| | | AGGCCGCGACGACGGTAAAGACAAA |
| | | GGCGAACACTCCGCTATCTTCTACC |
| | | GCACCGACAAATTCGACATCGTAGA |
| | | AAAAGGAGATTTCTGGCTGTCGGAA |
| | | ACTCCGGACGTGCCGAGCAAAGGCT |
| | | GGGATGCCGTATTGCCTCGTATTTG |
| | | CAGCTGGGGGCACTTCAAATGCAAA |
| | | GATACCGGTTTCGAGTTTCTGTTCT |
| | | TCAATCTCCACATGGACCACATCGG |
| | | CAAGAAAGCCCGTGTGGAGAGCGCT |
| | | TTCCTCGTACAGGAAAAGATGAAAG |
| | | AGCTGGGAAGAGGCAAGAATCTGCC |
| | | GGCTATCCTGACGGGAGACTTCAAC |
| | | GTCGACCAGACCCACCAGTCCTACG |
| | | ACGCATTTGTCAGCAAAGGCGTCCT |
| | | CTGTGATTCTTACGAGAAGTGCGAC |
| | | TACCGATATGCGCTCAACGGAACTT |
| | | TCAACAACTTCGATCCGAACAGTTT |
| | | TACCGAAAGCCGCATCGACCATATC |
| | | TTCGTTTCACCTTCTTTCCACGTCA |
| | | AGAGATACGGTGTGCTGACAGATAC |
| | | CTATCGGAGTGTACGGGAAAACAGT |
| | | AAAAAGGAGGACGTGAGAGATTGTC |
| | | CGGAAGAGATCACCATTAAGGCTTA |
| | | TGAAGCACGTACACCATCCGACCAT |
| | | TTCCCTGTAAAAGTGGAACTGGTGT |
| | | TTGACCAACGTCAGCAAAAATAA |
| 33 | BT3784 | ATGAAAACACATTTTTCATAAACAC |
| | | CTGTTATTTATTGGAGGTGCGGTGT |
| | | TGTACAGCATGCAAATTTCTGCCGT |
| | | CAAGAATCCGGTAGACTATGTCAGC |
| | | ACGCTGGTAGGAACGCAGTCCAAGT |
| | | TTGAGTTATCGACCGGAAATACCTA |
| | | TCCGGCTACGGCACTGCCGTGGGGA |
| | | ATGAACTTCTGGACACCGCAAACCG |
| | | GTAAAATGGGCGACGGTTGGGCATA |
| | | TACCTACAATGCCGACAAAATCCGG |
| | | GGCTTCAAACAAACACATCAACCCA |
| | | GCCCGTGGATGAACGACTACGGTCA |
| | | GTTTTCCATCATGCCGATCACAGGC |
| | | GGACTGGTATTCGACCAGGACCAAC |
| | | GTGCCAGCTGGTTCTCGCACAAGGC |
| | | GGAGGTTGCCAAACCTTATTATTAT |
| | | AAGGTATATCTCGCAGACCACGACG |
| | | TTACTACGGAACTCGTTCCGACGGA |
| | | GCGTGCCGCTATGTTCCGTTTCACG |
| | | TATCCGGAAACCAAGAACGCTTATG |
| | | TCGTTATCGACGCATTCGACAAAGG |
| | | CTCTTATGTAAAGGTGATTCCGGAA |
| | | GAAAACAAGATCATCGGTTATTCTA |
| | | CCAAGAACAGCGGCGGAGTGCCGGA |
| | | GAACTTCAAGAATTATTTTGTCATC |
| | | CAGTTCGACAAGCCGTTTACCTTTA |
| | | CTTCCGGCGTGAAAGAGAACAACAT |
| | | TCTCCCGAACGAAACAGAAGTTCAG |
| | | GGCAACCATACCGGAGCGATCATCG |
| | | GATTCGCTACCCAGAAAGGGGAGAT |
| | | CGTTCACGCACGTGTAGCTTCTTCT |
| | | TTTATCAGTTATGAGCAGGCGGAAC |
| | | TGAATCTCAAAGAATTGGGCAAGGA |
| | | TAGTTTCGACCAGCTGGTCACTAAA |
| | | GGAAAAGACATCTGGAACCGTGAAA |
| | | TGAGCAAAGTAGATGTGGAAGACGA |
| | | TAATATCGACAATCTGCGCACTTTC |
| | | TATTCCTGCCTCTATCGTTCGATGC |
| | | TGTTCCCACGCAGCTTTTATGAAAT |
| | | AGACGCCAAAGGACAGGTCGTACAC |
| | | TACAGCCCTTACAACGGAAAAGTGC |
| | | TACCGGGCTATATGTTTACGGATAC |
| | | CGGCTTCTGGGATACGTTCCGCTGT |
| | | CTGTTCCCATTCTTGAACCTGATGT |
| | | ATCCGTCCATGAATCAGAAGATGCA |
| | | GGAAGGACTGGTCAATGCGTACCTT |
| | | GAAAGCGGATTCCTTCCGGAATGGG |
| | | CAAGTCCCGGACACCGTGACTGTAT |
| | | GGTCGGCAACAACTCCGCTTCCGTA |
| | | GTAGCCGACGCCTATATCAAAGGAC |
| | | TGCGCGGATATGACATCGAAACACT |
| | | TTGGGAAGCATTGAAACATGACGCA |
| | | AACGCCCATCTCCGCGGCACAGCTT |
| | | CGGGCCGCCTTGCATACGACGCCTA |
| | | CAACAAACTGGGTTATGTCCCCAAC |
| | | AATATCGGTATAGGACAGAATGTTG |
| | | CCCGTACGCTGGAATATGCGTACAA |
| | | CGACTGGACCATCTACACGCTAGGC |
| | | AAGAAACTGGGCAAACCGGCAAGCG |
| | | AAATCGACATCTTCAAACAACGTGC |
| | | ACTCAACTACAAGAACGTCTACCAC |
| | | CCGAAACGCAAACTGATGGTAGGCA |
| | | AAGACGACAAAGGTGTGTTCAACCC |
| | | CAAATTTGATGCAGTAGACTGGAGC |
| | | GGCGAGTTCTGCGAAGGTAACAGCT |
| | | GGCACTGGAGTTTCTGCGTATTCCA |
| | | TGATCCGCAAGGACTGATCGACCTG |
| | | ATGGGAGGCAAGAAAGAATTCAACA |
| | | ACATGATGGATTCCGTCTTTGTCAT |
| | | TCCGGGCAAACAGGGTATGGAAAGC |
| | | CGTGGCATGATCCACGAAATGCGTG |
| | | AAATGCAGGTAATGAACATGGGACA |
| | | GTACGCTCACGGCAACCAGCCTATC |
| | | CAGCACATGGTTTATCTTTACAACT |
| | | ATTCGGGAGAACCGTGGAAGGCCCA |
| | | GCATTGGGTTCGTGAAATCATGGAC |
| | | AAGCTCTACACGGCAGGCCCCGACG |
| | | GATATTGCGGTGACGAAGACAACGG |
| | | TCAGACTTCTGCCTGGTATGTCTTC |
| | | TCGGCTTTAGGATTCTACCCCGTTT |
| | | GTCCGGGAACAGATCAGTACATTCT |
| | | GGGAACTCCCCTTTTCAAGTCAGCC |
| | | AAGCTGCATCTGGAAAATGGAAAAA |
| | | CCGTCACAATAAAAGCAAGCAACAA |
| | | TAACACCGACAACCGTTATGTGAAG |
| | | GATATGAAGGTAAATGGCAAGGCAT |
| | | TCACCCGCAATTATCTGACGCACGA |
| | | CCAATTACTGAAAGGAGCGAATATC |
| | | CAGTATCAGATGAGTCCTACGCCGA |
| | | ACAAACAGCGGGGAACGACTGAAAA |
| | | AGATATTCCCTATTCCCTTTCATTT |
| | | GAATAA |
| 34 | BT3788 | ATGAAAAATACTCATATTTCATATT |
| | | ACTTTTGATGTTGATATTACTTGTT |
| | | CCAAGCAATATATGGGGACAAGAAA |
| | | CAAAAAAGGAAATTATAGTCAAAGG |
| | | TGTAGTGGAAGATGATTTAGGGCCG |
| | | ATAATTGGTGCGTCAGTCGTTGCTA |
| | | AAAACCAGGCAGGTGTGGGAGTAAT |
| | | CACAAATACTGAAGGTAAGTTTTCT |
| | | TTGAAAGTGGGACCTTATGATGTAT |
| | | TGGTAGTGACTTTTGTTGGTTATCA |
| | | GCCATATGAGCTGCCTGTTCTGAAA |
| | | ATGAATGATCCCAATAATGTAACTA |
| | | TAAAGTTATTGGAAGATGTTGGCAA |
| | | AATTGATGAAGTGGTAATTACAGCC |
| | | AGTGGACTTCAACAAAAGAAAACTC |
| | | TGACTGGGGCAATAACCAATGTTGA |
| | | TGTAAAACAGTTGAATGCTGTAGGA |
| | | AGTAGTAGTCTTTCTAATTCATTGG |
| | | CTGGTGTGGTTCCCGGTATTATAGC |
| | | CATGCAGCGTAGTGGTGAACCGGGT |
| | | GAAAATACATCTGAATTCTGGATTC |
| | | GAGGTATTAGTACCTTTGGTGCAAA |
| | | ATCAGGAGCCTTAGTTCTTATCGAC |
| | | GGAGTAGAACGAAATTTTGATGAGA |
| | | TTTTGCCGCAAGACATTGAATCGTT |
| | | CTCAGTACTGAAAGATGCATCAGCA |
| | | ACTGCAATATATGGTCAGCGCGGTG |
| | | CAAATGGAGTTATCTTAATTACCAC |
| | | CAAGCGTGGGGAAAAAGGTAAGGTG |
| | | AAAATTAATGTAAAAGCAGGATTTG |
| | | ACTGGAATACTCCTGTAAAAGTGCC |
| | | AGAGTATGCAAGTGGTTATGATTGG |
| | | GCGCGTTTAGCCAATGAGGCTCGGT |
| | | TAGGACGCTATGATTCCCCGATTTA |
| | | TACTCCTGAAGAATTGGAGATAATT |
| | | AGATCAGGTTTAGATACTGATTTAT |
| | | ATCCTAATATTGATTGGAGGGATTT |
| | | AATGTTGAAGAGTGGTGCACCTCGC |
| | | TATTATGCTAATATTAGTTTTTCAG |
| | | GTGGTAGTGATAATGTACGTTATTA |
| | | TGTCTCTGGACAATATACCAGTGAA |
| | | CAAGGACGTTACAAAACGTTTAGCT |
| | | CTGAAAATAAGTACAATACCAATAC |
| | | GACTTATGAACGATATAATTATCGT |
| | | GCTAACGTAGACATGAACATAACTA |
| | | AGACAACAGTACTGAAAGTTAGTGT |
| | | AGGTGGATGGTTGGTGAATAGGACT |
| | | ACGCCTACTAGAAGTACTGGTGACA |
| | | TATGGGAGGATTTTGCCAAATTTAC |
| | | TCCTTTGTCTACTCCTCGTAAATGG |
| | | TCTACAGGACAATGGCCGAGAGTGG |
| | | ATGGGCAAGATACTCCTGAATATCA |
| | | TATGACACAAAGAGGATATCATACG |
| | | AAATGGGAGAGTAAGGTGGAAACTA |
| | | GTGTAAAGTTAGAGCAGGATCTTAA |
| | | GTTTATTACGCCCGGTTTGAAGTTT |
| | | GAAGGAGTATTTGCTTTTGATACTT |
| | | ATAATGAGAATATAATAAAACGGGA |
| | | GAAAAAAGAGGAAGTATGGGAAGCC |
| | | CAAAAATATAGAGATGAAAATGGTA |
| | | AATTGATTTTGAAAAGAGTGGTCAA |
| | | TAGAAGTCCGATGAATCAAAATAAG |
| | | GAAGTTAGGGGTGATAAACGATACT |
| | | ATTTTCAGGCGTCATTAGATTATAA |
| | | CCGTTTATTTGCTCATGCACATCGT |
| | | GTCGGTGTTTTTGGCATGGTATACC |
| | | AAGAGGAAAAGACGGATGTTAATTT |
| | | CGACTCCAGTGATTTGATTGGTTCT |
| | | ATTCCTCGTCGTAATTTGGCTTATT |
| | | CCGGTCGTTTTACTTATGCTTATAA |
| | | AGATAAATACCTTGCTGAATTTAAC |
| | | TGGGGATGTACTGGTTCAGAGAATT |
| | | TTGAACATGGAAAACAATTTGGTTT |
| | | CTTTCCTGCTGTTTCTGCCGGTTAT |
| | | GTAATTTCTGAAGAGGCTTTTATGA |
| | | AAAAAGCATTGCCATGGATAGATCA |
| | | ATTTAAGATCAGAGCTTCTTATGGT |
| | | GAGGTAGGTAATGATGTATTGGATG |
| | | GTCGTCGATTCCCTTATGTGTCTCT |
| | | TATAGATACTGATGATGGAGGATCA |
| | | TATTCATTTGGGGAATTTGGAACAA |
| | | ATAGAGTGCAAGCCTACCGTATTAG |
| | | AACTTTGGGGACTCCTAATTTGACT |
| | | TGGGAGATAGCTAAGAAATATGATG |
| | | TAGGTGTTGACTTTTCTTTTTTTAA |
| | | TGGGAAAATTAGTGGTGCTTTAGAT |
| | | TGGTTTTTGGATAAACGTGATGACA |
| | | TCTTTATGCAGCGTAAACATATGCC |
| | | ATTGACTACCGGGCTTGCTGATCAG |
| | | ACTCCAATGGCCAATGTCGGAAAGA |
| | | TGAAGTCTTATGGATGGGAAGGAAA |
| | | TATAGGATTTACTCAATCTATTGGT |
| | | CAGGTGAATCTCCAACTTCGTGCCA |
| | | ACTTTACTTATCAGACTACTGATAT |
| | | CATAGATAAGGATGAAGCAGCCAAT |
| | | GAGTTATGGTATAAAATGGATAAAG |
| | | GCTTTCAGTTAAATCAATCGCGTGG |
| | | ATTGATTGCTTTAGGATTATTTAAA |
| | | GATCAAGATGAAATAGACCGTAGTC |
| | | CGAAACAGACAAGTAACAGACCTAT |
| | | CCTTCCCGGTGATATTAAATACAAA |
| | | GATGTAAATGGTGATGGAGTTATTA |
| | | ATGATGATGATATTGTGCCTTTAGG |
| | | ATATCGGGAAGTTCCGGGATTACAG |
| | | TATGGTGTCGGTTTAAGTGCTAATT |
| | | GGAGAAATTGGAATTTGAGTGTACT |
| | | TTTCCAAGGAACAGGTAAATGTGAT |
| | | TTCTTTATTGGCGGTAATGGGCCTC |
| | | ATGCTTTCCGTAGTGAACGTTATGG |
| | | TAATATTTTACAGGCAATGGTCGAT |
| | | GGTAATCGTTGGATACCCAAAGAAA |
| | | TATCAGGCACGACTGCTACTGAAAA |
| | | TCCAAATGCGGATTGGCCGCTTTTG |
| | | ACATATGGCAATAATGATAATAATA |
| | | ATAGAAAATCAACATTTTGGTTGTA |
| | | TGAAAGAAAATATTTGCGATTACGG |
| | | AATGTTGAAGTCAGCTATGATTTTC |
| | | CACAAACTTGGACGCGTAAATTTTT |
| | | TGTAAGTAACTTACGTCTAGGCTTT |
| | | GTTGGACAAAATTTGTTGACATGGG |
| | | CTCCTTTTAAAATGTGGGATCCGGA |
| | | AGGGACTAGAGAGGACGGATCTAAC |
| | | TATCCGATAAATAAAACATTCTCAT |
| | | GTTATCTTCAAATAAGCTTTTAA |
| 35 | BT3791 | ATGAAAATTGTGAAGTATATAGTTA |
| | | TCGTATCATTATTTAGTATTTCTGC |
| | | ATGTAGTGATGATGATGATAAAAAA |
| | | AACAATGAGCGACCTGGGAATCTTG |
| | | TAGAGTTACAGGTTGATGTAAATGA |
| | | GATTAATATTGCGCAAGGAGATACC |
| | | CGTACTGTAAACATTACGTCAGGTA |
| | | ATGGGGAATATGTTGCGACTTCGGC |
| | | TAATGAAGAAGTAGTTGTCGCAGAA |
| | | ATAGATGGAAATGTGGTGAAACTAA |
| | | CCGCTGTTGAGGGGCATAATAATGC |
| | | TCAAGGAGTTGTTTATGTTAGCGAT |
| | | AAGTATTTCCAACGCACTAAAATTC |
| | | TAGTTAATACGGCGGCAGAATTTGA |
| | | ATTGAAGTTGAATAAAACTTTGTTT |
| | | ACGCTTTATTCTCAAGTGGAAGGAT |
| | | CTGATGAAGCTCTCATCAAGATCTA |
| | | TACAGGAAATGGAGGTTATTCTCTT |
| | | GAAGTGATTGATGATAAAAATTGTA |
| | | TTGAAGTTGATCAATCTACGCTTGA |
| | | AGACACAGAATCATTTATGGTGAAA |
| | | GGCATTGCTCAAGGTAATGCTGAGA |
| | | TTAAGATTACTGACCAAAAAGGAAA |
| | | AGAAGCTTTTGTGAATCTGAATGTA |
| | | ATTGCTCCTAAGCAAATTACGACTG |
| | | ATGCTGACGAAAAGGGCGTTCTGAT |
| | | AAATTCTAATCAAGGATCACAACAA |
| | | GTGAAGATTCTTACAGGTAATGGAG |
| | | AATATAAGGTTCTTGATGCTGGTGA |
| | | TGCAAAGATCATTCGTTTGGAAGTT |
| | | TATGGTAATGTGGTAACGGTGACCG |
| | | GAAGAAAGGCCGGAGAGACTTCATT |
| | | TACTTTGACTGATGCAAAAGGACAA |
| | | GTTTCACAGACTATTCATGTAAAGA |
| | | TCGCTCCTGAGAAGCGTTGGTATAT |
| | | GAATTTAGGAAAAGAGTATGCAGTT |
| | | TGGACTCACTTTGCAGAGATGACTG |
| | | GTGAGGGACTAGAGGCTGTGAAAGT |
| | | TGAAACTAACGGCTTTAAACTTAAA |
| | | AAAATGACTTGGGAGCTAGTTGCTC |
| | | GTATCGATGGAACTAATTGGCTACA |
| | | GACCTTTATGGGTAAGGAAGGCTAT |
| | | TTTATTCTTCGCGGTGGTGATTGGG |
| | | AAAATAATAAGGGTAGACAGATGGA |
| | | GTTGGTAGGTATAGATGATAAACTA |
| | | AAACTGAGAACTGGACATGGAGCCT |
| | | TTGAACTCGGAAAATGGTCTCATAT |
| | | TGCTTTAGTTGTAGATTGTTCGAAA |
| | | GGTAAGGATGATTACAATGAAAAAT |
| | | ACAAGCTTTATGTTAATGGTAAACA |
| | | AGTAAAGTGGGACGATAGCCGCAAA |
| | | ACCGATATGGACTATTCTGAGATTG |
| | | ATCTTTGTGCAGGTAATGACGGGGG |
| | | TAGAGTATCAATCGGAAGAGCTAGT |
| | | GACAACAGATGCTTTCTTGATGGTG |
| | | CTATACTCGAAGCACGTATCTGGAC |
| | | GGTTTGTCGTACAGAGGAACAACTT |
| | | AAGGCTAATGCATGGGAGCTTCATG |
| | | AACAAAATCCCGAAGGGTTATTAGG |
| | | GCGCTGGGATTTCTCGGCTGGAGCT |
| | | CCGACATCTTATATTGAGGATGGTA |
| | | CCAATTCGGATCATGAGTTGCTGAT |
| | | GCATATTTCGAAGTATGATAGCTGG |
| | | AATGCCACAGAATTTCCTATGAGCA |
| | | GATTTGGGGAAGCTCCCATTGAAGT |
| | | ACCTTTTAAATAA |
| 36 | BT3792 | ATGAAAGCAATATTCAAGCTGTTGA |
| | | TATTGAACTTTTTGACTCTGTTTAT |
| | | CTTTCCGTCTTGCAGTGATGATGAT |
| | | AAGTCAAAGTCTGAATTGAATGACC |
| | | CCATCAGTGGCAATATTTCTCCGGT |
| | | AGGTTCATTTGCGGTAGAAGCTACC |
| | | AATAACGAGAATGAACTTCTGGTGA |
| | | AATGGACCAATCCCAGTAATCGCGA |
| | | CGTGGATATGGTAGAACTCTCTTAC |
| | | AGGGACGTGGAAGCGAGTTTGTCTC |
| | | GTGCTACCGACTTCTCGCCGGGACA |
| | | TATCATAATACAAGTAGAGCGTGAT |
| | | GTCACACAGGAATATATGTTGAAGG |
| | | TTCCTTATTTTGCTACTTACGAAGT |
| | | TTCTGCCGTAGCTATCAGCAAAGCC |
| | | GGCAAGCGATCGGTACCCGAAAGCC |
| | | GTGTGGTGATGCCTTATCATGAAAA |
| | | GGTGGACGAGCCGGAACTGAAACTG |
| | | CCGGAAATGCTGGACCGTGCACATT |
| | | CTTACATGACTTCTGTCATTGGATA |
| | | TTATTTCGGCAAGAGTTCCAGAAGC |
| | | TGCTGGCGTAGTAATTATCCTTATG |
| | | ATGGAAAAGGTTATTGGGATGGTGA |
| | | TGCGTTGGTCTGGGGACAAGGCGGT |
| | | GGGCTTTCGGCATTTGTTGCTATGC |
| | | GTGATGCAACCAAAGAGAGCGAAGT |
| | | GGAGAATCTTTACGGTGCAATGGAT |
| | | GATATGATGTTCAAAGGAATACAGT |
| | | ATTTCTGTCAGCTGGATCGTGGAAT |
| | | CCTGGCTTATTCCTGCTACCCGGCT |
| | | GCCGGTAACGAACGTTTTTACGATG |
| | | ATAACGTATGGATCGGGCTCGATAT |
| | | GGTCGACTGGTATACGGAAACGAAA |
| | | GAGATGCGTTATCTGACACAGGCAA |
| | | AGGTGGTATGGCGCTACCTGATCGA |
| | | TCACGGTTGGGATGAGACTTGCGGA |
| | | GGAGGTGTACACTGGAGGGAGTTGA |
| | | ACGAACACACTACCAGCAAGCACTC |
| | | TTGCTCTACCGGACCTACTGCTGTG |
| | | ATGGGCTGTAAGATGTATCTGGCAA |
| | | CTCAGGAACAGGAATATCTCGACTG |
| | | GGCGATCAAATGTTACGACTATATG |
| | | CTGGATGTATTGCAAGACAAGTCCG |
| | | ATCATTTATTCTATGACAATGTACG |
| | | CCCGAATAAGGATGATCCCAATCTG |
| | | CCGGGTGATCTTGAAAAGAACAAGT |
| | | ATTCCTACAACTCCGGACAACCATT |
| | | GCAGGCGGCCTGTCTCTTATATAAG |
| | | ATTACCGGCGAACAGAAATATCTGG |
| | | ATGAAGCGTATGCGATTGCTGAAAG |
| | | CTGTCATAAGAAATGGTTTATGCCC |
| | | TATCGTTCCAAAGAGCTGAATCTTA |
| | | CTTTCAATATCCTTGCTCCGGGACA |
| | | CGCTTGGTTCAATACGATCATGTGC |
| | | CGTGGATTCTTTGAACTTTATTCTA |
| | | TAGACAATGACCGTAAATATATCGA |
| | | TGATATCGAAAAGTCAATGATTCAT |
| | | GCGTGGAGCAGTAGCTGTCATCAGG |
| | | GTAATAACTTGCTGAATGACGATGA |
| | | TCTGAGAGGGGGAACTACCAAGACC |
| | | GGTTGGGAAATACTCCATCAGGGAG |
| | | CATTGGTTGAATTGTATGCCCGGTT |
| | | GGCAGTATTGGAACGTGAAAACCGA |
| | | TAG |
| 37 | BT3792 | ATGAAAGCCATTTTTAACTTCTAAT |
| | (codon | AAATTTCTTAACTCTTTTCATTTTC |
| | optimized) | CCATCCTGTTCTGATGATGATAAAT |
| | | CCAAATCTGAATTGAACGATCCTAT |
| | | TTCTGGCAATATTTCTCCCGTAGGA |
| | | AGTTTTGCTGTCGAGGCTACAAACA |
| | | ATGAAAATGAGCTTCTTGTCAAGTG |
| | | GACCAATCCCAGTAACCGTGATGTG |
| | | GACATGGTAGAGCTTAGTTACAGAG |
| | | ACGTCGAAGCATCTCTTTCCCGTGC |
| | | AACTGACTTCAGTCCCGGACACATC |
| | | ATCATACAAGTTGAAAGGGATGTAA |
| | | CACAAGAATATATGCTTAAGGTTCC |
| | | CTATTTTGCTACCTATGAGGTCTCC |
| | | GCAGTTGCAATAAGTAAGGCCGGAA |
| | | AGAGGTCCGTTCCCGAAAGTAGGGT |
| | | AGTCATGCCTTATCACGAGAAGGTG |
| | | GATGAACCTGAGTTGAAGCTGCCCG |
| | | AGATGCTGGACAGAGCACATTCCTA |
| | | CATGACATCTGTAATAGGATACTAC |
| | | TTTGGTAAAAGTAGTCGTTCCTGTT |
| | | GGCGTTCTAACTATCCATATGACGG |
| | | TAAGGGCTACTGGGACGGAGATGCT |
| | | TTAGTGTGGGGTCAGGGAGGAGGAT |
| | | TAAGTGCATTTGTAGCAATGCGTGA |
| | | TGCTACCAAGGAATCAGAGGTAGAG |
| | | AATCTATATGGTGCTATGGACGATA |
| | | TGATGTTCAAGGGTATCCAATACTT |
| | | CTGTCAACTAGATAGAGGTATACTG |
| | | GCATATTCTTGTTATCCTGCCGCTG |
| | | GAAATGAGAGGTTTTACGATGATAA |
| | | TGTTTGGATTGGTCTAGATATGGTG |
| | | GACTGGTATACGGAAACCAAAGAGA |
| | | TGAGATACCTTACGCAAGCAAAGGT |
| | | TGTATGGCGTTATTTAATTGATCAC |
| | | GGATGGGACGAGACATGCGGTGGCG |
| | | GCGTACATTGGAGAGAACTGAATGA |
| | | ACATACTACTTCAAAACACTCATGC |
| | | AGTACTGGCCCCACTGCTGTAATGG |
| | | GTTGCAAGATGTATCTTGCTACGCA |
| | | GGAACAAGAATACTTGGACTGGGCA |
| | | ATTAAGTGTTACGATTATATGTTGG |
| | | ACGTACTACAAGATAAATCAGACCA |
| | | CTTGTTTTATGACAACGTCAGGCCA |
| | | AATAAAGATGATCCTAATTTACCAG |
| | | GCGACCTAGAGAAGAATAAGTACAG |
| | | TTATAATTCCGGCCAACCTCTGCAG |
| | | GCCGCTTGTTTACTATATAAAATTA |
| | | CGGGTGAGCAAAAGTACTTGGATGA |
| | | AGCTTATGCAATCGCCGAAAGTTGT |
| | | CACAAGAAATGGTTTATGCCATATA |
| | | GAAGTAAAGAGCTAAATCTAACTTT |
| | | CAACATCCTTGCCCCCGGACATGCT |
| | | TGGTTTAATACTATCATGTGCCGTG |
| | | GCTTTTTCGAACTATATTCAATAGA |
| | | TAATGATCGTAAATACATTGATGAC |
| | | ATAGAAAAATCAATGATACACGCCT |
| | | GGAGTTCCTCCTGCCACCAGGGAAA |
| | | CAATCTGTTAAATGACGACGACCTG |
| | | AGGGGTGGTACGACCAAGACGGGCT |
| | | GGGAAATTCTTCACCAAGGAGCACT |
| | | GGTCGAGTTATACGCAAGACTGGCA |
| | | GTTCTTGAGAGGGAGAACCGATAG |
| 38 | BT3858 | ATGATGATGAACAGATTGAATATAA |
| | | AAAGAACAGTCGGCTCCTGTTTGAT |
| | | GGCGATGGCGTTTTTTTCGTGTACC |
| | | CATACGGATCAGACGCCCACGAAAG |
| | | ACTTTGTCGATTATGTAAATCCATA |
| | | TATCGGCAATATCAGCCATCTGCTG |
| | | GTGCCTACTTACCCAACCGTACATC |
| | | TGCCGAACTCGATGCTCAGGGTCTA |
| | | TCCGGAAAGGGGAGACTATACATCG |
| | | GACAGGGTAAACGGCCTTCCGGTCG |
| | | TGGTGACCAGTCATAGAGGCAGCTC |
| | | GGCTTTTAACCTGAGTCCGGTGCAG |
| | | GGAGAGGTATCCCGACCGATTGTAT |
| | | CTTACTCCTATGATTTGGAGAATAT |
| | | TACCCCCTATAGTTATTCCGTATAC |
| | | CTGGATGAGGCTGATATACAGGTTG |
| | | AGTATGCCCCTTCACATCAGGCTGG |
| | | TATTTATCATATCAGTTTTGGGACG |
| | | GAAGGTGATAATGCTCTGGTGGTGA |
| | | ATACGAAGAACGGAAAGCTGGTCGC |
| | | TGAAGAAAAAGGAGTCAGTGGCTAT |
| | | CAGGTTATTGACAACACTCCTACCA |
| | | AAATCTATCTGTATCTCGAAACCAG |
| | | TCAACTACCTTTACGCAAAGGGGTA |
| | | CTGGCAGATGGAAAAGTTGATATGG |
| | | AAAGTAAGGAAGGCAGTGCCATCGC |
| | | TTTGTATTATGGAAGCGAGAAGAAC |
| | | CTGAATCTACGTTACGGAATTTCCT |
| | | TTATCAGTGCCGAGCAGGCAAAGAA |
| | | GAATCTGCAACGTGACATCACCACC |
| | | TATGATGTAAAGGCGGTGGCGGATG |
| | | CCGGACGCAGGATATGGAACAAGAC |
| | | ATTGGGCAAGATTGTGATAGAAGGC |
| | | GGTTCGGAAGACGAAAAAGAAATCT |
| | | TCTACACTTCCCTTTATCGTACCTA |
| | | CGAACGCATGATCAATCTTTCGGAG |
| | | GACGGGAAATATTACAGTGCTTTCG |
| | | ATGGCAAGATTCATGAAGATGGCGG |
| | | AGTACCTTTTTATACAGATGACTGG |
| | | ATATGGGATACTTACCGGGCTACAC |
| | | ATCCGTTGCGTATCTTGATAGAACC |
| | | GCAGAAGGAACTCGATATGATTCGT |
| | | TCATATATACGGATGGCAGAACAGT |
| | | CGGACAGAAGATGGATGCCTACCTT |
| | | CCCCGAGGTGACCGGAGACAGTCAC |
| | | CGGATGAATGGCAATCATGCAGTGG |
| | | CAGTTATCTGGGATGCTTATTGCAA |
| | | AGGATTGAAAGACTTTGATCTGGAG |
| | | GCTGCTTATGAAGCCTGCAAAGGAG |
| | | CGATTACAGAAAAAACGTTGTTGCC |
| | | CTGGCTGAGATGTCCGTTGACGGAG |
| | | CTCGATAAGTTCTATCAGGAAAAAG |
| | | GATTTTTCCCTGCACTGAACCCTGG |
| | | CGAAGAAGAAACTTGCAAGGCTGTT |
| | | CATTCGTTCGAGAGACGACAAGCGG |
| | | TTGCGGTTATGTTGGGTAACTGTTA |
| | | CGATAATTGGTGTCTGGCACAGATA |
| | | GCCAGAACATTAAACAAGACCGATG |
| | | ACTATAAGAAGTTTATGAGGATGTC |
| | | TTATACGTACCGGAATGTTTATAAT |
| | | GCGGAAACGGGTTTCTTTCATCCCA |
| | | AGAACAAGGACGGAAAGTTTATCGA |
| | | ACCGTTTGACTATCGATATTCGGGA |
| | | GGACAGGGGGCACGTGGCTATTATG |
| | | GTGAAAACAACGGTTGGATCTATCG |
| | | TTGGGATGTGCAGCACAATCCGGCG |
| | | GATTTGATTGCCTTGATGGGTGGAC |
| | | AGGCTTCATTTATCGAGAGATTGAA |
| | | TCAGACATTCAATGAACCGTTGGGG |
| | | CGGAGCAAGTTTGATTTCTATCATC |
| | | AGTTGCCGGACCATACCGGTAATGT |
| | | CGGCCAGTTCTCTATGGCAAATGAA |
| | | CCTTGTCTGCATATTCCTTATTTGT |
| | | ATAACTATGCCGGTCAGCCGTGGAT |
| | | GACACAAAAAAGGATTCGCGTTTTG |
| | | CTGAACCAGTGGTTCCGTAATGACT |
| | | TGATGGGCGTTCCCGGTGATGAAGA |
| | | CGGAGGTGGAATGACTGCATTTGTG |
| | | GTATTCTCCATGATGGGCTTTTATC |
| | | CGGTAACTCCCGGTTCTCCAACTTA |
| | | TAATATCGGCAGTCCGGTATTCCAA |
| | | TCCGCAAAGATGGAGGTAGGTGACG |
| | | GACATTATTTTGAGATCATAGCGGA |
| | | GAATTATGCGCCGGACCATAAGTAC |
| | | ATCCAGTCGGCTACCTTGAATGGAA |
| | | CGCCGTGGAATAAGCCGTGGTTCAG |
| | | CCATGCGGATATTCAAAACGGCGGA |
| | | CGTCTGGTTTTGCAGATGGGAGATA |
| | | AGCCCAATAAGAAGTGGGGGATAGC |
| | | TTCGGATGCCGTGCCGCCCTCTTCA |
| | | GAGAGTTTGCCGGAATAA |
| 39 | BT3862 | ATGAGGAAAGAACTTGTTTTTGTTT |
| | | TATTGGCATTATTTCTGTGTGCCGG |
| | | CTGTAACGGTAACAAAAAGAAAATG |
| | | AACGGTGAACACGATTTGGATGCGG |
| | | CAAACATTACGTTGGATGACCATAC |
| | | GATCAGTTTTTATTATAATTGGTAT |
| | | GGAAATCCGTCAGTGGATGGAGAAA |
| | | TGAAGCACTGGATGCACCCGATAGC |
| | | CCTTGCTCCGGGACATTCGGGAGAT |
| | | GTCGGTGCCATATCCGGACTTAATG |
| | | ATGACATCGCCTGTAATTTTTATCC |
| | | GGAGCTCGGAACGTACAGCAGCAAT |
| | | GATCCTGAAATCATTCGGAAACATA |
| | | TCCGGATGCATATAAAAGCGAATGT |
| | | CGGTGTACTGTCTGTCACTTGGTGG |
| | | GGAGAAAGCGATTATGGCAACCAAA |
| | | GTGTGTCTCTCCTGCTGGATGAGGC |
| | | TGCAAAAGTAGGGGCAAAGGTGTGC |
| | | TTTCATATAGAGCCTTTTAATGGAC |
| | | GCAGCCCGCAAACGGTAAGGGAGAA |
| | | TATTCAATATATAGTGGATACTTAT |
| | | GGTGATCATCCGGCTTTTTACCGTA |
| | | CGCACGGCAAACCTCTTTTCTTTAT |
| | | CTATGATTCTTATCTGATCAAACCT |
| | | GCCGAGTGGGCGAAGTTGTTTGCTG |
| | | CCGGGGGAGAGATAAGTGTGCGTAA |
| | | TACCAAGTACGACGGTCTTTTTATT |
| | | GGTCTGACATTGAAGGAAAGCGAGT |
| | | TGCCCGACATTGAGACAGCGTGCAT |
| | | GGATGGCTTTTACACTTACTTTGCC |
| | | GCAACAGGTTTCACAAATGCTTCTA |
| | | CTCCGGCCAACTGGAAATCCATGCA |
| | | GCAATGGGCAAAGGCACATAATAAA |
| | | TTGTTTATTCCGAGTGTCGGTCCGG |
| | | GATATATTGATACCCGGATTCGTCC |
| | | TTGGAACGGAAGTACCACCCGAGAC |
| | | CGTGAGAATGGAAAATATTACGATG |
| | | ATATGTATAAAGCTGCCATAGAAAG |
| | | CGGTGCTTCTTATATTTCGATTACG |
| | | TCTTTCAATGAATGGCATGAAGGAA |
| | | CTCAGATAGAGCCGGCTGTCTCAAA |
| | | GAAGTGCGATGCTTTTGAATATTTG |
| | | GATTATAAACCATTGGCTGATGATT |
| | | ACTATTTGATAAGAACTGCCTATTG |
| | | GGTAGATGAATTCCGGAAAGCAAGA |
| | | TCTGCTTCGGAAGATGTTCAATAA |
| 40 | BT3862 | ATGAGGAAAGAACTTGTTTTTGTTT |
| | (codon | TATTGGCATTATTTCTGTGTGCCGG |
| | optimized) | CTGCAATGGAAATAAAAAAAAAATG |
| | | AATGGCGAGCACGACTTGGACGCTG |
| | | CCAATATTACGCTTGATGACCATAC |
| | | AATCTCTTTTTATTACAATTGGTAC |
| | | GGTAACCCATCAGTTGACGGCGAGA |
| | | TGAAGCACTGGATGCACCCCATAGC |
| | | ACTGGCCCCCGGTCACTCCGGAGAT |
| | | GTTGGTGCAATATCTGGTTTGAATG |
| | | ATGATATTGCATGCAACTTCTACCC |
| | | TGAACTAGGAACATACTCCTCTAAC |
| | | GATCCTGAAATTATTCGTAAACACA |
| | | TTAGAATGCATATAAAGGCTAATGT |
| | | AGGCGTGCTATCTGTTACCTGGTGG |
| | | GGCGAGTCCGACTATGGAAATCAGT |
| | | CCGTTAGTCTACTATTAGATGAAGC |
| | | TGCCAAGGTAGGTGCCAAAGTATGC |
| | | TTCCACATAGAACCATTCAACGGAC |
| | | GTTCCCCCCAAACGGTGCGTGAGAA |
| | | CATCCAATACATAGTAGACACCTAT |
| | | GGTGACCACCCCGCCTTTTATCGTA |
| | | CTCACGGCAAACCTTTATTTTTCAT |
| | | TTACGACTCTTATTTGATCAAACCC |
| | | GCAGAATGGGCCAAATTGTTTGCCG |
| | | CCGGCGGTGAAATATCTGTTCGTAA |
| | | TACGAAGTATGATGGCTTGTTTATC |
| | | GGCCTTACATTAAAAGAATCTGAGC |
| | | TACCCGATATAGAAACTGCCTGCAT |
| | | GGACGGATTCTACACCTACTTCGCA |
| | | GCTACTGGATTTACGAATGCTTCAA |
| | | CGCCAGCCAATTGGAAAAGTATGCA |
| | | ACAGTGGGCTAAAGCACACAACAAA |
| | | CTTTTCATCCCTTCTGTTGGCCCAG |
| | | GATACATAGACACAAGGATAAGGCC |
| | | ATGGAACGGTTCTACAACTCGTGAC |
| | | AGAGAGAACGGAAAGTACTACGATG |
| | | ATATGTACAAAGCTGCCATAGAGTC |
| | | CGGAGCCTCTTATATATCTATCACC |
| | | TCCTTTAATGAATGGCATGAGGGCA |
| | | CACAAATAGAGCCTGCCGTATCCAA |
| | | GAAGTGCGACGCTTTCGAGTACCTT |
| | | GACTACAAACCTTTGGCCGATGACT |
| | | ACTATCTAATAAGGACCGCTTACTG |
| | | GGTGGATGAATTTAGGAAAGCCAGG |
| | | TCTGCCTCCGAGGATGTGCAGTAA |

TABLE 3
Exemplary advantageous proteins of interest (Amino Acid)

| SEQ ID | Sequence | |
|---|---|---|
| NO. | Info | Amino Acid sequence |
| 41 | BT2623 | MKKVIKKYFFLALAIIMYSCNEDEKYDILERYTPETITSDEIAPV |
| | Bacteroides | LNLQAQYMDSNSEIVLVTWMNPEDDFLSKVEISCCSANDNLLGEP |
| | thetaiotao- | VLLDAVSTKVGSYQTSLSVEERGYVKIVAINEKGVRSEARTAEIL |
| | micron | SSQQDFVYRADCLMSSVIELFFGGRYNAWNENYPNATGPYWDGIA |
| | mannan | AVWGQGAAYSGFVTMYKVTKETNNEKLRAKYAEKEETFLNSIDIF |
| | utilization | LNNGSGRKSFAYGTYIGPNDERYYDDNVWIGIEMANLYELTGNEV |
| | genes | YLQHANTVWNFILEGIDDVTGGGVYWKEGAVSKHTCSTAPAAVMA |
| | | LKLYQLSKNESYLEIAKSLYSYCKDVLQDPNDYLFYDNVRLSDPS |
| | | DKNSELKVSKDKFTYNSGQPMLAAAMLYRITKEEQFLKDAQNIAQ |
| | | SIYKKWFKNYHSSILDRDIMILSDPNTWFNAVMFRGFVELYKIDK |
| | | NDVYVKAVKNTMEHAWQSNCRNRLTNLMSDDYAGDKKEGKWNIKT |
| | | QGAFVEIFSLIGELEQLGCFQE |
| 42 | BT2629 | MKTHFSFKHLLFLGGAVLYSLOSSAVKNPVDYVSTLIGTQSKFEL |
| | | STGNTYPATALPWGMNFWTPQTGKMGDGWAYTYDADKIRGFKQTH |
| | | QPSPWMNDYGQFAIMPITGGLVFDQDRRASWFSHKAEVAKPYYYK |
| | | VYLADHDVTTELAPTERAVMFRFTYPETKNAYVIVDAFDKGSYVK |
| | | VIPEENKIIGYSTKNSGGVPENFKNYFVIQFDKPFTFVSTVFENN |
| | | ILPNETEAKGNHTGAVIGFATKKGEIVHARVASSFISPEQAELNL |
| | | KELGKNSFDQLVANGREIWNREMSKIEIEDDNIDNLRTFYSCLYR |
| | | SMLFPRSFYEIDAKGQVMHYSPYNGEVRPGYMFTDTGFWDTFRCL |
| | | FPFLNLMYPSMNQKMQEGLVNTYKESGFLPEWASPGHRDCMVGNN |
| | | SASVVADAYIKGLRGYDIETLWEALKHGANAHLRGTASGRLGYES |
| | | YNQLGYVANNIGIGQNVARTLEYAYNDWAIYTLGKKLGKPENEID |
| | | IYKKHALNYKNVYHPERKLMVGKDNKGVFNPNFDAVDWSGEFCEG |
| | | NSWHWSFCVFHDPQGLINLMGGKKEFNAMMDSVFVISGKLGMESR |
| | | GMIHEMREMQVMNMGQYAHGNQPIQHMVYLYNYSSEPWKAQYWIR |
| | | EIMNKLYTAGPDGYCGDEDNGQTSAWYVFSALGFYPVCPGTDEYI |
| | | IGTPLFKSAKLHLENGKTITIKADNNQLDNRYIKEMKVNGKSQTR |
| | | NFLTHDQLIKGANIQFQMSPVPNKQRGTTEKDVPYSLSFE |
| 43 | BT2630 | MKIKNLLLIALVAIVECGCQSNYQPTSITVASYNLRNANGGDSIN |
| | | GNGWGQRYPVIAQIVQYHDFDIFGTQECFIHQLKDMKEALPGYDY |
| | | IGVGRDDGKEKGEHSAIFYRTDKFDVIEKGDFWLSETPDVPSKGW |
| | | DAVLPRICSWGHFKCKDTGFEFLFFNLHMDHIGKKARVESAFLVQ |
| | | DKMKELGKGKELPAILTGDFNVDQTHQSYDAFVSKGVLCDSYEKA |
| | | GFRYAINGTENDFDPNSFTESRIDHIFVSPSFQVKRYGVLTDTYR |
| | | SIVGKGEKKQANDCPEEIDIKTYQARTPSDHFPVKVELEFDQRQQ |
| | | K |
| 44 | BT2631 | MRNICEVACMILFCLTSAVGKTPGNTRYLSIADSILSNVLNLYQT |
| | | NDGLLTETYPVNPDQKITYLAGGTQQNGTLKASFLWPYSGMMSGC |
| | | VALYKATGNKKYKKILEKRILPGMEQYWDNSRLPACYQSYPTKYG |
| | | QHGRYYDDNIWIALDYCDYYQLTHKPASLEKAVALYQYIYSGWSD |
| | | EIGGGIFWCEQQKEAKHTCSNAPSTVLGVKLYRLTKDAKYLEKAK |
| | | ETYAWTKKHLCDPTDHLYWDNINLKGKVSKEKYAYNSGQMIQAGV |
| | | LLYEETGDEQYLRDAQQTAAGTDAFFRTKADKKDPTVKVHKDMAW |
| | | ENVILFRGLKALYKIDKNPAYVNAMVENALHAWENYRDENGLLGR |
| | | DWSGHNKEQYKWLLDNACLIEFFAEI |
| 45 | BT2632 | MNITKAFCLSIALLGASNMQAITNSDFVIQQDNTKINNYQTNRPE |
| | | TSKRLFVSQAVEQQIAHIKQLLTNARLAWMFENCFPNTLDTTVHF |
| | | DGKDDTFVYTGDIHAMWLRDSGAQVWPYVQLANKDAELKKMLAGV |
| | | IKRQFKCINIDPYANAFNMNSEGGEWMSDLTDMKPELHERKWEID |
| | | SLCYPIRLAYHYWKTTGDASIFSDEWLTAIAKVLKTFKEQQRKED |
| | | PKGPYRFORKTERALDTMTNDGWGNPVKPVGLIASAFRPSDDATT |
| | | FQFLVPSNFFAVTSLRKAAEILNTVNKKPDLAKECTTLSNEVETA |
| | | LKKYAVYNHPKYGKIYAFEVDGFGNQLLMDDANVPSLIALPYLGD |
| | | VKVNDPIYQNTRKFVWSEDNPYFFKGTAGEGIGGPHIGYDMIWPM |
| | | SIMMKAFTSQNDAEIKTCIKMLMDTDAGTGFMHESFHKNDPKNFT |
| | | RSWFAWQNTLFGELILKLVNEGKVDLLNSIQ |
| 46 | BT3774 | MNKKVIAVALALALAGGSYAQDDTAKKKVKAYMVSDAHLDTQWNW |
| | | DIQTTINEYVWNTISQNLFLLKKYPEYVFNFEGGVKYAWMKEYYP |
| | | EQYEEMKKFIEEGRWHIAGSSWEASDVLVPSVEASIRNIMLGQTY |
| | | YRQEFGKEGTDIFLPDCFGFGWTLPTIAAHCGLIGFSSQKLDWRN |
| | | HPFYGKSKHPFTIGLWKGIDGKQVMLAHGYDYGRKWNNEDLSKNK |
| | | DLEKLAQRTPLNTVYRYYGTGDIGGSPTLGSVRSVEQGIKGDGPV |
| | | EVISATSDQLFKDYLPFNNHPELPVFDGELLMDVHGTGCYTSQAA |
| | | MKLYNRQNEQLGDAAERAAVAAEWLGTASYPQHTLTEAWKRFIFH |
| | | QFHDDLTGTSIPRAYEFSWNDELISLKQFSQVLTSSVNAIAGQMD |
| | | TRVKGTPVVLYNANAFPVSDLTEIILEQPKTPKGFTVYNAQGKKV |
| | | ASQMIGYENGRAHILVAASLPANSYAVYDVRTGGSEKTISPSAAS |
| | | AIENSVYKITLDKNGDIISLTDKRNNKELVKDGKAIRLALFTENK |
| | | SYAWPAWEILKETIDREPVSITDGAKITLVENGALRKALCIEKKY |
| | | GKSLFKQYIRLYEGSRADRIDFYNEIDWQSTNTLLKAEFPLNIEN |
| | | EKATYDLGIGSVERGNNVQTAYEVYAQQWADLTDKNNSYGVSILN |
| | | DSKYGWDKPDNNTIRLTLLHTPETKGNYAYQDHQDFGFHTFTYSL |
| | | TGHNGALDKPATAIKAEILNQPIKAFSSPKHAGTLGKEFAFVRSS |
| | | NDQVVIKALKKAEVSDEYVVRVYETGGAAPQQAAITFAGEIEKAV |
| | | LADGTEKEIGSADFNKNQLNVSIAPYSIQTFKVKLKKKADLQAPA |
| | | CAYLPLDYDRRCFSWNAFRKEGNFESGNSYAAELLPDSILKADGI |
| | | PFRLGEKEIANGLTCKGNVLQLPTGHSYNRIYFLAASAGEDAVAT |
| | | FSTGNNSQEITVPSYTGFIGQWEHLGHTEGFLKDAEIAYVGTHRH |
| | | ASDKDEAYEFTYMFKFGMDIPKGATTVTLPDHADIVLFAATLVNE |
| | | KYPAVTPASELFRTALKADNGEEATTKTNLLKQAKLIKCSGETNE |
| | | KEVARYAVDGDVKTKWCDTSTAPNYIDFDFGKEQTIRGWKLVNAG |
| | | NEGSVFITHTCFLQGRNSPDEEWKTIDELSDNKKNTVVRQFKPTS |
| | | VRYVRLLVTQSTQNNSLKAARIYELEVY |
| 47 | BT3780 | MKSTFLELVTTTMMTCTALGQPSNDKKNVLPDWAFGGFERPQGAN |
| | | PVISPIENTKFYCPMTQDYVAWESNDTFNPAATLHDGKIVVLYRA |
| | | EDKSGVGIGHRTSRLGYATSSDGIHFKREKTPVFYPDNDTQKKLE |
| | | WPGGCEDPRIAVTAEGLYVMTYTQWNRHIPRLAIATSRNLKDWTK |
| | | HGPAFAKAYDGKFFNLGCKSGSILTEVVNGKQVIKKIDGKYFMYW |
| | | GEEHVFAATSEDLVNWTPYVNTDGSLRKLFSPRDGHFDSQLTECG |
| | | PPAIYTPKGIVLLYNGKNSASRGDKRYTANVYAAGQALFDANDPT |
| | | RFITRLDEPFFRPMDSFEKSGQYVDGTVFIEGMVYYKDKWYLYYG |
| | | CADSKVGMAIYNPKKPAAADPLP |
| 48 | BT3781 | MNITKTLCLCAALSGAAGVQAMENREFVTQQDNTRVNNYQTNRPE |
| | | ASKRLFVSQEVERQIDHIKQLLTNAKLAWMFENCFPNTLDTTVHF |
| | | DGKEDTFVYTGDIHAMWLRDSGAQVWPYVQLANKDPELKKMLAGV |
| | | INRQFKCINIDPYANAFNMNSEGGEWMSDLTDMKPELHERKWEID |
| | | SLCYPIRLAYHYWKTTGDASVFSDEWLQAIANVLKTFKEQQRKDD |
| | | AKGPYRFQRKTERALDTMTNDGWGNPVKPVGLIASAFRPSDDATT |
| | | FQFLVPSNFFAVTSLRKAAEILNTVNRKPALAKECTALADEVEKA |
| | | LKKYAVCNHPKYGKIYAFEVDGFGNQLLMDDANVPSLIALPYLGD |
| | | VKVTDPIYQNTRKFVWSEDNPYFFKGSAGEGIGGPHIGYDMIWPM |
| | | SIMMKAFTSQNDAEIKTCIKMLMDTDAGTGFMHESFNKNDPKNFT |
| | | RAWFAWQNTLFGELILKLVNEGKVDLLNSIQ |
| 49 | BT3782 | MRNICEVACMLFCLASASGKTVKNHPFVSIADSILDNVLNLYQTE |
| | | DGLLTETYPVNPDQKITYLAGGAQQNGTLKASFLWPYSGMMSGCV |
| | | AMYQATGDKKYKTILEKRILPGLEQYWDGERLPACYQSYPVKYGQ |
| | | HGRYYDDNIWIALDYCDYYRLTKKADYLKKAIALYEYIYSGWSDE |
| | | LGGGIFWCEQQKEAKHTCSNAPSTVLGVKLYRLTKDKKYLNKAKE |
| | | TYAWTRKHLCDPDDFLYWDNINLKGKVSKDKYAYNSGQMIQAGVL |
| | | LYEETGDKDYLRDAQKTAAGTDAFFRSKADKKDPSVKVHKDMSWF |
| | | NVILFRGFKALEKIDHNPTYVRAMAENALHAWRNYRDANGLLGRD |
| | | WSGHNEEPYKWLLDNACLIELFAEIEK |
| 50 | BT3783 | MKLRNLLEIVLAAIVFCNCQSYQPTSLTVASYNLRNANGSDSARG |
| | | DGWGQRYPVIAQMVQYHDFDIFGTQECFLHQLKDMKEALPGYDYI |
| | | GVGRDDGKDKGEHSAIFYRTDKFDIVEKGDFWLSETPDVPSKGWD |
| | | AVLPRICSWGHFKCKDTGFEFLFFNLHMDHIGKKARVESAFLVQE |
| | | KMKELGRGKNLPAILTGDFNVDQTHQSYDAFVSKGVLCDSYEKCD |
| | | YRYALNGTFNNFDPNSFTESRIDHIFVSPSFHVKRYGVLTDTYRS |
| | | VRENSKKEDVRDCPEEITIKAYEARTPSDHFPVKVELVFDQRQQK |
| 51 | BT3784 | MKTHESEKHLLFIGGAVLYSMOISAVKNPVDYVSTLVGTQSKFEL |
| | | STGNTYPATALPWGMNFWTPQTGKMGDGWAYTYNADKIRGFKQTH |
| | | QPSPWMNDYGQFSIMPITGGLVFDQDQRASWFSHKAEVAKPYYYK |
| | | VYLADHDVTTELVPTERAAMFRFTYPETKNAYVVIDAFDKGSYVK |
| | | VIPEENKIIGYSTKNSGGVPENFKNYFVIQFDKPFTFTSGVKENN |
| | | ILPNETEVQGNHTGAIIGFATQKGEIVHARVASSFISYEQAELNL |
| | | KELGKDSFDQLVTKGKDIWNREMSKVDVEDDNIDNLRTFYSCLYR |
| | | SMLFPRSFYEIDAKGQVVHYSPYNGKVLPGYMFTDTGFWDTFRCL |
| | | FPFLNLMYPSMNQKMQEGLVNAYLESGFLPEWASPGHRDCMVGNN |
| | | SASVVADAYIKGLRGYDIETLWEALKHDANAHLRGTASGRLAYDA |
| | | YNKLGYVPNNIGIGQNVARTLEYAYNDWTIYTLGKKLGKPASEID |
| | | IFKQRALNYKNVYHPKRKLMVGKDDKGVFNPKFDAVDWSGEFCEG |
| | | NSWHWSFCVFHDPQGLIDLMGGKKEFNNMMDSVFVIPGKQGMESR |
| | | GMIHEMREMQVMNMGQYAHGNQPIQHMVYLYNYSGEPWKAQHWVR |
| | | EIMDKLYTAGPDGYCGDEDNGQTSAWYVFSALGFYPVCPGTDQYI |
| | | LGTPLFKSAKLHLENGKTVTIKASNNNTDNRYVKDMKVNGKAFTR |
| | | NYLTHDQLLKGANIQYQMSPTPNKQRGTTEKDIPYSLSFE |
| 52 | BT3788 | MKNTQKYFILLLMLILLVPSNIWQQETKKEIIVKGVVEDDLGPII |
| | | GASVVAKNQAGVGVITNTEGKFSLKVGPYDVLVVTFVGYQPYELP |
| | | VLKMNDPNNVTIKLLEDVGKIDEVVITASGLQQKKTLTGAITNVD |
| | | VKQLNAVGSSSLSNSLAGVVPGIIAMQRSGEPGENTSEFWIRGIS |
| | | TFGAKSGALVLIDGVERNFDEILPQDIESESVLKDASATAIYGQR |
| | | GANGVILITTKRGEKGKVKINVKAGFDWNTPVKVPEYASGYDWAR |
| | | LANEARLGRYDSPIYTPEELEIIRSGLDTDLYPNIDWRDLMLKSG |
| | | APRYYANISFSGGSDNVRYYVSGQYTSEQGRYKTFSSENKYNTNT |
| | | TYERYNYRANVDMNITKTTVLKVSVGGWLVNRTTPTRSTGDIWED |
| | | FAKFTPLSTPRKWSTGQWPRVDGQDTPEYHMTQRGYHTKWESKVE |
| | | TSVKLEQDLKFITPGLKFEGVFAFDTYNENIIKREKKEEVWEAQK |
| | | YRDENGKLILKRVVNRSPMNQNKEVRGDKRYYFQASLDYNRLFAH |
| | | AHRVGVFGMVYQEEKTDVNFDSSDLIGSIPRRNLAYSGRFTYAYK |
| | | DKYLAEFNWGCTGSENFEHGKQFGFFPAVSAGYVISEEAFMKKAL |
| | | PWIDQFKIRASYGEVGNDVLDGRRFPYVSLIDTDDGGSYSFGEFG |
| | | TNRVQAYRIRTLGTPNLTWEIAKKYDVGVDFSFFNGKISGALDWF |
| | | LDKRDDIFMQRKHMPLTTGLADQTPMANVGKMKSYGWEGNIGFTQ |
| | | SIGQVNLQLRANFTYQTTDIIDKDEAANELWYKMDKGFQLNQSRG |
| | | LIALGLFKDQDEIDRSPKQTSNRPILPGDIKYKDVNGDGVINDDD |
| | | IVPLGYREVPGLQYGVGLSANWRNWNLSVLFQGTGKCDFFIGGNG |
| | | PHAFRSERYGNILQAMVDGNRWIPKEISGTTATENPNADWPLLTY |
| | | GNNDNNNRKSTFWLYERKYLRLRNVEVSYDFPQTWTRKFFVSNLR |
| | | LGFVGQNLLTWAPFKMWDPEGTREDGSNYPINKTFSCYLQISF |
| 53 | BT3791 | MKIVKYIVIVSLESISACSDDDDKKNNERPGNLVELQVDVNEINI |
| | | AQGDTRTVNITSGNGEYVATSANEEVVVAEIDGNVVKLTAVEGHN |
| | | NAQGVVYVSDKYFQRTKILVNTAAEFELKLNKTLFTLYSQVEGSD |
| | | EALIKIYTGNGGYSLEVIDDKNCIEVDQSTLEDTESFMVKGIAQG |
| | | NAEIKITDQKGKEAFVNLNVIAPKQITTDADEKGVLINSNQGSQQ |
| | | VKILTGNGEYKVLDAGDAKIIRLEVYGNVVTVTGRKAGETSFTLT |
| | | DAKGQVSQTIHVKIAPEKRWYMNLGKEYAVWTHFAEMTGEGLEAV |
| | | KVETNGFKLKKMTWELVARIDGTNWLQTFMGKEGYFILRGGDWEN |
| | | NKGRQMELVGIDDKLKLRTGHGAFELGKWSHIALVVDCSKGKDDY |
| | | NEKYKLYVNGKQVKWDDSRKTDMDYSEIDLCAGNDGGRVSIGRAS |
| | | DNRCFLDGAILEARIWTVCRTEEQLKANAWELHEQNPEGLLGRWD |
| | | FSAGAPTSYIEDGTNSDHELLMHISKYDSWNATEFPMSRFGEAPI |
| | | EVPFK |
| 54 | BT3792 | MKAIFKLLILNFLTLFIFPSCSDDDKSKSELNDPISGNISPVGSF |
| | | AVEATNNENELLVKWTNPSNRDVDMVELSYRDVEASLSRATDFSP |
| | | GHIIIQVERDVTQEYMLKVPYFATYEVSAVAISKAGKRSVPESRV |
| | | VMPYHEKVDEPELKLPEMLDRAHSYMTSVIGYYFGKSSRSCWRSN |
| | | YPYDGKGYWDGDALVWGQGGGLSAFVAMRDATKESEVENLYGAMD |
| | | DMMFKGIQYFCQLDRGILAYSCYPAAGNERFYDDNVWIGLDMVDW |
| | | YTETKEMRYLTQAKVVWRYLIDHGWDETCGGGVHWRELNEHTTSK |
| | | HSCSTGPTAVMGCKMYLATQEQEYLDWAIKCYDYMLDVLQDKSDH |
| | | LFYDNVRPNKDDPNLPGDLEKNKYSYNSGQPLQAACLLYKITGEQ |
| | | KYLDEAYALAESCHKKWFMPYRSKELNLTFNILAPGHAWFNTIMC |
| | | RGFFELYSIDNDRKYIDDIEKSMIHAWSSSCHQGNNLLNDDDLRG |
| | | GTTKTGWEILHQGALVELYARLAVLERENR |
| 55 | BT3858 | MMMNRLNIKRTYGSCLMAMAFFSCTHTDQTPTKDFVDYVNPYIGN |
| | | ISHLLVPTYPTVHLPNSMLRVYPERGDYTSDRVNGLPVVVTSHRG |
| | | SSAFNLSPVQGEVSRPIVSYSYDLENITPYSYSVYLDEADIQVEY |
| | | APSHQAGIYHISFGTEGDNALVVNTKNGKLVAEEKGVSGYQVIDN |
| | | TPTKIYLYLETSQLPLRKGVLADGKVDMESKEGSAIALYYGSEKN |
| | | LNLRYGISFISAEQAKKNLQRDITTYDVKAVADAGRRIWNKTLGK |
| | | IVIEGGSEDEKEIFYTSLYRTYERMINLSEDGKYYSAFDGKIHED |
| | | GGVPFYTDDWIWDTYRATHPLRILIEPQKELDMIRSYIRMAEQSD |
| | | RRWMPTFPEVTGDSHRMNGNHAVAVIWDAYCKGLKDFDLEAAYEA |
| | | CKGAITEKTLLPWLRCPLTELDKFYQEKGFFPALNPGEEETCKAV |
| | | HSFERRQAVAVMLGNCYDNWCLAQIARTLNKTDDYKKFMRMSYTY |
| | | RNVYNAETGFFHPKNKDGKFIEPFDYRYSGGQGARGYYGENNGWI |
| | | YRWDVQHNPADLIALMGGQASFIERLNQTFNEPLGRSKFDFYHQL |
| | | PDHTGNVGQFSMANEPCLHIPYLYNYAGQPWMTQKRIRVLLNQWF |
| | | RNDLMGVPGDEDGGGMTAFVVFSMMGFYPVTPGSPTYNIGSPVFQ |
| | | SAKMEVGDGHYFEIIAENYAPDHKYIQSATLNGTPWNKPWFSHAD |
| | | IQNGGRLVLQMGDKPNKKWGIASDAVPPSSESLPE |
| 56 | BT3862 | MRKELVFVLLALFLCAGCNGNKKKMNGEHDLDAANITLDDHTISF |
| | | YYNWYGNPSVDGEMKHWMHPIALAPGHSGDVGAISGLNDDIACNF |
| | | YPELGTYSSNDPEIIRKHIRMHIKANVGVLSVTWWGESDYGNQSV |
| | | SLLLDEAAKVGAKVCFHIEPFNGRSPQTVRENIQYIVDTYGDHPA |
| | | FYRTHGKPLFFIYDSYLIKPAEWAKLFAAGGEISVRNTKYDGLFI |
| | | GLTLKESELPDIETACMDGFYTYFAATGFTNASTPANWKSMQQWA |
| | | KAHNKLFIPSVGPGYIDTRIRPWNGSTTRDRENGKYYDDMYKAAI |
| | | ESGASYISITSFNEWHEGTQIEPAVSKKCDAFEYLDYKPLADDYY |
| | | LIRTAYWVDEFRKARSASEDVQ |
| 86 | Erp1 | MLLTSLLQVFACCLVLPAQVTAFYYYTSGAERKCFHKELSKGTLF |
| | | QATYKAQIYDDQLQNYRDAGAQDFGVLIDIEETFDDNHLVVHQKG |
| | | SASGDLTFLASDSGEHKICIQPEAGGWLIKAKTKIDVEFQVGSDE |
| | | KLDSKGKATIDILHAKVNVLNSKIGEIRREQKLMRDREATFRDAS |
| | | EAVNSRAMWWIVIQLIVLAVTCGWQMKHLGKFFVKQKIL |
| 87 | Erp2 | MIKSTIALPSFFIVLILALVNSVAASSSYAPVAISLPAFSKECLY |
| | | YDMVTEDDSLAVGYQVLTGGNFEIDFDITAPDGSVITSEKQKKYS |
| | | FDLLKSFGVGKYTFCFSNNYGTALKKVEITLEKEKTLTDEHEADV |
| | | NNDDIIANNAVEEIDRNLNKITKTLNYLRAREWRNMSTVNSTESR |
| | | LTWLSILIIIIIAVISIAQVLLIQFLFTGRQKNYV |
| 88 | Emp24 | MASFATKFVIACFLFFSASAHNVLLPAYGRRCFFEDLSKGDELSI |
| | | SFQFGDRNPQSSSQLTGDFIIYGPERHEVLKTVRDTSHGEITLSA |
| | | PYKGHFQYCFLNENTGIETKDVTFNIHGVVYVDLDDPNTNTLDSA |
| | | VRKLSKLTREVKDEQSYIVIRERTHRNTAESTNDRVKWWSIFQLG |
| | | VVIANSLFQIYYLRRFFEVTSLV |
| 89 | Erv25 | MQVLQLWLTTLISLVVAVQGLHFDIAASTDPEQVCIRDFVTEGQL |
| | | VVADIHSDGSVGDGQKLNLFVRDSVGNEYRRKRDFAGDVRVAFTA |
| | | PSSTAFDVCFENQAQYRGRSLSRAIELDIESGAEARDWNKISANE |
| | | KLKPIEVELRRVEEITDEIVDELTYLKNREERLRDTNESTNRRVR |
| | | NFSILVIIVLSSLGVWQVNYLKNYFKTKHII |
| 90 | Erp3 | MSNLCVLFFQFFFLAQFFAEASPLTFELNKGRKECLYTLTPEIDC |
| | | TISYYFAVQQGESNDFDVNYEIFAPDDKNKPIIERSGERQGEWSF |
| | | IGQHKGEYAICFYGGKAHDKIVDLDFKYNCERQDDIRNERRKARK |
| | | AQRNLRDSKTDPLQDSVENSIDTIERQLHVLERNIQYYKSRNTRN |
| | | HHTVCSTEHRIVMFSIYGILLIIGMSCAQIAILEFIFRESRKHN |
| | | V* |
| 91 | Erp5 | MKYNIVHGICLLFAITQAVGAVHFYAKSGETKCFYEHLSRGNLLI |
| | | GDLDLYVEKDGLFEEDPESSLTITVDETFDNDHRVLNQKNSHTGD |
| | | VTFTALDTGEHRFCFTPFYSKKSATLRVFIELEIGNVEALDSKKK |
| | | EDMNSLKGRVGQLTQRLSSIRKEQDAIREKEAEFRNQSESANSKI |
| | | MTWSVFQLLILLGTCAFQLRYLKNFFVKQKVV |

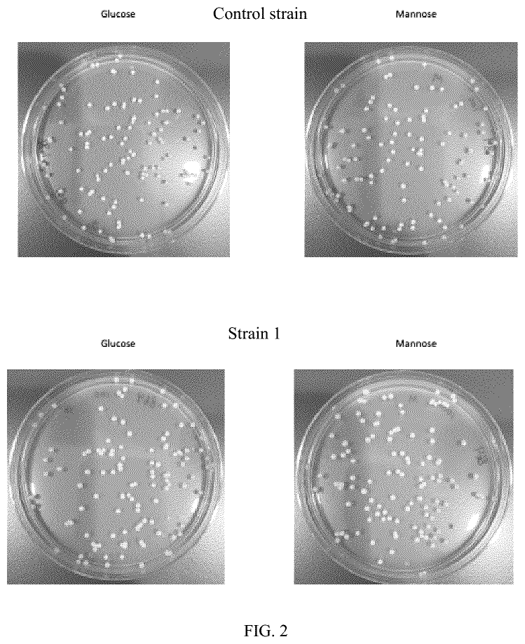

TABLE 4
Exemplary Surface Display Molecules

| SEQ ID | Sequence | |
|---|---|---|
| NO. | Info | Sequence |
| 57 | Surface | MREPSIFTAVLEAASSALAAPVNTTTEDETAOIPAEAVIGYSDLE |
| | display | GDEDVAVLPESNSTNNGLLFINTTIASIAAKBEGVSLDKREAEA |
| | molecule | (alpha factor) |
| | | MKKVIKKYFFLALAIIMYSCNEDEKYDILERYTPETITSDEIAPV |
| | | LNLQAQYMDSNSEIVLVTWMNPEDDELSKVEISCCSANDNLLGEP |
| | | VLLDAVSTKVGSYQTSLSVEERGYVKIVAINEKGVRSEARTABIL |
| | | SSQQDFVYRADCLMSSVIELFFGGRYNAWNENYPNATGPYWDGIA |
| | | AVWGQGAAYSGFVTMYKVTKETNNEKLRAKYABKEETELNSIDIF |
| | | LNNGSQRKSFAYQTYIGPNDERYYDDNVWIGIEMANLYELTQNEV |
| | | YLQHANTVWNFILEGIDDVTGQGVYWKEGAVSKHTCSTAPAAVMA |
| | | LKLYQLSKNESYLEIAKSLYSYCKDVLQDPNDYLFYDNVRLSDPS |
| | | DKNSBLKVSKDKFTYNSGQPMLAAAMLYRITKEBQFLKDAQNIAQ |
| | | SIYKKWEKNYHSSILDRDIMILSDPNTWENAVMFRQFVELYKIDK |
| | | NDVYVKAVKNTMEHAWQSNCRNRLTNLMSDDYAGDKKEGKWNIKT |
| | | QGAFVEIFSLIGELEQLGCFQE (codon optimized |
| | | BT2623) |
| | | EAAAREAAAREAAARBAAAR (alpha-helix linker) |
| | | GGGGSGGGGSGGGGS (linker) |
| | | QFSNSTSASSTDVTSSSSISTSSQSVTITSSEAPESDNGTSTAAP |
| | | TETSTEAPTTAIPTNQTSTEAPTTAIPTNGTSTEAPTDTPTTALP |
| | | TNGTSTEAPTDTTTEAPTTGLFINGTTSAFPPTTSLPITTTTPPY |
| | | NPSTDYTTDYTVVTEYTTYCPEPTTFTTNQKTYTVTEPTTLTITD |
| | | CPCTIEKPTTTSVVTEYTTYCPEPTTFTTNGKTYVTEPTTLTITD |
| | | CPCTIEKSEAPESSVPVTESKGTTTKETGVTTKQTTANPSLTVST |
| | | VVPVSSSASSHSVVINSN (Mature Sed1) |
| | | GANVVVPGALGLAGVAMLFL (Sed1 propeptide) |
| 58 | Tir4 from | QINELNVVLDDVKTNIADYITLSYTPNSGFSLDQMPAGIMDIAAQ |
| | Saccharomyces | LVANPSDDSYTTLYSEVDFSAVEHMLTMVPWYSSRLLPELEAMDA |
| | cerevisiae | SLTTSSSAATSSSEVASSSIASSTSSSVAPSSSEVVSSSVASSSS |
| | | EVASSSVASTSEATSSSAVTSSSAVSSSTESVSSSSVSSSSAVSS |
| | | SEAVSSSPVSSVVSSSAGPASSSVAPYNSTIASSSSTAQTSISTI |
| | | APYNSTTTTTPASSASSVIISTRNGTTVTETDNTLVTKETTVCDY |
| | | SSTSAVPASTTGYNNSTKVSTATICSTCKEGTSTATDFSTLKTTV |
| | | TVCDSACQAKKSATVVSVQSKTTGIVEQTENGAAKAVIGMGAGAL |
| | | AAVAAMLL |
| 59 | Tir4 from | MAYSKITLLAALAAIAYAQTQAQINELNVVLDDVKTNIADYITLS |
| | Saccharomyces | YTPNSGFSLDQMPAGIMDIAAQLVANPSDDSYTTLYSEVDFSAVE |
| | cerevisiae | HMLTMVPWYSSRLLPELEAMDASLTTSSSAATSSSEVASSSIASS |
| | (underlined | TSSSVAPSSSEVVSSSVASSSSEVASSSVASTSEATSSSAVTSSS |
| | is signal | AVSSSTESVSSSSVSSSSAVSSSEAVSSSPVSSVVSSSAGPASSS |
| | peptide, | VAPYNSTIASSSSTAQTSISTIAPYNSTTTTTPASSASSVIISTR |
| | which may | NGTTVTETDNTLVTKETTVCDYSSTSAVPASTTGYNNSTKVSTAT |
| | not be | ICSTCKEGTSTATDFSTLKTTVTVCDSACQAKKSATVVSVQSKTT |
| | utilized | GIVEQTENGAAKAVIGMGAGALAAVAAMLL |
| | in design) | |
| 60 | Tir4 | QINELNVVLDDVKTNIADYITLSYTPNSGFSLDQMPAGIMDIAAQ |
| | (NP_014652.1) | LVANPSDDSYTTLYSEVDFSAVEHMLTMVPWYSSRLLPELEAMDA |
| | from | SLTTSSSAATSSSEVASSSIASSTSSSVAPSSSEVVSSSVAPSSS |
| | Saccharomyces | EVVSSSVAPSSSEVVSSSVASSSSEVASSSVAPSSSEVVSSSVAS |
| | cerevisiae | SSSEVASSSVAPSSSEVVSSSVAPSSSEVVSSSVASSSSEVASSS |
| | | VAPSSSEVVSSSVASSTSEATSSSAVTSSSAVSSSTESVSSSSVS |
| | | SSSAVSSSEAVSSSPVSSVVSSSAGPASSSVAPYNSTIASSSSTA |
| | | QTSISTIAPYNSTTTTTPASSASSVIISTRNGTTVTETDNTLVTK |
| | | ETTVCDYSSTSAVPASTTGYNNSTKVSTATICSTCKEGTSTATDF |
| | | STLKTTVTVCDSACQAKKSATVVSVQSKTTGIVEQTENGAAKAVI |
| | | GMGAGALAAVAAMLL |
| 61 | Tir4 | MAYSKITLLAALAAIAYAQTQAQINELNVVLDDVKTNIADYITLS |
| | (NP_014652.1) | YTPNSGFSLDQMPAGIMDIAAQLVANPSDDSYTTLYSEVDFSAVE |
| | from | HMLTMVPWYSSRLLPELEAMDASLTTSSSAATSSSEVASSSIASS |
| | Saccharomyces | TSSSVAPSSSEVVSSSVAPSSSEVVSSSVAPSSSEVVSSSVASSS |
| | cerevisiae | SEVASSSVAPSSSEVVSSSVASSSSEVASSSVAPSSSEVVSSSVA |
| | (underlined | PSSSEVVSSSVASSSSEVASSSVAPSSSEVVSSSVASSTSEATSS |
| | is signal | SAVTSSSAVSSSTESVSSSSVSSSSAVSSSEAVSSSPVSSVVSSS |
| | peptide, | AGPASSSVAPYNSTIASSSSTAQTSISTIAPYNSTTTTTPASSAS |
| | which may | SVIISTRNGTTVTETDNTLVTKETTVCDYSSTSAVPASTTGYNNS |
| | not be | TKVSTATICSTCKEGTSTATDFSTLKTTVTVCDSACQAKKSATVV |
| | utilized | SVQSKTTGIVEQTENGAAKAVIGMGAGALAAVAAMLL |
| | in design) | |
| 62 | Dan1 from | ASVTTTLSPYDERVNLIELAVYVSDIGAHLSEYYAFQALHKTETY |
| | Saccharomyces | PPEIAKAVFAGGDFTTMLTGISGDEVTRMITGVPWYSTRLMGAIS |
| | cerevisiae | EALANEGIATAVPASTTEASSTSTSEASSAATESSSSSESSAETS |
| | | SNAASTQATVSSESSSAASTIASSAESSVASSVASSVASSASFAN |
| | | TTAPVSSTSSISVTPVVQNGTDSTVTKTQASTVETTITSCSNNVC |
| | | STVTKPVSSKAQSTATSVTSSASRVIDVTTNGANKENNGVFGAAA |
| | | IAGAAALLL |
| 63 | Dan1 from | MSRISILAVAAALVASATAASVTTTLSPYDERVNLIELAVYVSDI |
| | Saccharomyces | GAHLSEYYAFQALHKTETYPPEIAKAVFAGGDFTTMLTGISGDEV |
| | cerevisiae | TRMITGVPWYSTRLMGAISEALANEGIATAVPASTTEASSTSTSE |
| | (underlined | ASSAATESSSSSESSAETSSNAASTQATVSSESSSAASTIASSAE |
| | is signal | SSVASSVASSVASSASFANTTAPVSSTSSISVTPVVQNGTDSTVT |
| | peptide, | KTQASTVETTITSCSNNVCSTVTKPVSSKAQSTATSVTSSASRVI |
| | which may | DVTTNGANKENNGVFGAAAIAGAAALLL |
| | not be | |
| | utilized | |
| | in design) | |
| 64 | Sed1 from | QFSNSTSASSTDVTSSSSISTSSGSVTITSSEAPESDNGTSTAAP |
| | Saccharomyces | TETSTEAPTTAIPTNGTSTEAPTTAIPTNGTSTEAPTDTTTEAPT |
| | cerevisiae | TALPTNGTSTEAPTDTTTEAPTTGLPTNGTTSAFPPTTSLPPSNT |
| | | TTTPPYNPSTDYTTDYTVVTEYTTYCPEPTTFTTNGKTYTVTEPT |
| | | TLTITDCPCTIEKPTTTSTTEYTVVTEYTTYCPEPTTFTTNGKTY |
| | | TVTEPTTLTITDCPCTIEKSEAPESSVPVTESKGTTTKETGVTTK |
| | | QTTANPSLTVSTVVPVSSSASSHSVVINSNGANVVVPGALGLAGV |
| | | AMLFL |
| 65 | Sed1 from | MKLSTVLLSAGLASTTLAQFSNSTSASSTDVTSSSSISTSSGSVT |
| | Saccharomyces | ITSSEAPESDNGTSTAAPTETSTEAPTTAIPTNGTSTEAPTTAIP |
| | cerevisiae | TNGTSTEAPTDTTTEAPTTALPTNGTSTEAPTDTTTEAPTTGLPT |
| | (which may | NGTTSAFPPTTSLPPSNTTTTPPYNPSTDYTTDYTVVTEYTTYCP |
| | not be | EPTTFTTNGKTYTVTEPTTLTITDCPCTIEKPTTTSTTEYTVVTE |
| | utilized | YTTYCPEPTTFTTNGKTYTVTEPTTLTITDCPCTIEKSEAPESSV |
| | in design) | PVTESKGTTTKETGVTTKQTTANPSLTVSTVVPVSSSASSHSVVI |
| | | NSNGANVVVPGALGLAGVAMLFL |
| 66 | Dan4 from | ITATTTLSPYDERVNLIELAVYVSDIRAHIFQYYSFRNHHKTETY |
| | Saccharomyces | PSEIAAAVFDYGDFTTRLTGISGDEVTRMITGVPWYSTRLKPAIS |
| | cerevisiae | SALSKDGIYTAIPTSTSTTTTKSSTSTTPTTTITSTTSTTSTTPT |
| | | TSTTSTTPTTSTTSTTPTTSTTSTTPTTSTTSTTPTTSTTSTTPT |
| | | TSTTSTTPTTSTTSTTPTTSTTSTTPTTSTTPTTSTTSTTSQTST |
| | | KSTTPTTSSTSTTPTTSTTPTTSTTSTAPTTSTTSTTSTTSTIST |
| | | APTTSTTSSTESTSSASASSVISTTATTSTTFASLTTPATSTAST |
| | | DHTTSSVSTTNAFTTSATTTTTSDTYISSSSPSQVTSSAEPTTVS |
| | | EVTSSVEPTRSSQVTSSAEPTTVSEFTSSVEPTRSSQVTSSAEPT |
| | | TVSEFTSSVEPTRSSQVTSSAEPTTVSEFTSSVEPTRSSQVTSSA |
| | | EPTTVSEFTSSVEPTRSSQVTSSAEPTTVSEFTSSVEPIRSSQVT |
| | | SSAEPTTVSEVTSSVEPIRSSQVTTTEPVSSFGSTFSEITSSAEP |
| | | LSFSKATTSAESISSNQITISSELIVSSVITSSSEIPSSIEVLTS |
| | | SGISSSVEPTSLVGPSSDESISSTESLSATSTFTSAVVSSSKAAD |
| | | FFTRSTVSAKSDVSGNSSTQSTTFFATPSTPLAVSSTVVTSSTDS |
| | | VSPNIPFSEISSSPESSTAITSTSTSFIAERTSSLYLSSSNMSSF |
| | | TLSTFTVSQSIVSSFSMEPTSSVASFASSSPLLVSSRSNCSDARS |
| | | SNTISSGLFSTIENVRNATSTFTNLSTDEIVITSCKSSCTNEDSV |
| | | LTKTQVSTVETTITSCSGGICTTLMSPVTTINAKANTLTTTETST |
| | | VETTITTCPGGVCSTLTVPVTTITSEATTTATISCEDNEEDITST |
| | | ETELLTLETTITSCSGGICTTLMSPVTTINAKANTLTTTETSTVE |
| | | TTITTCSGGVCSTLTVPVTTITSEATTTATISCEDNEEDVASTKT |
| | | ELLTMETTITSCSGGICTTLMSPVSSFNSKATTSNNAESTIPKAI |
| | | KVSCSAGACTTLTTVDAGISMFTRTGLSITQTTVTNCSGGTCTML |
| | | TAPIATATSKVISPIPKASSATSIAHSSASYTVSINTNGAYNFDK |
| | | DNIFGTAIVAVVALLLL |
| 67 | Dan4 | MVNISIVAGIVALATSAAAITATTTLSPYDERVNLIELAVYVSDI |
| | Saccharomyces | RAHIFQYYSFRNHHKTETYPSEIAAAVFDYGDFTTRLTGISGDEV |
| | cerevisiae | TRMITGVPWYSTRLKPAISSALSKDGIYTAIPTSTSTTTTKSSTS |
| | (underlined | TTPTTTITSTTSTTSTTPTTSTTSTTPTTSTTSTTPTTSTTSTTP |
| | is signal | TTSTTSTTPTTSTTSTTPTTSTTSTTPTTSTTSTTPTTSTTSTTP |
| | peptide, | TTSTTPTTSTTSTTSQTSTKSTTPTTSSTSTTPTTSTTPTTSTTS |
| | which may | TAPTTSTTSTTSTTSTISTAPTTSTTSSTESTSSASASSVISTTA |
| | not be | TTSTTFASLTTPATSTASTDHTTSSVSTTNAFTTSATTTTTSDTY |
| | utilized | ISSSSPSQVTSSAEPTTVSEVTSSVEPTRSSQVTSSAEPTTVSEF |
| | in design) | TSSVEPTRSSQVTSSAEPTTVSEFTSSVEPTRSSQVTSSAEPTTV |
| | | SEFTSSVEPTRSSQVTSSAEPTTVSEFTSSVEPTRSSQVTSSAEP |
| | | TTVSEFTSSVEPIRSSQVTSSAEPTTVSEVTSSVEPIRSSQVTTT |
| | | EPVSSFGSTFSEITSSAEPLSFSKATTSAESISSNQITISSELIV |
| | | SSVITSSSEIPSSIEVLTSSGISSSVEPTSLVGPSSDESISSTES |
| | | LSATSTFTSAVVSSSKAADFFTRSTVSAKSDVSGNSSTQSTTFFA |
| | | TPSTPLAVSSTVVTSSTDSVSPNIPFSEISSSPESSTAITSTSTS |
| | | FIAERTSSLYLSSSNMSSFTLSTFTVSQSIVSSFSMEPTSSVASF |
| | | ASSSPLLVSSRSNCSDARSSNTISSGLFSTIENVRNATSTFTNLS |
| | | TDEIVITSCKSSCTNEDSVLTKTQVSTVETTITSCSGGICTTLMS |
| | | PVTTINAKANTLTTTETSTVETTITTCPGGVCSTLTVPVTTITSE |
| | | ATTTATISCEDNEEDITSTETELLTLETTITSCSGGICTTLMSPV |
| | | TTINAKANTLTTTETSTVETTITTCSGGVCSTLTVPVTTITSEAT |
| | | TTATISCEDNEEDVASTKTELLTMETTITSCSGGICTTLMSPVSS |
| | | FNSKATTSNNAESTIPKAIKVSCSAGACTTLTTVDAGISMFTRTG |
| | | LSITQTTVTNCSGGTCTMLTAPIATATSKVISPIPKASSATSIAH |
| | | SSASYTVSINTNGAYNFDKDNIFGTAIVAVVALLLL |
| 68 | Sag1 from | ININDITFSNLEITPLTANKQPDQGWTATFDFSIADASSIREGDE |
| | Saccharomyces | FTLSMPHVYRIKLLNSSQTATISLADGTEAFKCYVSQQAAYLYEN |
| | cerevisiae | TTFTCTAQNDLSSYNTIDGSITESLNFSDGGSSYEYELENAKFFK |
| | | SGPMLVKLGNQMSDVVNFDPAAFTENVFHSGRSTGYGSFESYHLG |
| | | MYCPNGYFLGGTEKIDYDSSNNNVDLDCSSVQVYSSNDFNDWWFP |
| | | QSYNDTNADVTCFGSNLWITLDEKLYDGEMLWVNALQSLPANVNT |
| | | IDHALEFQYTCLDTIANTTYATQFSTTREFIVYQGRNLGTASAKS |
| | | SFISTTTTDLTSINTSAYSTGSISTVETGNRTTSEVISHVVTTST |
| | | KLSPTATTSLTIAQTSIYSTDSNITVGTDIHTTSEVISDVETISR |
| | | ETASTVVAAPTSTTGWTGAMNTYISQFTSSSFATINSTPIISSSA |
| | | VFETSDASIVNVHTENITNTAAVPSEEPTFVNATRNSLNSFCSSK |
| | | QPSSPSSYTSSPLVSSLSVSKTLLSTSFTPSVPTSNTYIKTKNTG |
| | | YFEHTALTTSSVGLNSFSETAVSSQGTKIDTFLVSSLIAYPSSAS |
| | | GSQLSGIQQNFTSTSLMISTYEGKASIFFSAELGSIIFLLLSYLL |
| | | F |
| 69 | Sag1 from | MFTFLKIILWLFSLALASAININDITFSNLEITPLTANKQPDQGW |
| | Saccharomyces | TATFDFSIADASSIREGDEFTLSMPHVYRIKLLNSSQTATISLAD |
| | cerevisiae | GTEAFKCYVSQQAAYLYENTTFTCTAQNDLSSYNTIDGSITESLN |
| | (underlined | FSDGGSSYEYELENAKFFKSGPMLVKLGNQMSDVVNFDPAAFTEN |
| | is signal | VFHSGRSTGYGSFESYHLGMYCPNGYFLGGTEKIDYDSSNNNVDL |
| | peptide, | DCSSVQVYSSNDFNDWWFPQSYNDTNADVTCFGSNLWITLDEKLY |
| | which may | DGEMLWVNALQSLPANVNTIDHALEFQYTCLDTIANTTYATQFST |
| | not be | TREFIVYQGRNLGTASAKSSFISTTTTDLTSINTSAYSTGSISTV |
| | utilized | ETGNRTTSEVISHVVTTSTKLSPTATTSLTIAQTSIYSTDSNITV |
| | in design) | GTDIHTTSEVISDVETISRETASTVVAAPTSTTGWTGAMNTYISQ |
| | | FTSSSFATINSTPIISSSAVFETSDASIVNVHTENITNTAAVPSE |
| | | EPTFVNATRNSLNSFCSSKQPSSPSSYTSSPLVSSLSVSKTLLST |
| | | SFTPSVPTSNTYIKTKNTGYFEHTALTTSSVGLNSFSETAVSSQG |
| | | TKIDTFLVSSLIAYPSSASGSQLSGIQQNFTSTSLMISTYEGKAS |
| | | IFFSAELGSIIFLLLSYLLF |
| 70 | FIG. 2 from | QIVFYQNSSTSLPVPTLVSTSIADFHESSSTGEVQYSSSYSYVQP |
| | Saccharomyces | SIDSFTSSSFLTSFEAPTETSSSYAVSSSLITSDTFSSYSDIFDE |
| | cerevisiae | ETSSLISTSAASSEKASSTLSSTAQPHRTSHSSSSFELPVTAPSS |
| | | SSLPSSTSLTFTSVNPSQSWTSFNSEKSSALSSTIDFTSSEISGS |
| | | TSPKSLESFDTTGTITSSYSPSPSSKNSNQTSLLSPLEPLSSSSG |
| | | DLILSSTIQATTNDQTSKTIPTLVDATSSLPPTLRSSSMAPTSGS |
| | | DSISHNFTSPPSKTSGNYDVLTSNSIDPSLFTTTSEYSSTQLSSL |
| | | NRASKSETVNFTASIASTPFGTDSATSLIDPISSVGSTASSFVGI |
| | | STANFSTQGNSNYVPESTASGSSQYQDWSSSSLPLSQTTWVVINT |
| | | TNTQGSVTSTTSPAYVSTATKTVDGVITEYVTWCPLTQTKSQAIG |
| | | VSSSISSVPQASSFSGSSILSSNSSTLAASNNVPESTASGSSQYQ |
| | | DWSSSSLPLSQTTWVVINTTNTQGSVTSTTSPAYVSTATKTVDGV |
| | | ITEYVTWCPLTQTKSQAIGISSSTISATQTSKPSSILTLGISTLQ |
| | | LSDATFKGTETINTHLMTESTSITEPTYFSGTSDSFYLCTSEVNL |
| | | ASSLSSYPNFSSSEGSTATITNSTVTFGSTSKYPSTSVSNPTEAS |
| | | QHVSSSVNSLTDFTSNSTETIAVISNIHKTSSNKDYSLTTTQLKT |
| | | SGMQTLVLSTVTTTVNGAATEYTTWCPASSIAYTTSISYKTLVLT |
| | | TEVCSHSECTPTVITSVTATSSTIPLLSTSSSTVLSSTVSEGAKN |
| | | PAASEVTINTQVSATSEATSTSTQVSATSATATASESSTTSQVST |
| | | ASETISTLGTQNFTTTGSLLFPALSTEMINTTVVSRKTLIISTEV |
| | | CSHSKCVPTVITEVVTSKGTPSNGHSSQTLQTEAVEVTLSSHQTV |
| | | TMSTEVCSNSICTPTVITSVQMRSTPFPYLTSSTSSSSLASTKKS |
| | | SLEASSEMSTFSVSTQSLPLAFTSSEKRSTTSVSQWSNTVLTNTI |
| | | MSSSSNVISTNEKPSSTTSPYNFSSGYSLPSSSTPSQYSLSTATT |
| | | TINGIKTVYTTWCPLAEKSTVAASSQSSRSVDRFVSSSKPSSSLS |
| | | QTSIQYTLSTATTTISGLKTVYTTWCPLTSKSTLGATTQTSSTAK |
| | | VRITSASSATSTSISLSTSTESESSSGYLSKGVCSGTECTQDVPT |
| | | QSSSPASTLAYSPSVSTSSSSSFSTTTASTLTSTHTSVPLLPSSS |
| | | SISASSPSSTSLLSTSLPSPAFTSSTLPTATAVSSSTFIASSLPL |
| | | SSKSSLSLSPVSSSILMSQFSSSSSSSSSLASLPSLSISPTVDTV |
| | | SVLQPTTSIATLTCTDSQCQQEVSTICNGSNCDDVTSTATTPPST |
| | | VTDTMTCTGSECQKTTSSSCDGYSCKVSETYKSSATISACSGEGC |
| | | QASATSELNSQYVTMTSVITPSAITTTSVEVHSTESTISITTVKP |
| | | VTYTSSDTNGELITITSSSQTVIPSVTTIITRTKVAITSAPKPTT |
| | | TTYVEQRLSSSGIATSFVAAASSTWITTPIVSTYAGSASKFLCSK |
| | | FFMIMVMVINFI |
| 71 | FIG. 2 from | MNSFASLGLIYSVVNLLTRVEAQIVFYQNSSTSLPVPTLVSTSIA |
| | Saccharomyces | DFHESSSTGEVQYSSSYSYVQPSIDSFTSSSFLTSFEAPTETSSS |
| | cerevisiae | YAVSSSLITSDTFSSYSDIFDEETSSLISTSAASSEKASSTLSST |
| | (underlined | AQPHRTSHSSSSFELPVTAPSSSSLPSSTSLTFTSVNPSQSWTSF |
| | is signal | NSEKSSALSSTIDFTSSEISGSTSPKSLESFDTTGTITSSYSPSP |
| | peptide, | SSKNSNQTSLLSPLEPLSSSSGDLILSSTIQATTNDQTSKTIPTL |
| | which may | VDATSSLPPTLRSSSMAPTSGSDSISHNFTSPPSKTSGNYDVLTS |
| | not be | NSIDPSLFTTTSEYSSTQLSSLNRASKSETVNFTASIASTPFGTD |
| | utilized | SATSLIDPISSVGSTASSFVGISTANFSTQGNSNYVPESTASGSS |
| | in design) | QYQDWSSSSLPLSQTTWVVINTTNTQGSVTSTTSPAYVSTATKTV |
| | | DGVITEYVTWCPLTQTKSQAIGVSSSISSVPQASSFSGSSILSSN |
| | | SSTLAASNNVPESTASGSSQYQDWSSSSLPLSQTTWVVINTTNTQ |
| | | GSVTSTTSPAYVSTATKTVDGVITEYVTWCPLTQTKSQAIGISSS |
| | | TISATQTSKPSSILTLGISTLQLSDATFKGTETINTHLMTESTSI |
| | | TEPTYFSGTSDSFYLCTSEVNLASSLSSYPNFSSSEGSTATITNS |
| | | TVTFGSTSKYPSTSVSNPTEASQHVSSSVNSLTDFTSNSTETIAV |
| | | ISNIHKTSSNKDYSLTTTQLKTSGMQTLVLSTVTTTVNGAATEYT |
| | | TWCPASSIAYTTSISYKTLVLTTEVCSHSECTPTVITSVTATSST |
| | | IPLLSTSSSTVLSSTVSEGAKNPAASEVTINTQVSATSEATSTST |
| | | QVSATSATATASESSTTSQVSTASETISTLGTQNFTTTGSLLFPA |
| | | LSTEMINTTVVSRKTLIISTEVCSHSKCVPTVITEVVTSKGTPSN |
| | | GHSSQTLQTEAVEVTLSSHQTVTMSTEVCSNSICTPTVITSVQMR |
| | | STPFPYLTSSTSSSSLASTKKSSLEASSEMSTFSVSTQSLPLAFT |
| | | SSEKRSTTSVSQWSNTVLTNTIMSSSSNVISTNEKPSSTTSPYNF |
| | | SSGYSLPSSSTPSQYSLSTATTTINGIKTVYTTWCPLAEKSTVAA |
| | | SSQSSRSVDRFVSSSKPSSSLSQTSIQYTLSTATTTISGLKTVYT |
| | | TWCPLTSKSTLGATTQTSSTAKVRITSASSATSTSISLSTSTESE |
| | | SSSGYLSKGVCSGTECTQDVPTQSSSPASTLAYSPSVSTSSSSSF |
| | | STTTASTLTSTHTSVPLLPSSSSISASSPSSTSLLSTSLPSPAFT |
| | | SSTLPTATAVSSSTFIASSLPLSSKSSLSLSPVSSSILMSQFSSS |
| | | SSSSSSLASLPSLSISPTVDTVSVLQPTTSIATLTCTDSQCQQEV |
| | | STICNGSNCDDVTSTATTPPSTVTDTMTCTGSECQKTTSSSCDGY |
| | | SCKVSETYKSSATISACSGEGCQASATSELNSQYVTMTSVITPSA |
| | | ITTTSVEVHSTESTISITTVKPVTYTSSDTNGELITITSSSQTVI |
| | | PSVTTIITRTKVAITSAPKPTTTTYVEQRLSSSGIATSFVAAASS |
| | | TWITTPIVSTYAGSASKFLCSKFFMIMVMVINFI |

TABLE 5
Exemplary Proteins of Interest

| | SEQ | |
|---|---|---|
| | ID | |
| Sequence Info | NO: | Sequence |
| Ovomucoid | 92 | AEVDCSRFPNATDKEGKDVLVCNKDLRPICGTDGVTYTNDCL |
| (canonical) | | LCAYSIEFGTNISKEHDGECKETVPMNCSSYANTTSEDGKVM |
| | | VLCNRAFNPVCGTDGVTYDNECLLCAHKVEQGASVDKRHDGG |
| | | CRKELAAVSVDCSEYPKPDCTAEDRPLCGSDNKTYGNKCNFC |
| | | NAVVESNGTLTLSHFGKC* |
| Ovomucoid | 93 | AEVDCSRFPNATDMEGKDVLVCNKDLRPICGTDGVTYTNDCL |
| | | LCAYSVEFGTNISKEHDGECKETVPMNCSSYANTTSEDGKVM |
| | | VLCNRAFNPVCGTDGVTYDNECLLCAHKVEQGASVDKRHDGG |
| | | CRKELAAVSVDCSEYPKPDCTAEDRPLCGSDNKTYGNKCNFC |
| | | NAVVESNGTLTLSHFGKC* |
| Ovomucoid | 94 | AEVDCSRFPNATDMEGKDVLVCNKDLRPICGTDGVTYTNDCL |
| G162M F167A | | LCAYSVEFGTNISKEHDGECKETVPMNCSSYANTTSEDGKVM |
| | | VLCNRAFNPVCGTDGVTYDNECLLCAHKVEQGASVDKRHDGG |
| | | CRKELAAVSVDCSEYPKPDCTAEDRPLCGSDNKTYMNKCNAC |
| | | NAVVESNGTLTLSHFGKC* |
| Ovomucoid | 95 | MAMAGVFVLFSFVLCGFLPDAAFGAEVDCSRFPNATDKEGKD |
| isoform 1 | | VLVCNKDLRPICGTDGVTYTNDCLLCAYSIEFGTNISKEHDG |
| precursor full | | ECKETVPMNCSSYANTTSEDGKVMVLCNRAFNPVCGTDGVTY |
| length | | DNECLLCAHKVEQGASVDKRHDGGCRKELAAVSVDCSEYPKP |
| | | DCTAEDRPLCGSDNKTYGNKCNFCNAVVESNGTLTLSHFGKC |
| Ovomucoid | 96 | MAMAGVFVLFSFVLCGFLPDAVFGAEVDCSRFPNATDMEGKD |
| [Gallus gallus] | | VLVCNKDLRPICGTDGVTYTNDCLLCAYSVEFGTNISKEHDG |
| | | ECKETVPMNCSSYANTTSEDGKVMVLCNRAFNPVCGTDGVTY |
| | | DNECLLCAHKVEQGASVDKRHDGGCRKELAAVSVDCSEYPKP |
| | | DCTAEDRPLCGSDNKTYGNKCNFCNAVVESNGTLTLSHFGKC |
| Ovomucoid | 97 | MAMAGVFVLFSFVLCGFLPDAAFGAEVDCSRFPNATDKEGKD |
| isoform 2 | | VLVCNKDLRPICGTDGVTYTNDCLLCAYSIEFGTNISKEHDG |
| precursor | | ECKETVPMNCSSYANTTSEDGKVMVLCNRAFNPVCGTDGVTY |
| [Gallus gallus] | | DNECLLCAHKVEQGASVDKRHDGGCRKELAAVDCSEYPKPDC |
| | | TAEDRPLCGSDNKTYGNKCNFCNAVVESNGTLTLSHFGKC |
| Ovomucoid | 98 | AEVDCSRFPNATDKEGKDVLVCNKDLRPICGTDGVTYNNECL |
| [Gallus gallus] | | LCAYSIEFGTNISKEHDGECKETVPMNCSSYANTTSEDGKVM |
| | | VLCNRAFNPVCGTDGVTYDNECLLCAHKVEQGASVDKRHDGE |
| | | CRKELAAVSVDCSEYPKPDCTAEDRPLCGSDNKTYGNKCNFC |
| | | NAVVESNGTLTLSHFGKC |
| Ovomucoid | 99 | MAMAGVFVLFSFALCGFLPDAAFGVEVDCSRFPNATNEEGKD |
| [Numida | | VLVCTEDLRPICGTDGVTYSNDCLLCAYNIEYGTNISKEHDG |
| meleagris] | | ECREAVPVDCSRYPNMTSEEGKVLILCNKAFNPVCGTDGVTY |
| | | DNECLLCAHNVEQGTSVGKKHDGECRKELAAVDCSEYPKPAC |
| | | TMEYRPLCGSDNKTYDNKCNFCNAVVESNGTLTLSHFGKC |
| PREDICTED: | 100 | MQTITWRQPQGDHLRSRAPAATCRAGQYLTMAMAGIFVLFSF |
| Ovomucoid | | ALCGFLPDAAFGVEVDCSRFPNTTNEEGKDVLVCTEDLRPIC |
| isoform X1 | | GTDGVTHSECLLCAYNIEYGTNISKEHDGECREAVPMDCSRY |
| [Meleagris | | PNTTNEEGKVMILCNKALNPVCGTDGVTYDNECVLCAHNLEQ |
| gallopavo] | | GTSVGKKHDGGCRKELAAVSVDCSEYPKPACTLEYRPLCGSD |
| | | NKTYGNKCNFCNAVVESNGTLTLSHFGKC |
| Ovomucoid | 101 | VEVDCSRFPNTTNEEGKDVLVCTEDLRPICGTDGVTHSECLL |
| [Meleagris | | CAYNIEYGTNISKEHDGECREAVPMDCSRYPNTTSEEGKVMI |
| gallopavo] | | LCNKALNPVCGTDGVTYDNECVLCAHNLEQGTSVGKKHDGEC |
| | | RKELAAVSVDCSEYPKPACTLEYRPLCGSDNKTYGNKCNFCN |
| | | AVVESNGTLTLSHFGKC |
| PREDICTED: | 102 | MQTITWRQPQGDHLRSRAPAATCRAGQYLTMAMAGIFVLFSF |
| Ovomucoid | | ALCGFLPDAAFGVEVDCSRFPNTTNEEGKDVLVCTEDLRPIC |
| isoform X2 | | GTDGVTHSECLLCAYNIEYGTNISKEHDGECREAVPMDCSRY |
| [Meleagris | | PNTTNEEGKVMILCNKALNPVCGTDGVTYDNECVLCAHNLEQ |
| gallopavo] | | GTSVGKKHDGGCRKELAAVDCSEYPKPACTLEYRPLCGSDNK |
| | | TYGNKCNFCNAVVESNGTLTLSHFGKC |
| Ovomucoid | 103 | EYGTNISIKHNGECKETVPMDCSRYANMTNEEGKVMMPCDRT |
| [Bambusicola | | YNPVCGTDGVTYDNECQLCAHNVEQGTSVDKKHDGVCGKELA |
| thoracicus] | | AVSVDCSEYPKPECTAEERPICGSDNKTYGNKCNFCNAVVYV |
| | | QP |
| Ovomucoid | 104 | VDCSRFPNTTNEEGKDVLACTKELHPICGTDGVTYSNECLLC |
| [Callipepla | | YYNIEYGTNISKEHDGECTEAVPVDCSRYPNTTSEEGKVLIP |
| squamata] | | CNRDFNPVCGSDGVTYENECLLCAHNVEQGTSVGKKHDGGCR |
| | | KEFAAVSVDCSEYPKPDCTLEYRPLCGSDNKTYASKCNFCNA |
| | | VVIWEQEKNTRHHASHSVFFISARLVC |
| Ovomucoid | 105 | MLPLGLREYGTNTSKEHDGECTEAVPVDCSRYPNTTSEEGKV |
| [Colinus | | RILCKKDINPVCGTDGVTYDNECLLCSHSVGQGASIDKKHDG |
| virginianus] | | GCRKEFAAVSVDCSEYPKPACMSEYRPLCGSDNKTYVNKCNF |
| | | CNAVVYVQPWLHSRCRLPPTGTSFLGSEGRETSLLTSRATDL |
| | | QVAGCTAISAMEATRAAALLGLVLLSSFCELSHLCFSQASCD |
| | | VYRLSGSRNLACPRIFQPVCGTDNVTYPNECSLCRQMLRSRA |
| | | VYKKHDGRCVKVDCTGYMRATGGLGTACSQQYSPLYATNGVI |
| | | YSNKCTFCSAVANGEDIDLLAVKYPEEESWISVSPTPWRMLS |
| | | AGA |
| Ovomucoid-like | 106 | MSWWGIKPALERPSQEQSTSGQPVDSGSTSTTTMAGIFVLLS |
| isoform X2 | | LVLCCFPDAAFGVEVDCSRFPNTTNEEGKEVLLCTKDLSPIC |
| [Anser | | GTDGVTYSNECLLCAYNIEYGTNISKDHDGECKEAVPVDCST |
| cygnoides | | YPNMTNEEGKVMLVCNKMFSPVCGTDGVTYDNECMLCAHNVE |
| domesticus] | | QGTSVGKKYDGKCKKEVATVDCSDYPKPACTVEYMPLCGSDN |
| | | KTYDNKCNFCNAVVDSNGTLTLSHFGKC |
| Ovomucoid-like | 107 | MSSQNQLHRRRRPLPGGQDLNKYYWPHCTSDRFSWLLHVTAE |
| isoform X1 | | QFRHCVCIYLQPALERPSQEQSTSGQPVDSGSTSTTTMAGIF |
| [Anser | | VLLSLVLCCFPDAAFGVEVDCSRFPNTTNEEGKEVLLCTKDL |
| cygnoides | | SPICGTDGVTYSNECLLCAYNIEYGTNISKDHDGECKEAVPV |
| domesticus] | | DCSTYPNMTNEEGKVMLVCNKMFSPVCGTDGVTYDNECMLCA |
| | | HNVEQGTSVGKKYDGKCKKEVATVDCSDYPKPACTVEYMPLC |
| | | GSDNKTYDNKCNFCNAVVDSNGTLTLSHFGKC |
| Ovomucoid | 108 | VEVDCSRFPNTTNEEGKDEVVCPDELRLICGTDGVTYNHECM |
| [Coturnix | | LCFYNKEYGTNISKEQDGECGETVPMDCSRYPNTTSEDGKVT |
| japonica] | | ILCTKDFSFVCGTDGVTYDNECMLCAHNVVQGTSVGKKHDGE |
| | | CRKELAAVSVDCSEYPKPACPKDYRPVCGSDNKTYSNKCNFC |
| | | NAVVESNGTLTLNHFGKC |
| Ovomucoid | 109 | MAMAGVFLLFSFALCGFLPDAAFGVEVDCSRFPNTTNEEGKD |
| [Coturnix | | EVVCPDELRLICGTDGVTYNHECMLCFYNKEYGTNISKEQDG |
| japonica] | | ECGETVPMDCSRYPNTTSEDGKVTILCTKDFSFVCGTDGVTY |
| | | DNECMLCAHNIVQGTSVGKKHDGECRKELAAVSVDCSEYPKP |
| | | ACPKDYRPVCGSDNKTYSNKCNFCNAVVESNGTLTLNHFGKC |
| Ovomucoid | 110 | MAGVFVLLSLVLCCFPDAAFGVEVDCSRFPNTTNEEGKDVLL |
| [Anas | | CTKELSPVCGTDGVTYSNECLLCAYNIEYGTNISKDHDGECK |
| platyrhynchos] | | EAVPADCSMYPNMTNEEGKMTLLCNKMFSPVCGTDGVTYDNE |
| | | CMLCAHNVEQGTSVGKKYDGKCKKEVATVDCSGYPKPACTME |
| | | YMPLCGSDNKTYGNKCNFCNAVVDSNGTLTLSHFGEC |
| Ovomucoid, | 111 | QVDCSRFPNTTNEEGKEVLLCTKELSPVCGTDGVTYSNECLL |
| partial [Anas | | CAYNIEYGTNISKDHDGECKEAVPADCSMYPNMTNEEGKMTL |
| platyrhynchos] | | LCNKMFSPVCGTDGVTYDNECMLCAHNVEQGTSVGKKYDGKC |
| | | KKEVATVSVDCSGYPKPACTMEYMPLCGSDNKTYGNKCNFCN |
| | | AVV |
| Ovomucoid-like | 112 | MTMPGAFVVLSFVLCCFPDATFGVEVDCSTYPNTTNEEGKEV |
| [Tyto alba] | | LVCSKILSPICGTDGVTYSNECLLCANNIEYGTNISKYHDGE |
| | | CKEFVPVNCSRYPNTTNEEGKVMLICNIKDLSPVCGTDGVTY |
| | | DNECLLCAHNLEPGTSVGKKYDGECKKEIATVDCSDYPKPVC |
| | | SLESMPLCGSDNKTYSNKCNFCNAVVDSNETLTLSHFGKC |
| Ovomucoid | 113 | MTMAGVFVLLSFALCCFPDAAFGVEVDCSTYPNTTNEEGKEV |
| [Balearica | | LVCTKILSPICGTDGVTYSNECLLCAYNIEYGTNVSKDHDGE |
| regulorum | | CKEVVPVDCSRYPNSTNEEGKVVMLCSKDLNPVCGTDGVTYD |
| gibbericeps] | | NECVLCAHNVESGTSVGKKYDGECKKETATVDCSDYPKPACT |
| | | LEYMPFCGSDSKTYSNKCNFCNAVVDSNGTLTLSHFGKC |
| Turkey vulture | 114 | MTTAGVFVLLSFALCSFPDAAFGVEVDCSTYPNTTNEEGKEV |
| [Cathartes aura] | | LVCTKILSPICGTDGVTYSNECLLCAYNIEYGTNVSKDHDGE |
| OVD (native | | CKEFVPVDCSRYPNTTNEDGKVVLLCNKDLSPICGTDGVTYD |
| sequence) | | NECLLCARNLEPGTSVGKKYDGECKKEIATVDCSDYPKPVCS |
| bolded is native | | LEYMPLCGSDSKTYSNKCNFCNAVVDSNGTLTLSHFGKC |
| signal sequence | | |
| Ovomucoid-like | 115 | MTTAGVFVLLSFTLCSFPDAAFGVEVDCSPYPNTTNEEGKEV |
| [Cuculus | | LVCNKILSPICGTDGVTYSNECLLCAYNLEYGTNISKDYDGE |
| canorus] | | CKEVAPVDCSRHPNTTNEEGKVELLCNKDLNPICGTNGVTYD |
| | | NECLLCARNLESGTSIGKKYDGECKKEIATVDCSDYPKPVCT |
| | | LEEMPLCGSDNKTYGNKCNFCNAVVDSNGTLTLSHFGKC |
| Ovomucoid | 116 | MTTAVVFVLLSFALCCFPDAAFGVEVDCSTYPNSTNEEGKDV |
| [Antrostomus | | LVCPKILGPICGTDGVTYSNECLLCAYNIQYGTNVSKDHDGE |
| carolinensis] | | CKEIVPVDCSRYPNTTNEEGKVVFLCNKNFDPVCGTDGDTYD |
| | | NECMLCARSLEPGTTVGKKHDGECKREIATVDCSDYPKPTCS |
| | | AEDMPLCGSDSKTYSNKCNFCNAVVDSNGTLTLSRFGKC |
| Ovomucoid | 117 | MTMTGVFVLLSFAICCFPDAAFGVEVDCSTYPNTTNEEGKEV |
| [Cariama | | LVCTKILSPICGTDGVTYSNECLLCAYNIEYGTNVSKDHDGE |
| cristata] | | CKEVVPVDCSKYPNTTNEEGKVVLLCSKDLSPVCGTDGVTYD |
| | | NECLLCARNLEPGSSVGKKYDGECKKEIATIDCSDYPKPVCS |
| | | LEYMPLCGSDSKTYDNKCNFCNAVVDSNGTLTLSHFGKC |
| Ovomucoid-like | 118 | MTTAGVFVLLSFVLCCFPDAVFGVEVDCSTYPNTTNEEGKEV |
| isoform X2 | | LVCTKILSPICGTDGVTYSNECLLCAYNIEYGTNVSKDHDGE |
| [Pygoscelis | | CKEVVPVNCSRYPNTTNEEGKVVLRCSKDLSPVCGTDGVTYD |
| adeliae] | | NECLMCARNLEPGAVVGKNYDGECKKEIATVDCSDYPKPVCS |
| | | LEYMPLCGSDSKTYSNKCNFCNAVVDSNGTLTLSHFGKC |
| Ovomucoid-like | 119 | MTTAGVFVLLSIALCCFPDAAFGVEVDCSAYSNTTSEEGKEV |
| [Nipponia | | LSCTKILSPICGTDGVTYSNECLLCAYNIEYGTNISKDHDGE |
| nippon] | | CKEVVSVDCSRYPNTTNEEGKAVLLCNKDLSPVCGTDGVTYD |
| | | NECLLCAHNLEPGTSVGKKYDGACKKEIATVDCSDYPKPVCT |
| | | LEYLPLCGSDSKTYSNKCDFCNAVVDSNGTLTLSHFGKC |
| Ovomucoid-like | 120 | MTTAGVFVLLSFALCCFPDAAFGVEVDCSTYPNTTNEEGKEV |
| [Phaethon | | LVCTKILSPICGTDGTTYSNECLLCAYNIEYGTNVSKDHDGE |
| lepturus] | | CKVVPVDCSKYPNTTNEDGKVVLLCNKALSPICGTDRVTYDN |
| | | ECLMCAHNLEPGTSVGKKHDGECQKEVATVDCSDYPKPVCSL |
| | | EYMPLCGSDGKTYSNKCNFCNAVVNSNGTLTLSHFEKC |
| Ovomucoid-like | 121 | MTTAGVFVLLSFVLCCFFPDAAFGVEVDCSTYPNTTNEEGKE |
| isoform X1 | | VLVCAKILSPVCGTDGVTYSNECLLCAHNIENGTNVGKDHDG |
| [Melopsittacus | | KCKEAVPVDCSRYPNTTDEEGKVVLLCNKDVSPVCGTDGVTY |
| undulatus] | | DNECLLCAHNLEAGTSVDKKNDSECKTEDTTLAAVSVDCSDY |
| | | PKPVCTLEYLPLCGSDNKTYSNKCRFCNAVVDSNGTLTLSRF |
| | | GKC |
| Ovomucoid | 122 | MTTAGVFVLLSFALCCSPDAAFGVEVDCSTYPNTTNEEGKEV |
| [Podiceps | | LACTKILSPICGTDGVTYSNECLLCAYNMEYGTNVSKDHDGK |
| cristatus] | | CKEVVPVDCSRYPNTTNEEGKVVLLCNKDLSPVCGTDGVTYD |
| | | NECLLCARNLEPGASVGKKYDGECKKEIATVDCSDYPKPVCS |
| | | LEHMPLCGSDSKTYSNKCTFCNAVVDSNGTLTLSHFGKC |
| Ovomucoid-like | 123 | MTTAGVFVLLSFALCCFPDAAFGVEVDCSTYPNTTNEEGREV |
| [Fulmarus | | LVCTKILSPICGTDGVTYSNECLLCAYNIEYGTNVSKDHDGE |
| glacialis] | | CKEVAPVGCSRYPNTTNEEGKVVLLCNKDLSPVCGTDGVTYD |
| | | NECLLCARHLEPGTSVGKKYDGECKKEIATVDCSDYPKPVCS |
| | | LEYMPLCGSDSKTYSNKCNFCNAVLDSNGTLTLSHFGKC |
| Ovomucoid | 124 | MTTAGVFVLLSFALCCFPDAVFGVEVDCSTYPNTTNEEGKEV |
| [Aptenodytes | | LVCTKILSPICGTDGVTYSNECLLCAYNIEYGTNVSKDHDGE |
| forsteri] | | CKEVVPVDCSRYPNTTNEEGKVVLRCNKDLSPVCGTDGVTYD |
| | | NECLMCARNLEPGAIVGKKYDGECKKEIATVDCSDYPKPVCS |
| | | LEYMPLCGSDSKTYSNKCNFCNAVVDSNGTLILSHFGKC |
| Ovomucoid-like | 125 | MTTAGVFVLLSFVLCCFPDAVFGVEVDCSTYPNTTNEEGKEV |
| isoform X1 | | LVCTKILSPICGTDGVTYSNECLLCAYNIEYGTNVSKDHDGE |
| [Pygoscelis | | CKEVVPVDCSRYPNTTNEEGKVVLRCSKDLSPVCGTDGVTYD |
| adeliae] | | NECLMCARNLEPGAVVGKNYDGECKKEIATVDCSDYPKPVCS |
| | | LEYMPLCGSDSKTYSNKCNFCNAVVDSNGTLTLSHFGKC |
| Ovomucoid | 126 | MSSQNQLPSRCRPLPGSQDLNKYYQPHCTGDRFCWLFYVTVE |
| isoform X1 | | QFRHCICIYLQLALERPSHEQSGQPADSRNTSTMTTAGVFVL |
| [Aptenodytes | | LSFALCCFPDAVFGVEVDCSTYPNTTNEEGKEVLVCTKILSP |
| forsteri] | | ICGTDGVTYSNECLLCAYNIEYGTNVSKDHDGECKEVVPVDC |
| | | SRYPNTTNEEGKVVLRCNKDLSPVCGTDGVTYDNECLMCARN |
| | | LEPGAIVGKKYDGECKKEIATVDCSDYPKPVCSLEYMPLCGS |
| | | DSKTYSNKCNFCNAVVDSNGTLILSHFGKC |
| Ovomucoid, | 127 | MTTAVVFVLLSFALCCFPDAAFGVEVDCSTYPNSTNEEGKDV |
| partial | | LVCPKILGPICGTDGVTYSNECLLCAYNIQYGTNVSKDHDGE |
| [Antrostomus | | CKEIVPVDCSRYPNTTNEEGKVVFLCNKNFDPVCGTDGDTYD |
| carolinensis] | | NECMLCARSLEPGTTVGKKHDGECKREIATVDCSDYPKPTCS |
| | | AEDMPLCGSDSKTYSNKCNFCNAVV |
| rOVD as | 128 | EAEAAEVDCSRFPNATDKEGKDVLVCNKDLRPICGTDGVTYT |
| expressed in | | NDCLLCAYSIEFGTNISKEHDGECKETVPMNCSSYANTTSED |
| pichia secreted | | GKVMVLCNRAFNPVCGTDGVTYDNECLLCAHKVEQGASVDKR |
| form 1 | | HDGGCRKELAAVSVDCSEYPKPDCTAEDRPLCGSDNKTYGNK |
| | | CNFCNAVVESNGTLTLSHFGKC |
| rOVD as | 129 | EEGVSLEKREAEAAEVDCSRFPNATDKEGKDVLVCNKDLRPI |
| expressed in | | CGTDGVTYTNDCLLCAYSIEFGTNISKEHDGECKETVPMNCS |
| pichia secreted | | SYANTTSEDGKVMVLCNRAFNPVCGTDGVTYDNECLLCAHKV |
| form 2 | | EQGASVDKRHDGGCRKELAAVSVDCSEYPKPDCTAEDRPLCG |
| | | SDNKTYGNKCNFCNAVVESNGTLTLSHFGKC |
| rOVD [gallus] | 130 | MRFPSIFTAVLFAASSALAAPVNTTTEDETAQIPAEAVIGYS |
| coding | | DLEGDFDVAVLPFSNSTNNGLLFINTTIASIAAKEEGVSLEK |
| sequence | | REAEAAEVDCSRFPNATDKEGKDVLVCNKDLRPICGTDGVTY |
| containing an | | TNDCLLCAYSIEFGTNISKEHDGECKETVPMNCSSYANTTSE |
| alpha mating | | DGKVMVLCNRAFNPVCGTDGVTYDNECLLCAHKVEQGASVDK |
| factor signal | | RHDGGCRKELAAVSVDCSEYPKPDCTAEDRPLCGSDNKTYGN |
| sequence | | KCNFCNAVVESNGTLTLSHFGKC |
| (bolded) as | | |
| expressed in | | |
| pichia | | |
| Turkey vulture | 131 | MRFPSIFTAVLFAASSALAAPVNTTTEDETAQIPAEAVIGYS |
| OVD coding | | DLEGDFDVAVLPFSNSTNNGLLFINTTIASIAAKEEGVSLEK |
| sequence | | REAEAVEVDCSTYPNTTNEEGKEVLVCTKILSPICGTDGVTY |
| containing | | SNECLLCAYNIEYGTNVSKDHDGECKEFVPVDCSRYPNTTNE |
| secretion | | DGKVVLLCNKDLSPICGTDGVTYDNECLLCARNLEPGTSVGK |
| signals as | | KYDGECKKEIATVDCSDYPKPVCSLEYMPLCGSDSKTYSNKC |
| expressed in | | NFCNAVVDSNGTLTLSHFGKC |
| pichia | | |
| bolded is an | | |
| alpha mating | | |
| factor signal | | |
| sequence | | |
| Turkey vulture | 132 | EAEAVEVDCSTYPNTTNEEGKEVLVCTKILSPICGTDGVTYS |
| OVD in | | NECLLCAYNIEYGTNVSKDHDGECKEFVPVDCSRYPNTTNED |
| secreted form | | GKVVLLCNKDLSPICGTDGVTYDNECLLCARNLEPGTSVGKK |
| expressed in | | YDGECKKEIATVDCSDYPKPVCSLEYMPLCGSDSKTYSNKCN |
| Pichia | | FCNAVVDSNGTLTLSHFGKC |
| Humming bird | 133 | MTMAGVFVLLSFILCCFPDTAFGVEVDCSIYPNTTSEEGKEV |
| OVD (native | | LVCTETLSPICGSDGVTYNNECQLCAYNVEYGTNVSKDHDGE |
| sequence) | | CKEIVPVDCSRYPNTTEEGRVVMLCNKALSPVCGTDGVTYDN |
| bolded is the | | ECLLCARNLESGTSVGKKFDGECKKEIATVDCTDYPKPVCSL |
| native signal | | DYMPLCGSDSKTYSNKCNFCNAVMDSNGTLTLNHFGKC |
| sequence | | |
| Humming bird | 134 | MRFPSIFTAVLFAASSALAAPVNTTTEDETAQIPAEAVIGYS |
| OVD coding | | DLEGDFDVAVLPFSNSTNNGLLFINTTIASIAAKEEGVSLDK |
| sequence as | | REAEAVEVDCSIYPNTTSEEGKEVLVCTETLSPICGSDGVTY |
| expressed in | | NNECQLCAYNVEYGTNVSKDHDGECKEIVPVDCSRYPNTTEE |
| Pichia | | GRVVMLCNKALSPVCGTDGVTYDNECLLCARNLESGTSVGKK |
| bolded is an | | FDGECKKEIATVDCTDYPKPVCSLDYMPLCGSDSKTYSNKCN |
| alpha mating | | FCNAVMDSNGTLTLNHFGKC |
| factor signal | | |
| sequence | | |
| Humming bird | 135 | EAEAVEVDCSIYPNTTSEEGKEVLVCTETLSPICGSDGVTYN |
| OVD in | | NECQLCAYNVEYGTNVSKDHDGECKEIVPVDCSRYPNTTEEG |
| secreted form | | RVVMLCNKALSPVCGTDGVTYDNECLLCARNLESGTSVGKKE |
| from Pichia | | DGECKKEIATVDCTDYPKPVCSLDYMPLCGSDSKTYSNKCNF |
| | | CNAVMDSNGTLTLNHFGKC |
| Ovalbumin | 136 | MFFYNTDFRMGSISAANAEFCFDVFNELKVQHTNENILYSPL |
| related protein | | SIIVALAMVYMGARGNTEYQMEKALHFDSIAGLGGSTQTKVQ |
| X | | KPKCGKSVNIHLLFKELLSDITASKANYSLRIANRLYAEKSR |
| | | PILPIYLKCVKKLYRAGLETVNFKTASDQARQLINSWVEKQT |
| | | EGQIKDLLVSSSTDLDTTLVLVNAIYFKGMWKTAFNAEDTRE |
| | | MPFHVTKEESKPVQMMCMNNSFNVATLPAEKMKILELPFASG |
| | | DLSMLVLLPDEVSGLERIEKTINFEKLTEWTNPNTMEKRRVK |
| | | VYLPQMKIEEKYNLTSVLMALGMTDLFIPSANLTGISSAESL |
| | | KISQAVHGAFMELSEDGIEMAGSTGVIEDIKHSPELEQFRAD |
| | | HPFLFLIKHNPTNTIVYFGRYWSP* |
| Ovalbumin | 137 | MDSISVTNAKFCFDVFNEMKVHHVNENILYCPLSILTALAMV |
| related protein | | YLGARGNTESQMKKVLHFDSITGAGSTTDSQCGSSEYVHNLF |
| Y | | KELLSEITRPNATYSLEIADKLYVDKTFSVLPEYLSCARKFY |
| | | TGGVEEVNFKTAAEEARQLINSWVEKETNGQIKDLLVSSSID |
| | | FGTTMVFINTIYFKGIWKIAFNTEDTREMPFSMTKEESKPVQ |
| | | MMCMNNSFNVATLPAEKMKILELPYASGDLSMLVLLPDEVSG |
| | | LERIEKTINFDKLREWTSTNAMAKKSMKVYLPRMKIEEKYNL |
| | | TSILMALGMTDLFSRSANLTGISSVDNLMISDAVHGVFMEVN |
| | | EEGTEATGSTGAIGNIKHSLELEEFRADHPFLFFIRYNPTNA |
| | | ILFFGRYWSP* |
| Ovalbumin | 138 | MGSIGAASMEFCFDVFKELKVHHANENIFYCPIAIMSALAMV |
| | | YLGAKDSTRTQINKVVRFDKLPGFGDSIEAQCGTSVNVHSSL |
| | | RDILNQITKPNDVYSFSLASRLYAEERYPILPEYLQCVKELY |
| | | RGGLEPINFQTAADQARELINSWVESQTNGIIRNVLQPSSVD |
| | | SQTAMVLVNAIVFKGLWEKAFKDEDTQAMPFRVTEQESKPVQ |
| | | MMYQIGLFRVASMASEKMKILELPFASGTMSMLVLLPDEVSG |
| | | LEQLESIINFEKLTEWTSSNVMEERKIKVYLPRMKMEEKYNL |
| | | TSVLMAMGITDVFSSSANLSGISSAESLKISQAVHAAHAEIN |
| | | EAGREVVGSAEAGVDAASVSEEFRADHPFLFCIKHIATNAVL |
| | | FFGRCVSP* |
| Chicken | 139 | MRFPSIFTAVLFAASSALAAPVNTTTEDETAQIPAEAVIGYS |
| Ovalbumin with | | DLEGDFDVAVLPFSNSTNNGLLFINTTIASIAAKEEGVSLDK |
| bolded signal | | REAEAGSIGAASMEFCFDVFKELKVHHANENIFYCPIAIMSA |
| sequence | | LAMVYLGAKDSTRTQINKVVRFDKLPGFGDSIEAQCGTSVNV |
| | | HSSLRDILNQITKPNDVYSFSLASRLYAEERYPILPEYLQCV |
| | | KELYRGGLEPINFQTAADQARELINSWVESQINGIIRNVLQP |
| | | SSVDSQTAMVLVNAIVFKGLWEKAFKDEDTQAMPFRVTEQES |
| | | KPVQMMYQIGLFRVASMASEKMKILELPFASGTMSMLVLLPD |
| | | EVSGLEQLESIINFEKLTEWTSSNVMEERKIKVYLPRMKMEE |
| | | KYNLTSVLMAMGITDVESSSANLSGISSAESLKISQAVHAAH |
| | | AEINEAGREVVGSAEAGVDAASVSEEFRADHPFLFCIKHIAT |
| | | NAVLFFGRCVSP |
| Chicken OVA | 140 | EAEAGSIGAASMEFCFDVFKELKVHHANENIFYCPIAIMSAL |
| sequence as | | AMVYLGAKDSTRTQINKVVRFDKLPGFGDSIEAQCGTSVNVH |
| secreted from | | SSLRDILNQITKPNDVYSFSLASRLYAEERYPILPEYLQCVK |
| pichia | | ELYRGGLEPINFQTAADQARELINSWVESQTNGIIRNVLQPS |
| | | SVDSQTAMVLVNAIVFKGLWEKAFKDEDTQAMPFRVTEQESK |
| | | PVQMMYQIGLFRVASMASEKMKILELPFASGTMSMLVLLPDE |
| | | VSGLEQLESIINFEKLTEWTSSNVMEERKIKVYLPRMKMEEK |
| | | YNLTSVLMAMGITDVFSSSANLSGISSAESLKISQAVHAAHA |
| | | EINEAGREVVGSAEAGVDAASVSEEFRADHPFLFCIKHIATN |
| | | AVLFFGRCVSP |
| Predicted | 141 | MRVPAQLLGLLLLWLPGARCGSIGAASMEFCFDVFKELKVHH |
| Ovalbumin | | ANENIFYCPIAIMSALAMVYLGAKDSTRTQINKVVRFDKLPG |
| [Achromobacter | | FGDSIEAQCGTSVNVHSSLRDILNQITKPNDVYSFSLASRLY |
| denitrificans] | | AEERYPILPEYLQCVKELYRGGLEPINFQTAADQARELINSW |
| | | VESQTNGIIRNVLQPSSVDSQTAMVLVNAIVFKGLWEKAFKD |
| | | EDTQAMPFRVTEQESKPVQMMYQIGLFRVASMASEKMKILEL |
| | | PFASGTMSMLVLLPDEVSGLEQLESIINFEKLTEWTSSNVME |
| | | ERKIKVYLPRMKMEEKYNLTSVLMAMGITDVFSSSANLSGIS |
| | | SAESLKISQAVHAAHAEINEAGREVVGSAEAGVDAASVSEEF |
| | | RADHPFLFCIKHIATNAVLFFGRCVSPLEIKRAAAHHHHHH |
| OLLAS | 142 | MTSGFANELGPRLMGKLTMGSIGAASMEFCFDVFKELKVHHA |
| epitope-tagged | | NENIFYCPIAIMSALAMVYLGAKDSTRTQINKVVRFDKLPGF |
| ovalbumin | | GDSIEAQCGTSVNVHSSLRDILNQITKPNDVYSFSLASRLYA |
| | | EERYPILPEYLQCVKELYRGGLEPINFQTAADQARELINSWV |
| | | ESQTNGIIRNVLQPSSVDSQTAMVLVNAIVFKGLWEKTFKDE |
| | | DTQAMPFRVTEQESKPVQMMYQIGLFRVASMASEKMKILELP |
| | | FASGTMSMLVLLPDEVSGLEQLESIINFEKLTEWTSSNVMEE |
| | | RKIKVYLPRMKMEEKYNLTSVLMAMGITDVFSSSANLSGISS |
| | | AESLKISQAVHAAHAEINEAGREVVGSAEAGVDAASVSEEFR |
| | | ADHPFLFCIKHIATNAVLFFGRCVSPSR |
| Serpin family | 143 | MGGRRVRWEVYISRAGYVNRQIAWRRHHRSLTMRVPAQLLGL |
| protein | | LLLWLPGARCGSIGAASMEFCFDVFKELKVHHANENIFYCPI |
| [Achromobacter | | AIMSALAMVYLGAKDSTRTQINKVVRFDKLPGFGDSIEAQCG |
| denitrificans] | | TSVNVHSSLRDILNQITKPNDVYSFSLASRLYAEERYPILPE |
| | | YLQCVKELYRGGLEPINFQTAADQARELINSWVESQTNGIIR |
| | | NVLQPSSVDSQTAMVLVNAIVFKGLWEKAFKDEDTQAMPFRV |
| | | TEQESKPVQMMYQIGLFRVASMASEKMKILELPFASGTMSML |
| | | VLLPDEVSGLEQLESIINFEKLTEWTSSNVMEERKIKVYLPR |
| | | MKMEEKYNLTSVLMAMGITDVFSSSANLSGISSAESLKISQA |
| | | VHAAHAEINEAGREVVGSAEAGVDAASVSEEFRADHPFLFCI |
| | | KHIATNAVLFFGRCVSPLEIKRAAAHHHHHH |
| PREDICTED: | 144 | MGSIGAVSMEFCFDVFKELKVHHANENIFYSPFTIISALAMV |
| ovalbumin | | YLGAKDSTRTQINKVVRFDKLPGFGDSVEAQCGTSVNVHSSL |
| isoform X1 | | RDILNQITKPNDVYSFSLASRLYAEETYPILPEYLQCVKELY |
| [Meleagris | | RGGLESINFQTAADQARGLINSWVESQTNGMIKNVLQPSSVD |
| gallopavo] | | SQTAMVLVNAIVFKGLWEKAFKDEDTQAIPFRVTEQESKPVQ |
| | | MMYQIGLFKVASMASEKMKILELPFASGTMSMWVLLPDEVSG |
| | | LEQLETTISFEKMTEWISSNIMEERRIKVYLPRMKMEEKYNL |
| | | TSVLMAMGITDLFSSSANLSGISSAGSLKISQAVHAAYAEIY |
| | | EAGREVIGSAEAGADATSVSEEFRVDHPFLYCIKHNLTNSIL |
| | | FFGRCISP |
| Ovalbumin | 145 | MGSIGAVSMEFCFDVFKELKVHHANENIFYSPFTIISALAMV |
| precursor | | YLGAKDSTRTQINKVVRFDKLPGFGDSVEAQCGTSVNVHSSL |
| [Meleagris | | RDILNQITKPNDVYSFSLASRLYAEETYPILPEYLQCVKELY |
| gallopavo] | | RGGLESINFQTAADQARGLINSWVESQTNGMIKNVLQPSSVD |
| | | SQTAMVLVNAIVFKGLWEKAFKDEDTQAIPFRVTEQESKPVQ |
| | | MMYQIGLFKVASMASEKMKILELPFASGTMSMWVLLPDEVSG |
| | | LEQLETTISFEKMTEWISSNIMEERRIKVYLPRMKMEEKYNL |
| | | TSVLMAMGITDLFSSSANLSGISSAGSLKISQAAHAAYAEIY |
| | | EAGREVIGSAEAGADATSVSEEFRVDHPFLYCIKHNLTNSIL |
| | | FFGRCISP |
| Hypothetical | 146 | YYRVPCMVLCTAFHPYIFIVLLFALDNSEFTMGSIGAVSMEF |
| protein | | CFDVFKELRVHHPNENIFFCPFAIMSAMAMVYLGAKDSTRTQ |
| [Bambusicola | | INKVIRFDKLPGFGDSTEAQCGKSANVHSSLKDILNQITKPN |
| thoracicus] | | DVYSFSLASRLYADETYSIQSEYLQCVNELYRGGLESINFQT |
| | | AADQARELINSWVESQTNGIIRNVLQPSSVDSQTAMVLVNAI |
| | | VFRGLWEKAFKDEDTQTMPFRVTEQESKPVQMMYQIGSFKVA |
| | | SMASEKMKILELPLASGTMSMLVLLPDEVSGLEQLETTISFE |
| | | KLTEWTSSNVMEERKIKVYLPRMKMEEKYNLTSVLMAMGITD |
| | | LFRSSANLSGISLAGNLKISQAVHAAHAEINEAGRKAVSSAE |
| | | AGVDATSVSEEFRADRPFLFCIKHIATKVVFFFGRYTSP |
| Egg albumin | 147 | MGSIGAASMEFCFDVFKELKVHHANDNMLYSPFAILSTLAMV |
| | | FLGAKDSTRTQINKVVHFDKLPGFGDSIEAQCGTSVNVHSSL |
| | | RDILNQITKQNDAYSFSLASRLYAQETYTVVPEYLQCVKELY |
| | | RGGLESVNFQTAADQARGLINAWVESQTNGIIRNILQPSSVD |
| | | SQTAMVLVNAIAFKGLWEKAFKAEDTQTIPFRVTEQESKPVQ |
| | | MMYQIGSFKVASMASEKMKILELPFASGTMSMLVLLPDDVSG |
| | | LEQLESIISFEKLTEWTSSSIMEERKVKVYLPRMKMEEKYNL |
| | | TSLLMAMGITDLFSSSANLSGISSVGSLKISQAVHAAHAEIN |
| | | EAGRDVVGSAEAGVDATEEFRADHPFLFCVKHIETNAILLFG |
| | | RCVSP |
| Ovalbumin | 148 | MASIGAVSTEFCVDVYKELRVHHANENIFYSPFTIISTLAMV |
| isoform X2 | | YLGAKDSTRTQINKVVRFDKLPGFGDSIEAQCGTSVNVHSSL |
| [Numida | | RDILNQITKPNDVYSFSLASRLYAEETYPILPEYLQCVKELY |
| meleagris] | | RGGLESINFQTAADQARELINSWVESQTSGIIKNVLQPSSVN |
| | | SQTAMVLVNAIYFKGLWERAFKDEDTQAIPFRVTEQESKPVQ |
| | | MMSQIGSFKVASVASEKVKILELPFVSGTMSMLVLLPDEVSG |
| | | LEQLESTISTEKLTEWTSSSIMEERKIKVFLPRMRMEEKYNL |
| | | TSVLMAMGMTDLFSSSANLSGISSAESLKISQAVHAAYAEIY |
| | | EAGREVVSSAEAGVDATSVSEEFRVDHPFLLCIKHNPTNSIL |
| | | FFGRCISP |
| Ovalbumin | 149 | MALCKAFHPYIFIVLLFDVDNSAFTMASIGAVSTEFCVDVYK |
| isoform X1 | | ELRVHHANENIFYSPFTIISTLAMVYLGAKDSTRTQINKVVR |
| [Numida | | FDKLPGFGDSIEAQCGTSVNVHSSLRDILNQITKPNDVYSFS |
| meleagris] | | LASRLYAEETYPILPEYLQCVKELYRGGLESINFQTAADQAR |
| | | ELINSWVESQTSGIIKNVLQPSSVNSQTAMVLVNAIYFKGLW |
| | | ERAFKDEDTQAIPFRVTEQESKPVQMMSQIGSFKVASVASEK |
| | | VKILELPFVSGTMSMLVLLPDEVSGLEQLESTISTEKLTEWT |
| | | SSSIMEERKIKVFLPRMRMEEKYNLTSVLMAMGMTDLFSSSA |
| | | NLSGISSAESLKISQAVHAAYAEIYEAGREVVSSAEAGVDAT |
| | | SVSEEFRVDHPFLLCIKHNPTNSILFFGRCISP |
| PREDICTED: | 150 | MGSIGAASMEFCFDVFKELKVHHANDNMLYSPFAILSTLAMV |
| Ovalbumin | | FLGAKDSTRTQINKVVHFDKLPGFGDSIEAQCGTSANVHSSL |
| isoform X2 | | RDILNQITKQNDAYSFSLASRLYAQETYTVVPEYLQCVKELY |
| [Coturnix | | RGGLESVNFQTAADQARGLINAWVESQTNGIIRNILQPSSVD |
| japonica] | | SQTAMVLVNAIAFKGLWEKAFKAEDTQTIPFRVTEQESKPVQ |
| | | MMHQIGSFKVASMASEKMKILELPFASGTMSMLVLLPDDVSG |
| | | LEQLESTISFEKLTEWTSSSIMEERKVKVYLPRMKMEEKYNL |
| | | TSLLMAMGITDLFSSSANLSGISSVGSLKISQAVHAAYAEIN |
| | | EAGRDVVGSAEAGVDATEEFRADHPFLFCVKHIETNAILLFG |
| | | RCVSP |
| PREDICTED: | 151 | MGLCTAFHPYIFIVLLFALDNSEFTMGSIGAASMEFCFDVFK |
| ovalbumin | | ELKVHHANDNMLYSPFAILSTLAMVFLGAKDSTRTQINKVVH |
| isoform X1 | | FDKLPGFGDSIEAQCGTSANVHSSLRDILNQITKQNDAYSFS |
| [Coturnix | | LASRLYAQETYTVVPEYLQCVKELYRGGLESVNFQTAADQAR |
| japonica] | | GLINAWVESQTNGIIRNILQPSSVDSQTAMVLVNAIAFKGLW |
| | | EKAFKAEDTQTIPFRVTEQESKPVQMMHQIGSFKVASMASEK |
| | | MKILELPFASGTMSMLVLLPDDVSGLEQLESTISFEKLTEWT |
| | | SSSIMEERKVKVYLPRMKMEEKYNLTSLLMAMGITDLFSSSA |
| | | NLSGISSVGSLKISQAVHAAYAEINEAGRDVVGSAEAGVDAT |
| | | EEFRADHPFLFCVKHIETNAILLFGRCVSP |
| Egg albumin | 152 | MGSIGAASMEFCFDVFKELKVHHANDNMLYSPFAILSTLAMV |
| | | FLGAKDSTRTQINKVVHFDKLPGFGDSIEAQCGTSANVHSSL |
| | | RDILNQITKQNDAYSFSLASRLYAQETYTVVPEYLQCVKELY |
| | | RGGLESVNFQTAADQARGLINAWVESQINGIIRNILQPSSVD |
| | | SQTAMVLVNAIAFKGLWEKAFKAEDTQTIPFRVTEQESKPVQ |
| | | MMHQIGSFKVASMASEKMKILELPFASGTMSMLVLLPDDVSG |
| | | LEQLESTISFEKLTEWTSSSIMEERKVKVYLPRMKMEEKYNL |
| | | TSLLMAMGITDLFSSSANLSGISSVGSLKIPQAVHAAYAEIN |
| | | EAGRDVVGSAEAGVDATEEFRADHPFLFCVKHIETNAILLFG |
| | | RCVSP |
| ovalbumin | 153 | MGSIGAASTEFCFDVFRELRVQHVNENIFYSPFSIISALAMV |
| [Anas | | YLGARDNTRTQIDKVVHFDKLPGFGESMEAQCGTSVSVHSSL |
| platyrhynchos] | | RDILTQITKPSDNFSLSFASRLYAEETYAILPEYLQCVKELY |
| | | KGGLESISFQTAADQARELINSWVESQINGIIKNILQPSSVD |
| | | SQTTMVLVNAIYFKGMWEKAFKDEDTQAMPFRMTEQESKPVQ |
| | | MMYQVGSFKVAMVTSEKMKILELPFASGMMSMFVLLPDEVSG |
| | | LEQLESTISFEKLTEWTSSTMMEERRMKVYLPRMKMEEKYNL |
| | | TSVFMALGMTDLFSSSANMSGISSTVSLKMSEAVHAACVEIF |
| | | EAGRDVVGSAEAGMDVTSVSEEFRADHPFLFFIKHNPTNSIL |
| | | FFGRWMSP |
| PREDICTED: | 154 | MGSIGAASTEFCFDVFRELKVQHVNENIFYSPLSIISALAMV |
| ovalbumin-like | | YLGARDNTRTQIDQVVHFDKIPGFGESMEAQCGTSVSVHSSL |
| [Anser | | RDILTEITKPSDNFSLSFASRLYAEETYTILPEYLQCVKELY |
| cygnoides | | KGGLESISFQTAADQARELINSWVESQTNGIIKNILQPSSVD |
| domesticus] | | SQTTMVLVNAIYFKGMWEKAFKDEDTQTMPFRMTEQESKPVQ |
| | | MMYQVGSFKLATVTSEKVKILELPFASGMMSMCVLLPDEVSG |
| | | LEQLETTISFEKLTEWTSSTMMEERRMKVYLPRMKMEEKYNL |
| | | TSVFMALGMTDLFSSSANMSGISSTVSLKMSEAVHAACVEIF |
| | | EAGRDVVGSAEAGMDVTSVSEEFRADHPFLFFIKHNPSNSIL |
| | | FFGRWISP |
| PREDICTED: | 155 | MGSIGAASTEFCFDVFKELKVQHVNENIFYSPLTIISALSMV |
| Ovalbumin-like | | YLGARENTRAQIDKVLHFDKMPGFGDTIESQCGTSVSIHTSL |
| [Aquila | | KDMFTQITKPSDNYSLSFASRLYAEETYPILPEYLQCVKELY |
| chrysaetos | | KGGLETISFQTAAEQARELINSWVESQTNGMIKNILQPSSVD |
| canadensis] | | PQTKMVLVNAIYFKGVWEKAFKDEDTQEVPFRVTEQESKPVQ |
| | | MMYQIGSFKVAVMASEKMKILELPYASGQLSMLVLLPDDVSG |
| | | LEQLESAITFEKLMAWTSSTTMEERKMKVYLPRMKIEEKYNL |
| | | TSVLMALGVTDLFSSSANLSGISSAESLKISKAVHEAFVEIY |
| | | EAGSEVVGSTEAGMEVTSVSEEFRADHPFLFLIKHNPTNSIL |
| | | FFGRCFSP |
| PREDICTED: | 156 | MGSIGAASTEFCFDVFKELKVQHVNENIFYSPLTIISALSMV |
| Ovalbumin-like | | YLGARENTRTQIDKVLHFDKMTGFGDTVESQCGTSVSIHTSL |
| [Haliaeetus | | KDIFTQITKPSDNYSLSLASRLYAEETYPILPEYLQCVKELY |
| albicilla] | | KGGLETVSFQTAAEQARELINSWVESQTNGMIKNILQPSSVD |
| | | PQTKMVLVNAIYFKGVWEKAFKDEDTQEVPFRVTEQESKPVQ |
| | | MMYQIGSFKVAVMASEKMKILELPYASGQLSMLVLLPDDVSG |
| | | LEQLESAITSEKLMEWTSSTTMEERKMKVYLPRMKIEEKYNL |
| | | TSVLMALGVTDLFSSSADLSGISSAESLKISKAVHEAFVEIY |
| | | EAGSEVVGSTEGGMEVTSVSEEFRADHPFLFLIKHKPTNSIL |
| | | FFGRCFSP |
| PREDICTED: | 157 | MGSIGAASTEFCFDVFKELKVQHVNENIFYSPLTIISALSMV |
| Ovalbumin-like | | YLGARENTRTQIDKVLHFDKMTGFGDTVESQCGTSVSIHTSL |
| [Haliaeetus | | KDIFTQITKPSDNYSLSLASRLYAEETYPILPEYLQCVKELY |
| leucocephalus] | | KGGLETVSFQTAAEQARELINSWVESQTNGMIKNILQPSSVD |
| | | PQTKMVLVNAIYFKGVWEKAFKDEDTQEVPFRVTEQESKPVQ |
| | | MMYQIGSFKVAVMASEKMKILELPYASGQLSMLVLLPDDVSG |
| | | LEQLESAITSEKLMEWTSSTTMEERKMKVYLPRMKIEEKYNL |
| | | TSVLMALGVTDLFSSSADLSGISSAESLKISKAVHEAFVEIY |
| | | EAGSEVVGSTEGGMEVTSFSEEFRADHPFLFLIKHKPTNSIL |
| | | FFGRCFSP |
| PREDICTED: | 158 | MGSIGAASTEFCFDVFKELKVQHVNENIFYSPLSIISALSMV |
| Ovalbumin | | YLGARENTRAQIDKVVHFDKITGFGETIESQCGTSVSVHTSL |
| [Fulmarus | | KDMFTQITKPSDNYSLSFASRLYAEETYPILPEYLQCVKELY |
| glacialis] | | KGGLETTSFQTAADQARELINSWVESQTNGMIKNILQPGSVD |
| | | PQTEMVLVNAIYFKGMWEKAFKDEDTQAVPFRMTEQESKTVQ |
| | | MMYQIGSFKVAVMASEKMKILELPYASGELSMLVMLPDDVSG |
| | | LEQLETAITFEKLMEWTSSNMMEERKMKVYLPRMKMEEKYNL |
| | | TSVLMALGVTDLFSSSANLSGISSAESLKMSEAVHEAFVEIY |
| | | EAGSEVVGSTGAGMEVTSVSEEFRADHPFLFLIKHNPTNSIL |
| | | FFGRCFSP |
| PREDICTED: | 159 | MGSIGAASTEFCFDVFKELRVQHVNENVCYSPLIIISALSLV |
| Ovalbumin-like | | YLGARENTRAQIDKVVHFDKITGFGESIESQCGTSVSVHTSL |
| [Chlamydotis | | KDMENQITKPSDNYSLSVASRLYAEERYPILPEYLQCVKELY |
| macqueenii] | | KGGLESISFQTAADQAREAINSWVESQTNGMIKNILQPSSVD |
| | | PQTEMVLVNAIYFKGMWQKAFKDEDTQAVPFRISEQESKPVQ |
| | | MMYQIGSFKVAVMAAEKMKILELPYASGELSMLVLLPDEVSG |
| | | LEQLENAITVEKLMEWTSSSPMEERIMKVYLPRMKIEEKYNL |
| | | TSVLMALGITDLFSSSANLSGISAEESLKMSEAVHQAFAEIS |
| | | EAGSEVVGSSEAGIDATSVSEEFRADHPFLFLIKHNATNSIL |
| | | FFGRCFSP |
| PREDICTED: | 160 | MGSISAASTEFCFDVFKELKVQHVNENIFYSPLSIISALSMV |
| Ovalbumin like | | YLGARENTRAQIEKVVHFDKITGFGESIESQCSTSVSVHTSL |
| [Nipponia | | KDMFTQITKPSDNYSLSFASRFYAEETYPILPEYLQCVKELY |
| nippon] | | KGGLETINFRTAADQARELINSWVESQTNGMIKNILQPGSVD |
| | | PQTDMVLVNAIYFKGMWEKAFKDEDTQALPFRVTEQESKPVQ |
| | | MMYQIGSFKVAVLASEKVKILELPYASGQLSMLVLLPDDVSG |
| | | LEQLETAITVEKLMEWTSSNNMEERKIKVYLPRIKIEEKYNL |
| | | TSVLMALGITDLFSSSANLSGISSAESLKVSEAIHEAFVEIY |
| | | EAGSEVAGSTEAGIEVTSVSEEFRADHPFLFLIKHNATNSIL |
| | | FFGRCFSP |
| PREDICTED: | 161 | MVSIGAASTEFCFDVFKELKVQHVNENIFYSPLSIISALSMV |
| Ovalbumin-like | | YLGARENTRAQIDKVVHFDKITGFEETIESQCSTSVSVHTSL |
| isoform X2 | | KDMFTQITKPSDNYSLSFASRLYAEETYPILPEYLQCVKELY |
| [Gavia stellata] | | KGGLETISFQTAADQARELINSWVESQTDGMIKNILQPGSVD |
| | | PQTEMVLVNAIYFKGMWEKAFKDEDTQAVPFRMTEQESKPVQ |
| | | MMYQIGSFKVAVMASEKMKILELPYASGGMSMLVMLPDDVSG |
| | | LEQLETAITFEKLMEWTSSNMMEERKMKVYLPRMKMEEKYNL |
| | | TSVLMALGMTDLFSSSANLSGISSAESLKMSEAVHEAFVEIY |
| | | EAGSEAVGSTGAGMEVTSVSEEFRADHPFLFLIKHNPTNSIL |
| | | FFGRCFSP |
| PREDICTED: | 162 | MGSIGAASTEFCFDVFKELKVQHVNENIFYSPLSIISALSMV |
| Ovalbumin | | YLGARENTRAQIDKVVHFDKITGFGEPIESQCGISVSVHTSL |
| [Pelecanus | | KDMITQITKPSDNYSLSFASRLYAEETYPILPEYLQCVKELY |
| crispus] | | KGGLETISFQTAADQARELINSWVENQTNGMIKNILQPGSVD |
| | | PQTEMVLVNAVYFKGMWEKAFKDEDTQAVPFRMTEQESKPVQ |
| | | MMYQIGSFKVAVMASEKIKILELPYASGELSMLVLLPDDVSG |
| | | LEQLETAITLDKLTEWTSSNAMEERKMKVYLPRMKIEKKYNL |
| | | TSVLIALGMTDLFSSSANLSGISSAESLKMSEAIHEAFLEIY |
| | | EAGSEVVGSTEAGMEVTSVSEEFRADHPFLFLIKHNPTNSIL |
| | | FFGRCLSP |
| PREDICTED: | 163 | MGSIGAASTEFCFDVFKELKVQHVNENIFYSPLTIISALSMV |
| Ovalbumin-like | | YLGARENTRAQIDKVVHFDKIPGFGDTTESQCGTSVSVHTSL |
| [Charadrius | | KDMFTQITKPSDNYSVSFASRLYAEETYPILPEFLECVKELY |
| vociferus] | | KGGLESISFQTAADQARELINSWVESQTNGMIKNILQPGSVD |
| | | SQTEMVLVNAIYFKGMWEKAFKDEDTQTVPFRMTEQETKPVQ |
| | | MMYQIGTFKVAVMPSEKMKILELPYASGELCMLVMLPDDVSG |
| | | LEELESSITVEKLMEWTSSNMMEERKMKVFLPRMKIEEKYNL |
| | | TSVLMALGMTDLFSSSANLSGISSAEPLKMSEAVHEAFIEIY |
| | | EAGSEVVGSTGAGMEITSVSEEFRADHPFLFLIKHNPTNSIL |
| | | FFGRCVSP |
| PREDICTED: | 164 | MGSIGAVSTEFCFDVFKELKVQHVNENIFYSPLSIISALSMV |
| Ovalbumin-like | | YLGARENTRAQIDKVVHFDKITGSGETIEAQCGTSVSVHTSL |
| [Eurypyga | | KDMFTQITKPSENYSVGFASRLYADETYPIIPEYLQCVKELY |
| helias] | | KGGLEMISFQTAADQARELINSWVESQTNGMIKNILQPGSVD |
| | | PQTEMILVNAIYFKGVWEKAFKDEDTQAVPFRMTEQESKPVQ |
| | | MMYQFGSFKVAAMAAEKMKILELPYASGALSMLVLLPDDVSG |
| | | LEQLESAITFEKLMEWTSSNMMEEKKIKVYLPRMKMEEKYNF |
| | | TSVLMALGMTDLFSSSANLSGISSADSLKMSEVVHEAFVEIY |
| | | EAGSEVVGSTGSGMEAASVSEEFRADHPFLFLIKHNPTNSIL |
| | | FFGRCFSP |
| PREDICTED: | 165 | MVSIGAASTEFCFDVFKELKVQHVNENIFYSPLSIISALSMV |
| Ovalbumin-like | | YLGARENTRAQIDKVVHFDKITGFEETIESQVQKKQCSTSVS |
| isoform X1 | | VHTSLKDMFTQITKPSDNYSLSFASRLYAEETYPILPEYLQC |
| [Gavia stellata] | | VKELYKGGLETISFQTAADQARELINSWVESQTDGMIKNILQ |
| | | PGSVDPQTEMVLVNAIYFKGMWEKAFKDEDTQAVPFRMTEQE |
| | | SKPVQMMYQIGSFKVAVMASEKMKILELPYASGGMSMLVMLP |
| | | DDVSGLEQLETAITFEKLMEWTSSNMMEERKMKVYLPRMKME |
| | | EKYNLTSVLMALGMTDLFSSSANLSGISSAESLKMSEAVHEA |
| | | FVEIYEAGSEAVGSTGAGMEVTSVSEEFRADHPFLFLIKHNP |
| | | TNSILFFGRCFSP |
| PREDICTED: | 166 | MGSIGAASGEFCFDVFKELKVQHVNENIFYSPLSIISALSMV |
| Ovalbumin-like | | YLGARENTRAQIDKVVHFDKIIGFGESIESQCGTSVSVHTSL |
| [Egretta | | KDMFAQITKPSDNYSLSFASRLYAEETFPILPEYLQCVKELY |
| garzetta] | | KGGLETLSFQTAADQARELINSWVESQTNGMIKDILQPGSVD |
| | | PQTEMVLVNAIYFKGVWEKAFKDEDTQTVPFRMTEQESKPVQ |
| | | MMYQIGSFKVAVVAAEKIKILELPYASGALSMLVLLPDDVSS |
| | | LEQLETAITFEKLTEWTSSNIMEERKIKVYLPRMKIEEKYNL |
| | | TSVLMDLGITDLFSSSANLSGISSAESLKVSEAIHEAIVDIY |
| | | EAGSEVVGSSGAGLEGTSVSEEFRADHPFLFLIKHNPTSSIL |
| | | FFGRCFSP |
| PREDICTED: | 167 | MGSIGAASTEFCFDVFKELKVQHVNENIFYSPLSIISALSMV |
| Ovalbumin-like | | YLGARENTRAQIDKVVHFDKITGSGEAIESQCGTSVSVHISL |
| [Balearica | | KDMFTQITKPSDNYSLSFASRLYAEETYPILPEYLQCVKELY |
| regulorum | | KEGLATISFQTAADQAREFINSWVESQTNGMIKNILQPGSVD |
| gibbericeps] | | PQTQMVLVNAIYFKGVWEKAFKDEDTQAVPFRMTKQESKPVQ |
| | | MMYQIGSFKVAVMASEKMKILELPYASGQLSMLVMLPDDVSG |
| | | LEQIENAITFEKLMEWTNPNMMEERKMKVYLPRMKMEEKYNL |
| | | TSVLMALGMTDLFSSSANLSGISSAESLKMSEAVHEAFVEIY |
| | | EAGSEVVGSTGAGIEVTSVSEEFRADHPFLFLIKHNPTNSIL |
| | | FFGRCFSP |
| PREDICTED: | 168 | MGSIGEASTEFCIDVFRELKVQHVNENIFYSPLSIISALSMV |
| Ovalbumin-like | | YLGARENTRAQIDQVVHFDKITGFGDTVESQCGSSLSVHSSL |
| [Nestor | | KDIFAQITQPKDNYSLNFASRLYAEETYPILPEYLQCVKELY |
| notabilis] | | KGGLETISFQTAADQARELINSWVESQTNGMIKNILQPSSVD |
| | | PQTEMVLVNAIYFKGVWEKAFKDEETQAVPFRITEQENRPVQ |
| | | IMYQFGSFKVAVVASEKIKILELPYASGQLSMLVLLPDEVSG |
| | | LEQLENAITFEKLTEWTSSDIMEEKKIKVFLPRMKIEEKYNL |
| | | TSVLVALGIADLFSSSANLSGISSAESLKMSEAVHEAFVEIY |
| | | EAGSEVVGSSGAGIEAASDSEEFRADHPFLFLIKHKPTNSIL |
| | | FFGRCFSP |
| PREDICTED: | 169 | MGSIGAASTEFCFDIFNELKVQHVNENIFYSPLSIISALSMV |
| Ovalbumin-like | | YLGARENTKAQIDKVVHFDKITGFGESIESQCSTSASVHTSF |
| [Pygoscelis | | KDMFTQITKPSDNYSLSFASRLYAEETYPILPEYSQCVKELY |
| adeliae] | | KGGLESISFQTAADQARELINSWVESQTNGMIKNILQPGSVD |
| | | PQTELVLVNAIYFKGTWEKAFKDKDTQAVPFRVTEQESKPVQ |
| | | MMYQIGSYKVAVIASEKMKILELPYASGELSMLVLLPDDVSG |
| | | LEQLETAITFEKLMEWTSSNMMEERKVKVYLPRMKIEEKYNL |
| | | TSVLMALGMTDLFSPSANLSGISSAESLKMSEAIHEAFVEIY |
| | | EAGSEVVGSTEAGMEVTSVSEEFRADHPFLFLIKCNLTNSIL |
| | | FFGRCFSP |
| Ovalbumin-like | 170 | MGSISTASTEFCFDVFKELKVQHVNENIFYSPLSIISALSMV |
| [Athene | | YLGARENTRAQIEKVVHFDKITGFGESIESQCGTSVSVHTSL |
| cunicularia] | | KDMLIQISKPSDNYSLSFASKLYAEETYPILPEYLQCVKELY |
| | | KGGLESINFQTAADQARQLINSWVESQTNGMIKDILQPSSVD |
| | | PQTEMVLVNAIYFKGIWEKAFKDEDTQEVPFRITEQESKPVQ |
| | | MMYQIGSFKVAVIASEKIKILELPYASGELSMLIVLPDDVSG |
| | | LEQLETAITFEKLIEWTSPSIMEERKTKVYLPRMKIEEKYNL |
| | | TSVLMALGMTDLFSPSANLSGISSAESLKMSEAIHEAFVEIY |
| | | EAGSEVVGSAEAGMEATSVSEFRVDHPFLFLIKHNPANIILF |
| | | FGRCVSP |
| PREDICTED: | 171 | MGSIGAASTEFCFDVFKELKVQHVNENIFYSPLTIISALSLV |
| Ovalbumin-like | | YLGARENTRAQIDKVFHFDKISGFGETTESQCGTSVSVHTSL |
| [Calidris | | KEMFTQITKPSDNYSVSFASRLYAEDTYPILPEYLQCVKELY |
| pugnax] | | KGGLETISFQTAADQAREVINSWVESQTNGMIKNILQPGSVD |
| | | SQTEMVLVNAIYFKGMWEKAFKDEDTQTMPFRITEQERKPVQ |
| | | MMYQAGSFKVAVMASEKMKILELPYASGEFCMLIMLPDDVSG |
| | | LEQLENSFSFEKLMEWTTSNMMEERKMKVYIPRMKMEEKYNL |
| | | TSVLMALGMTDLFSSSANLSGISSAETLKMSEAVHEAFMEIY |
| | | EAGSEVVGSTGSGAEVTGVYEEFRADHPFLFLVKHKPTNSIL |
| | | FFGRCVSP |
| PREDICTED: | 172 | MGSIGAASTEFCFDIFNELKVQHVNENIFYSPLSIISALSMV |
| Ovalbumin | | YLGARENTKAQIDKVVHFDKITGFGETIESQCSTSVSVHTSL |
| [Aptenodytes | | KDTFTQITKPSDNYSLSFASRLYAEETYPILPEYSQCVKELY |
| forsteri] | | KGGLETISFQTAADQARELINSWVESQTNGMIKNILQPGSVD |
| | | PQTELVLVNAIYFKGTWEKAFKDKDTQAVPFRVTEQESKPVQ |
| | | MMYQIGSYKVAVIASEKMKILELPYASRELSMLVLLPDDVSG |
| | | LEQLETAITFEKLMEWTSSNMMEERKVKVYLPRMKIEEKYNL |
| | | TSVLMALGMTDLFSPSANLSGISSAESLKMSEAVHEAFVEIY |
| | | EAGSEVVGSTGAGMEVTSVSEEFRADHPFLFLIKCNPTNSIL |
| | | FFGRCFSP |
| PREDICTED: | 173 | MGSISAASAEFCLDVFKELKVQHVNENIFYSPLSIISALSMV |
| Ovalbumin-like | | YLGARENTRAQIDKVVHFDKITGSGETIEFQCGTSANIHPSL |
| [Pterocles | | KDMFTQITRLSDNYSLSFASRLYAEERYPILPEYLQCVKELY |
| gutturalis] | | KGGLETISFQTAADQARELINSWVESQTNGMIKNILQPGSVN |
| | | PQTEMVLVNAIYFKGLWEKAFKDEDTQTVPFRMTEQESKPVQ |
| | | MMYQVGSFKVAVMASDKIKILELPYASGELSMLVLLPDDVTG |
| | | LEQLETSITFEKLMEWTSSNVMEERTMKVYLPHMRMEEKYNL |
| | | TSVLMALGVTDLFSSSANLSGISSAESLKMSEAVHEAFVEIY |
| | | ESGSQVVGSTGAGTEVTSVSEEFRVDHPFLFLIKHNPTNSIL |
| | | FFGRCFSP |
| Ovalbumin-like | 174 | MGSIGAASVEFCFDVFKELKVQHVNENIFYSPLSIISALSMV |
| [Falco | | YLGARENTKAQIDKVVHFDKIAGFGEAIESQCVTSASIHSLK |
| peregrinus] | | DMFTQITKPSDNYSLSFASRLYAEEAYSILPEYLQCVKELYK |
| | | GGLETISFQTAADQARDLINSWVESQTNGMIKNILQPGAVDL |
| | | ETEMVLVNAIYFKGMWEKAFKDEDTQTVPFRMTEQESKPVQM |
| | | MYQVGSFKVAVMASDKIKILELPYASGQLSMVVVLPDDVSGL |
| | | EQLEASITSEKLMEWTSSSIMEEKKIKVYFPHMKIEEKYNLT |
| | | SVLMALGMTDLFSSSANLSGISSAEKLKVSEAVHEAFVEISE |
| | | AGSEVVGSTEAGTEVTSVSEEFKADHPFLFLIKHNPTNSILF |
| | | FGRCFSP |
| PREDICTED: | 175 | MGSIGAASSEFCFDIFKELKVQHVNENIFYSPLSIISALSMV |
| Ovalbumin-like | | YLGARENTRAQIDKVVPFDKITASGESIESQCSTSVSVHTSL |
| isoform X2 | | KDIFTQITKSSDNHSLSFASRLYAEETYPILPEYLQCVKELY |
| [Phalacrocorax | | EGGLETISFQTAADQARELINSWIESQTNGRIKNILQPGSVD |
| carbo] | | PQTEMVLVNAIYFKGMWEKAFKDEDTQAVPFRMTEQESKPVQ |
| | | VMHQIGSFKVAVLASEKIKILELPYASGELSMLVLLPDDVSG |
| | | LEQLETAITFEKLMEWTSPNIMEERKIKVFLPRMKIEEKYNL |
| | | TSVLMALGITDLFSPLANLSGISSAESLKMSEAIHEAFVEIS |
| | | EAGSEVIGSTEAEVEVINDPEEFRADHPFLFLIKHNPTNSIL |
| | | FFGRCFSP |
| PREDICTED: | 176 | MGSIGAASTEFCFDVFKELKAQYVNENIFYSPMTIITALSMV |
| Ovalbumin-like | | YLGSKENTRAQIAKVAHFDKITGFGESIESQCGASASIQFSL |
| [Merops | | KDLFTQITKPSGNHSLSVASRIYAEETYPILPEYLECMKELY |
| nubicus] | | KGGLETINFQTAANQARELINSWVERQTSGMIKNILQPSSVD |
| | | SQTEMVLVNAIYFRGLWEKAFKVEDTQATPFRITEQESKPVQ |
| | | MMHQIGSFKVAVVASEKIKILELPYASGRLTMLVVLPDDVSG |
| | | LKQLETTITFEKLMEWTTSNIMEERKIKVYLPRMKIEEKYNL |
| | | TSVLMALGLTDLESSSANLSGISSAESLKMSEAVHEAFVEIY |
| | | EAGSEVVASAEAGMDATSVSEEFRADHPFLFLIKDNTSNSIL |
| | | FFGRCFSP |
| PREDICTED: | 177 | MGSIGAASTEFCFDVFKELKGQHVNENIFFCPLSIVSALSMV |
| Ovalbumin-like | | YLGARENTRAQIVKVAHFDKIAGFAESIESQCGTSVSIHTSL |
| [Tauraco | | KDMFTQITKPSDNYSLNFASRLYAEETYPIIPEYLQCVKELY |
| erythrolophus] | | KGGLETISFQTAADQAREIINSWVESQTNGMIKNILRPSSVH |
| | | PQTELVLVNAVYFKGTWEKAFKDEDTQAVPFRITEQESKPVQ |
| | | MMYQIGSFKVAAVTSEKMKILEVPYASGELSMLVLLPDDVSG |
| | | LEQLETAITAEKLIEWTSSTVMEERKLKVYLPRMKIEEKYNL |
| | | TTVLTALGVTDLFSSSANLSGISSAQGLKMSNAVHEAFVEIY |
| | | EAGSEVVGSKGEGTEVSSVSDEFKADHPFLFLIKHNPTNSIV |
| | | FFGRCFSP |
| PREDICTED: | 178 | MGSIGAASTEFCFDVFKELKVHHVNENILYSPLAIISALSMV |
| Ovalbumin - | | YLGAKENTRDQIDKVVHFDKITGIGESIESQCSTAVSVHTSL |
| like [Cuculus | | KDVFDQITRPSDNYSLAFASRLYAEKTYPILPEYLQCVKELY |
| canorus] | | KGGLETIDFQTAADQARQLINSWVEDETNGMIKNILRPSSVN |
| | | PQTKIILVNAIYFKGMWEKAFKDEDTQEVPFRITEQETKSVQ |
| | | MMYQIGSFKVAEVVSDKMKILELPYASGKLSMLVLLPDDVYG |
| | | LEQLETVITVEKLKEWTSSIVMEERITKVYLPRMKIMEKYNL |
| | | TSVLTAFGITDLFSPSANLSGISSTESLKVSEAVHEAFVEIH |
| | | EAGSEVVGSAGAGIEATSVSEEFKADHPFLFLIKHNPTNSIL |
| | | FFGRCFSP |
| Ovalbumin | 179 | MGSIGAASTEFCLDVFKELKVQHVNENIFYSPLSIISALSMV |
| [Antrostomus | | YLGARENTRAQIDKVVHFDKITGFEDSIESQCGTSVSVHTSL |
| carolinensis] | | KDMFTQITKPSDNYSVGFASRLYAAETYQILPEYSQCVKELY |
| | | KGGLETINFQKAADQATELINSWVESQTNGMIKNILQPSSVD |
| | | PQTQIFLVNAIYFKGMWQRAFKEEDTQAVPFRISEKESKPVQ |
| | | MMYQIGSFKVAVIPSEKIKILELPYASGLLSMLVILPDDVSG |
| | | LEQLENAITLEKLMQWTSSNMMEERKIKVYLPRMRMEEKYNL |
| | | TSVFMALGITDLFSSSANLSGISSAESLKMSDAVHEASVEIH |
| | | EAGSEVVGSTGSGTEASSVSEEFRADHPYLFLIKHNPTDSIV |
| | | FFGRCFSP |
| PREDICTED: | 180 | MGSIGAASTEFCFDVFKELKFQHVDENIFYSPLTIISALSMV |
| Ovalbumin-like | | YLGARENTRAQIDKVVHFDKIAGFEETVESQCGTSVSVHTSL |
| [Opisthocomus | | KDMFAQITKPSDNYSLSFASRLYAEETYPILPEYLQCVKELY |
| hoazin] | | KGGLETISFQTAADQARDLINSWVESQTNGMIKNILQPSSVG |
| | | PQTELILVNAIYFKGMWQKAFKDEDTQEVPFRMTEQQSKPVQ |
| | | MMYQTGSFKVAVVASEKMKILALPYASGQLSLLVMLPDDVSG |
| | | LKQLESAITSEKLIEWTSPSMMEERKIKVYLPRMKIEEKYNL |
| | | TSVLMALGITDLFSPSANLSGISSAESLKMSQAVHEAFVEIY |
| | | EAGSEVVGSTGAGMEDSSDSEEFRVDHPFLFFIKHNPTNSIL |
| | | FFGRCFSP |
| PREDICTED: | 181 | MGSIGPLSVEFCCDVFKELRIQHPRENIFYSPVTIISALSMV |
| Ovalbumin-like | | YLGARDNTKAQIEKAVHFDKIPGFGESIESQCGTSLSIHTSL |
| [Lepidothrix | | KDIFTQITKPSDNYTVGIASRLYAEEKYPILPEYLQCIKELY |
| coronata] | | KGGLEPINFQTAAEQARELINSWVESQTNGMIKNILQPSSVN |
| | | PETDMVLVNAIYFKGLWEKAFKDEDIQTVPFRITEQESKPVQ |
| | | MMFQIGSFRVAEITSEKIRILELPYASGQLSLWVLLPDDISG |
| | | LEQLETAITFENLKEWTSSTKMEERKIKVYLPRMKIEEKYNL |
| | | TSVLTSLGITDLFSSSANLSGISSAESLKVSSAFHEASVEIY |
| | | EAGSKVVGSTGAEVEDTSVSEEFRADHPFLFLIKHNPSNSIF |
| | | FFGRCFSP |
| PREDICTED: | 182 | MGSIGTASAEFCFDVFKELKVHHVNENIFYSPLSIISALSMV |
| Ovalbumin | | YLGARENTKTQMEKVIHFDKITGLGESMESQCGTGVSIHTAL |
| [Struthio | | KDMLSEITKPSDNYSLSLASRLYAEQTYAILPEYLQCIKELY |
| camelus | | KESLETVSFQTAADQARELINSWIESQTNGVIKNFLQPGSVD |
| australis] | | SQTELVLVNAIYFKGMWEKAFKDEDTQEVPFRITEQESRPVQ |
| | | MMYQAGSFKVATVAAEKIKILELPYASGELSMLVLLPDDISG |
| | | LEQLETTISFEKLTEWTSSNMMEDRNMKVYLPRMKIEEKYNL |
| | | TSVLIALGMTDLFSPAANLSGISAAESLKMSEAIHAAYVEIY |
| | | EADSEIVSSAGVQVEVTSDSEEFRVDHPFLFLIKHNPTNSVL |
| | | FFGRCISP |
| PREDICTED: | 183 | MGSIGAVSTEFSCDVFKELRIHHVQENIFYSPVTIISALSMI |
| Ovalbumin-like | | YLGARDSTKAQIEKAVHFDKIPGFGESIESQCGTSLSIHTSI |
| [Acanthisitta | | KDMFTKITKASDNYSIGIASRLYAEEKYPILPEYLQCVKELY |
| chloris] | | KGGLESISFQTAAEQAREIINSWVESQTNGMIKNILQPSSVD |
| | | PQTDIVLVNAIYFKGLWEKAFRDEDTQTVPFKITEQESKPVQ |
| | | MMYQIGSFKVAEITSEKIKILEVPYASGQLSLWVLLPDDISG |
| | | LEKLETAITFENLKEWTSSTKMEERKIKVYLPRMKIEEKYNL |
| | | TSVLTALGITDLFSSSANLSGISSAESLKVSEAFHEAIVEIS |
| | | EAGSKVVGSVGAGVDDTSVSEEFRADHPFLFLIKHNPTSSIF |
| | | FFGRCFSP |
| PREDICTED: | 184 | MGSIGAASTEFCFDVFKELKVQHVNENIFYSPLSIISALSMV |
| Ovalbumin-like | | YLGARENTRAQIDKVVHFDKIAGFGESTESQCGTSVSAHTSL |
| [Tyto alba] | | KDMSNQITKLSDNYSLSFASRLYAEETYPILPEYSQCVKELY |
| | | KGGLESISFQTAAYQARELINAWVESQTNGMIKDILQPGSVD |
| | | SQTKMVLVNAIYFKGIWEKAFKDEDTQEVPFRMTEQETKPVQ |
| | | MMYQIGSFKVAVIAAEKIKILELPYASGQLSMLVILPDDVSG |
| | | LEQLETAITFEKLTEWTSASVMEERKIKVYLPRMSIEEKYNL |
| | | TSVLIALGVTDLESSSANLSGISSAESLRMSEAIHEAFVETY |
| | | EAGSTESGTEVTSASEEFRVDHPFLFLIKHKPTNSILFFGRC |
| | | FSP |
| PREDICTED: | 185 | MGSIGAASSEFCFDIFKELKVQHVNENIFYSPLSIISALSMV |
| Ovalbumin-like | | YLGARENTRAQIDKVVPFDKITASGESIESQVQKIQCSTSVS |
| isoform X1 | | VHTSLKDIFTQITKSSDNHSLSFASRLYAEETYPILPEYLQC |
| [Phalacrocorax | | VKELYEGGLETISFQTAADQARELINSWIESQTNGRIKNILQ |
| carbo] | | PGSVDPQTEMVLVNAIYFKGMWEKAFKDEDTQAVPFRMTEQE |
| | | SKPVQVMHQIGSFKVAVLASEKIKILELPYASGELSMLVLLP |
| | | DDVSGLEQLETAITFEKLMEWTSPNIMEERKIKVFLPRMKIE |
| | | EKYNLTSVLMALGITDLFSPLANLSGISSAESLKMSEAIHEA |
| | | FVEISEAGSEVIGSTEAEVEVTNDPEEFRADHPFLFLIKHNP |
| | | TNSILFFGRCFSP |
| Ovalbumin-like | 186 | MGSIGPLSVEFCCDVFKELRIQHARENIFYSPVTIISALSMV |
| [Pipra filicauda] | | YLGARDNTKAQIEKAVHFDKIPGFGESIESQCGTSLSIHTSL |
| | | KDIFTQITKPSDNYTVGIASRLYAEEKYPILPEYLQCIKELY |
| | | KGGLEPISFQTAAEQARELINSWVESQTNGIIKNILQPSSVN |
| | | PETDMVLVNAIYFKGLWEKAFKDEGTQTVPFRITEQESKPVQ |
| | | MMFQIGSFRVAEIASEKIRILELPYASGQLSLWVLLPDDISG |
| | | LEQLETAITFENLKEWTSSTKMEERKIKVYLPRMKIEEKYNL |
| | | TSVLTSLGITDLFSSSANLSGISSAERLKVSSAFHEASMEIN |
| | | EAGSKVVGAGVDDTSVSEEFRVDRPFLFLIKHNPSNSIFFFG |
| | | RCFSP |
| Ovalbumin | 187 | MGSIGAASTEFCFDMFKELKVHHVNENIIYSPLSIISILSMV |
| [Dromaius | | FLGARENTKTQMEKVIHFDKITGFGESLESQCGTSVSVHASL |
| novaehollandiae] | | KDILSEITKPSDNYSLSLASKLYAEETYPVLPEYLQCIKELY |
| | | KGSLETVSFQTAADQARELINSWVETQTNGVIKNFLQPGSVD |
| | | PQTEMVLVDAIYFKGTWEKAFKDEDTQEVPFRITEQESKPVQ |
| | | MMYQAGSFKVATVAAEKMKILELPYASGELSMFVLLPDDISG |
| | | LEQLETTISIEKLSEWTSSNMMEDRKMKVYLPHMKIEEKYNL |
| | | TSVLVALGMTDLFSPSANLSGISTAQTLKMSEAIHGAYVEIY |
| | | EAGSEMATSTGVLVEAASVSEEFRVDHPFLFLIKHNPSNSIL |
| | | FFGRCIFP |
| Chain A, | 188 | MGSIGAASTEFCFDMFKELKVHHVNENIIYSPLSIISILSMV |
| Ovalbumin | | FLGARENTKTQMEKVIHFDKITGFGESLESQCGTSVSVHASL |
| | | KDILSEITKPSDNYSLSLASKLYAEETYPVLPEYLQCIKELY |
| | | KGSLETVSFQTAADQARELINSWVETQTNGVIKNFLQPGSVD |
| | | PQTEMVLVDAIYFKGTWEKAFKDEDTQEVPFRITEQESKPVQ |
| | | MMYQAGSFKVATVAAEKMKILELPYASGELSMFVLLPDDISG |
| | | LEQLETTISIEKLSEWTSSNMMEDRKMKVYLPHMKIEEKYNL |
| | | TSVLVALGMTDLFSPSANLSGISTAQTLKMSEAIHGAYVEIY |
| | | EAGSEMATSTGVLVEAASVSEEFRVDHPFLFLIKHNPSNSIL |
| | | FFGRCIFPHHHHHH |
| Ovalbumin-like | 189 | MGSIGPLSVEFCCDVFKELRIQHARENIFYSPVTIISALSMV |
| [Corapipo | | YLGARDNTKAQIEKAVHFDKIPGFGESIESQCGTSLSIHTSL |
| altera] | | KDIFTQITKPSDNYTVGIASRLYAEEKYPILPEYLQCIKELY |
| | | KGGLEPISFQTAAEQARELINSWVESQTNGMIKNILQPSAVN |
| | | PETDMVLVNAIYFKGLWEKAFKDEGTQTVPFRITEQESKPVQ |
| | | MMFQIGSFRVAEITSEKIRILELPYASGQLSLWVLLPDDISG |
| | | LEQLETAITFENLKEWTSSTKMEERKIKVYLPRMKIEEKYNL |
| | | TSVLTSLGITDLFSSSANLSGISSAERLKVSSAFHEASMEIY |
| | | EAGSKVVGSTGAGVDDTSVSEEFRVDRPFLFLIKHNPSNSIF |
| | | FFGRCFSP |
| Ovalbumin-like | 190 | MEDQRGNTGFTMGSIGAASTEFCIDVFRELRVQHVNENIFYS |
| protein | | PLTIISALSMVYLGARENTRAQIDQVVHFDKIAGFGDTVESQ |
| [Amazona | | CGSSPSVHNSLKTVXAQITQPRDNYSLNLASRLYAEESYPIL |
| aestiva] | | PEYLQCVKELYNGGLETVSFQTAADQARELINSWVESQINGI |
| | | IKNILQPSSVDPQTEMVLVNAIYFKGLWEKAFKDEETQAVPF |
| | | RITEQENRPVQMMYQFGSFKVAXVASEKIKILELPYASGQLS |
| | | MLVLLPDEVSGLEQNAITFEKLTEWTSSDLMEERKIKVFFPR |
| | | VKIEEKYNLTAVLVSLGITDLFSSSANLSGISSAENLKMSEA |
| | | VHEAXVEIYEAGSEVAGSSGAGIEVASDSEEFRVDHPFLFLI |
| | | XHNPTNSILFFGRCFSP |
| PREDICTED: | 191 | MGSIGAASTEFCIDVFRELRVQHVNENIFYSPLSIISALSMV |
| Ovalbumin-like | | YLGARENTRAQIDEVFHFDKIAGFGDTVDPQCGASLSVHKSL |
| [Melopsittacus | | QNVFAQITQPKDNYSLNLASRLYAEESYPILPEYLQCVKELY |
| undulatus] | | NEGLETVSFQTGADQARELINSWVENQTNGVIKNILQPSSVD |
| | | PQTEMVLVNAIYFKGLWQKAFKDEETQAVPFRITEQENRPVQ |
| | | MMYQFGSFKVAVVASEKVKILELPYASGQLSMWVLLPDEVSG |
| | | LEQLENAITFEKLTEWTSSDLTEERKIKVFLPRVKIEEKYNL |
| | | TAVLMALGVTDLFSSSANFSGISAAENLKMSEAVHEAFVEIY |
| | | EAGSEVVGSSGAGIEAPSDSEEFRADHPFLFLIKHNPTNSIL |
| | | FFGRCFSP |
| Ovalbumin-like | 192 | MGSIGPLSVEFCCDVFKELRIQHARDNIFYSPVTIISALSMV |
| [Neopelma | | YLGARDNTKAQIEKAVHFDKIPGFGESIESQCGTSLSVHTSL |
| chrysocephalum] | | KDIFTQITKPRENYTVGIASRLYAEEKYPILPEYLQCIKELY |
| | | KGGLEPISFQTAAEQARELINSWVESQTNGMIKNILQPSSVN |
| | | PETDMVLVNAIYFKGLWKKAFKDEGTQTVPFRITEQESKPVQ |
| | | MMFQIGSFRVAEITSEKIRILELPYASGQLSLWVLLPDDISG |
| | | LEQLESAITFENLKEWTSSTKMEERKIKVYLPRMKIEEKYNL |
| | | TSVLTSLGITDLFSSSANLSGISSAEKLKVSSAFHEASMEIY |
| | | EAGNKVVGSTGAGVDDTSVSEEFRVDRPFLFLIKHNPSNSIF |
| | | FFGRCFSP |
| PREDICTED: | 193 | MGSIGAASAEFCVDVFKELKDQHVNNIVFSPLMIISALSMVN |
| Ovalbumin-like | | IGAREDTRAQIDKVVHFDKITGYGESIESQCGTSIGIYFSLK |
| [Buceros | | DAFTQITKPSDNYSLSFASKLYAEETYPILPEYLKCVKELYK |
| rhinoceros | | GGLETISFQTAADQARELINSWVESQTNGMIKNILQPSSVDP |
| silvestris] | | QTEMVLVNAIYFKGLWEKAFKDEDTQAVPFRITEQESKPVQM |
| | | MYQIGSFKVAVIASEKIKILELPYASGQLSLLVLLPDDVSGL |
| | | EQLESAITSEKLLEWTNPNIMEERKTKVYLPRMKIEEKYNLT |
| | | SVLVALGITDLFSSSANLSGISSAEGLKLSDAVHEAFVEIYE |
| | | AGREVVGSSEAGVEDSSVSEEFKADRPFIFLIKHNPTNGILY |
| | | FGRYISP |
| PREDICTED: | 194 | MGSIGAANTDFCFDVFKELKVHHANENIFYSPLSIVSALAMV |
| Ovalbumin-like | | YLGARENTRAQIDKALHFDKILGFGETVESQCDTSVSVHTSL |
| [Cariama | | KDMLIQITKPSDNYSFSFASKIYTEETYPILPEYLQCVKELY |
| cristata] | | KGGVETISFQTAADQAREVINSWVESHTNGMIKNILQPGSVD |
| | | PQTKMVLVNAVYFKGIWEKAFKEEDTQEMPFRINEQESKPVQ |
| | | MMYQIGSFKLTVAASENLKILEFPYASGQLSMMVILPDEVSG |
| | | LKQLETSITSEKLIKWTSSNTMEERKIRVYLPRMKIEEKYNL |
| | | KSVLMALGITDLESSSANLSGISSAESLKMSEAVHEAFVEIY |
| | | EAGSEVTSSTGTEMEAENVSEEFKADHPFLFLIKHNPTDSIV |
| | | FFGRCMSP |
| Ovalbumin | 195 | MGSIGPLSVEFCCDVFKELRIQHARENIFYSPVTIISALSMV |
| [Manacus | | YLGARDNTKAQIEKAVHFDKIPGFGESIESQCGTSLSIHTSL |
| vitellinus] | | KDIFTQITKPSDNYTVGIASRLYAEEKYPILPEYLQCIKELY |
| | | KGGLEPISFQTAAEQARELINSWVESQTNGMIKNILQPSSVN |
| | | PETDMVLVNAIYFKGLWEKAFKDESTQTVPFRITEQESKPVQ |
| | | MMFQIGSFRVAEIASEKIRILELPYASGQLSLWVLLPDDISG |
| | | LEQLETAITFENLKEWTSSTKMEERKIKVYLPRMKIEEKYNL |
| | | TSVLTSLGITDLESSSANLSGISSAERLKVSSAFHEASMEIY |
| | | EAGSRVVEAGVDDTSVSEEFRVDRPFLFLIKHNPSNSIFFFG |
| | | RCFSP |
| Ovalbumin-like | 196 | MGSIGPVSTEFCCDIFKELRIQHARENIIYSPVTIISALSMV |
| [Empidonax | | YLGARDNTKAQIEKAVHFDKIPGFGESIESQCGTSLSIHTSL |
| traillii] | | KDILTQITKPSDNYTVGIASRLYAEEKYPILSEYLQCIKELY |
| | | KGGLEPISFQTAAEQARELINSWVESQTNGMIKNILQPSSVN |
| | | PETDMVLVNAIYFKGLWEKAFKDEGTQTVPFRITEQESKPVQ |
| | | MMFQIGSFKVAEITSEKIRILELPYASGKLSLWVLLPDDISG |
| | | LEQLETAITFENLKEWTSSTRMEERKIKVYLPRMKIEEKYNL |
| | | TSVLTSLGITDLFSSSANLSGISSAERLKVSSAFHEVFVEIY |
| | | EAGSKVEGSTGAGVDDTSVSEEFRADHPFLFLVKHNPSNSII |
| | | FFGRCYLP |
| PREDICTED: | 197 | MGSTGAASMEFCFALFRELKVQHVNENIFFSPVTIISALSMV |
| Ovalbumin-like | | YLGARENTRAQLDKVAPFDKITGFGETIGSQCSTSASSHTSL |
| [Leptosomus | | KDVFTQITKASDNYSLSFASRLYAEETYPILPEYLQCVKELY |
| discolor] | | KGGLESISFQTAADQARELINSWVESQTNGMIKDILRPSSVD |
| | | PQTKIILITAIYFKGMWEKAFKEEDTQAVPFRMTEQESKPVQ |
| | | MMYQIGSFKVAVIPSEKLKILELPYASGQLSMLVILPDDVSG |
| | | LEQLETAITTEKLKEWTSPSMMKERKMKVYFPRMRIEEKYNL |
| | | TSVLMALGITDLFSPSANLSGISSAESLKVSEAVHEASVDID |
| | | EAGSEVIGSTGVGTEVTSVSEEIRADHPFLFLIKHKPTNSIL |
| | | FFGRCFSP |
| Hypothetical | 198 | MEHAQLTQLVNSNMTSNTCHEADEFENIDFRMDSISVTNTKF |
| protein | | CFDVFNEMKVHHVNENILYSPLSILTALAMVYLGARGNTESQ |
| H355_008077 | | MKKALHFDSITGAGSTTDSQCGSSEYIHNLFKEFLTEITRTN |
| [Colinus | | ATYSLEIADKLYVDKTFTVLPEYINCARKFYTGGVEEVNFKT |
| virginianus] | | AAEEARQLINSWVEKETNGQIKDLLVPSSVDFGTMMVFINTI |
| | | YFKGIWKTAFNTEDTREMPFSMTKQESKPVQMMCLNDTFNMA |
| | | TLPAEKMRILELPYASGELSMLVLLPDEVSGLEQIEKAINFE |
| | | KLREWTSTNAMEKKSMKVYLPRMKIEEKYNLTSTLMALGMTD |
| | | LFSRSANLTGISSVENLMISDAVHGAFMEVNEEGTEAAGSTG |
| | | AIGNIKHSVEFEEFRADHPFLFLIRYNPTNVILFFDNSEFTM |
| | | GSIGAVSTEFCFDVFKELRVHHANENIFYSPFTVISALAMVY |
| | | LGAKDSTRTQINKVVRFDKLPGFGDSIEAQCGTSANVHSSLR |
| | | DILNQITKPNDIYSFSLASRLYADETYTILPEYLQCVKELYR |
| | | GGLESINFQTAADQARELINSWVESQTSGIIRNVLQPSSVDS |
| | | QTAMVLVNAIYFKGLWEKGFKDEDTQAMPFRVTEQENKSVOM |
| | | MYQIGTFKVASVASEKMKILELPFASGTMSMWVLLPDEVSGL |
| | | EQLETTISIEKLTEWTSSSVMEERKIKVFLPRMKMEEKYNLT |
| | | SVLMAMGMTDLFSSSANLSGISSTLQKKGFRSQELGDKYAKP |
| | | MLESPALTPQVTAWDNSWIVAHPAAIEPDLCYQIMEQKWKPF |
| | | DWPDFRLPMRVSCRFRTMEALNKANTSFALDFFKHECQEDDD |
| | | ENILFSPFSISSALATVYLGAKGNTADQMAKTEIGKSGNIHA |
| | | GFKALDLEINQPTKNYLLNSVNQLYGEKSLPFSKEYLQLAKK |
| | | YYSAEPQSVDFLGKANEIRREINSRVEHQTEGKIKNLLPPGS |
| | | IDSLTRLVLVNALYFKGNWATKFEAEDTRHRPFRINMHTTKQ |
| | | VPMMYLRDKFNWTYVESVQTDVLELPYVNNDLSMFILLPRDI |
| | | TGLQKLINELTFEKLSAWTSPELMEKMKMEVYLPRFTVEKKY |
| | | DMKSTLSKMGIEDAFTKVDSCGVTNVDEITTHIVSSKCLELK |
| | | HIQINKKLKCNKAVAMEQVSASIGNFTIDLFNKLNETSRDKN |
| | | IFFSPWSVSSALALTSLAAKGNTAREMAEDPENEQAENIHSG |
| | | FKELMTALNKPRNTYSLKSANRIYVEKNYPLLPTYIQLSKKY |
| | | YKAEPYKVNFKTAPEQSRKEINNWVEKQTERKIKNFLSSDDV |
| | | KNSTKSILVNAIYFKAEWEEKFQAGNTDMQPFRMSKNKSKLV |
| | | KMMYMRHTFPVLIMEKLNFKMIELPYVKRELSMFILLPDDIK |
| | | DSTTGLEQLERELTYEKLSEWADSKKMSVTLVDLHLPKFSME |
| | | DRYDLKDALKSMGMASAFNSNADFSGMTGFQAVPMESLSAST |
| | | NSFTLDLYKKLDETSKGQNIFFASWSIATALAMVHLGAKGDT |
| | | ATQVAKGPEYEETENIHSGFKELLSAINKPRNTYLMKSANRL |
| | | FGDKTYPLLPKFLELVARYYQAKPQAVNFKTDAEQARAQINS |
| | | WVENETESKIQNLLPAGSIDSHTVLVLVNAIYFKGNWEKRFL |
| | | EKDTSKMPFRLSKTETKPVQMMFLKDTFLIHHERTMKFKIIE |
| | | LPYVGNELSAFVLLPDDISDNTTGLELVERELTYEKLAEWSN |
| | | SASMMKAKVELYLPKLKMEENYDLKSVLSDMGIRSAFDPAQA |
| | | DFTRMSEKKDLFISKVIHKAFVEVNEEDRIVQLASGRLTGRC |
| | | RTLANKELSEKNRTKNLFFSPFSISSALSMILLGSKGNTEAQ |
| | | IAKVLSLSKAEDAHNGYQSLLSEINNPDTKYILRTANRLYGE |
| | | KTFEFLSSFIDSSQKFYHAGLEQTDFKNASEDSRKQINGWVE |
| | | EKTEGKIQKLLSEGIINSMTKLVLVNAIYFKGNWQEKFDKET |
| | | TKEMPFKINKNETKPVQMMFRKGKYNMTYIGDLETTVLEIPY |
| | | VDNELSMIILLPDSIQDESTGLEKLERELTYEKLMDWINPNM |
| | | MDSTEVRVSLPRFKLEENYELKPTLSTMGMPDAFDLRTADFS |
| | | GISSGNELVLSEVVHKSFVEVNEEGTEAAAATAGIMLLRCAM |
| | | IVANFTADHPFLFFIRHNKTNSILFCGRFCSP |
| PREDICTED: | 199 | MGSIGTASTEFCFDMFKEMKVQHANQNIIFSPLTIISALSMV |
| Ovalbumin | | YLGARDNTKAQMEKVIHFDKITGFGESVESQCGTSVSIHTSL |
| isoform X2 | | KDMLSEITKPSDNYSLSLASRLYAEETYPILPEYLQCMKELY |
| [Apteryx | | KGGLETVSFQTAADQARELINSWVESQTNGVIKNFLQPGSVD |
| australis | | PQTEMVLVNAIYFKGMWEKAFKDEDTQEVPFRITEQESKPVQ |
| mantelli] | | MMYQVGSFKVATVAAEKMKILEIPYTHRELSMFVLLPDDISG |
| | | LEQLETTISFEKLTEWTSSNMMEERKVKVYLPHMKIEEKYNL |
| | | TSVLMALGMTDLFSPSANLSGISTAQTLMMSEAIHGAYVEIY |
| | | EAGREMASSTGVQVEVTSVLEEVRADKPFLFFIRHNPTNSMV |
| | | VFGRYMSP |
| Hypothetical | 200 | MTSNTCHEADEFENIDFRMDSISVTNTKFCFDVFNEMKVHHV |
| protein | | NENILYSPLSILTALAMVYLGARGNTESQMKKALHFDSITGG |
| ASZ78_006007 | | GSTTDSQCGSSEYIHNLFKEFLTEITRTNATYSLEIADKLYV |
| [Callipepla | | DKTFTVLPEYINCARKFYTGGVEEVNFKTAAEEARQLMNSWV |
| squamata] | | EKETNGQIKDLLVPSSVDFGTMMVFINTIYFKGIWKTAFNTE |
| | | DTREMPFSMTKQESKPVQMMCLNDTFNMVTLPAEKMRILELP |
| | | YASGELSMLVLLPDEVSGLERIEKAINFEKLREWTSTNAMEK |
| | | KSMKVYLPRMKIEEKYNLTSTLMALGMTDLFSRSANLTGISS |
| | | VDNLMISDAVHGAFMEVNEEGTEAAGSTGAIGNIKHSVEFEE |
| | | FRADHPFLFLIRYNPTNVILFFDNSEFTMGSIGAVSTEFCFD |
| | | VFKELRVHHANENIFYSPFTIISALAMVYLGAKDSTRTQINK |
| | | VVRFDKLPGFGDSIEAQCGTSANVHSSLRDILNQITKPNDIY |
| | | SFSLASRLYADETYTILPEYLQCVKELYRGGLESINFQTAAD |
| | | QARELINSWVESQTSGIIRNVLQPSSVDSQTAMVLVNAIYFK |
| | | GLWEKGFKDEDTQAIPFRVTEQENKSVQMMYQIGTFKVASVA |
| | | SEKMKILELPFASGTMSMWVLLPDEVSGLEQLETTISIEKLT |
| | | EWTSSSVMEERKIKVFLPRMKMEEKYNLTSVLMAMGMTDLFS |
| | | SSANLSGISSTLQKKGFRSQELGDKYAKPMLESPALTPQATA |
| | | WDNSWIVAHPPAIEPDLYYQIMEQKWKPFDWPDFRLPMRVSC |
| | | RFRTMEALNKANTSFALDFFKHECQEDDSENILFSPFSISSA |
| | | LATVYLGAKGNTADQMAKVLHFNEAEGARNVTTTIRMQVYSR |
| | | TDQQRLNRRACFQKTEIGKSGNIHAGFKGLNLEINQPTKNYL |
| | | LNSVNQLYGEKSLPFSKEYLQLAKKYYSAEPQSVDFVGTANE |
| | | IRREINSRVEHQTEGKIKNLLPPGSIDSLTRLVLVNALYFKG |
| | | NWATKFEAEDTRHRPFRINTHTTKQVPMMYLSDKFNWTYVES |
| | | VQTDVLELPYVNNDLSMFILLPRDITGLQKLINELTFEKLSA |
| | | WTSPELMEKMKMEVYLPRFTVEKKYDMKSTLSKMGIEDAFTK |
| | | VDNCGVTNVDEITIHVVPSKCLELKHIQINKELKCNKAVAME |
| | | QVSASIGNFTIDLFNKLNETSRDKNIFFSPWSVSSALALTSL |
| | | AAKGNTAREMAEDPENEQAENIHSGFNELLTALNKPRNTYSL |
| | | KSANRIYVEKNYPLLPTYIQLSKKYYKAEPHKVNFKTAPEQS |
| | | RKEINNWVEKQTERKIKNFLSSDDVKNSTKLILVNAIYFKAE |
| | | WEEKFQAGNTDMQPFRMSKNKSKLVKMMYMRHTFPVLIMEKL |
| | | NFKMIELPYVKRELSMFILLPDDIKDSTTGLEQLERELTYEK |
| | | LSEWADSKKMSVTLVDLHLPKFSMEDRYDLKDALRSMGMASA |
| | | FNSNADFSGMTGERDLVISKVCHQSFVAVDEKGTEAAAATAV |
| | | IAEAVPMESLSASTNSFTLDLYKKLDETSKGQNIFFASWSIA |
| | | TALTMVHLGAKGDTATQVAKGPEYEETENIHSGFKELLSALN |
| | | KPRNTYSMKSANRLFGDKTYPLLPTKTKPVQMMFLKDTFLIH |
| | | HERTMKFKIIELPYMGNELSAFVLLPDDISDNTTGLELVERE |
| | | LTYEKLAEWSNSASMMKVKVELYLPKLKMEENYDLKSALSDM |
| | | GIRSAFDPAQADFTRMSEKKDLFISKVIHKAFVEVNEEDRIV |
| | | QLASGRLTGNTEAQIAKVLSLSKAEDAHNGYQSLLSEINNPD |
| | | TKYILRTANRLYGEKTFEFLSSFIDSSQKFYHAGLEQTDFKN |
| | | ASEDSRKQINGWVEEKTEGKIQKLLSEGIINSMTKLVLVNAI |
| | | YFKGNWQEKFDKETTKEMPFKINKNETKPVQMMFRKGKYNMT |
| | | YIGDLETTVLEIPYVDNELSMIILLPDSIQDESTGLEKLERE |
| | | LTYEKLMDWINPNMMDSTEVRVSLPRFKLEENYELKPTLSTM |
| | | GMPDAFDLRTADESGISSGNELVLSEVVHKSFVEVNEEGTEA |
| | | AAATAGIMLLRCAMIVANFTADHPFLFFIRHNKTNSILFCGR |
| | | FCSP |
| PREDICTED: | 201 | MASIGAASTEFCFDVFKELKTQHVKENIFYSPMAIISALSMV |
| Ovalbumin-like | | YIGARENTRAEIDKVVHFDKITGFGNAVESQCGPSVSVHSSL |
| [Mesitornis | | KDLITQISKRSDNYSLSYASRIYAEETYPILPEYLQCVKEVY |
| unicolor] | | KGGLESISFQTAADQARENINAWVESQTNGMIKNILQPSSVN |
| | | PQTEMVLVNAIYLKGMWEKAFKDEDTQTMPFRVTQQESKPVQ |
| | | MMYQIGSFKVAVIASEKMKILELPYTSGQLSMLVLLPDDVSG |
| | | LEQVESAITAEKLMEWTSPSIMEERTMKVYLPRMKMVEKYNL |
| | | TSVLMALGMTDLFTSVANLSGISSAQGLKMSQAIHEAFVEIY |
| | | EAGSEAVGSTGVGMEITSVSEEFKADLSFLFLIRHNPTNSII |
| | | FFGRCISP |
| Ovalbumin, | 202 | MGSIGAASTEFCFDVFRELRVQHVNENIFYSPFSIISALAMV |
| partial [Anas | | YLGARDNTRTQIDKISQFQALSDEHLVLCIQQLGEFFVCTNR |
| platyrhynchos] | | ERREVTRYSEQTEDKTQDQNTGQIHKIVDTCMLRQDILTQIT |
| | | KPSDNFSLSFASRLYAEETYAILPEYLQCVKELYKGGLESIS |
| | | FQTAADQARELINSWVESQTNGIIKNILQPSSVDSQTTMVLV |
| | | NAIYFKGMWEKAFKDEDTQAMPFRMTEQESKPVQMMYQVGSF |
| | | KVAMVTSEKMKILELPFASGMMSMFVLLPDEVSGLEQLESTI |
| | | SFEKLTEWTSSTMMEERRMKVYLPRMKMEEKYNLTSVFMALG |
| | | MTDLFSSSANMSGISSTVSLKMSEAVHAACVEIFEAGRDVVG |
| | | SAEAGMDVTSVSEEFRADHPFLFFIKHNPTNSILFFGRWMSP |
| PREDICTED: | 203 | MGSIGAASAEFCLDIFKELKVQHVNENIIFSPMTIISALSLV |
| Ovalbumin-like | | YLGAKEDTRAQIEKVVPFDKIPGFGEIVESQCPKSASVHSSI |
| [Chaetura | | QDIFNQIIKRSDNYSLSLASRLYAEESYPIRPEYLQCVKELD |
| pelagica] | | KEGLETISFQTAADQARQLINSWVESQTNGMIKNILQPSSVN |
| | | SQTEMVLVNAIYFRGLWQKAFKDEDTQAVPFRITEQESKPVQ |
| | | MMQQIGSFKVAEIASEKMKILELPYASGQLSMLVLLPDDVSG |
| | | LEKLESSITVEKLIEWTSSNLTEERNVKVYLPRLKIEEKYNL |
| | | TSVLAALGITDLFSSSANLSGISTAESLKLSRAVHESFVEIQ |
| | | EAGHEVEGPKEAGIEVTSALDEFRVDRPFLFVTKHNPTNSIL |
| | | FLGRCLSP |
| PREDICTED: | 204 | MGSISAASGEFCLDIFKELKVQHVNENIFYSPMVIVSALSLV |
| Ovalbumin-like | | YLGARENTRAQIDKVIPFDKITGSSEAVESQCGTPVGAHISL |
| [Apaloderma | | KDVFAQIAKRSDNYSLSFVNRLYAEETYPILPEYLQCVKELY |
| vittatum] | | KGGLETISFQTAADQAREIINSWVESQTDGKIKNILQPSSVD |
| | | PQTKMVLVSAIYFKGLWEKSFKDEDTQAVPFRVTEQESKPVQ |
| | | MMYQIGSFKVAAIAAEKIKILELPYASEQLSMLVLLPDDVSG |
| | | LEQLEKKISYEKLTEWTSSSVMEEKKIKVYLPRMKIEEKYNL |
| | | TSILMSLGITDLFSSSANLSGISSTKSLKMSEAVHEASVEIY |
| | | EAGSEASGITGDGMEATSVFGEFKVDHPFLFMIKHKPTNSIL |
| | | FFGRCISP |
| Ovalbumin-like | 205 | MGSIGPVSTEVCCDIFRELRSQSVQENVCYSPLLIISTLSMV |
| [Corvus cornix | | YIGAKDNTKAQIEKAIHFDKIPGFGESTESQCGTSVSIHTSL |
| cornix] | | KDIFTQITKPSDNYSISIARRLYAEEKYPILPEYIQCVKELY |
| | | KGGLESISFQTAAEKSRELINSWVESQTNGTIKNILQPSSVS |
| | | SQTDMVLVSAIYFKGLWEKAFKEEDTQTIPFRITEQESKPVQ |
| | | MMSQIGTFKVAEIPSEKCRILELPYASGRLSLWVLLPDDISG |
| | | LEQLETAITFENLKEWTSSSKMEERKIRVYLPRMKIEEKYNL |
| | | TSVLKSLGITDLFSSSANLSGISSAESLKVSAAFHEASVEIY |
| | | EAGSKGVGSSEAGVDGTSVSEEIRADHPFLFLIKHNPSDSIL |
| | | FFGRCFSP |
| PREDICTED: | 206 | MGSIGAASTEFCFDVFKELKVQHVNENIIISPLSIISALSMV |
| Ovalbumin-like | | YLGAREDTRAQIDKVVHFDKITGFGEAIESQCPTSESVHASL |
| [Calypte anna] | | KETFSQLTKPSDNYSLAFASRLYAEETYPILPEYLQCVKELY |
| | | KGGLETINFQTAAEQARQVINSWVESQTDGMIKSLLQPSSVD |
| | | PQTEMILVNAIYFRGLWERAFKDEDTQELPFRITEQESKPVQ |
| | | MMSQIGSFKVAVVASEKVKILELPYASGQLSMLVLLPDDVSG |
| | | LEQLESSITVEKLIEWISSNTKEERNIKVYLPRMKIEEKYNL |
| | | TSVLVALGITDLESSSANLSGISSAESLKISEAVHEAFVEIQ |
| | | EAGSEVVGSPGPEVEVTSVSEEWKADRPFLFLIKHNPTNSIL |
| | | FFGRYISP |
| PREDICTED: | 207 | MGSIGPVSTEVCCDIFRELRSQSVQENVCYSPLLIISTLSMV |
| Ovalbumin | | YIGAKDNTKAQIEKAIHFDKIPGFGESTESQCGTSVSIHTSL |
| [Corvus | | KDIFTQITKPSDNYSISIARRLYAEEKYPILQEYIQCVKELY |
| brachyrhynchos] | | KGGLESISFQTAAEKSRELINSWVESQTNGTIKNILQPSSVS |
| | | SQTDMVLVSAIYFKGLWEKAFKEEDTQTIPFRITEQESKPVQ |
| | | MMSQIGTFKVAEIPSEKCRILELPYASGRLSLWVLLPDDISG |
| | | LEQLETSITFENLKEWTSSSKMEERKIRVYLPRMKIEEKYNL |
| | | TSVLKSLGITDLFSSSANLSGISSAESLKVSAVFHEASVEIY |
| | | EAGSKGVGSSEAGVDGTSVSEEIRADHPFLFLIKHNPSDSIL |
| | | FFGRCFSP |
| Hypothetical | 208 | MLNLMHPKQFCCTMGSIGPVSTEVCCDIFRELRSQSVQENVC |
| protein | | YSPLLIISTLSMVYIGAKDNTKAQIEKAIHFDKIPGFGESTE |
| DUI87_08270 | | SQCGTSVSIHTSLKDIFTQITKPSDNYSISIASRLYAEEKYP |
| [Hirundo | | ILPEYIQCVKELYKGGLESISFQTAAEKSRELINSWVESQTN |
| rustica rustica] | | GTIKNILQPSSVSSQTDMVLVSAIYFKGLWEKAFKEEDTQTV |
| | | PFRITEQESKPVQMMSQIGTFKVAEIPSEKCRILELPYASGR |
| | | LSLWVLLPDDISGLEQLETAITSENLKEWTSSSKMEERKIKV |
| | | YLPRMKIEEKYNLTSVLKSLGITDLFSSSANLSGISSAESLK |
| | | VSGAFHEAFVEIYEAGSKAVGSSGAGVEDTSVSEEIRADHPF |
| | | LFFIKHNPSDSILFFGRCFSP |
| Ostrich OVA | 209 | EAEAGSIGTASAEFCFDVFKELKVHHVNENIFYSPLSIISAL |
| sequence as | | SMVYLGARENTKTQMEKVIHFDKITGLGESMESQCGTGVSIH |
| secreted from | | TALKDMLSEITKPSDNYSLSLASRLYAEQTYAILPEYLQCIK |
| pichia | | ELYKESLETVSFQTAADQARELINSWIESQTNGVIKNFLQPG |
| | | SVDSQTELVLVNAIYFKGMWEKAFKDEDTQEVPFRITEQESR |
| | | PVQMMYQAGSFKVATVAAEKIKILELPYASGELSMLVLLPDD |
| | | ISGLEQLETTISFEKLTEWTSSNMMEDRNMKVYLPRMKIEEK |
| | | YNLTSVLIALGMTDLFSPAANLSGISAAESLKMSEAIHAAYV |
| | | EIYEADSEIVSSAGVQVEVTSDSEEFRVDHPFLFLIKHNPTN |
| | | SVLFFGRCISP |
| Ostrich | 300 | MRFPSIFTAVLFAASSALAAPVNTTTEDETAQIPAEAVIGYS |
| construct | | DLEGDFDVAVLPFSNSTNNGLLFINTTIASIAAKEEGVSLEK |
| (secretion | | REAEAGSIGTASAEFCFDVFKELKVHHVNENIFYSPLSIISA |
| signal + mature | | LSMVYLGARENTKTQMEKVIHFDKITGLGESMESQCGTGVSI |
| protein) | | HTALKDMLSEITKPSDNYSLSLASRLYAEQTYAILPEYLQCI |
| | | KELYKESLETVSFQTAADQARELINSWIESQTNGVIKNFLQP |
| | | GSVDSQTELVLVNAIYFKGMWEKAFKDEDTQEVPFRITEQES |
| | | RPVQMMYQAGSFKVATVAAEKIKILELPYASGELSMLVLLPD |
| | | DISGLEQLETTISFEKLTEWTSSNMMEDRNMKVYLPRMKIEE |
| | | KYNLTSVLIALGMTDLFSPAANLSGISAAESLKMSEAIHAAY |
| | | VEIYEADSEIVSSAGVQVEVTSDSEEFRVDHPFLFLIKHNPT |
| | | NSVLFFGRCISP |
| Duck OVA | 301 | EAEAGSIGAASTEFCFDVFRELRVQHVNENIFYSPFSIISAL |
| sequence as | | AMVYLGARDNTRTQIDKVVHFDKLPGFGESMEAQCGTSVSVH |
| secreted from | | SSLRDILTQITKPSDNFSLSFASRLYAEETYAILPEYLQCVK |
| pichia | | ELYKGGLESISFQTAADQARELINSWVESQTNGIIKNILQPS |
| | | SVDSQTTMVLVNAIYFKGMWEKAFKDEDTQAMPFRMTEQESK |
| | | PVQMMYQVGSFKVAMVTSEKMKILELPFASGMMSMFVLLPDE |
| | | VSGLEQLESTISFEKLTEWTSSTMMEERRMKVYLPRMKMEEK |
| | | YNLTSVFMALGMTDLFSSSANMSGISSTVSLKMSEAVHAACV |
| | | EIFEAGRDVVGSAEAGMDVTSVSEEFRADHPFLFFIKHNPTN |
| | | SILFFGRWMSP |
| Duck construct | 302 | MRFPSIFTAVLFAASSALAAPVNTTTEDETAQIPAEAVIGYS |
| (secretion | | DLEGDFDVAVLPFSNSTNNGLLFINTTIASIAAKEEGVSLEK |
| signal + mature | | REAEAGSIGAASTEFCFDVFRELRVQHVNENIFYSPFSIISA |
| protein) | | LAMVYLGARDNTRTQIDKVVHFDKLPGFGESMEAQCGTSVSV |
| | | HSSLRDILTQITKPSDNFSLSFASRLYAEETYAILPEYLQCV |
| | | KELYKGGLESISFQTAADQARELINSWVESQTNGIIKNILQP |
| | | SSVDSQTTMVLVNAIYFKGMWEKAFKDEDTQAMPFRMTEQES |
| | | KPVQMMYQVGSFKVAMVTSEKMKILELPFASGMMSMFVLLPD |
| | | EVSGLEQLESTISFEKLTEWTSSTMMEERRMKVYLPRMKMEE |
| | | KYNLTSVFMALGMTDLFSSSANMSGISSTVSLKMSEAVHAAC |
| | | VEIFEAGRDVVGSAEAGMDVTSVSEEFRADHPFLFFIKHNPT |
| | | NSILFFGRWMSP |
| Ovoglobulin G2 | 303 | TRAPDCGGILTPLGLSYLAEVSKPHAEVVLRQDLMAQRASDL |
| | | FLGSMEPSRNRITSVKVADLWLSVIPEAGLRLGIEVELRIAP |
| | | LHAVPMPVRISIRADLHVDMGPDGNLQLLTSACRPTVQAQST |
| | | REAESKSSRSILDKVVDVDKLCLDVSKLLLFPNEQLMSLTAL |
| | | FPVTPNCQLQYLPLAAPVFSKQGIALSLQTTFQVAGAVVPVP |
| | | VSPVPFSMPELASTSTSHLILALSEHFYTSLYFTLERAGAFN |
| | | MTIPSMLTTATLAQKITQVGSLYHEDLPITLSAALRSSPRVV |
| | | LEEGRAALKLFLTVHIGAGSPDFQSFLSVSADVTAGLQLSVS |
| | | DTRMMISTAVIEDAELSLAASNVGLVRAALLEELFLAPVCQQ |
| | | VPAWMDDVLREGVHLPHLSHFTYTDVNVVVHKDYVLVPCKLK |
| | | LRSTMA* |
| Ovoglobulin G3 | 304 | MDSISVTNAKFCFDVFNEMKVHHVNENILYCPLSILTALAMV |
| | | YLGARGNTESQMKKVLHFDSITGAGSTTDSQCGSSEYVHNLF |
| | | KELLSEITRPNATYSLEIADKLYVDKTFSVLPEYLSCARKFY |
| | | TGGVEEVNFKTAAEEARQLINSWVEKETNGQIKDLLVSSSID |
| | | FGTTMVFINTIYFKGIWKIAFNTEDTREMPFSMTKEESKPVQ |
| | | MMCMNNSFNVATLPAEKMKILELPYASGDLSMLVLLPDEVSG |
| | | LERIEKTINFDKLREWTSTNAMAKKSMKVYLPRMKIEEKYNL |
| | | TSILMALGMTDLFSRSANLTGISSVDNLMISDAVHGVFMEVN |
| | | EEGTEATGSTGAIGNIKHSLELEEFRADHPFLFFIRYNPTNA |
| | | ILFFGRYWSP* |
| β-ovomucin | 305 | CSTWGGGHFSTFDKYQYDFTGTCNYIFATVCDESSPDFNIQF |
| | | RRGLDKKIARIIIELGPSVIIVEKDSISVRSVGVIKLPYASN |
| | | GIQIAPYGRSVRLVAKLMEMELVVMWNNEDYLMVLTEKKYMG |
| | | KTCGMCGNYDGYELNDFVSEGKLLDTYKFAALQKMDDPSEIC |
| | | LSEEISIPAIPHKKYAVICSQLLNLVSPTCSVPKDGFVTRCQ |
| | | LDMQDCSEPGQKNCTCSTLSEYSRQCAMSHQVVFNWRTENFC |
| | | SVGKCSANQIYEECGSPCIKTCSNPEYSCSSHCTYGCFCPEG |
| | | TVLDDISKNRTCVHLEQCPCTLNGETYAPGDTMKAACRTCKC |
| | | TMGQWNCKELPCPGRCSLEGGSFVTTFDSRSYRFHGVCTYIL |
| | | MKSSSLPHNGTLMAIYEKSGYSHSETSLSAIIYLSTKDKIVI |
| | | SQNELLTDDDELKRLPYKSGDITIFKQSSMFIQMHTEFGLEL |
| | | VVQTSPVFQAYVKVSAQFQGRTLGLCGNYNGDTTDDFMTSMD |
| | | ITEGTASLFVDSWRAGNCLPAMERETDPCALSQLNKISAETH |
| | | CSILTKKGTVFETCHAVVNPTPFYKRCVYQACNYEETFPYIC |
| | | SALGSYARTCSSMGLILENWRNSMDNCTITCTGNQTFSYNTQ |
| | | ACERTCLSLSNPTLECHPTDIPIEGCNCPKGMYLNHKNECVR |
| | | KSHCPCYLEDRKYILPDQSTMTGGITCYCVNGRLSCTGKLQN |
| | | PAESCKAPKKYISCSDSLENKYGATCAPTCOMLATGIECIPT |
| | | KCESGCVCADGLYENLDGRCVPPEECPCEYGGLSYGKGEQIQ |
| | | TECEICTCRKGKWKCVQKSRCSSTCNLYGEGHITTFDGQRFV |
| | | FDGNCEYILAMDGCNVNRPLSSFKIVTENVICGKSGVTCSRS |
| | | ISIYLGNLTIILRDETYSISGKNLQVKYNVKKNALHLMFDII |
| | | IPGKYNMTLIWNKHMNFFIKISRETQETICGLCGNYNGNMKD |
| | | DFETRSKYVASNELEFVNSWKENPLCGDVYFVVDPCSKNPYR |
| | | KAWAEKTCSIINSQVFSACHNKVNRMPYYEACVRDSCGCDIG |
| | | GDCECMCDAIAVYAMACLDKGICIDWRTPEFCPVYCEYYNSH |
| | | RKTGSGGAYSYGSSVNCTWHYRPCNCPNQYYKYVNIEGCYNC |
| | | SHDEYFDYEKEKCMPCAMQPTSVTLPTATQPTSPSTSSASTV |
| | | LTETTNPPV* |
| Lysozyme | 306 | KVFGRCELAAAMKRHGLDNYRGYSLGNWVCAAKFESNENTQA |
| | | TNRNTDGSTDYGILQINSRWWCNDGRTPGSRNLCNIPCSALL |
| | | SSDITASVNCAKKIVSDGNGMNAWVAWRNRCKGTDVQAWIRG |
| | | CRL* |
| Lysozyme | 307 | KVFGRCELAAAMKRHGLDNYRGYSLGNWVCVAKFESNENTQA |
| | | TNRNTDGSTDYGILQINSRWWCNDGRTPGSRNLCNIPCSALL |
| | | SSDITASVNCAKKIVSDGNGMSAWVAWRNRCKGTDVQAWIRG |
| | | CRL* |
| Lysozyme C | 308 | KVFERCELARTLKRLGMDGYRGISLANWMCLAKWESGYNTRA |
| (Human) | | TNYNAGDRSTDYGIFQINSRYWCNDGKTPGAVNACHLSCSAL |
| | | LQDNIADAVACAKRVVRDPQGIRAWVAWRNRCQNRDVRQYVQ |
| | | GCGV* |
| Lysozyme C | 309 | KVFERCELARTLKKLGLDGYKGVSLANWLCLTKWESSYNTKA |
| (Bos taurus) | | TNYNPSSESTDYGIFQINSKWWCNDGKTPNAVDGCHVSCREL |
| | | MENDIAKAVACAKHIVSEQGITAWVAWKSHCRDHDVSSYVEG |
| | | CTL* |
| Ovoinhibitor | 310 | IEVNCSLYASGIGKDGTSWVACPRNLKPVCGTDGSTYSNECG |
| | | ICLYNREHGANVEKEYDGECRPKHVMIDCSPYLQVVRDGNTM |
| | | VACPRILKPVCGSDSFTYDNECGICAYNAEHHTNISKLHDGE |
| | | CKLEIGSVDCSKYPSTVSKDGRTLVACPRILSPVCGTDGFTY |
| | | DNECGICAHNAEQRTHVSKKHDGKCRQEIPEIDCDQYPTRKT |
| | | TGGKLLVRCPRILLPVCGTDGFTYDNECGICAHNAQHGTEVK |
| | | KSHDGRCKERSTPLDCTQYLSNTQNGEAITACPFILQEVCGT |
| | | DGVTYSNDCSLCAHNIELGTSVAKKHDGRCREEVPELDCSKY |
| | | KTSTLKDGRQVVACTMIYDPVCATNGVTYASECTLCAHNLEQ |
| | | RTNLGKRKNGRCEEDITKEHCREFQKVSPICTMEYVPHCGSD |
| | | GVTYSNRCFFCNAYVQSNRTLNLVSMAAC* |
| Cystatin | 311 | MAGARGCVVLLAAALMLVGAVLGSEDRSRLLGAPVPVDENDE |
| | | GLQRALQFAMAEYNRASNDKYSSRVVRVISAKRQLVSGIKYI |
| | | LQVEIGRTTCPKSSGDLQSCEFHDEPEMAKYTTCTFVVYSIP |
| | | WLNQIKLLESKCQ* |
| Porcine Lipase | 312 | SEVCFPRLGCFSDDAPWAGIVQRPLKILPWSPKDVDTRFLLY |
| | | TNQNQNNYQELVADPSTITNSNFRMDRKTRFIIHGFIDKGEE |
| | | DWLSNICKNLFKVESVNCICVDWKGGSRTGYTQASQNIRIVG |
| | | AEVAYFVEVLKSSLGYSPSNVHVIGHSLGSHAAGEAGRRTNG |
| | | TIERITGLDPAEPCFQGTPELVRLDPSDAKFVDVIHTDAAPI |
| | | IPNLGFGMSQTVGHLDFFPNGGKQMPGCQKNILSQIVDIDGI |
| | | WEGTRDFVACNHLRSYKYYADSILNPDGFAGFPCDSYNVFTA |
| | | NKCFPCPSEGCPQMGHYADRFPGKTNGVSQVFYLNTGDASNF |
| | | ARWRYKVSVTLSGKKVTGHILVSLFGNEGNSRQYEIYKGTLQ |
| | | PDNTHSDEFDSDVEVGDLQKVKFIWYNNNVINPTLPRVGASK |
| | | ITVERNDGKVYDFCSQETVREEVLLTLNPC* |
| Kid Lipase | 313 | GLVAADRITGGKDFRDIESKFALRTPEDTAEDTCHLIPGVTE |
| | | SVANCHENHSSKTFVVIHGWTVTGMYESWVPKLVAALYKREP |
| | | DSNVIVVDWLSRAQQHYPVSAGYTKLVGQDVAKFMNWMADEF |
| | | NYPLGNVHLLGYSLGAHAAGIAGSLTSKKVNRITGLDPAGPN |
| | | FEYAEAPSRLSPDDADFVDVLHTFTRGSPGRSIGIQKPVGHV |
| | | DIYPNGGTFQPGCNIGEALRVIAERGLGDVDQLVKCSHERSV |
| | | HLFIDSLLNEENPSKAYRCNSKEAFEKGLCLSCRKNRCNNMG |
| | | YEINKVRAKRSSKMYLKTRSQMPYKVFHYQVKIHFSGTESNT |
| | | YTNQAFEISLYGTVAESENIPFTLPEVSTNKTYSFLLYTEVD |
| | | IGELLMLKLKWISDSYFSWSNWWSSPGFDIGKIRVKAGETQK |
| | | KVIFCSREKMSYLQKGKSPVIFVKCHDKSLNRKSG* |
| Porcine | 314 | APKKGVRWCVISTAEYSKCRQWQSKIRRTNPMFCIRRASPTD |
| Lactoferrin | | CIRAIAAKRADAVTLDGGLVFEADQYKLRPVAAEIYGTEENP |
| | | QTYYYAVAVVKKGFNFQLNQLQGRKSCHTGLGRSAGWNIPIG |
| | | LLRRFLDWAGPPEPLQKAVAKFFSQSCVPCADGNAYPNLCQL |
| | | CIGKGKDKCACSSQEPYFGYSGAFNCLHKGIGDVAFVKESTV |
| | | FENLPQKADRDKYELLCPDNTRKPVEAFRECHLARVPSHAVV |
| | | ARSVNGKENSIWELLYQSQKKFGKSNPQEFQLFGSPGQQKDL |
| | | LFRDATIGFLKIPSKIDSKLYLGLPYLTAIQGLRETAAEVEA |
| | | RQAKVVWCAVGPEELRKCRQWSSQSSQNLNCSLASTTEDCIV |
| | | QVLKGEADAMSLDGGFIYTAGKCGLVPVLAENQKSRQSSSSD |
| | | CVHRPTQGYFAVAVVRKANGGITWNSVRGTKSCHTAVDRTAG |
| | | WNIPMGLLVNQTGSCKFDEFFSQSCAPGSQPGSNLCALCVGN |
| | | DQGVDKCVPNSNERYYGYTGAFRCLAENAGDVAFVKDVTVLD |
| | | NINGQNTEEWARELRSDDFELLCLDGTRKPVTEAQNCHLAVA |
| | | PSHAVVSRKEKAAQVEQVLLTEQAQFGRYGKDCPDKFCLFRS |
| | | ETKNLLFNDNTEVLAQLQGKTTYEKYLGSEYVTAIANLKQCS |
| | | VSPLLEACAFMMR* |
| Bovine | 315 | APRKNVRWCTISQPEWFKCRRWQWRMKKLGAPSITCVRRAFA |
| Lactoferrin | | LECIRAIAEKKADAVTLDGGMVFEAGRDPYKLRPVAAEIYGT |
| | | KESPQTHYYAVAVVKKGSNFQLDQLQGRKSCHTGLGRSAGWI |
| | | IPMGILRPYLSWTESLEPLQGAVAKFFSASCVPCIDRQAYPN |
| | | LCQLCKGEGENQCACSSREPYFGYSGAFKCLQDGAGDVAFVK |
| | | ETTVFENLPEKADRDQYELLCLNNSRAPVDAFKECHLAQVPS |
| | | HAVVARSVDGKEDLIWKLLSKAQEKFGKNKSRSFQLFGSPPG |
| | | QRDLLFKDSALGFLRIPSKVDSALYLGSRYLTTLKNLRETAE |
| | | EVKARYTRVVWCAVGPEEQKKCQQWSQQSGQNVTCATASTTD |
| | | DCIVLVLKGEADALNLDGGYIYTAGKCGLVPVLAENRKSSKH |
| | | SSLDCVLRPTEGYLAVAVVKKANEGLTWNSLKDKKSCHTAVD |
| | | RTAGWNIPMGLIVNQTGSCAFDEFFSQSCAPGADPKSRLCAL |
| | | CAGDDQGLDKCVPNSKEKYYGYTGAFRCLAEDVGDVAFVKND |
| | | TVWENTNGESTADWAKNLNREDFRLLCLDGTRKPVTEAQSCH |
| | | LAVAPNHAVVSRSDRAAHVKQVLLHQQALFGKNGKNCPDKFC |
| | | LFKSETKNLLFNDNTECLAKLGGRPTYEEYLGTEYVTAIANL |
| | | KKCSTSPLLEACAFLTR* |

TABLE 6
Exemplary Linkers

| Sequence | SEQ | |
|---|---|---|
| Info | ID NO: | Amino Acid sequence |
| GGGS | SEQ | GGGGS |
| linker | ID NO: | |
| | 316 | |
| GSS | SEQ | GSS |
| linker | ID NO: | |
| | 317 | |
| A rigid | SEQ | EAAAREAAAREAAAREAAAR |
| linker | ID NO: | |
| that | 318 | |
| forms | | |
| 4 turns | | |
| of an | | |
| alpha | | |
| helix | | |
| Full | SEQ | GSSGSSGSSGSSGSSGSSGSSGSSEAAAREAAAREAAAREAAARGGG |
| linker | ID NO: | GSGGGGSGGGGS |
| | 319 | |
| A | SEQ | GSSGSSGSSGSSGSSGSSGSSGSS |
| flexible | ID NO: | |
| GS | 320 | |
| linker | | |
| with | | |
| higher | | |
| S | | |
| content | | |
| A | SEQ | GGGGSGGGGSGGGGS |
| flexible | ID NO: | |
| GS | 321 | |
| linker | | |
| with | | |
| much | | |
| higher | | |
| G | | |
| content | | |

TABLE 7
ALG/OST PAthway knockouts

| Sequence | SEQ ID | |
|---|---|---|
| Info | NO: | Amino Acid sequence |
| ALG6 | SEQ ID | MPHKRTPSSSLLYARIPGISFENSPVFDFLSPFGPAPNQWVARYIIII |
| (GS115-GQ68_ | NO: 322 | FAILIRLAVGLGSYSGFNTPPMYGDFEAQRHWMEITQHLSIEKWY |
| 00786T0/ | | FYDLQYWGLDYPPLTAFHSYFFGKLGSFINPAWFALDVSRGFESV |
| XP_002491463.1) | | DLKSYMRATAILSELLCFIPAVIWYCRWMGLNYFNQNAIEQTIIAS |
| | | AILFNPSLIIIDHGHFQYNSVMLGFALLSILNLLYDNFALAAIFFVLS |
| | | ISFKQMALYYSPIMFFYMLSVSCWPLKNFNLLRLATISIAVLLTFA |
| | | TLLLPFVLVDGMSQIGQILFRVFPFSRGLFEDKVANFWCTTNILVK |
| | | YKQLFTDKTLTRISLVATLIAISPSCFIIFTHPKKVLLPWAFAACSW |
| | | AFYLFSFQVHEKSVLVPLMPTTLLLVEKDLDIISMVCWISNIAFFS |
| | | MWPLLKRDGLALEYFVLGILSNWLIGNLNWISKWLVPSFLIPGPT |
| | | LSKKVPKRDTKTVVHTHWFWGSVTFVSYLGATVIQFVDWLYLPP |
| | | AKYPDLWVILNTTLSFACFGLFWLWINYNLYILRDFKLKDA* |
| STT3 | SEQ ID | MVTINDQGYITVNDRVLKLIKSLLIVLIFISITIAAVSSRLFSVIRFESI |
| (GS115-Q68_ | NO: 323 | IHEFDPWFNFRATKYLVHNGFYKFLNWFDDKTWYPLGRVTGGTL |
| 01669T0/ | | YPGLMVTSAVIHNLLAKIGLPIDIRNICVMLAPAFSSLTAIAMYFLT |
| XP_002490630.1) | | LELTNDSESIANGTAKATAALFSAIFMGITPGYISRSVAGSYDNEAI |
| | | AITLLMVTFYFWIKAVKLGSIFYSSVTALFYFYMVSAWGGYVFIT |
| | | NLIPLHVFVLLLMGRFTHKIYVSYTTWYVLGTLMSMQIPFVGFLPI |
| | | RSNDHMAPLGVFGLIQLVLIGDFFKSQLSRKVFIKLAIASGVVIGIL |
| | | GVVGLVLATKIGLIAPWTGRFYSLWDTNYAKIHIPIIASVSEHQPTP |
| | | WASFFFDLNFLIWLFPVGVWFCFQELTDGAVFVIIYSVLASYFAG |
| | | VMVRLILTLAPIVCVCGAIAITKLFEVYSDFTDVVKGKSGNFFTLF |
| | | SKLAVLGSFGFYLFFYVKHCTWVTENAYSSPSVVLASHAADGSQI |
| | | LIDDYREAYYWLRMNTPEDAKVMAWWDYGYQIGGMADRTTFV |
| | | DNNTWNNTHIATVGKAMAVSEEKSEVIMRQLGVDYILVIFGGVL |
| | | GYSGDDINKFLWMVRISEGIWPEEVSERGYFTPRGEYKIDDNAAQ |
| | | AMKDSMLYKMSFYRFGELFPSGDAIDRVRGQRLSRSYAESIDLNI |
| | | VEEVFTSENWLVRLYKLKEPDNLGRSLLTLKDNEKKLATKKGRR |
| | | LRVNKKPSLDLRV* |

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Expression Constructs, Transformation, Protein Purification and Processing

Constructs may be designed to disrupt beta-mannosyl transferases BMT1 and BMT2 genes (XP_002493882.1 and XP_002493883.1 respectively). Additionally, expression constructs may be designed to express one or more proteins of interest, such as nutritional proteins. The constructs may be transformed into a host cell such as Pichia pastoris.

In one example, another expression construct expressing a mannosidase may be designed and transformed into the host cell. In this example, the disruption of BMT1 and BMT2 would lead to the production of a smaller exopolysaccharide. Additionally, the mannosidase production would be expected to further hydrolyze the exopolysaccharide to mannose which can be used by the host cell as a carbon source. It would be expected that the host cell produces a reduced level of exopolysaccharides thereby reducing the impurities to be separated from the recombinantly produced nutritional protein.

The nutritional protein may be secreted from the host cell and purified using conventional methods of purification.

### Example 2: Expression Constructs, Transformation, Protein Purification and Processing

Constructs were designed to disrupt beta-mannosyl transferases BMT1 and BMT2 genes (XP_002493882.1 and XP_002493883.1 respectively) in a Pichia pastoris strain. Knockouts were performed via standard Homologous Recombination (HR) methods in yeast. In summary, genes of interest (GOIs) were deleted by using linearized plasmids that had homology to genomic regions that surround the GOIs, which were transformed into yeast via standard electroporation techniques. The native HR machinery replaces the GOI with the linearized plasmid. The plasmid with antibiotic resistance can eventually be removed using the Cre/lox recombinase system leaving only a small insertion scar where the GOI initially was found.

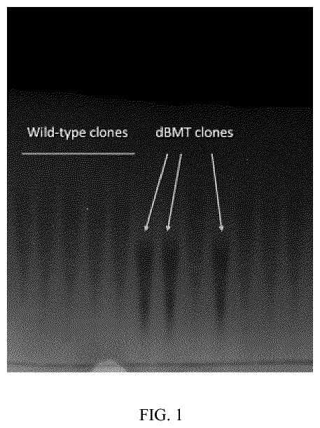

In this example, the disruption of BMT1 and BMT2 lead to the production of a smaller exopolysaccharide. Using gel electrophoresis and the cationic dye Alcian blue (which binds to the phospho-mannan moiety via the phosphodiester bond) it is shown in FIG. 1 that disrupting the BMT1 and BMT2 genes (AT250_GQ6804781 and AT250_GQ6804782) produces a noticeable shift in the size of EPS, which strongly suggests that the EPS byproduct is a form of mannan polysaccharide.

It is also shown in FIG. 2 that Pichia species can grow with mannose as a sole carbon source, illustrating that production strains will be able to recover carbon from the EPS/mannan that is broken down.

### Example 3: Expression Constructs, Transformation, Protein Purification and Processing

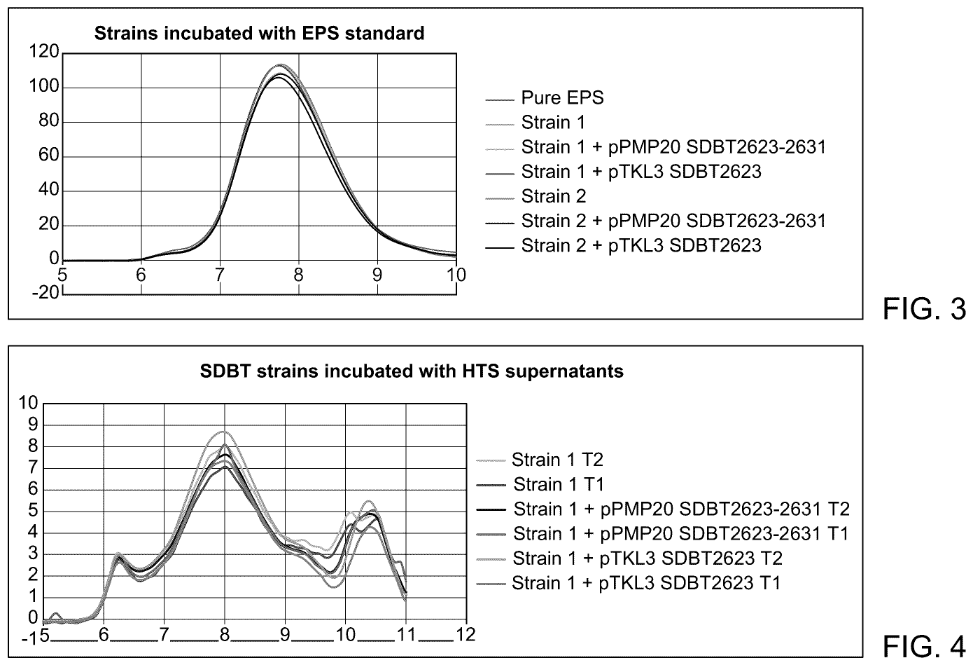

Several Pichia pastoris strains which were previously transformed to express a glycoprotein (ovomucoid) and a transcription factor (HAC1) were cultured. The supernatant from that culture contained exopolysaccharides (EPS). The EPS was filter-purified and analyzed. Additionally, Strain 1 and Strain 2 were transformed with a mannosidase expressing constructs (pPMP20 SDBT2623-2631 vs pTKL3 SDBT2623). The EPS produced by these strains were analyzed and as is shown in FIG. 3, the size of the EPS byproduct is unchanged when strains are incubated with purified EPS. The Sed1 display construct found in the strain uses the PMP20 promoter from Pichia pastoris and TDH3 terminator.

The cells were also incubated with their own culture supernatant to see if increasing the time spent with substrate would allow for hydrolysis of the polysaccharide byproduct. FIG. 4 shows that regardless of the expressed mannosidase (pPMP20 SDBT2623-2631 vs pTKL3 SDBT2623), there is no activity for the enzymes against the wild-type mannan, which is highly branched and ends in terminal beta anomers of mannose.

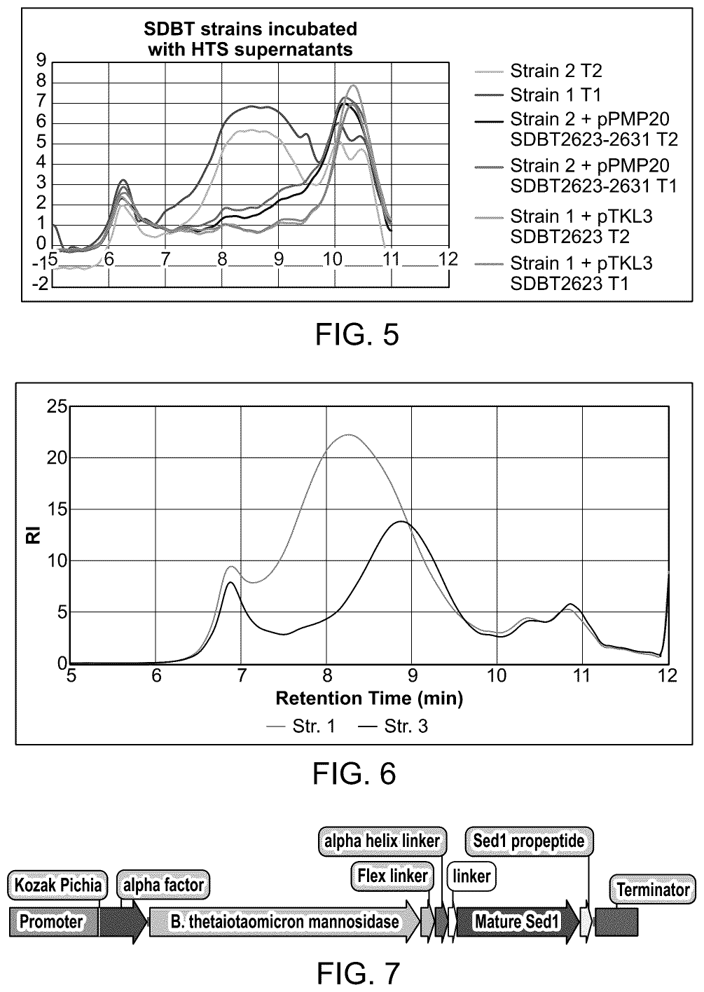

While the mannosidases were not able to act on the “wild-type” EPS produced in Strain 1 cells or the purified product, FIG. 5 shows that when the enzymes are coupled with mannosyltransferase deletions, they do indeed use EPS as a substrate. Strain 2 has had the genes responsible for producing terminal beta mannose anomers (BMT1 and BMT2, GQ6804782 and GQ6804781, respectively), and an alpha-1,2 branching enzyme (MNN2 family protein, GQ6802166), which already produces a right shift in the elution profile of the EPS it produces. When this deletion mutant is coupled with the expression of different mannosidase constructs, it produces a right shift in the elution time of the EPS byproduct, suggesting that the enzymes display activity against the simplified structure of mannan following the deletion of native mannan mannosyltransferases.

### Example 4: Surface Display of Mannosidases

Mannan has been identified using gel electrophoresis and mass spectrometry as the polysaccharide impurity (known as EPS—extracellular polysaccharide) found in supernatants from P. pastoris strains that secrete Proteins of Interest (POIs). Mannan is produced by the sequential action of many mannosyltransferases in the Golgi apparatus. Following the attachment of the core glycan moiety to an asparagine residue, mannan polymerase I (M-pol I) extend the core structure with ˜10 alpha-1,6 mannose units using the Mnn9 catalytic subunit. Next the M-pol II complex (catalytic subunits Mnn10 and Mnn11) extends by another ˜50-100 alpha-1,6 mannose units, which creates a long, linear mannan backbone composed of alpha-1,6-linked sugars. The linear mannan backbone is the extensively decorated with alpha-1,2- and phospho-mannose branch points. These decorations are carried out by members of the MNN and KTR families of proteins—of which there are a total of 10 known in P. pastoris. Finally, some species of yeast (including C. albicans and P. pastoris) produce terminal beta-1,2-linked mannose units to “cap” the mannan molecule (opposed to the terminal alpha-1,3-mannose units found in S. cerevisiae mannan), and these reactions are carried out by the BMT family of mannosyltransferases (four of these family members are found in P. pastoris, two of which have been determined to be catalytically active—BMT1/2). Following the identification of the mannosyltransferases discussed in Example 2, they were deleted to reduce the size and complexity of the mannan/EPS molecule. As is shown in the chromatogram in FIG. 6, the deletion of multiple native mannosyltransferases indeed increased the retention time of eluted EPS using size exclusion chromatography (SEC) (indicative of a decrease in the size of the molecule). Strain 3 was built from Strain 1 by the sequential deletion of five native mannosyltransferases (BMT1 (SEQ ID NO: 12), BMT2 (SEQ ID NO: 13), MNN2 (SEQ ID NO: 1), MNNF1 (SEQ ID NO: 2), MNNF2 (SEQ ID NO: 3)), causing the noticeable right-shift in the EPS peak between 8 and 9 minutes.

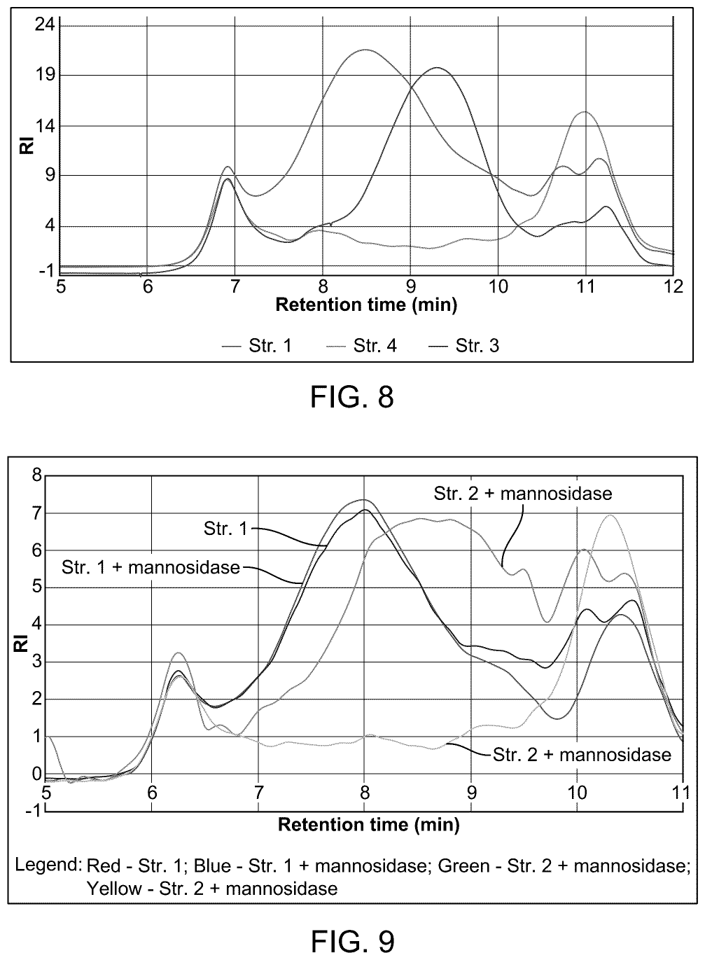

The strain was also modified to express mannan hydrolytic enzymes (mannanases/mannosidases) which are normally expressed by the common human gut microbe Bacteroides thetaiotaomicron. Most yeasts are not known to produce enzymes that breakdown their own cell wall material, however B. theta has been shown to scavenge carbon in the form of mannose from yeast cell wall material in the human gut. Using a surface-display approach (FIG. 7) this example demonstrates that these enzymes can used to breakdown the EPS molecule produced by P. Pastoris (following the deletion of select native mannosyltransferases), once again evidenced by shifts in the elution profile of EPS following SEC analysis (FIG. 8).

Some mannosyltransferase deletions are required for B. theta mannosidases to recognize EPS as a substrate for cleavage. In FIG. 9, it is shown that when Strain 1 and Strain 2 (Strain 1+3 deleted mannosyltransferases) express the exact same mannosidase construct, only the Strain 2+ mannosidase build produces EPS which the surface-displayed enzyme can use as a substrate. The disruption of native mannosyltransferases are important for B. theta enzymes to recognize mannan as a substrate for cleavage. Only the strain with deletions and mannosidase elicits the right-shift in the EPS elution profile.

1. A recombinant host cell for manufacturing a heterologous protein of interest, wherein the host cell is a yeast and is engineered to underexpress two mannosyl transferases: beta-mannosyl transferase 1 (BMT1) and beta-mannosyl transferase 2 (BMT2) or functional homologues thereof wherein the underexpression is compared to the host cell prior to genetic manipulation to achieve underexpression, wherein the host cell is engineered to express a heterologous protein of interest and a heterologous mannosidase.

2. The recombinant host cell of claim 1, wherein underexpression is achieved by independently for each mannosyl transferase protein knocking-out the polynucleotide encoding the mannosyl transferase protein or a homologue thereof from the genome of said host cell, disrupting the polynucleotide encoding the mannosyl transferase protein or a homologue thereof in the host cell, disrupting a promoter which is operably linked with said polynucleotide encoding the mannosyl transferase protein or a homologue thereof, replacing the promoter which is operably linked with said polynucleotide encoding the mannosyl transferase protein or a homologue thereof with another promoter which has lower promoter activity, or disrupting expression control sequences of the mannosyl transferase protein or a homologue thereof, wherein the functional homologue has at least 70% sequence identity to an amino acid sequence of a mannosyl transferase.

3. The recombinant host cell of claim 1, wherein the host cell is Pichia pastoris.

4. The recombinant host cell of claim 1, wherein the BMT1 protein comprises an amino acid sequence that is at least 70% identical, at least 75% identical, at least 80% identical, at least 85% identical, at least 90% identical, or at least 95% identical, to SEQ ID NO: 12.

5. The recombinant host cell of claim 1, wherein the BMT2 protein comprises an amino acid sequence that is at least 70% identical, at least 75% identical, at least 80% identical, at least 85% identical, at least 90% identical, or at least 95% identical, to SEQ ID NO: 13.

6. The recombinant host cell of claim 1, wherein the recombinant host cell is engineered to express at least 10% less BMT1 relative to a host cell which has not been engineered to underexpress BMT1.

7. The recombinant host cell of claim 1, wherein the recombinant host cell is engineered to express at least 10%, 15%, 20%, 30%, 40%, 50%, 60%, 70%, 80%, 90%, or 95% less BMT1 relative to a host cell which has not been engineered to underexpress BMT1.

8. The recombinant host cell of claim 1, wherein the recombinant host cell is engineered to knock out BMT1, wherein the knockout leads to no activity of BMT1 in the recombinant host cell.

9. The recombinant host cell of claim 1, wherein the recombinant host cell is engineered to express at least 10% less BMT2 relative to a host cell which has not been engineered to underexpress BMT2.

10. The recombinant host cell of claim 1, wherein the recombinant host cell is engineered to express at least 10%, 15%, 20%, 30%, 40%, 50%, 60%, 70%, 80%, 90%, or 95% less BMT2 relative to a host cell which has not been engineered to underexpress BMT2.

11. The recombinant host cell of claim 1, wherein the recombinant host cell is engineered to knock out BMT2, wherein the knockout leads to no activity of BMT2 in the recombinant host cell.

12. The recombinant host cell of claim 1, wherein the recombinant host cell produces a reduced size of exopolysaccharides relative to a host cell not engineered to underexpress BMT1 and BMT2.

13. The recombinant host cell of claim 1, wherein the recombinant host cell is further engineered to underexpress alpha-1,2-mannosyltransferase MNN2.

14. The recombinant host cell of claim 13, wherein the MNN2 protein comprises an amino acid sequence that is at least 70% identical, at least 75% identical, at least 80% identical, at least 85% identical, at least 90% identical, or at least 95% identical, to one of SEQ ID NO: 1.

15. The recombinant host cell of claim 13, wherein the recombinant host cell is engineered to express at least 10%, 15%, 20%, 30%, 40%, 50%, 60%, 70%, 80%, 90%, or 95% less MNN2 relative to a host cell which has not been engineered to underexpress MNN2.

16. The recombinant host cell of claim 1, wherein the recombinant host cell is further engineered to underexpress MNNF1.

17. The recombinant host cell of claim 16, wherein the MNNF1 protein comprises an amino acid sequence that is at least 70% identical, at least 75% identical, at least 80% identical, at least 85% identical, at least 90% identical, or at least 95% identical, to one of SEQ ID NO: 2.

18. The recombinant host cell of claim 16, wherein the recombinant host cell is engineered to express at least 10%, 15%, 20%, 30%, 40%, 50%, 60%, 70%, 80%, 90%, or 95% less MNNF1 relative to a host cell which has not been engineered to underexpress MNNF1.

19. The recombinant host cell of claim 1, wherein the recombinant host cell is further engineered to underexpress MNNF2.

20. The recombinant host cell of claim 19, wherein the MNNF2 protein comprises an amino acid sequence that is at least 70% identical, at least 75% identical, at least 80% identical, at least 85% identical, at least 90% identical, or at least 95% identical, to one of SEQ ID NO: 3.

21. The recombinant host cell of claim 19, wherein the recombinant host cell is engineered to express at least 10%, 15%, 20%, 30%, 40%, 50%, 60%, 70%, 80%, 90%, or 95% less MNNF2 relative to a host cell which has not been engineered to underexpress MNNF2.

22. The recombinant host cell of claim 1, wherein the recombinant host cell is further engineered to underexpress one or more enzymes in addition to BMT1 and BMT2.

23. The recombinant host cell of claim 22, wherein the one or more enzyme comprises an amino acid sequence that is at least 70% identical, at least 75% identical, at least 80% identical, at least 85% identical, at least 90% identical, or at least 95% identical, to one of SEQ ID NOs: 4-11, 14-15, and 72-85.

24. The recombinant host cell of claim 22, wherein the recombinant host cell is engineered to express at least 10%, 15%, 20%, 30%, 40%, 50%, 60%, 70%, 80%, 90%, or 95% less one or more enzymes relative to a host cell which has not been engineered to underexpress said one or more enzymes.

25. The recombinant host cell of claim 1, wherein the recombinant host cell recombinantly expresses a mannosidase from a species different from the recombinant host cell.

26. The recombinant host cell of claim 25, wherein the mannosidase is from a genus different from the recombinant host cell.

27. The recombinant host cell of claim 25, wherein the mannosidase comprises an amino acid sequence that is at least 70% identical, at least 75% identical, at least 80% identical, at least 85% identical, at least 90% identical, or at least 95% identical, to one of SEQ ID NOs: 41-56.

28. The recombinant host cell of claim 25, wherein the mannosidase is expressed on the surface of the recombinant host cell.

29. The recombinant host cell of claim 25, wherein the recombinant host cell expresses a surface-displayed fusion protein comprising a catalytic domain of a mannosidase and an anchoring domain of a glycosylphosphatidylinositol (GPI)-anchored protein, wherein the anchoring domain comprises at least about 200 amino acids and/or at least about 30% of the residues in the anchoring domain are serines or threonines.

30. The recombinant host cell of claim 29, wherein the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

31. The recombinant host cell of claim 29, wherein at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

32. The recombinant host cell of claim 29, wherein the serines or threonines in the anchoring domain are capable of being O-mannosylated.

33. The recombinant host cell of claim 29, wherein a fusion protein having an anchoring domain comprising at least about 325 amino acids provides greater enzymatic activity relative to a fusion protein having an anchoring domain comprising less than about 300 amino acids.

34. The recombinant host cell of claim 29, wherein a fusion protein having an anchoring domain comprising at least about 300 amino acids provides greater enzymatic activity relative to a fusion protein having an anchoring domain comprising less than about 250 amino acids.

35. The recombinant host cell of claim 29, wherein the fusion protein comprises the anchoring domain of the GPI anchored protein.

36. The recombinant host cell of claim 29, wherein the fusion protein comprises the GPI anchored protein without its native signal peptide.

37. The recombinant host cell of claim 29, wherein the GPI anchored protein is not native to the recombinant host cell.

38. The recombinant host cell of claim 29, wherein the GPI anchored protein is naturally expressed by a S. cerevisiae cell and the recombinant host cell is not a S. cerevisiae cell.

39. The recombinant host cell of claim 29, wherein the GPI anchored protein is selected from Tir4, Dan1, Dan4, Sag1, Fig2, and Sed1.

40. The recombinant host cell of claim 29, wherein the anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 57 to SEQ ID NO: 71.

41. The recombinant host cell of claim 29, wherein the anchoring domain of the GPI anchored protein comprises an amino acid sequence of one of SEQ ID NO: 57 to SEQ ID NO: 71.

42. The recombinant host cell of claim 29, wherein the recombinant host cell comprises a genomic modification that expresses the fusion protein and/or comprises an extrachromosomal modification that expresses the fusion protein.

43. The recombinant host cell of claim 29, wherein the fusion protein comprises a portion of the mannosidase in addition to its catalytic domain.

44. The recombinant host cell of claim 29, wherein the fusion protein comprises substantially the entire amino acid sequence of the mannosidase.

45. The recombinant host cell of claim 29, wherein in the fusion protein, the catalytic domain is N-terminal to the anchoring domain.

46. The recombinant host cell of claim 29, wherein the fusion protein comprises a linker between the catalytic domain and the anchoring domain.

47. The recombinant host cell of claim 29, wherein the fusion protein comprises a linker having an amino acid sequence that is at least 95% identical to any one of SEQ ID NOs: 316-321.

48. The recombinant host cell of claim 29, wherein, upon translation, the fusion protein comprises a signal peptide and/or a secretory signal.

49. The recombinant host cell of claim 29, wherein the recombinant host cell comprises two or more fusion proteins, three or more fusion proteins, or four fusion proteins.

50. The recombinant host cell of claim 1, wherein the recombinant host cell comprises a mutation in its AOX1 gene and/or its AOX2 gene.

51. The recombinant host cell of claim 1, wherein the recombinant host cell comprises a genomic modification that overexpresses a secreted heterologous protein of interest and/or comprises an extrachromosomal modification that overexpresses a secreted protein of interest.

52. The recombinant host cell of claim 1, wherein the secreted protein of interest is an animal protein.

53. The recombinant host cell of claim 52, wherein the animal protein is an egg protein.

54. The recombinant host cell of claim 53, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

55. The recombinant host cell of claim 52, wherein the genomic modification and/or the extrachromosomal modification that overexpresses the secreted recombinant protein comprises an inducible promoter.

56. The recombinant host cell of claim 55, wherein the inducible promoter is an AOX1, DAK2, PEX11, FLD1, FGH1, DAS1, DAS2, CAT1, MDH3, HAC1, BIP, RAD30, RVS161-2, MPP10, THP3, TLR, GBP2, PMP20, SHB17, PEX8, PEX4, or TKL3 promoter.

57. The recombinant host cell of claim 52, wherein the genomic modification and/or the extrachromosomal modification that overexpresses a secreted recombinant protein comprises an AOX1, TDH3, MOX, RPS25A, or RPL2A terminator.

58. The recombinant host cell of claim 52, wherein the genomic modification and/or the extrachromosomal modification that overexpresses a secreted recombinant protein encodes a signal peptide and/or a secretory signal.

59. The recombinant host cell of claim 52, wherein the genomic modification and/or the extrachromosomal modification that overexpresses a secreted recombinant protein comprises codons that are optimized for the species of the recombinant host cell.

60. The recombinant host cell of claim 52, wherein the secreted recombinant protein is designed to be secreted from the cell and/or is capable of being secreted from the cell.

61. The recombinant host cell of claim 56, wherein the additional genomic modification reduces the number of native cell wall proteins expressed by the recombinant host cell, thereby allowing additional space for localization of the surface-displayed fusion protein.

62. The recombinant host cell of claim 1, wherein the recombinant host cell comprises a further genomic modification that overexpresses a protein related to the p24 complex.

63. The recombinant host cell of claim 62, wherein the recombinant host cell comprises a further genomic modification comprising that overexpresses more than one protein related to the p24 complex.

64. The recombinant host cell of claim 62, wherein the protein related to the p24 complex is selected from Erp1, Erp2, Erp3, Erp5, Emp24, and Erv25.

65. The recombinant host cell of claim 62, wherein the protein related to the p24 complex comprises the amino acid sequence of any one of SEQ ID NO: 86 to SEQ ID NO: 91.

66. A method for expressing a heterologous protein of interest, the method comprising obtaining a recombinant host cell of claim 1 and culturing the recombinant host cell under conditions that allow expression of the heterologous protein of interest.

67. An isolated heterologous protein of interest expressed according to the method of claim 66.

68. Use of the isolated heterologous protein of interest of claim 67 in the manufacture of a nutritional, dietary, digestive, supplements, such as in food products, feed products, or cosmetic products.

69. A method for expressing a heterologous protein of interest having of a reduced level of exopolysaccharides, the method comprising obtaining a recombinant host cell of claim 1 and culturing the recombinant host cell under conditions that allow expression of the heterologous protein of interest.

70. An isolated heterologous protein of interest expressed according to the method of claim 69.

71. Use of the isolated heterologous protein of interest of claim 70 in the manufacture of a nutritional, dietary, digestive, supplements, such as in food products, feed products, or cosmetic products.

72. A method for expressing a heterologous protein of interest having of a reduced level of exopolysaccharides, the method comprising:
obtaining a host cell that is a yeast and is engineered to underexpress two mannosyl transferases: beta-mannosyl transferase 1 (BMT1) and beta-mannosyl transferase 2 (BMT2) or functional homologues thereof wherein the underexpression is compared to the host cell prior to genetic manipulation, wherein the host cell is engineered to express a heterologous protein of interest and a heterologous mannosidase; and
culturing the recombinant host cell under conditions that allow expression of the heterologous protein of interest.

73. The method of claim 72, wherein the BMT1 protein comprises an amino acid sequence that is at least 70% identical, at least 75% identical, at least 80% identical, at least 85% identical, at least 90% identical, or at least 95% identical, to SEQ ID NO: 12 and the BMT2 protein comprises an amino acid sequence that is at least 70% identical, at least 75% identical, at least 80% identical, at least 85% identical, at least 90% identical, or at least 95% identical, to SEQ ID NO: 13.

74. The method of claim 72, wherein the recombinant host cell is further engineered to underexpress one or more enzymes comprising an amino acid sequence of one of SEQ ID NOs: 1-11, 14-15, and 72-85.

75. The method of claim 72, wherein the recombinant host cell recombinantly expresses a mannosidase from a species different than from the recombinant host cell.

76. The method of claim 75, wherein the mannosidase comprises an amino acid sequence that is at least 70% identical, at least 75% identical, at least 80% identical, at least 85% identical, at least 90% identical, or at least 95% identical, to one of SEQ ID NOs: 41-56.

77. The method of claim 75, wherein the mannosidase is expressed on the surface of the recombinant host cell.

78. The method of claim 72, wherein the recombinant host cell expresses a surface-displayed fusion protein comprising a catalytic domain of a mannosidase and an anchoring domain of a glycosylphosphatidylinositol (GPI)-anchored protein, wherein the anchoring domain comprises at least about 200 amino acids and/or at least about 30% of the residues in the anchoring domain are serines or threonines.

79. The method of claim 72, wherein the heterologous protein of interest is secreted from the recombinant host cell.

80. The method of claim 79, wherein the secreted heterologous protein of interest is an animal protein.

81. The method of claim 80, wherein the animal protein is an egg protein.

82. The method of claim 81, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

83. The method of claim 72, wherein the recombinant host cell comprises a further genomic modification that overexpresses a protein related to the p24 complex.

84. An isolated heterologous protein of interest expressed according to the method of claim 72.

85. Use of the isolated heterologous protein of interest of claim 84 in the manufacture of a nutritional, dietary, digestive, supplements, such as in food products, feed products, or cosmetic products.

86. A method for manufacturing a recombinant host cell for manufacturing a heterologous protein of interest having of a reduced level of exopolysaccharides, the method comprising:
obtaining a yeast cell engineered to express a heterologous protein of interest and/or a heterologous mannosidase; and
modifying the yeast cell to underexpress two mannosyl transferases: beta-mannosyl transferase 1 (BMT1) and beta-mannosyl transferase 2 (BMT2) or functional homologues thereof. A method for manufacturing a recombinant host cell for manufacturing a heterologous protein of interest having of a reduced level of exopolysaccharides, the method comprising:
obtaining a yeast cell engineered to underexpress two mannosyl transferases: beta-mannosyl transferase 1 (BMT1) and beta-mannosyl transferase 2 (BMT2) or functional homologues thereof and engineered to express a heterologous mannosidase; and
modifying the yeast cell to express a heterologous protein of interest.

87. A method for manufacturing a recombinant host cell for manufacturing a heterologous protein of interest having of a reduced level of exopolysaccharides, the method comprising:
obtaining a yeast cell engineered to underexpress two mannosyl transferases: beta-mannosyl transferase 1 (BMT1) and beta-mannosyl transferase 2 (BMT2) or functional homologues thereof and engineered to express a heterologous protein of interest; and
modifying the yeast cell to express a heterologous mannosidase.

88. A method for manufacturing a recombinant host cell for manufacturing a heterologous protein of interest having of a reduced level of exopolysaccharides, the method comprising:
obtaining a yeast cell
modifying the yeast cell engineered to underexpress two mannosyl transferases: beta-mannosyl transferase I (BMT1) and beta-mannosyl transferase 2 (BMT2) or functional homologues thereof and engineered to express a heterologous protein of interest;
modifying the yeast cell to express a heterologous protein of interest; and
modifying the yeast cell to express a heterologous mannosidase.
